(19) United States
(12) Patent Application Publication
(10) Pub. No.: US 2024/0382432 A1
(43) Pub. Date: Nov. 21, 2024

(54) SINGLE CHAIN VARIABLE FRAGMENT (SCFV) MODIFIED LIPID NANOPARTICLE COMPOSITIONS AND USES THEREOF

(71) Applicant: Generation Bio Co., Cambridge, MA (US)

(72) Inventors: Phillip Samayoa, Cambridge, MA (US); Nathaniel Silver, Cambridge, MA (US); Prudence Yui Tung Li, Cambridge, MA (US); Randall Newton Toy, Cambridge, MA (US); Birte Nolting, Cambridge, MA (US); Lalita Oonthonpan, Cambridge, MA (US)

(21) Appl. No.: 18/578,073

(22) PCT Filed: Jul. 13, 2022
(86) PCT No.: PCT/US2022/036930

§ 371 Date: Jan. 10, 2024

Related U.S. Application Data
(60) Provisional application No. 63/221,290, filed on Jul. 13, 2021.

(51) Int. Cl. A61K 9/51; A61K 47/68; A61K 47/69; A61K 48/00; C12N 15/88

(52) U.S. Cl. CPC A61K 9/5123; A61K 9/5169; A61K 47/6855; A61K 47/6931; A61K 48/0041; C12N 15/88

(57) ABSTRACT

Provided herein are pharmaceutical compositions comprising a lipid nanoparticle (LNP) and a therapeutic nucleic acid (TNA), wherein the LNP comprises a single-chain variable fragment (scFv) linked to the LNP, and at least one pharmaceutically acceptable excipient. The scFv is capable of binding an antigen present on the surface of a cell, advantageously providing LNP compositions that target only those cells or tissues expressing the receptor.

### RELATED APPLICATIONS

This application claims priority to U.S. Provisional Application No. 63/221,290, filed on Jul. 13, 2021, the contents of which is hereby incorporated by reference in its entirety.

### BACKGROUND

Ionizable lipid nanoparticles (LNPs) have been widely used for the systemic delivery of RNA therapeutics. Various types of ionizable lipid materials have been previously reported for LNP formulations, such as C12-200, cKK-E12, and DLin-MC3-DMA, and efficient gene silencing in the liver at a dosing level of 0.002 mg of siRNA/kg has been demonstrated (Dong, et al., Proc. Natl. Acad. Sci. U.S.A. 111, 3955-3960 (2014)). Although the inclusion of targeting ligands has been shown to enhance the delivery and therapeutic efficiency of mRNA-LNPs, it has been recognized that attaching targeting moieties may add complexity, cost, and regulatory difficulties to the process of manufacturing LNP systems (Cheng et al., Science. 2012 Nov. 16; 338(6109):903-10). In addition, it has been demonstrated that the targeting specificity of some targeting ligands may disappear when lipid nanoparticles are exposed to biological fluids where interaction with proteins in the media and the consequent formation of protein corona takes place (Salvati et al., Nat Nanotechnol. 2013 February; 8(2):137-43). Therefore, a trade-off exists between the possible clinical benefits and the complexity and cost of the targeted RNA-LNP manufacture.

Antibodies function by targeting specific antigens that are expressed only on the surface of diseased cells, or heavily overexpressed on these cells relative to healthy cells. As these antigens are present solely, or abundantly, on the surface of the target diseased cells, antibodies can conceptually be exploited to carry nanoparticles and their cargo (e.g., therapeutic agents) through the body and enable selective delivery/targeting. While this approach was first explored in the 1980s, there were considerable limitations such as insufficient methods for generating and evaluating antibody-decorated nanoparticles, which prevented significant progress in the area. Advancements in both antibody expression techniques and nanoparticle design over the past few decades have enabled a more thorough exploration of nanoparticle-antibody conjugates, which has resulted in a rapid expansion of the field. Early developments focused almost entirely on using full antibodies as targeting ligands, primarily due to the wealth of available information on both their generation and modification. However, several issues associated with the use of full antibody ligands emerged, such as immunogenicity, rapid elimination, poor stability, and lower than expected efficacy.

The modular nature of antibodies, both structurally and functionally, allows for the generation of smaller antigen binding fragments, such as fragment antigen binding (Fab), the single chain fragment variable (scFv), single-domain antibodies, and the fragment crystallizable (Fc) domain, through molecular cloning, antibody engineering, and even enzymatic methods. Antigen-binding fragments of antibodies have a considerable potential to overcome the disadvantages of conventional mAbs, such as poor penetration into solid tumors and Fc-mediated bystander activation of the immune system. Antibody fragments can be used on their own or linked to other molecules to generate numerous possibilities for bispecific, multi-specific, multimeric, or multifunctional molecules, and to achieve a variety of biological effects. Antibody fragments can offer several advantages over the use of conventional antibodies. For example, they can be produced easily, generally using microbial expression systems, which results in faster cultivation, higher yields, and lower production costs (Fernandes J C, Drug Discov Today. 2018 December; 23(12):1996-2002). Their small size allows access to challenging, cryptic epitopes, and tumour penetration, they have reduced immunogenicity, and the lack of Fc limits bystander activation of the immune system (Kholodenko et al. Curr Med Chem. 2019; 26(3):396-426). On the other hand, their smaller size results in faster renal excretion, which may require higher doses and/or more frequent dosing regimens in vivo.

Although LNPs have been shown to be advantageous for in vivo delivery, systemic delivery of RNA therapeutics other than liver hepatocytes remains highly challenging. The relatively large size of these LNPs reduces the therapeutic index for liver indications by several mechanisms: (1) larger LNPs are unable to efficiently bypass the fenestrae of the endothelial cells that line liver sinusoids, preventing access to target cells (hepatocytes); (2) larger LNPs are unable to be efficiently internalized by hepatocytes via clathrin-mediated endocytosis with several different receptors (e.g. asialoglycoprotein receptor (ASGPR), low-density lipoprotein (LDL) receptor); and (3) LNPs above a certain threshold size are prone to preferential uptake by cells of the reticuloendothelial system, which can provoke dose-limiting immune responses. Despite these advances, LNP-mediated delivery of larger, rigid polynucleotide cargos (e.g., double stranded linear DNA, plasmid DNA, closed-ended double stranded DNA (ceDNA)) presents additional challenges relative to the smaller and/or flexible cargos (e.g., siRNA). One such challenge involves the size of the resulting LNP when large, rigid cargo is encapsulated.

To fully realize the potential of LNP-targeted nucleic acid therapeutics, an efficient in vivo delivery system is needed.

### SUMMARY

The present disclosure provides a pharmaceutical composition comprising a lipid nanoparticle (LNP), a therapeutic nucleic acid (TNA), and at least one pharmaceutically acceptable excipient, wherein the LNP comprises a single chain fragment variable (scFv) linked to the LNP, and wherein the scFv is directed against an antigen present on the surface of a cell (e.g., a tumor cell). The LNP compositions described herein advantageously provide efficient, covalent conjugation with minimal effects on particle size and stability. It is a finding of the present disclosure that maleimide conjugation of scFv to LNP resulted in robust conjugation to the LNP along with other thiol based cross-linking methods and importantly, maintained LNP size and integrity.

According to a first aspect, the disclosure provides a pharmaceutical composition comprising a lipid nanoparticle (LNP), a therapeutic nucleic acid (TNA), and at least one pharmaceutically acceptable excipient, wherein the LNP comprises a single-chain variable fragment (scFv) linked to the LNP, and wherein the scFv is directed against an antigen present on the surface of a cell. In some embodiments, the scFV is covalently linked to the LNP. In some embodiments, the scFV is chemically conjugated to the LNP. In some embodiments, the scFV is chemically conjugated to the LNP via a non-cleavable linker. In some embodiments, the non-cleavable linker is a maleimide-containing linker. In some embodiments, the scFV is chemically conjugated to the LNP via a cleavable linker. In some embodiments, the cleavable linker is a pyridyldisulfide (PDS)-containing linker. In some embodiments, the scFv is linked to the LNP via transglutaminase-mediated conjugation. In some embodiments of any of the above aspects and embodiments, the antigen is a tumor-associated antigen (TAA) or a tumor-specific antigen (TSA). In a further embodiments the antigen is human epidermal growth factor receptor 2 (HER2). In some embodiments of any of the above aspects and embodiments, thescFv is bivalent. In some embodiments of any of the above aspects and embodiments, the LNP is capable of being internalized into the cell. In some embodiments of any of the above aspects and embodiments, the scFV comprises an amino acid sequence of SEQ ID NO:2 or has a sequence similarity of at least 99% to the amino acid sequence set forth in SEQ ID NO:2. In some embodiments of any of the above aspects and embodiments, the scFV comprises an amino acid sequence of SEQ ID NO:3 or has a sequence similarity of at least 99% to the amino acid sequence set forth in SEQ ID NO:3. In some embodiments of any of the above aspects and embodiments, the LNP comprises a lipid selected from the group consisting of: a cationic lipid, a sterol or a derivative thereof, a non-cationic lipid, and a PEGylated lipid. In some embodiments of any of the above aspects and embodiments, the TNA is encapsulated in the LNP. In some embodiments of any of the above aspects and embodiments, the TNA is selected from the group consisting of minigenes, plasmids, minicircles, small interfering RNA (siRNA), microRNA (miRNA), antisense oligonucleotides (ASO), ribozymes, closed-ended (ceDNA), ministring, doggybone™ protelomere closed ended DNA, or dumbbell linear DNA, dicer-substrate dsRNA, small hairpin RNA (shRNA), asymmetrical interfering RNA (aiRNA), microRNA (miRNA), mRNA, tRNA, rRNA, DNA viral vectors, viral RNA vector, non-viral vector and any combination thereof. In some embodiments, the TNA is ceDNA. In some embodiments, the ceDNA is linear duplex DNA. In some embodiments, the TNA is mRNA. In some embodiments, the TNA is siRNA. In some embodiments, the TNA is a plasmid. In some embodiments of any of the above aspects and embodiments, the pharmaceutical composition is administered to a subject. In some embodiments, the subject is a human patient in need of treatment with LNP encapsulated with TNA. In some embodiments of any of the above aspects and embodiments, the composition is targeted to a cell expressing the cell-surface antigen for which the scFv is directed. In some embodiments of any of the above aspects and embodiments, the composition is targeted to tumor cells. In some embodiments of any of the above aspects and embodiments, the composition is targeted to liver cells. In some embodiments of any of the above aspects and embodiments, the composition is targeted to hepatocytes in the liver.

In some embodiments of any of the above aspects and embodiments, the cationic lipid is represented by Formula (I):

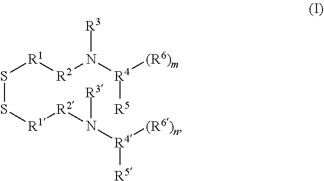

or a pharmaceutically acceptable salt thereof, wherein:
R¹ and R^{1′} are each independently optionally substituted linear or branched C₁₋₃ alkylene;
R² and R^{2′} are each independently optionally substituted linear or branched C₁₋₆ alkylene;
R³ and R^{3′} are each independently optionally substituted linear or branched C₁₋₆ alkyl;
or alternatively, when R² is optionally substituted branched C₁₋₆ alkylene, R² and R³, taken together with their intervening N atom, form a 4- to 8-membered heterocyclyl;
or alternatively, when R^{2′} is optionally substituted branched C₁₋₆ alkylene, R^{2′} and R³, taken together with their intervening N atom, form a 4- to 8-membered heterocyclyl;
R⁴ and R^{4′} are each independently —CR^{a}, —C(R^{a})₂CR^{a}, or —[C(R^{a})₂]₂CR^{a};
R^{a}, for each occurrence, is independently H or C₁₋₃ alkyl;
or alternatively, when R⁴ is —C(R^{a})₂CR^{a}, or —[C(R^{a})₂]₂CR^{a} and when R^{a} is C₁₋₃ alkyl, R³ and R⁴, taken together with their intervening N atom, form a 4- to 8-membered heterocyclyl;
or alternatively, when R^{4′} is —C(R^{a})₂CR^{a}, or —[C(R^{a})₂]₂CR^{a} and when R^{a} is C₁₋₃ alkyl, R^{3′} and
R^{4′}, taken together with their intervening N atom, form a 4- to 8-membered heterocyclyl;
R⁵ and R^{5′} are each independently hydrogen, C₁₋₂₀ alkylene or C₂₋₂₀ alkenylene; R⁶ and R^{6′}, for each occurrence, are independently C₁₋₂₀ alkylene, C₃₋₂₀ cycloalkylene, or C₂₋₂₀ alkenylene; and
m and n are each independently an integer selected from 1, 2, 3, 4, and 5.

In some embodiments of any of the above aspects and embodiments, the cationic lipid is represented by Formula (II):

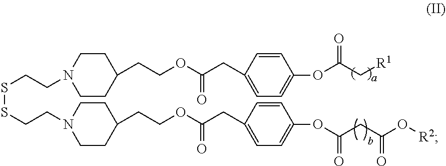

or a pharmaceutically acceptable salt thereof, wherein:
a is an integer ranging from 1 to 20;
b is an integer ranging from 2 to 10;
R¹ is absent or is selected from (C₂-C₂₀)alkenyl, —C(O)O(C₂-C₂₀)alkyl, and cyclopropyl substituted with (C₂-C₂₀)alkyl; and
R² is (C₂-C₂₀)alkyl.

In some embodiments of any of the above aspects and embodiments, the lipid is represented by the Formula (V):

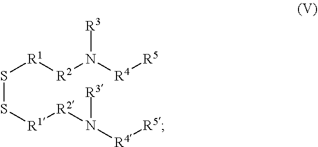

or a pharmaceutically acceptable salt thereof, wherein:
R¹ and R^{1′} are each independently (C₁-C₆)alkylene optionally substituted with one or more groups selected from R^{a};
R² and R^{2′} are each independently (C₁-C₂)alkylene;
R³ and R^{3′} are each independently (C₁-C₆)alkyl optionally substituted with one or more groups selected from R^{b};
or alternatively, R² and R³ and/or R^{2′} and R^{3′} are taken together with their intervening N atom to form a 4- to 7-membered heterocyclyl;
R⁴ and R^{4′} are each a (C₂-C₆)alkylene interrupted by —C(O)O—;
R⁵ and R^{1′} are each independently a (C₂-C₃₀)alkyl or (C₂-C₃₀)alkenyl, each of which are optionally interrupted with —C(O)O— or (C₃-C₆)cycloalkyl; and
R^{a} and R^{b} are each halo or cyano.

In some embodiments of any of the above aspects and embodiments, the cationic lipid is represented by Formula (XV):

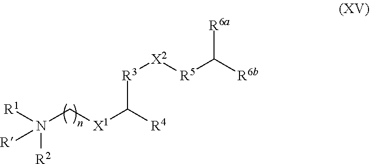

or a pharmaceutically acceptable salt thereof, wherein:
R′ is absent, hydrogen, or C₁-C₆ alkyl; provided that when R′ is hydrogen or C₁-C₆ alkyl, the nitrogen atom to which R′, R¹, and R² are all attached is protonated;
R¹ and R² are each independently hydrogen, C₁-C₆ alkyl, or C₂-C₆ alkenyl;
   R³ is C₁-C₁₂ alkylene or C₂-C₁₂ alkenylene;
R⁴ is C₁-C₁₆unbranched alkyl, C₂-C₁₆unbranched alkenyl, or

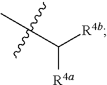

wherein:
R^{4a} and R^{4b} are each independently C₁-C₁₆unbranched alkyl or C₂-C₁₆unbranched alkenyl;
R⁵ is absent, C₁-C₅ alkylene, or C₂-C₅ alkenylene;
R^{6a} and R^{6b} are each independently C₇-C₁₆ alkyl or C₇-C₁₆ alkenyl; provided that the total number of carbon atoms in R^{6a} and R^{6b} as combined is greater than 15;
X¹ and X² are each independently —OC(═O)—, —SC(═O)—, —OC(═S)—, —C(═O)O—, —C(═O)S—, —S—S—, —C(R^{a})═N—, —N═C(R^{a})—, —C(R^{a})═NO—, —O—N═C(R^{a})—, —C(═O)NR^{a}—, —NR^{a}C(═O)—, —NR^{a}C(═O)NR^{a}—, —OC(═O)O—, —OSi(R^{a})₂O—, —C(═O)(CR^{a}₂)C(═O)O—, or OC(═O)(CR^{a}₂)C(═O)—; wherein:
   R^{a}, for each occurrence, is independently hydrogen or C₁-C₆ alkyl; and n is an integer selected from 1, 2, 3, 4, 5, and 6.

In some embodiments of any of the above aspects and embodiments, the cationic lipid is represented by Formula (XX):

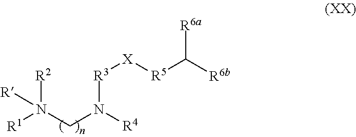

or a pharmaceutically acceptable salt thereof, wherein:
R′ is absent, hydrogen, or C₁-C₃ alkyl; provided that when R′ is hydrogen or C₁-C₃ alkyl, the nitrogen atom to which R′, R¹, and R² are all attached is protonated;
R¹ and R² are each independently hydrogen or C₁-C₃ alkyl;
R³ is C₃-C₁₀ alkylene or C₃-C₁₀ alkenylene;
R⁴ is C₁-C₁₆unbranched alkyl, C₂-C₁₆unbranched alkenyl, or

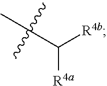

wherein:
R^{4a} and R^{4b} are each independently C₁-C₁₆unbranched alkyl or C₂-C₁₆unbranched alkenyl;
R⁵ is absent, C₁-C₆ alkylene, or C₂-C₆ alkenylene;
R^{6a} and R^{6b} are each independently C₇-C₁₄ alkyl or C₇-C₁₄ alkenyl;
X is —OC(═O)—, —SC(═O)—, —OC(═S)—, —C(═O)O—, —C(═O)S—, —S—S—, —C(R^{a})═N—, —N═C(R^{a})—, —C(R^{a})═NO—, —O—N═C(R^{a})—, —C(═O)NR^{a}—, —NR^{a}C(═O)—, —NR^{a}C(═O)NR^{a}—, —OC(═O)O—, —OSi(R^{a})₂O—, —C(═O)(CR^{a}₂)C(═O)O—, or OC(═O)(CR^{a}₂)C(═O)—; wherein:
   R^{a}, for each occurrence, is independently hydrogen or C₁-C₆ alkyl; and
n is an integer selected from 1, 2, 3, 4, 5, and 6.

In some embodiments of any of the above aspects and embodiments, the cationic lipid is selected from any lipid in Table 2, Table 5, Table 6, Table 7, or Table 8. In some embodiments, the cationic lipid is a lipid having the structure:

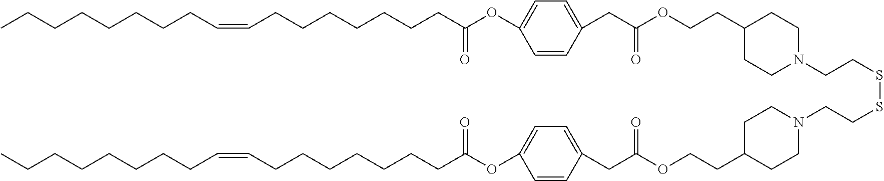 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCC/C=C\CCCCCCCC)cc4)CC3)CC2)cc1]

or a pharmaceutically acceptable salt thereof. In some embodiments, the cationic lipid is MC3 (6Z,9Z,28Z,31Z)-heptatriaconta-6,9,28,31-tetraen-19-yl-4-(dimethylamino) butanoate (DLin-MC3-DMA or MC3) having the following structure:

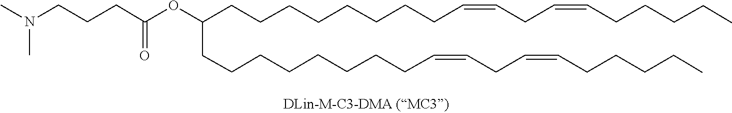 [SMILES: CCCCC/C=C\C/C=C\CCCCCCCCC(CCCCCCCC/C=C\C/C=C\CCCCC)OC(=O)CCCN(C)C]

or a pharmaceutically acceptable salt thereof.

In some embodiments of any of the above aspects and embodiments, the sterol or a derivative thereof is a cholesterol or a beta-sitosterol. In some embodiments, the non-cationic lipid is selected from the group consisting of distearoyl-sn-glycero-phosphoethanolamine (DSPE), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), 1,2-dilauroyl-sn-glycero-3-pho sphoethanolamine (DLPE); 1,2-diphytanoyl-sn-glycero-3-phosphoethanolamine (DPHyPE); lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid,cerebrosides, dicetylphosphate, lysophosphatidylcholine, dilinoleoylphosphatidylcholine, and mixtures thereof. In some embodiments, the non-cationic lipid is selected from the group consisting of dioleoylphosphatidylcholine (DOPC), distearoylphosphatidylcholine (DSPC), and dioleoyl-phosphatidylethanolamine (DOPE). In some embodiments, the PEGylated lipid is selected from the group consisting of PEG-dilauryloxypropyl; PEG-dimyristyloxypropyl; PEG-dipalmityloxypropyl, PEG-distearyloxypropyl; l-(monomethoxy-polyethyleneglycol)-2,3-dimyristoylglycerol (DMG-PEG); PEG-dilaurylglycerol; PEG-dipalmitoylglycerol; PEG-disterylglycerol; PEG-dilaurylglycamide; PEG-dimyristylglycamide; PEG-dipalmitoylglycamide; PEG-disterylglycamide; (1-[8′-(Cholest-5-en-3[beta]-oxy)carboxamido-3′,6′-dioxaoctanyl]carbamoyl-[omega]-methyl-poly(ethylene glycol) (PEG-cholesterol); 3,4-ditetradecoxylbenzyl-[omega]-methyl-poly(ethylene glycol) ether (PEG-DMB), and 1,2-dimyristoyl-sn-glycero-3-phosphoethanolamine-N-[methoxy(polyethylene glycol) (DSPE-PEG), and 1,2-distearoyl-sn-glycero-3-phosphoethanolamine-N-poly(ethylene glycol)-hydroxyl (DSPE-PEG-OH). In some embodiments, the PEGylated lipid is DMG-PEG, DSPE-PEG, DSPE-PEG-OH, or a combination thereof. In some embodiments of any of the above aspects and embodiments, the at least one PEGylated lipid is DMG-PEG2000, DSPE-PEG2000, DSPE-PEG2000-OH, DMG-PEG5000, DSPE-PEG5000, DSPE-PEG5000-OH, or a combination thereof. In some embodiments of any of the above aspects and embodiments, the scFv is chemically conjugated or covalently linked to a PEGylated lipid of the LNP to form a PEGylated lipid conjugate. In some embodiments of any of the above aspects and embodiments, the PEGylated lipid to which the scFv is chemically conjugated or covalently linked is DSPE-PEG. In some embodiments, the PEGylated lipid to which the scFv is chemically conjugated or covalently linked is DSPE-PEG2000. In some embodiments, the PEGylated lipid to which the scFv is chemically conjugated or covalently linked is DSPE-PEG5000. In some embodiments of any of the above aspects and embodiments, the cationic lipid is present at a molar percentage of about 30% to about 80%. In some embodiments, the sterol is present at a molar percentage of about 20% to about 50%. In some embodiments of any of the above aspects and embodiments, the non-cationic lipid is present at a molar percentage of about 2% to about 20%. In some embodiments of any of the above aspects and embodiments, the at least one PEGylated lipid is present at a molar percentage of about 2.1% to about 10%.

In some embodiments of any of the above aspects and embodiments, the scFv are present at a total amount of about 0.02 μg/μg of TNA to about 0.1 μg/μg of TNA.

In some embodiments of any of the above aspects and embodiments, the pharmaceutical composition further comprises dexamethasone palmitate.

In some embodiments of any of the above aspects and embodiments, the LNP has a total lipid to TNA ratio of about 10:1 to about 40:1.

In some embodiments of any of the above aspects and embodiments, the LNP has a diameter ranging from about 40 nm to about 120 nm.

In some embodiments of any of the above aspects and embodiments, the nanoparticle has a diameter of less than about 100 nm.

In some embodiments of any of the above aspects and embodiments, the nanoparticle has a diameter of about 60 nm to about 80 nm.

In some embodiments of any of the above aspects and embodiments, the ceDNA comprises an expression cassette, and wherein the expression cassette comprises a promoter sequence and a transgene. In some embodiments, the expression cassette comprises a polyadenylation sequence. In some embodiments of any of the above aspects and embodiments, the ceDNA comprises at least one inverted terminal repeat (ITR) flanking either 5′ or 3′ end of the expression cassette. In some embodiments, the expression cassette is flanked by two ITRs, wherein the two ITRs comprise one 5′ ITR and one 3′ ITR. In some embodiments, the expression cassette is connected to an ITR at 3′ end (3′ ITR). In some embodiments of any of the above aspects and embodiments, the expression cassette is connected to an ITR at 5′ end (5′ ITR).

In some embodiments of any of the above aspects and embodiments, the at least one ITR is an ITR derived from an AAV serotype, an ITR derived from an ITR of a goose virus, an ITR derived from a B19 virus ITR, or a wild-type ITR from a parvovirus. In some embodiments, said AAV serotype is selected from the group consisting of: AAV1, AAV2, AAV3, AAV4, AAV5, AAV6, AAV7, AAV8, AAV9, AAV10, AAV11 and AAV12.

In some embodiments of any of the above aspects and embodiments, the at least one of the 5′ ITR and the 3′ ITR is a wild-type AAV ITR.

In some embodiments of any of the above aspects and embodiments, the at least one of the 5′ ITR and the 3′ ITR is a modified or mutant ITR.

In some embodiments of any of the above aspects and embodiments, the 5′ ITR and the 3′ ITR are symmetrical.

In some embodiments of any of the above aspects and embodiments, the 5′ ITR and the 3′ ITR are asymmetrical.

In some embodiments of any of the above aspects and embodiments, the ceDNA further comprises a spacer sequence between a 5′ ITR and the expression cassette.

In some embodiments of any of the above aspects and embodiments, the ceDNA further comprises a spacer sequence between a 3′ ITR and the expression cassette. In some embodiments of any of the above aspects and embodiments, the spacer sequence is at least 5 base pairs long in length.

In some embodiments of any of the above aspects and embodiments, the ceDNA has a nick or a gap.

In some embodiments of any of the above aspects and embodiments, the ceDNA is a CELiD, DNA-based minicircle, a MIDGE, a ministring DNA, a dumbbell shaped linear duplex closed-ended DNA comprising two hairpin structures of ITRs in the 5′ and 3′ ends of an expression cassette, or a doggybone™ DNA.

In some aspects, the disclosure provides a method of treating a cancer in a subject, comprising administering to the subject an effective amount of the pharmaceutical composition of any one of the aspects and embodiments herein. In some embodiments, the subject is a human.

In some aspects, the disclosure provides a method of delivering a therapeutic nucleic acid (TNA) or increasing the concentration of the TNA to a tumor in a subject, comprising administering to the subject an effective amount of the pharmaceutical composition of any one of the aspects and embodiments herein.

In some aspects, the disclosure provides a method of delivering a therapeutic nucleic acid (TNA) or increasing the concentration of the TNA to the liver of a subject, comprising administering to the subject an effective amount of the pharmaceutical composition of any one of the aspects and embodiments herein.

According to some embodiments, the LNP is internalized into the cell. According to some embodiments of the above aspects and embodiments, the LNP comprises a cationic lipid, a sterol or a derivative thereof, a non-cationic lipid, or a PEGylated lipid. According to some embodiments of the above aspects and embodiments, the TNA is encapsulated in the lipid. According to some embodiments of the above aspects and embodiments, the TNA is selected from the group consisting of minigenes, plasmids, minicircles, small interfering RNA (siRNA), microRNA (miRNA), antisense oligonucleotides (ASO), ribozymes, closed-ended (ceDNA), ministring, doggybone™, protelomere closed ended DNA, or dumbbell linear DNA, dicer-substrate dsRNA, small hairpin RNA (shRNA), asymmetrical interfering RNA (aiRNA), microRNA (miRNA), mRNA, tRNA, rRNA, DNA viral vectors, viral RNA vector, non-viral vector and any combination thereof. According to some embodiments, the TNA is ceDNA. According to some embodiments, the ceDNA is linear duplex DNA. According to some embodiments, the TNA is mRNA. According to some embodiments, the TNA is siRNA. According to some embodiments, the TNA is a plasmid.

According to some embodiments, the LNP comprises a PEGylated lipid, wherein the PEGylated lipid is linked to the amino acid sequence encoding the scFv (the scFv polypeptide).

According to some embodiments of the above aspects and embodiments, the pharmaceutical composition is administered to a subject. According to some embodiments of the above aspects and embodiments, the subject is a human patient in need of treatment with LNP encapsulated with TNA.

According to some embodiments of the above aspects and embodiments, the composition is targeted to a cell or tissue expressing the target antigen via binding of the scFv in the LNP to the antigen target. According to some embodiments of the above aspects and embodiments, the composition is targeted to tumor cells. According to some embodiments, the tumor is a solid tumor. According to some embodiments, the tumor is a hematological tumor. According to some embodiments of the above aspects and embodiments, the composition is targeted to liver cells.

According to some embodiments of the above aspects and embodiments, the cationic lipid is represented by Formula (I):

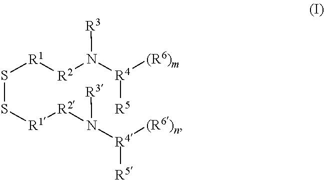

or a pharmaceutically acceptable salt thereof, wherein:
R¹ and R^{1′} are each independently optionally substituted linear or branched C₁ 3 alkylene;
R² and R^{2′} are each independently optionally substituted linear or branched C₁ 6 alkylene;
R³ and R^{3′} are each independently optionally substituted linear or branched C₁ 6 alkyl;
or alternatively, when R² is optionally substituted branched C₁ 6 alkylene, R² and R³, taken together with their intervening N atom, form a 4- to 8-membered heterocyclyl;
or alternatively, when R^{2′} is optionally substituted branched C₁ 6 alkylene, R^{2′} and R³, taken together with their intervening N atom, form a 4- to 8-membered heterocyclyl;
R⁴ and R^{4′} are each independently —CR^{a}, —C(R^{a})₂CR^{a}, or —[C(R^{a})₂]2CR^{a};
R^{a}, for each occurrence, is independently H or C₁₋₃ alkyl;
or alternatively, when R⁴ is —C(R^{a})₂CR^{a}, or —[C(R^{a})₂]2CR^{a} and when R^{a} is C₁₋₃ alkyl, R³ and R⁴, taken together with their intervening N atom, form a 4- to 8-membered heterocyclyl;
or alternatively, when R^{4′} is —C(R^{a})₂CR^{a}, or —[C(R^{a})₂]₂CR^{a} and when R^{a} is C₁₋₃ alkyl, R^{3′} and R^{4′}, taken together with their intervening N atom, form a 4- to 8-membered heterocyclyl;
R⁵ and R^{1′} are each independently hydrogen, C₁₋₂₀ alkylene or C₂₋₂₀ alkenylene;
R⁶ and R^{6′}, for each occurrence, are independently C₁₋₂₀ alkylene, C₃₋₂₀ cycloalkylene, or C₂₂o alkenylene; and
m and n are each independently an integer selected from 1, 2, 3, 4, and 5.

According to some embodiments of the above aspects and embodiments, the cationic lipid is represented by Formula (II):

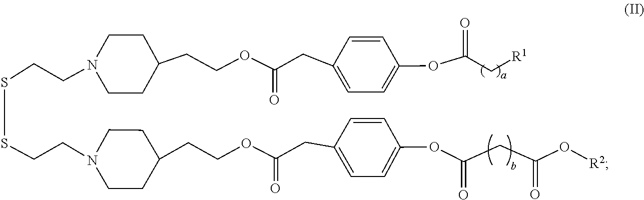

or a pharmaceutically acceptable salt thereof, wherein:
a is an integer ranging from 1 to 20;
b is an integer ranging from 2 to 10;
R¹ is absent or is selected from (C₂-C₂₀)alkenyl, —C(O)O(C₂-C₂₀)alkyl, and cyclopropyl substituted with (C₂-C₂₀)alkyl; and
R² is (C₂-C₂₀)alkyl.

According to some embodiments of the above aspects and embodiments, the lipid is represented by the Formula (V):

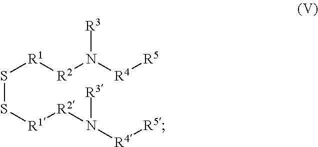

or a pharmaceutically acceptable salt thereof, wherein:
R¹ and R^{1′} are each independently (C₁-C₆)alkylene optionally substituted with one or more groups selected from R^{a};
R² and R^{2′} are each independently (C₁-C₂)alkylene;
R³ and R^{3′} are each independently (C₁-C₆)alkyl optionally substituted with one or more groups selected from R^{b};
or alternatively, R² and R³ and/or R^{2′} and R^{3′} are taken together with their intervening N atom to form a 4- to 7-membered heterocyclyl;
R⁴ and R^{4′} are each a (C₂-C₆)alkylene interrupted by —C(O)O—;
R⁵ and R^{5′} are each independently a (C₂-C₃₀)alkyl or (C₂-C₃₀)alkenyl, each of which are optionally interrupted with —C(O)O— or (C₃-C₆)cycloalkyl; and R^{a} and R^{b} are each halo or cyano.

According to some embodiments of the above aspects and embodiments, the cationic lipid is represented by Formula (XV):

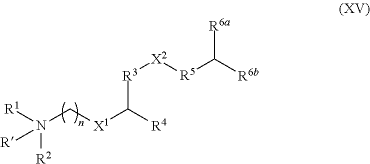

or a pharmaceutically acceptable salt thereof, wherein:
R′ is absent, hydrogen, or C₁-C₆ alkyl; provided that when R′ is hydrogen or C₁-C₆ alkyl, the nitrogen atom to which R′, R¹, and R² are all attached is protonated;
R¹ and R² are each independently hydrogen, C₁-C₆ alkyl, or C₂-C₆ alkenyl;
   R³ is C₁-C₁₂ alkylene or C₂-C₁₂ alkenylene;
R⁴ is C₁-C₁₆unbranched alkyl, C₂-C₁₆unbranched alkenyl, or

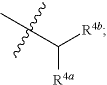

wherein:
R^{4a} and R^{4b} are each independently C₁-C₁₆unbranched alkyl or C₂-C₁₆unbranched alkenyl;
R⁵ is absent, C₁-C₅ alkylene, or C₂-C₅ alkenylene;
R^{6a} and R^{6b} are each independently C₇-C₁₆ alkyl or C₇-C₁₆ alkenyl; provided that the total number of carbon atoms in R^{6a} and R^{6b} as combined is greater than 15;
X¹ and X² are each independently —OC(═O)—, —SC(═O)—, —OC(═S)—, —C(═O)O—, —C(═O)S—, —S—S—, —C(R^{a})═N-, —N═C(R^{a})—, —C(R^{a})═NO—, —O—N═C(R^{a})—, —C(═O)NR^{a}—, —NR^{a}C(═O)—, —NR^{a}C(═O)NR^{a}—, —OC(═O)O—, —OSi(R^{a})₂O—, —C(═O)(CR^{a}₂)C(═O)O—, or OC(═O)(CR^{a}₂)C(═O)—; wherein:
   R^{a}, for each occurrence, is independently hydrogen or C₁-C₆ alkyl; and
n is an integer selected from 1, 2, 3, 4, 5, and 6.

According to some embodiments of the above aspects and embodiments, the cationic lipid is represented by Formula (XX):

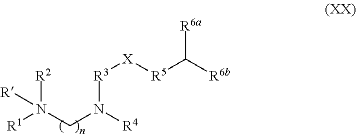

or a pharmaceutically acceptable salt thereof, wherein:
R′ is absent, hydrogen, or C₁-C₃ alkyl; provided that when R′ is hydrogen or C₁-C₃ alkyl, the nitrogen atom to which R′, R¹, and R² are all attached is protonated;
R¹ and R² are each independently hydrogen or C₁-C₃ alkyl;
R³ is C₃-C₁₀ alkylene or C₃-C₁₀ alkenylene;
R⁴ is C₁-C₁₆unbranched alkyl, C₂-C₁₆unbranched alkenyl, or

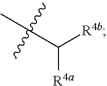

wherein:
R^{4a} and R^{4b} are each independently C₁-C₁₆unbranched alkyl or C₂-C₁₆unbranched alkenyl;
R⁵ is absent, C₁-C₆ alkylene, or C₂-C₆ alkenylene;
R^{6a} and R^{6b} are each independently C₇-C₁₄ alkyl or C₇-C₁₄ alkenyl;
X is —OC(═O)—, —SC(═O)—, —OC(═S)—, —C(═O)O—, —C(═O)S—, —S—S—, —C(R^{a})═N—, —N═C(R^{a})—, —C(R^{a})═NO—, —O—N═C(R^{a})—, —C(═O)NR^{a}—, —NR^{a}C(═O)—, —NR^{a}C(═O)NR^{a}—, —OC(═O)O—, —OSi(R^{a})₂O—, —C(═O)(CR^{a}₂)C(═O)O—, or OC(═O)(CR^{a}₂)C(═O)—; wherein:
   R^{a}, for each occurrence, is independently hydrogen or C₁-C₆ alkyl; and n is an integer selected from 1, 2, 3, 4, 5, and 6.

According to some embodiments of the above aspects and embodiments, the cationic lipid is selected from any lipid in Table 2, Table 5, Table 6, Table 7, or Table 8.

According to some embodiments of the above aspects and embodiments, the cationic lipid is a lipid having the structure:

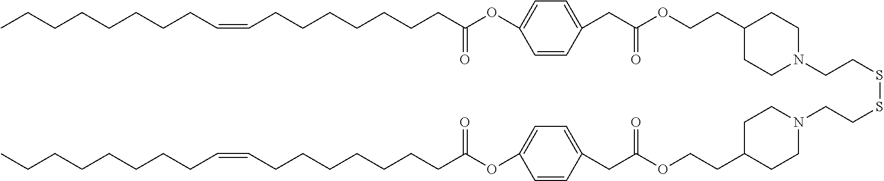 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCC/C=C\CCCCCCCC)cc4)CC3)CC2)cc1]

or a pharmaceutically acceptable salt thereof.

According to some embodiments of the above aspects and embodiments, the cationic lipid is MC3 (6Z,9Z,28Z,31Z)-heptatriaconta-6,9,28,31-tetraen-19-yl-4-(dimethylamino) butanoate (DLin-MC3-DMA or MC3) having the following structure:

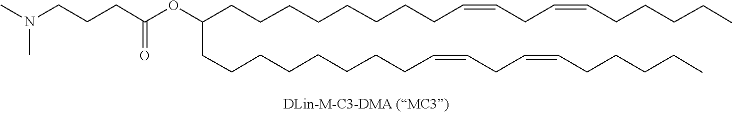 [SMILES: CCCCC/C=C\C/C=C\CCCCCCCCC(CCCCCCCC/C=C\C/C=C\CCCCC)OC(=O)CCCN(C)C]

or a pharmaceutically acceptable salt thereof.

According to some embodiments of the above aspects and embodiments, the sterol or a derivative thereof is a cholesterol or beta-sitosterol. According to some embodiments, the non-cationic lipid is selected from the group consisting of distearoyl-sn-glycero-phosphoethanolamine (DSPE), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), 1,2-dilauroyl-sn-glycero-3-pho sphoethanolamine (DLPE); 1,2-diphytanoyl-sn-glycero-3-phosphoethanolamine (DPHyPE); lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid,cerebrosides, dicetylphosphate, lysophosphatidylcholine, dilinoleoylphosphatidylcholine, and mixtures thereof. According to some embodiments, the non-cationic lipid is selected from the group consisting of dioleoylphosphatidylcholine (DOPC), distearoylphosphatidylcholine (DSPC), and dioleoyl-phosphatidylethanolamine (DOPE). According to some embodiments, the PEGylated lipid is selected from the group consisting of PEG-dilauryloxypropyl; PEG-dimyristyloxypropyl; PEG-dipalmityloxypropyl, PEG-distearyloxypropyl; l-(monomethoxy-polyethyleneglycol)-2,3-dimyristoylglycerol (DMG-PEG); PEG-dilaurylglycerol; PEG-dipalmitoylglycerol; PEG-disterylglycerol; PEG-dilaurylglycamide; PEG-dimyristylglycamide; PEG-dipalmitoylglycamide; PEG-disterylglycamide; (1-[8′-(Cholest-5-en-3[beta]-oxy)carboxamido-3′,6′-dioxaoctanyl]carbamoyl-[omega]-methyl-poly(ethylene glycol) (PEG-cholesterol); 3,4-ditetradecoxylbenzyl-[omega]-methyl-poly(ethylene glycol) ether (PEG-DMB), and 1,2-dimyristoyl-sn-glycero-3-phosphoethanolamine-N-[methoxy(polyethylene glycol) (DSPE-PEG), and 1,2-distearoyl-sn-glycero-3-phosphoethanolamine-N-poly(ethylene glycol)-hydroxyl (DSPE-PEG-OH). According to some embodiments, the PEGylated lipid is DMG-PEG, DSPE-PEG, DSPE-PEG-OH, or a combination thereof. According to some embodiments, the at least one PEGylated lipid is DMG-PEG2000, DSPE-PEG2000, DSPE-PEG2000-OH, or a combination thereof.

According to some embodiments of the above aspects and embodiments, the scFv is chemically conjugated or covalently linked to a PEGylated lipid of the LNP to form a PEGylated lipid conjugate. According to some embodiments, the PEGylated lipid to which the scFv is chemically conjugated or covalently linked is DSPE-PEG. According to some embodiments of the above aspects and embodiments, the scFv is covalently linked to the LNP via a non-cleavable linker. According to some embodiments, the non-cleavable linker is a maleimide-containing linker.

According to some embodiments of the above aspects and embodiments, the scFv is covalently linked to the LNP via a cleavable linker.

According to some embodiments of the above aspects and embodiments, the scFv is covalently linked to the LNP via a pyridyldisulfide (PDS)-containing linker.

According to some embodiments of the above aspects and embodiments, the cationic lipid is present at a molar percentage of about 30% to about 80%. According to some embodiments, the sterol is present at a molar percentage of about 20% to about 50%. According to some embodiments of the above aspects and embodiments, the non-cationic lipid is present at a molar percentage of about 2% to about 20%. According to some embodiments of the above aspects and embodiments, the at least one PEGylated lipid is present at a molar percentage of about 2.1% to about 10%. According to some embodiments of the above aspects and embodiments, scFv polypeptide is present at a total amount of about 0.02 μg/μg of TNA to about 0.1 μg/μg of TNA.

According to some embodiments of the above aspects and embodiments, the pharmaceutical composition further comprises dexamethasone palmitate. According to some embodiments of the above aspects and embodiments, the LNP has a total lipid to TNA ratio of about 10:1 to about 40:1.

According to some embodiments of the above aspects and embodiments, the LNP has a diameter ranging from about 40 nm to about 120 nm. According to some embodiments of the above aspects and embodiments, the nanoparticle has a diameter of less than about 100 nm. According to some embodiments of the above aspects and embodiments, the nanoparticle has a diameter of about 60 nm to about 80 nm. According to some embodiments of the above aspects and embodiments, the ceDNA comprises an expression cassette, and wherein the expression cassette comprises a promoter sequence and a transgene. According to some embodiments, the expression cassette comprises a polyadenylation sequence.

According to some embodiments of the above aspects and embodiments, the ceDNA comprises at least one inverted terminal repeat (ITR) flanking either 5′ or 3′ end of the expression cassette. According to some embodiments, the expression cassette is flanked by two ITRs, wherein the two ITRs comprise one 5′ ITR and one 3′ ITR. According to some embodiments, the expression cassette is connected to an ITR at 3′ end (3′ ITR). According to some embodiments of the above aspects and embodiments, the expression cassette is connected to an ITR at 5′ end (5′ ITR). According to some embodiments of the above aspects and embodiments, the at least one ITR is an ITR derived from an AAV serotype, derived from an ITR of goose virus, derived from a B19 virus ITR, a wild-type ITR from a parvovirus. According to some embodiments of the above aspects and embodiments, the AAV serotype is selected from AAV1, AAV2, AAV3, AAV4, AAV5, AAV6, AAV7, AAV8, AAV9, AAV10, AAV11 and AAV12. According to some embodiments of the above aspects and embodiments, the at least one of the 5′ ITR and the 3′ ITR is a wild-type AAV ITR. According to some embodiments of the above aspects and embodiments, the at least one of the 5′ ITR and the 3′ ITR is a modified or mutant ITR. According to some embodiments of the above aspects and embodiments, the 5′ ITR and the 3′ ITR are symmetrical. According to some embodiments of the above aspects and embodiments, the 5′ ITR and the 3′ ITR are asymmetrical. According to some embodiments of the above aspects and embodiments, the ceDNA further comprises a spacer sequence between a 5′ ITR and the expression cassette. According to some embodiments of the above aspects and embodiments, the ceDNA further comprises a spacer sequence between a 3′ ITR and the expression cassette. According to some embodiments of the above aspects and embodiments, the spacer sequence is at least 5 base pairs long in length. According to some embodiments of the above aspects and embodiments, the ceDNA has a nick or a gap. According to some embodiments of the above aspects and embodiments, the ceDNA is a CELiD, DNA-based minicircle, a MIDGE, a ministring DNA, a dumbbell shaped linear duplex closed-ended DNA comprising two hairpin structures of ITRs in the 5′ and 3′ ends of an expression cassette, or a doggybone™ DNA.

According to another aspect, the disclosure features a method of treating e.g.

According to another aspect, the disclosure provides a method of delivering a therapeutic nucleic acid (TNA) or increasing the concentration of the TNA to a tumor of a subject, comprising administering to the subject an effective amount of the pharmaceutical composition of any one of the aspects and embodiments herein.

### BRIEF DESCRIPTION OF THE DRAWINGS

Embodiments of the present disclosure, briefly summarized above and discussed in greater detail below, can be understood by reference to the illustrative embodiments of the disclosure depicted in the appended drawings. However, the appended drawings illustrate only typical embodiments of the disclosure and are therefore not to be considered limiting of scope, for the disclosure may admit to other equally effective embodiments.

FIGS. 1A-1F show that trastuzumab-derived α-HER2 scFv exhibited clear HER2-specific membrane targeting and internalization in vitro. Alexa-fluor 488-(AF488) labeled anti-HER2 scFv was used to show HER2 receptor engagement in SkBR3 (FIG. 1A) and SkOV3 (FIG. 1B) Her2-expressing (HER2+) cell lines, but not in MCF7 cells (FIG. 1C), which do not express Her2 receptor (HER2-). A second immunofluorescent label (pHrhodo) was used to demonstrate ligand internalization. As shown in FIGS. 1D-1F, SkBR3 and SkOV3 cells that express the HER2 receptor showed ligand internalization (FIG. 1D and FIG. 1E), while the MCF7 HER2-cell line did not (FIG. 1F).

FIG. 2A and FIG. 2B show schematics of exemplary primary routes of conjugation using thiol-based crosslinking.

FIG. 3A and FIG. 3B show that the scFv-LNP conjugation process demonstrated excellent conjugation yield and LNP particle stability. The results of a conjugation process that included an initial TCEP reduction, fresh MAL-LNP (maleimide-conjugated LNP) preparation, 0.5% MAL-PEG2K, scFv:Mal molar equivalents of 0.5, 0.25, 0.1, and 0.05 are shown in FIG. 3A. Next, the PEG chain length was increased to PEG5K and a dialysis step was deployed to remove unreacted scFv without disrupting the particle size and stability. The results are shown in FIG. 3B.

FIG. 4A and FIG. 4B are graphs that show that LNP size and encapsulation efficiency were maintained post-scFv conjugation (±10 nm) with the conjugation process.

FIG. 5 shows that the maleimide conjugation process resulted in robust conjugation.

FIG. 6A and FIG. 6B are graphs that show that only Tras-scFv-conjugated LNPs (FIG. 6A) but not 0.5% DSPE control LNP (FIG. 6B) showed HER2 engagement, thereby confirming ligand function on the LNP.

FIG. 7 shows that maleimide-conjugated LNPs (MAL-LNPs) demonstrated Her2-specific, enhanced cell uptake, specifically demonstrating that the uptake of conjugated Tras-scFv Lipid A LNPs (mCherry) was mediated by HER2.

FIG. 8A and FIG. 8B shows that ligand presentation on the LNP surface significantly affected biological activity. The graph in FIG. 8A compares LNP uptake (mCherry) in maleimide-conjugated LNPs, where the PEG chain length was either 2000 Da (PEG2K) or 5000 Da (PEG5K), normalized to cell viability. As shown in FIG. 8A, maleimide-conjugated LNPs having PEG5K showed greater biological activity, as assessed by cellular uptake of LNPs. The graph in FIG. 8B shows that a dose-dependent decrease in LNP uptake (mCherry) was observed as the maleimide concentration (as conjugated to PEG5K) was increased from 0.5% to 1.25%.

### DETAILED DESCRIPTION

The present disclosure provides lipid nanoparticle (LNP) compositions (e.g., pharmaceutical compositions) comprising a therapeutic nucleic acid (TNA), wherein the LNP comprises a single chain fragment variable (scFv) linked to the LNP, and wherein the scFv is directed against an antigen present on the surface of a cell (e.g., a tumor cell). It is an advantageous feature of the present disclosure that any scFv may be linked to the LNPs, and are useful for targeting any cell or tissue that expresses antigen that the scFv is directed against. The LNP compositions described herein advantageously provide efficient, covalent conjugation with minimal effects on particle size and stability.

According to one aspect, the disclosure provides a pharmaceutical composition comprising a lipid nanoparticle (LNP),a therapeutic nucleic acid (TNA), and at least one pharmaceutically acceptable excipient, wherein the LNP comprises a single-chain variable fragment (scFv) linked to the LNP, wherein the scFv is directed against an antigen present on the surface of a cell. It is a finding of the present disclosure that maleimide conjugation of scFv to LNP resulted in robust conjugation to the LNP and importantly, maintained LNP size and integrity. According to some embodiments, the scFV is covalently linked to the LNP. As used herein, the term “covalent” refers to chemical bonds that involve the sharing of electron pairs between atoms. According to some embodiments, the scFV is chemically conjugated to the LNP. As used herein, the term “conjugation” when referring to conjugation chemistry or system, refers to a system of overlapping p orbitals with delocalized electrons from multiple atoms. According to some embodiments, the scFV is chemically conjugated to the LNP via a non-cleavable linker. According to some embodiments, the non-cleavable linker is a maleimide-containing linker. According to some embodiments, the scFV is chemically conjugated to the LNP via a cleavable linker. According to some embodiments, the cleavable linker is a pyridyl disulfide (PDS)-containing linker. According to some embodiments, the scFV is linked to to the LNP via transglutaminase-mediated conjugation. As used herein, “transglutaminase-mediated conjugation” refers to conjugation as defined herein that is mediated by microbial transglutaminase (MTGase). MTGase catalyzes site-specific modification (i.e., transpeptidation) between a primary amine within linkers and the side chain of a specific glutamine residue of an antibody or a single chain fragment variable (scFv), e.g., glutamine 295 within deglycosylated chimeric, humanized and human IgGI (see, e.g., Anami Y., Tsuchikama K. (2020) Transglutaminase-Mediated Conjugations. In: Tumey L. (eds) Antibody-Drug Conjugates. Methods in Molecular Biology, vol 2078. Humana, New York, NY., incorporated by reference in its entirety herein). This method can be empowered by mutation of asparagine 297, insertion of a glutamine-containing peptide tag, and the use of branched linkers. Such modifications facilitate the conjugation process and provide flexibility in adjusting the conjugation site and drug-to-antibody ratio (DAR) (Yasuaki Anami and Kyoji Tsuchikama “Transglutaminase-Mediated Conjugations” in Methods in Molecular Biology, Antibody Drug Conjugates (2020), incorporated by reference in its entirety herein). In some embodiments, the conjugation can be enhanced by insertion of a glutamine-containing peptide tag and/or the use of branched linkers. In one embodiment, the glutamine-containing peptide tag is LLQGA (Leu-Leu-Gln-Glu-Ala or SEQ ID NO:4). In some embodiments, the glutamine-containing peptide tag comprises SEQ IDNO: 4. In some embodiments, the glutamine-containing tag consists of SEQ ID NO: 4.

As a further advantage, the LNPs comprising described herein provide more efficient delivery of the therapeutic nucleic acid, better tolerability and an improved safety profile. Because the presently described therapeutic nucleic acid lipid particles (e.g., lipid nanoparticles) have no packaging constraints imposed by the space within the viral capsid, in theory, the only size limitation of the therapeutic nucleic acid lipid particles (e.g., lipid nanoparticles) resides in the DNA replication efficiency of the host cell. As described and exemplified herein, according to some embodiments, the therapeutic nucleic acid is a therapeutic nucleic acid (TNA) like double stranded DNA (e.g., ceDNA). Described and exemplified herein, according to some embodiments, the therapeutic nucleic acid is a ceDNA. As also described herein, according to some embodiments, the therapeutic nucleic acid is a mRNA.

### I. Definitions

Unless otherwise defined herein, scientific and technical terms used in connection with the present application shall have the meanings that are commonly understood by those of ordinary skill in the art to which this disclosure belongs. It should be understood that this disclosure is not limited to the particular methodology, protocols, and reagents, etc., described herein and as such can vary. The terminology used herein is for the purpose of describing particular embodiments only, and is not intended to limit the scope of the present disclosure, which is defined solely by the claims. Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), Fields Virology, 6th Edition, published by Lippincott Williams & Wilkins, Philadelphia, PA, USA (2013), Knipe, D. M. and Howley, P.M. (ed.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al. Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

As used in this specification and the appended claims, the singular forms “a”, “an” and “the” include plural references unless the content clearly dictates otherwise.

The abbreviation, “e.g.” is derived from the Latin exempli gratia and is used herein to indicate a non-limiting example. Thus, the abbreviation “e.g.” is synonymous with the term “for example.”

The use of the alternative (e.g., “or”) should be understood to mean either one, both, or any combination thereof of the alternatives.

As used herein, the term “about,” when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, any concentration range, percentage range, ratio range, or integer range is to be understood to include the value of any integer within the recited range and, when appropriate, fractions thereof (such as one tenth and one hundredth of an integer), unless otherwise indicated.

As used herein, “comprise,” “comprising,” and “comprises” and “comprised of” are meant to be synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

The term “consisting of” refers to compositions, methods, processes, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the disclosure.

As used herein, the terms “such as”, “for example” and the like are intended to refer to exemplary embodiments and not to limit the scope of the present disclosure.

Unless defined otherwise, all technical and scientific terms used herein have the same meaning as commonly understood by one of ordinary skill in the art to which the disclosure pertains. Although any methods and materials similar or equivalent to those described herein can be used in the practice for testing of the present disclosure, preferred materials and methods are described herein.

As used herein the terms, “administration,” “administering” and variants thereof refers to introducing a composition or agent (e.g., nucleic acids, in particular ceDNA) into a subject and includes concurrent and sequential introduction of one or more compositions or agents. “Administration” can refer, e.g., to therapeutic, pharmacokinetic, diagnostic, research, placebo, and experimental methods. “Administration” also encompasses in vitro and ex vivo treatments. The introduction of a composition or agent into a subject is by any suitable route, including orally, pulmonarily, intranasally, parenterally (intravenously, intramuscularly, intraperitoneally, or subcutaneously), rectally, intralymphatically, intratumorally, or topically. Administration includes self-administration and the administration by another. Administration can be carried out by any suitable route. A suitable route of administration allows the composition or the agent to perform its intended function. For example, if a suitable route is intravenous, the composition is administered by introducing the composition or agent into a vein of the subject.

The term “antibody” as used herein encompasses any naturally-occurring, recombinant, modified or engineered immunoglobulin or immunoglobulin-like structure or antigen-binding fragment or portion thereof, or derivative thereof, as further described elsewhere herein. Thus, the term refers to an immunoglobulin molecule that specifically binds to a target antigen, and includes, for instance, chimeric, humanized, fully human, and bispecific antibodies. An intact antibody will generally comprise at least two full-length heavy chains and two full-length light chains, but in some instances can include fewer chains such as antibodies naturally occurring in camelids which can comprise only heavy chains. Antibodies can be derived solely from a single source, or can be “chimeric,” that is, different portions of the antibody can be derived from two different antibodies.

Antibodies, or antigen binding portions thereof, can be produced in hybridomas, by recombinant DNA techniques, or by enzymatic or chemical cleavage of intact antibodies. The term antibodies, as used herein, includes monoclonal antibodies, bispecific antibodies, minibodies, domain antibodies, synthetic antibodies (sometimes referred to herein as “antibody mimetics”), chimeric antibodies, humanized antibodies, human antibodies, antibody fusions (sometimes referred to herein as “antibody conjugates”), respectively.

The terms “antigen-binding portion” or “antigen-binding fragment” of an antibody, as used herein, are meant to refer to one or more fragments of an antibody that retain the ability to specifically bind to an antigen (e.g., TGFβ1). Antigen binding portions include, but are not limited to, any naturally occurring, enzymatically obtainable, synthetic, or genetically engineered polypeptide or glycoprotein that specifically binds an antigen to form a complex. In some embodiments, an antigen-binding portion of an antibody may be derived, e.g., from full antibody molecules using any suitable standard techniques such as proteolytic digestion or recombinant genetic engineering techniques involving the manipulation and expression of DNA encoding antibody variable and optionally constant domains. Non-limiting examples of antigen-binding portions include: (i) Fab fragments, a monovalent fragment consisting of the VL, VH, CL and CHI domains; (ii) F(ab′)2 fragments, a bivalent fragment comprising two Fab fragments linked by a disulfide bridge at the hinge region; (iii) Fd fragments consisting of the VH and CHI domains; (iv) Fv fragments consisting of the VL and VH domains of a single arm of an antibody; (v) single-chain Fv (scFv) molecules (see, e.g., Bird et al. (1988) SCIENCE 242:423-426; and Huston et al. (1988) PROC. NAT'L. ACAD. SCI. USA 85:5879-5883); (vi) dAb fragments (see, e.g., Ward et al. (1989) NATURE 341: 544-546); and (vii) minimal recognition units consisting of the amino acid residues that mimic the hypervariable region of an antibody (e.g., an isolated complementarity determining region (CDR)). Other forms of single chain antibodies, such as diabodies are also encompassed. The term antigen binding portion of an antibody includes a “single chain Fab fragment” otherwise known as an “scFab,” comprising an antibody heavy chain variable domain (VH), an antibody constant domain 1 (CHI), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL; and wherein said linker is a polypeptide of at least 30 amino acids, preferably between 32 and 50 amino acids.

As used herein, the term “single-chain variable fragment” or “scFv” is a fusion protein of the variable regions of the heavy (VH) and light chains (VL) of an immunoglobulin covalently linked to form a VH::VL heterodimer. The heavy (VH) and light chains (VL) are either joined directly or joined by a peptide-encoding linker (e.g., 10, 15, 20, 25 amino acids), which connects the N-terminus of the VH with the C-terminus of the VL, or the C-terminus of the VH with the N-terminus of the VL. The linker is usually rich in glycine for flexibility, as well as serine or threonine for solubility. Despite removal of the constant regions and the introduction of a linker, scFv proteins retain the specificity of the original immunoglobulin. Single chain Fv polypeptide antibodies can be expressed from a nucleic acid including VH- and VL-encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. According to some embodiments, scFvs may be used that are derived from Fab's (instead of from an antibody, e.g., obtained from Fab libraries). In one embodiment, the scFv binds human epidermal growth factor receptor 2 (HER2).

As used herein, the term “antigen” is meant to refer to a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. Moreover, a skilled artisan will understand that an antigen need not be encoded by a “gene” at all. An antigen can be generated synthesized or can be derived from a biological sample. Such a biological sample can include, but is not limited to a tissue sample, a tumor sample, a cell or a biological fluid. In one embodiment, the antigen is a tumor-associated antigen (TAA) or a tumor-specific antigen (TSA). In one embodiment, the TAA or TSA is selected from the group consisting of: a glioma-associated antigen, a carcinoembryonic antigen (CEA), f-human chorionic gonadotropin, alphafetoprotein (AFP), lectin-reactive AFP, thyroglubilin, RAGE-1, MN-CA IX, human telomerase reverse transcriptase, RU1, RU2 (AS), intestinal carboxylesterase, mut hsp70-2, M-CSF, prostase, prostate-specific antigen (PSA), PAP, NY-ESO-1, LAGE-la, p53, prostein, PSMA, HER2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin, EphA2, HER2, GD2, Glypican-3, 5T4, 8H9, ανβ6 integrin, BCMA, B7-H3, B7-H6, CAIX, CA9, CD19, CD20, CD22, kappa light chain, CD30, CD33, CD38, CD44, CD44v6, CD44v7/8, CD70, CD123, CD138, CD171, CEA, CSPG4, EGFR, EGFRvIII, EGP2, EGP40, EPCAM, ERBB3, ERBB4, ErbB3/4, FAP, FAR, FBP, fetal AchR, Folate Receptor a, GD2, GD3, HLA-AI MAGE A1, HLA-A2, ILI1R^{a}, ILl3Ra2, KDR, Lambda, Lewis-Y, MCSP, Mesothelin, Mucd, Muc16, NCAM, NKG2D ligands, NY-ESO-1, PRAME, PSCA, PSCl, PSMA, RORI, SURVIVIN, TAG72, TEM1, TEM8, VEGRR2, HMW-MAA, and VEGF receptors. In some embodiments, the TAA or TSA is an antigen that is present within the extracellular matrix of tumors, such as oncofetal variants of fibronectin, tenascin, or necrotic regions of tumors. In some embodiments, the TAA or TSA is any membrane protein or biomarker that is expressed or overexpressed in a tumor cell including, but not limited to, integrins (e.g., integrin ανβ3, a51), EGF Receptor Family (e.g., EGFR2, Erbb2/HER2/neu, Erbb3, Erbb4), proteoglycans (e.g., heparan sulfate proteoglycans), disialogangliosides (e.g., GD2, GD3), B7-H3 (aka CD276), cancer antigen 125 (CA-125), epithelial cell adhesion molecule (EpCAM), vascular endothelial growth factor receptors 1 and 2 (VEGFR-1, VEGFR-2), CD52, carcinoembryonic antigen (CEA), tumor associated glycoproteins (e.g., TAG-72), cluster of differentiation 19 (CD19), CD20, CD22, CD30, CD33, CD40, CD44, CD74, CD152, mucin 1 (MUC1), tumor necrosis factor receptors (e.g., TRAIL-R2), insulin-like growth factor receptors, folate receptor a, transmembrane glycoprotein NMB (GPNMB), C-C chemokine receptors (e.g., CCR4), prostate specific membrane antigen (PSMA), recepteur d'origine nantais (RON) receptor, cytotoxic T-lymphocyte antigen 4 (CTLA4), and other tumor specific receptors or antigens.

In one embodiment, the antigen is human epidermal growth factor receptor 2 (HER2).

As used herein, the phrase “anti-therapeutic nucleic acid immune response”, “anti-transfer vector immune response”, “immune response against a therapeutic nucleic acid”, “immune response against a transfer vector”, or the like is meant to refer to any undesired immune response against a therapeutic nucleic acid, viral or non-viral in its origin. In some embodiments, the undesired immune response is an antigen-specific immune response against the viral transfer vector itself. In some embodiments, the immune response is specific to the transfer vector which can be double stranded DNA, single stranded RNA, or double stranded RNA. In other embodiments, the immune response is specific to a sequence of the transfer vector. In other embodiments, the immune response is specific to the CpG content of the transfer vector.

As used herein, the term “aqueous solution” is meant to refer to a composition comprising in whole, or in part, water.

As used herein, the term “bases” includes purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides.

As used herein, the terms “carrier” and “excipient” are meant to include any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutically active substances is well known in the art. Supplementary active ingredients can also be incorporated into the compositions. The phrase “pharmaceutically-acceptable” refers to molecular entities and compositions that do not produce a toxic, an allergic, or similar untoward reaction when administered to a host.

As used herein, the term “ceDNA” is meant to refer to capsid-free closed-ended linear double stranded (ds) duplex DNA for non-viral gene transfer, synthetic or otherwise. According to some embodiments, the ceDNA is a closed-ended linear duplex (CELiD) CELiD DNA. According to some embodiments, the ceDNA is a DNA-based minicircle. According to some embodiments, the ceDNA is a minimalistic immunological-defined gene expression (MIDGE)-vector. According to some embodiments, the ceDNA is a ministering DNA. According to some embodiments, the ceDNA is a dumbbell shaped linear duplex closed-ended DNA comprising two hairpin structures of ITRs in the 5′ and 3′ ends of an expression cassette. According to some embodiments, the ceDNA is a doggybone™ DNA. Detailed description of ceDNA is described in International Patent Application No. PCT/US2017/020828, filed Mar. 3, 2017, the entire contents of which are expressly incorporated herein by reference. Certain methods for the production of ceDNA comprising various inverted terminal repeat (ITR) sequences and configurations using cell-based methods are described in Example 1 of International Patent Application Nos. PCT/US18/49996, filed Sep. 7, 2018, and PCT/US2018/064242, filed Dec. 6, 2018 each of which is incorporated herein in its entirety by reference. Certain methods for the production of synthetic ceDNA vectors comprising various ITR sequences and configurations are described, e.g., in International application PCT/US2019/14122, filed Jan. 18, 2019, the entire content of which is incorporated herein by reference.

As used herein, the term “closed-ended DNA vector” refers to a capsid-free DNA vector with at least one covalently closed end and where at least part of the vector has an intramolecular duplex structure.

As used herein, the terms “ceDNA vector” and “ceDNA” are used interchangeably and refer to a closed-ended DNA vector comprising at least one terminal palindrome. In some embodiments, the ceDNA comprises two covalently-closed ends.

As used herein, the term “ceDNA-bacmid” is meant to refer to an infectious baculovirus genome comprising a ceDNA genome as an intermolecular duplex that is capable of propagating in E. coli as a plasmid, and so can operate as a shuttle vector for baculovirus.

As used herein, the term “ceDNA-baculovirus” is meant to refer to a baculovirus that comprises a ceDNA genome as an intermolecular duplex within the baculovirus genome.

As used herein, the terms “ceDNA-baculovirus infected insect cell” and “ceDNA-BIIC” are used interchangeably, and are meant to refer to an invertebrate host cell (including, but not limited to an insect cell (e.g., an Sf9 cell)) infected with a ceDNA-baculovirus.

As used herein, the term “ceDNA genome” is meant to refer to an expression cassette that further incorporates at least one inverted terminal repeat (ITR) region. A ceDNA genome may further comprise one or more spacer regions. In some embodiments the ceDNA genome is incorporated as an intermolecular duplex polynucleotide of DNA into a plasmid or viral genome.

As used herein, the terms “DNA regulatory sequences,” “control elements,” and “regulatory elements,” are used interchangeably herein, and are meant to refer to transcriptional and translational control sequences, such as promoters, enhancers, polyadenylation signals, terminators, protein degradation signals, and the like, that provide for and/or regulate transcription of a non-coding sequence (e.g., DNA-targeting RNA) or a coding sequence (e.g., site-directed modifying polypeptide, or Cas9/Csnl polypeptide) and/or regulate translation of an encoded polypeptide.

As used herein, the term “terminal repeat” or “TR¹” includes any viral or non-viral terminal repeat or synthetic sequence that comprises at least one minimal required origin of replication and a region comprising a palindromic hairpin structure. A Rep-binding sequence (“RBS” or also referred to as Rep-binding element (RBE)) and a terminal resolution site (“TRS”) together constitute a “minimal required origin of replication” for an AAV and thus the TR comprises at least one RBS and at least one TRS. TRs that are the inverse complement of one another within a given stretch of polynucleotide sequence are typically each referred to as an “inverted terminal repeat” or “ITR”. In the context of a virus, ITRs plays a critical role in mediating replication, viral particle and DNA packaging, DNA integration and genome and provirus rescue. TRs that are not inverse complement (palindromic) across their full length can still perform the traditional functions of ITRs, and thus, the term ITR is used to refer to a TR in an viral or non-viral AAV vector that is capable of mediating replication of in the host cell. It will be understood by one of ordinary skill in the art that in a complex AAV vector configurations more than two ITRs or asymmetric ITR pairs may be present.

The “ITR¹” can be artificially synthesized using a set of oligonucleotides comprising one or more desirable functional sequences (e.g., palindromic sequence, RBS). The ITR sequence can be an AAV ITR, an artificial non-AAV ITR, or an ITR physically derived from a viral AAV ITR (e.g., ITR fragments removed from a viral genome). For example, the ITR can be derived from the family Parvoviridae, which encompasses parvoviruses and dependoviruses (e.g., canine parvovirus, bovine parvovirus, mouse parvovirus, porcine parvovirus, human parvovirus B-19), or the SV40 hairpin that serves as the origin of SV40 replication can be used as an ITR, which can further be modified by truncation, substitution, deletion, insertion and/or addition. Parvoviridae family viruses consist of two subfamilies: Parvovirinae, which infect vertebrates, and Densovirinae, which infect invertebrates. Dependoparvoviruses include the viral family of the adeno-associated viruses (AAV) which are capable of replication in vertebrate hosts including, but not limited to, human, primate, bovine, canine, equine and ovine species. Typically, ITR sequences can be derived not only from AAV, but also from Parvovirus, lentivirus, goose virus, B19, in the configurations of wildtype, “doggy bone” and “dumbbell shape”, symmetrical or even asymmetrical ITR orientation. Although the ITRs are typically present in both 5′ and 3′ ends of an AAV vector, ITR can be present in only one of end of the linear vector. For example, the ITR can be present on the 5′ end only. Some other cases, the ITR can be present on the 3′ end only in synthetic AAV vector. For convenience herein, an ITR located 5′ to (“upstream of”) an expression cassette in a synthetic AAV vector is referred to as a “5” ITR¹′ or a “left ITR”, and an ITR located 3′ to (“downstream of”) an expression cassette in a vector or synthetic AAV is referred to as a “3” ITR¹′ or a “right ITR”.

As used herein, a “wild-type ITR¹” or “WT-ITR¹” refers to the sequence of a naturally occurring ITR sequence in an AAV genome or other dependovirus that remains, e.g., Rep binding activity and Rep nicking ability. The nucleotide sequence of a WT-ITR from any AAV serotype may slightly vary from the canonical naturally occurring sequence due to degeneracy of the genetic code or drift, and therefore WT-ITR sequences encompasses for use herein include WT-ITR sequences as result of naturally occurring changes (e.g., a replication error).

As used herein, the term “substantially symmetrical WT-ITRs” or a “substantially symmetrical WT-ITR pair” refers to a pair of WT-ITRs within a synthetic AAV vector that are both wild type ITRs that have an inverse complement sequence across their entire length. For example, an ITR can be considered to be a wild-type sequence, even if it has one or more nucleotides that deviate from the canonical naturally occurring canonical sequence, so long as the changes do not affect the physical and functional properties and overall three-dimensional structure of the sequence (secondary and tertiary structures). In some aspects, the deviating nucleotides represent conservative sequence changes. As one non-limiting example, a sequence that has at least 95%, 96%, 97%, 98%, or 99% sequence identity to the canonical sequence (as measured, e.g., using BLAST at default settings), and also has a symmetrical three-dimensional spatial organization to the other WT-ITR such that their 3D structures are the same shape in geometrical space. The substantially symmetrical WT-ITR has the same A, C-C‘ and B-B’ loops in 3D space. A substantially symmetrical WT-ITR can be functionally confirmed as WT by determining that it has an operable Rep binding site (RBE or RBE′) and terminal resolution site (trs) that pairs with the appropriate Rep protein. One can optionally test other functions, including transgene expression under permissive conditions.

As used herein, the phrases of “modified ITR¹” or “mod-ITR¹” or “mutant ITR¹” are used interchangeably and refer to an ITR with a mutation in at least one or more nucleotides as compared to the WT-ITR from the same serotype. The mutation can result in a change in one or more of A, C, C′, B, B′ regions in the ITR, and can result in a change in the three-dimensional spatial organization (i.e. its 3D structure in geometric space) as compared to the 3D spatial organization of a WT-ITR of the same serotype.

As used herein, the term “asymmetric ITRs” also referred to as “asymmetric ITR pairs” refers to a pair of ITRs within a single synthetic AAV genome that are not inverse complements across their full length. As one non-limiting example, an asymmetric ITR pair does not have a symmetrical three-dimensional spatial organization to their cognate ITR such that their 3D structures are different shapes in geometrical space. Stated differently, an asymmetrical ITR pair have the different overall geometric structure, i.e., they have different organization of their A, C-C‘ and B-B’ loops in 3D space (e.g., one ITR may have a short C-C′ arm and/or short B-B′ arm as compared to the cognate ITR). The difference in sequence between the two ITRs may be due to one or more nucleotide addition, deletion, truncation, or point mutation. In one embodiment, one ITR of the asymmetric ITR pair may be a wild-type AAV ITR sequence and the other ITR a modified ITR as defined herein (e.g., a non-wild-type or synthetic ITR sequence). In another embodiment, neither ITRs of the asymmetric ITR pair is a wild-type AAV sequence and the two ITRs are modified ITRs that have different shapes in geometrical space (i.e., a different overall geometric structure). In some embodiments, one mod-ITRs of an asymmetric ITR pair can have a short C-C′ arm and the other ITR can have a different modification (e.g., a single arm, or a short B-B′ arm etc.) such that they have different three-dimensional spatial organization as compared to the cognate asymmetric mod-ITR.

As used herein, the term “symmetric ITRs” refers to a pair of ITRs within a single stranded AAV genome that are wild-type or mutated (e.g., modified relative to wild-type) dependoviral ITR sequences and are inverse complements across their full length. In one non-limiting example, both ITRs are wild type ITRs sequences from AAV2. In another example, neither ITRs are wild type ITR AAV2 sequences (i.e., they are a modified ITR, also referred to as a mutant ITR), and can have a difference in sequence from the wild type ITR due to nucleotide addition, deletion, substitution, truncation, or point mutation. For convenience herein, an ITR located 5′ to (upstream of) an expression cassette in a synthetic AAV vector is referred to as a “5” ITR¹′ or a “left ITR”, and an ITR located 3′ to (downstream of) an expression cassette in a synthetic AAV vector is referred to as a “3” ITR¹′ or a “right ITR”.

As used herein, the terms “substantially symmetrical modified-ITRs” or a “substantially symmetrical mod-ITR pair” refers to a pair of modified-ITRs within a synthetic AAV that are both that have an inverse complement sequence across their entire length. For example, the a modified ITR can be considered substantially symmetrical, even if it has some nucleotide sequences that deviate from the inverse complement sequence so long as the changes do not affect the properties and overall shape. As one non-limiting example, a sequence that has at least 85%, 90%, 95%, 96%, 97%, 98%, or 99% sequence identity to the canonical sequence (as measured using BLAST at default settings), and also has a symmetrical three-dimensional spatial organization to their cognate modified ITR such that their 3D structures are the same shape in geometrical space. Stated differently, a substantially symmetrical modified-ITR pair have the same A, C-C‘ and B-B’ loops organized in 3D space. In some embodiments, the ITRs from a mod-ITR pair may have different reverse complement nucleotide sequences but still have the same symmetrical three-dimensional spatial organization —that is both ITRs have mutations that result in the same overall 3D shape. For example, one ITR (e.g., 5′ ITR) in a mod-ITR pair can be from one serotype, and the other ITR (e.g., 3′ ITR) can be from a different serotype, however, both can have the same corresponding mutation (e.g., if the 5′ITR has a deletion in the C region, the cognate modified 3′ITR from a different serotype has a deletion at the corresponding position in the C′ region), such that the modified ITR pair has the same symmetrical three-dimensional spatial organization. In such embodiments, each ITR in a modified ITR pair can be from different serotypes (e.g., AAV1, 2, 3, 4, 5, 6, 7, 8, 9, 10, 11, and 12) such as the combination of AAV2 and AAV6, with the modification in one ITR reflected in the corresponding position in the cognate ITR from a different serotype. In one embodiment, a substantially symmetrical modified ITR pair refers to a pair of modified ITRs (mod-ITRs) so long as the difference in nucleotide sequences between the ITRs does not affect the properties or overall shape and they have substantially the same shape in 3D space. As a non-limiting example, a mod-ITR that has at least 95%, 96%, 97%, 98% or 99% sequence identity to the canonical mod-ITR as determined by standard means well known in the art such as BLAST (Basic Local Alignment Search Tool), or BLASTN at default settings, and also has a symmetrical three-dimensional spatial organization such that their 3D structure is the same shape in geometric space. A substantially symmetrical mod-ITR pair has the same A, C-C‘ and B-B’ loops in 3D space, e.g., if a modified ITR in a substantially symmetrical mod-ITR pair has a deletion of a C-C′ arm, then the cognate mod-ITR has the corresponding deletion of the C-C′ loop and also has a similar 3D structure of the remaining A and B-B′ loops in the same shape in geometric space of its cognate mod-ITR.

As used herein, the phrase an “effective amount” or “therapeutically effective amount” of an active agent or therapeutic agent, such as a therapeutic nucleic acid, is an amount sufficient to produce the desired effect, e.g., inhibition of expression of a target sequence in comparison to the expression level detected in the absence of a therapeutic nucleic acid. Suitable assays for measuring expression of a target gene or target sequence include, e.g., examination of protein or RNA levels using techniques known to those of skill in the art such as dot blots, northern blots, in situ hybridization, ELISA, immunoprecipitation, enzyme function, as well as phenotypic assays known to those of skill in the art.

As used herein, the term “expression” is meant to refer to the cellular processes involved in producing RNA and proteins and as appropriate, secreting proteins, including where applicable, but not limited to, for example, transcription, transcript processing, translation and protein folding, modification and processing. As used herein, the phrase “expression products” include RNA transcribed from a gene (e.g., transgene), and polypeptides obtained by translation of mRNA transcribed from a gene.

As used herein, the term “expression vector” is meant to refer to a vector that directs expression of an RNA or polypeptide from sequences linked to transcriptional regulatory sequences on the vector. The sequences expressed will often, but not necessarily, be heterologous to the host cell. An expression vector may comprise additional elements, for example, the expression vector may have two replication systems, thus allowing it to be maintained in two organisms, for example in human cells for expression and in a prokaryotic host for cloning and amplification. The expression vector may be a recombinant vector.

As used herein, the term “flanking” is meant to refer to a relative position of one nucleic acid sequence with respect to another nucleic acid sequence. Generally, in the sequence ABC, B is flanked by A and C. The same is true for the arrangement A×B×C. Thus, a flanking sequence precedes or follows a flanked sequence but need not be contiguous with, or immediately adjacent to the flanked sequence.

As used herein, the term “spacer region” is meant to refer to an intervening sequence that separates functional elements in a vector or genome. In some embodiments, spacer regions keep two functional elements at a desired distance for optimal functionality. In some embodiments, the spacer regions provide or add to the genetic stability of the vector or genome. In some embodiments, spacer regions facilitate ready genetic manipulation of the genome by providing a convenient location for cloning sites and a gap of design number of base pair.

As used herein, the terms “expression cassette” and “expression unit” are used interchangeably, and meant to refer to a heterologous DNA sequence that is operably linked to a promoter or other DNA regulatory sequence sufficient to direct transcription of a transgene of a DNA vector, e.g., synthetic AAV vector. Suitable promoters include, for example, tissue specific promoters. Promoters can also be of AAV origin.

As used herein, the phrase “genetic disease” or “genetic disorder” is meant to refer to a disease, partially or completely, directly or indirectly, caused by one or more abnormalities in the genome, including and especially a condition that is present from birth. The abnormality may be a mutation, an insertion or a deletion in a gene. The abnormality may affect the coding sequence of the gene or its regulatory sequence.

As used herein, the term “polypeptide” is meant to refer to a repeating sequence of amino acids.

As used herein, the term “lipid” is meant to refer to a group of organic compounds that include, but are not limited to, esters of fatty acids and are characterized by being insoluble in water, but soluble in many organic solvents. They are usually divided into at least three classes: (1) “simple lipids,” which include fats and oils as well as waxes; (2) “compound lipids,” which include phospholipids and glycolipids; and (3) “derived lipids” such as steroids.

Representative examples of phospholipids include, but are not limited to, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine, dipalmitoylphosphatidylcholine, dioleoylphosphatidylcholine, distearoylphosphatidylcholine, and dilinoleoylphosphatidylcholine. Other compounds lacking in phosphorus, such as sphingolipid, glycosphingolipid families, diacylglycerols, and O-acyloxyacids, are also within the group designated as amphipathic lipids. Additionally, the amphipathic lipids described above can be mixed with other lipids including triglycerides and sterols.

In one embodiment, the lipid compositions comprise one or more tertiary amino groups, one or more phenyl ester bonds, and a disulfide bond.

As used herein, the term “lipid conjugate” is meant to refer to a conjugated lipid that inhibits aggregation of lipid particles (e.g., lipid nanoparticles). Such lipid conjugates include, but are not limited to, PEGylated lipids such as, e.g., PEG coupled to dialkyloxypropyls (e.g., PEG-DAA conjugates), PEG coupled to diacylglycerols (e.g., PEG-DAG conjugates), PEG coupled to cholesterol, PEG coupled to phosphatidylethanolamines, and PEG conjugated to ceramides (see, e.g., U.S. Pat. No. 5,885,613), cationic PEG lipids, polyoxazoline (POZ)-lipid conjugates (e.g., POZ-DAA conjugates; see, e.g., U.S. Provisional Application No. 61/294,828, filed Jan. 13, 2010, and U.S. Provisional Application No. 61/295,140, filed Jan. 14, 2010), polyamide oligomers (e.g., ATTA-lipid conjugates), and mixtures thereof. Additional examples of POZ-lipid conjugates are described in International Patent Application Publication No. WO 2010/006282. PEG or POZ can be conjugated directly to the lipid or may be linked to the lipid via a linker moiety. Any linker moiety suitable for coupling the PEG or the POZ to a lipid can be used including, e.g., non-ester containing linker moieties and ester-containing linker moieties. In certain preferred embodiments, non-ester containing linker moieties, such as amides or carbamates, are used. The disclosures of each of the above patent documents are herein incorporated by reference in their entirety for all purposes.

As used herein, the term “lipid encapsulated” is meant to refer to a lipid particle that provides an active agent or therapeutic agent, such as a nucleic acid (e.g., a ceDNA), with full encapsulation, partial encapsulation, or both. In a preferred embodiment, the nucleic acid is fully encapsulated in the lipid particle (e.g., to form a nucleic acid containing lipid particle).

As used herein, the terms “lipid particle” or “lipid nanoparticle” is meant to refer to a lipid formulation that can be used to deliver a therapeutic agent such as nucleic acid therapeutics to a target site of interest (e.g., cell, tissue, organ, and the like). In one embodiment, the lipid particle of the disclosure is a nucleic acid containing lipid particle, which is typically formed from a cationic lipid, a non-cationic lipid, and optionally a conjugated lipid that prevents aggregation of the particle. In other preferred embodiments, a therapeutic agent such as a therapeutic nucleic acid may be encapsulated in the lipid portion of the particle, thereby protecting it from enzymatic degradation. In one embodiment, the lipid particle comprises a nucleic acid (e.g., ceDNA) and a lipid comprising one or more a tertiary amino groups, one or more phenyl ester bonds and a disulfide bond.

According to some embodiments, the lipid particles of the disclosure typically have a mean diameter of from about 20 nm to about 75 nm, about 20 nm to about 70 nm, about 25 nm to about 75 nm, about 25 nm to about 70 nm, from about 30 nm to about 75 nm, from about 30 nm to about 70 nm, from about 35 nm to about 75 nm, from about 35 nm to about 70 nm, from about 40 nm to about 75 nm, from about 40 nm to about 70 nm, from about 45 nm to about 75 nm, from about 50 nm to about 75 nm, from about 50 nm to about 70 nm, from about 60 nm to about 75 nm, from about 60 nm to about 70 nm, from about 65 nm to about 75 nm, from about 65 nm to about 70 nm, or about 20 nm, about 25 nm, about 30 nm, about 35 nm, about 40 nm, about 45 nm, about 50 nm, about 51 nm, about 52 nm, about 53 nm, about 54 nm, about 55 nm, about 56 nm, about 57 nm, about 58 nm, about 59 nm about 60 nm, about 61 nm, about 62 nm, about 63 nm, about 64 nm, about 65 nm, about 66 nm, about 67 nm, about 68 nm, about 69 nm, about 70 nm, about 71 nm, about 72 nm, about 73 nm, about 74 nm, or about 75 nm (±3 nm) in size.

Generally, the lipid particles (e.g., lipid nanoparticles) of the disclosure have a mean diameter selected to provide an intended therapeutic effect.

According to some embodiments, the lipid particles of the disclosure typically have a mean diameter of less than about 75 nm, less than about 70 nm, less than about 65 nm, less than about 60 nm, less than about 55 nm, less than about 50 nm, less than about 45 nm, less than about 40 nm, less than about 35 nm, less than about 30 nm, less than about 25 nm, less than about 20 nm in size.

As used herein, the term “cationic lipid” refers to any lipid that is positively charged at physiological pH. The cationic lipid in the lipid particles may comprise, e.g., one or more cationic lipids such as 1,2-dilinoleyloxy-N,N-dimethylaminopropane (DLinDMA), 1,2-dilinolenyloxy-N,N-dimethylaminopropane (DLenDMA), 1,2-di-γ-linolenyloxy-N,N-dimethylaminopropane (γ-DLenDMA), 2,2-dilinoleyl-4-(2-dimethylaminoethyl)-[1,3]-dioxolane (DLin-K-C2-DMA), 2,2-dilinoleyl-4-dimethylaminomethyl-[1,3]-dioxolane (DLin-K-DMA), “SS-cleavable lipid”, or a mixture thereof. In some embodiments, a cationic lipid is also an ionizable lipid, i.e., an ionizable cationic lipid. Corresponding quaternary lipids of all cationic lipids described herein (i.e., where the nitrogen atom in the cationic moiety is protonated and has four substituents) are contemplated within the scope of this disclosure. Any cationic lipid described herein may be converted to corresponding quaternary lipids, for example, by treatment with chloromethane (CH₃C₁) in acetonitrile (CH₃CN) and chloroform (CHCl₃).

As used herein, the term “anionic lipid” refers to any lipid that is negatively charged at physiological pH. These lipids include, but are not limited to, phosphatidylglycerols, cardiolipins, diacylphosphatidylserines, diacylphosphatidic acids, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), and other anionic modifying groups joined to neutral lipids.

As used herein, the term “hydrophobic lipid” refers to compounds having apolar groups that include, but are not limited to, long-chain saturated and unsaturated aliphatic hydrocarbon groups and such groups optionally substituted by one or more aromatic, cycloaliphatic, or heterocyclic group(s). Suitable examples include, but are not limited to, diacylglycerol, dialkylglycerol, N-N-dialkylamino, 1,2-diacyloxy-3-aminopropane, and 1,2-dialkyl-3-aminopropane.

As used herein, the term “ionizable lipid” is meant to refer to a lipid, e.g., cationic lipid, having at least one protonatable or deprotonatable group, such that the lipid is positively charged at a pH at or below physiological pH (e.g., pH 7.4), and neutral at a second pH, preferably at or above physiological pH. It will be understood by one of ordinary skill in the art that the addition or removal of protons as a function of pH is an equilibrium process, and that the reference to a charged or a neutral lipid refers to the nature of the predominant species and does not require that all of the lipid be present in the charged or neutral form. Generally, ionizable lipids have a pKa of the protonatable group in the range of about 4 to about 7. In some embodiments, ionizable lipid may include “cleavable lipid” or “SS-cleavable lipid”.

As used herein, the term “neutral lipid” is meant to refer to any of a number of lipid species that exist either in an uncharged or neutral zwitterionic form at a selected pH. At physiological pH, such lipids include, for example, diacylphosphatidylcholine, diacylphosphatidylethanolamine, ceramide, sphingomyelin, cephalin, cholesterol, cerebrosides, and diacylglycerols.

As used herein, the term “non-cationic lipid” is meant to refer to any amphipathic lipid as well as any other neutral lipid or anionic lipid.

As used herein, the term “cleavable lipid” or “SS-cleavable lipid” refers to a lipid comprising a disulfide bond cleavable unit. Cleavable lipids may include cleavable disulfide bond (“ss”) containing lipid-like materials that comprise a pH-sensitive tertiary amine and self-degradable phenyl ester. For example, a SS-cleavable lipid can be an ss-OP lipid (COATSOME® SS-OP), an ss-M lipid (COATSOME® SS-M), an ss-E lipid (COATSOME® SS-E), an ss-EC lipid (COATSOME® SS-EC), an ss-LC lipid (COATSOME® SS-LC), an ss-OC lipid (COATSOME® SS-OC), and an ss-PalmE lipid (see, for example, Formulae I-IV), or a lipid described by Togashi et al., (2018) Journal of Controlled Release “A hepatic pDNA delivery system based on an intracellular environment sensitive vitaminE-scaffold lipid-like material with the aid of an anti-inflammatory drug” 279:262-270.

Additional examples of cleavable lipids are described in U.S. Pat. Nos. 9,708,628, and 10,385,030, the entire contents of which are incorporated herein by reference. In one embodiment, cleavable lipids comprise a tertiary amine, which responds to an acidic compartment, e.g., an endosome or lysosome for membrane destabilization and a disulfide bond that can be cleaved in a reducing environment, such as the cytoplasm. In one embodiment, a cleavable lipid is a cationic lipid. In one embodiment, a cleavable lipid is an ionizable cationic lipid. Cleavable lipids are described in more detail herein.

As used herein, the term “organic lipid solution” is meant to refer to a composition comprising in whole, or in part, an organic solvent having a lipid.

As used herein, the term “liposome” is meant to refer to lipid molecules assembled in a spherical configuration encapsulating an interior aqueous volume that is segregated from an aqueous exterior. Liposomes are vesicles that possess at least one lipid bilayer. Liposomes are typical used as carriers for drug/therapeutic delivery in the context of pharmaceutical development. They work by fusing with a cellular membrane and repositioning its lipid structure to deliver a drug or active pharmaceutical ingredient. Liposome compositions for such delivery are typically composed of phospholipids, especially compounds having a phosphatidylcholine group, however these compositions may also include other lipids.

As used herein, the term “local delivery” is meant to refer to delivery of an active agent such as an interfering RNA (e.g., siRNA) directly to a target site within an organism. For example, an agent can be locally delivered by direct injection into a disease site such as a tumor or other target site such as a site of inflammation or a target organ such as the liver, heart, pancreas, kidney, and the like.

As used herein, the term “nucleic acid,” is meant to refer to a polymer containing at least two nucleotides (i.e., deoxyribonucleotides or ribonucleotides) in either single- or double-stranded form and includes DNA, RNA, and hybrids thereof. DNA may be in the form of, e.g., antisense molecules, plasmid DNA, DNA-DNA duplexes, pre-condensed DNA, PCR products, vectors (P1, PAC, BAC, YAC, artificial chromosomes), expression cassettes, chimeric sequences, chromosomal DNA, or derivatives and combinations of these groups. DNA may be in the form of minicircle, plasmid, bacmid, minigene, ministring DNA (linear covalently closed DNA vector), closed-ended linear duplex DNA (CELiD or ceDNA), doggybone™ DNA, dumbbell shaped DNA, minimalistic immunological-defined gene expression (MIDGE)-vector, viral vector or nonviral vectors. RNA may be in the form of small interfering RNA (siRNA), Dicer-substrate dsRNA, small hairpin RNA (shRNA), asymmetrical interfering RNA (aiRNA), microRNA (miRNA), mRNA, rRNA, tRNA, viral RNA (vRNA), and combinations thereof. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs and/or modified residues include, without limitation, phosphorothioates, phosphorodiamidate morpholino oligomer (morpholino), phosphoramidates, methyl phosphonates, chiral-methyl phosphonates, 2′-O-methyl ribonucleotides, locked nucleic acid (LNA™), and peptide nucleic acids (PNAs). Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated.

As used herein, the phrases “nucleic acid therapeutic”, “therapeutic nucleic acid” and “TNA” are used interchangeably and refer to any modality of therapeutic using nucleic acids as an active component of therapeutic agent to treat a disease or disorder. As used herein, these phrases refer to RNA-based therapeutics and DNA-based therapeutics. Non-limiting examples of RNA-based therapeutics include mRNA, antisense RNA and oligonucleotides, ribozymes, aptamers, interfering RNAs (RNAi), Dicer-substrate dsRNA, small hairpin RNA (shRNA), asymmetrical interfering RNA (aiRNA), microRNA (miRNA). Non-limiting examples of DNA-based therapeutics include minicircle DNA, minigene, viral DNA (e.g., Lentiviral or AAV genome) or non-viral synthetic DNA vectors, closed-ended linear duplex DNA (ceDNA/CELiD), plasmids, bacmids, DOGGYBONE™ DNA vectors, minimalistic immunological-defined gene expression (MIDGE)-vector, nonviral ministring DNA vector (linear-covalently closed DNA vector), or dumbbell-shaped DNA minimal vector (“dumbbell DNA”).

As used herein, “nucleotides” contain a sugar deoxyribose (DNA) or ribose (RNA), a base, and a phosphate group. Nucleotides are linked together through the phosphate groups.

As used herein, the term “pharmaceutically acceptable carrier” includes any of the standard pharmaceutical carriers, such as a phosphate buffered saline solution, water, emulsions such as an oil/water or water/oil, and various types of wetting agents. The term also encompasses any of the agents approved by a regulatory agency of the US Federal government or listed in the US Pharmacopeia for use in animals, including humans, as well as any carrier or diluent that does not cause significant irritation to a subject and does not abrogate the biological activity and properties of the administered compound.

As used herein, the term “gap” is meant to refer to a discontinued portion of synthetic DNA vector of the present disclosure, creating a stretch of single stranded DNA portion in otherwise double stranded ceDNA. The gap can be 1 base-pair to 100 base-pair long in length in one strand of a duplex DNA. Typical gaps, designed and created by the methods described herein and synthetic vectors generated by the methods can be, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 bp long in length. Exemplified gaps in the present disclosure can be 1 bp to 10 bp long, 1 to 20 bp long, 1 to 30 bp long in length.

As used herein, the term “nick” refers to a discontinuity in a double stranded DNA molecule where there is no phosphodiester bond between adjacent nucleotides of one strand typically through damage or enzyme action. It is understood that one or more nicks allow for the release of torsion in the strand during DNA replication and that nicks are also thought to play a role in facilitating binding of transcriptional machinery.

By “receptor” is meant a polypeptide, or portion thereof, present on a cell membrane that selectively binds one or more ligands. The term “receptor” as used herein is intended to encompass the entire receptor or ligand-binding portions thereof. These portions of the receptor particularly include those regions sufficient for specific binding of the ligand to occur.

As used herein, the term “cancer” as used herein refers to the physiological condition in multicellular eukaryotes that is typically characterized by unregulated cell proliferation and malignancy. Thus, the term broadly encompasses, solid tumors, blood cancers (e.g., leukemias), as well as myelofibrosis and multiple myeloma.

As used herein, the term “subject” is meant to refer to a human or animal, to whom treatment, including prophylactic treatment, with the therapeutic nucleic acid according to the present disclosure, is provided. Usually, the animal is a vertebrate such as, but not limited to a primate, rodent, domestic animal or game animal. Primates include but are not limited to, chimpanzees, cynomolgus monkeys, spider monkeys, and macaques, e.g., Rhesus. Rodents include mice, rats, woodchucks, ferrets, rabbits and hamsters. Domestic and game animals include, but are not limited to, cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In certain embodiments of the aspects described herein, the subject is a mammal, e.g., a primate or a human. A subject can be male or female. Additionally, a subject can be an infant or a child. In some embodiments, the subject can be a neonate or an unborn subject, e.g., the subject is in utero. Preferably, the subject is a mammal. The mammal can be a human, non-human primate, mouse, rat, dog, cat, horse, or cow, but is not limited to these examples. Mammals other than humans can be advantageously used as subjects that represent animal models of diseases and disorders. In addition, the methods and compositions described herein can be used for domesticated animals and/or pets. A human subject can be of any age, gender, race or ethnic group, e.g., Caucasian (white), Asian, African, black, African American, African European, Hispanic, Mideastern, etc. In some embodiments, the subject can be a patient or other subject in a clinical setting. In some embodiments, the subject is already undergoing treatment. In some embodiments, the subject is an embryo, a fetus, neonate, infant, child, adolescent, or adult. In some embodiments, the subject is a human fetus, human neonate, human infant, human child, human adolescent, or human adult. In some embodiments, the subject is an animal embryo, or non-human embryo or non-human primate embryo. In some embodiments, the subject is a human embryo.

As used herein, the phrase “subject in need” refers to a subject that (i) will be administered a ceDNA lipid particle (or pharmaceutical composition comprising a ceDNA lipid particle) according to the described disclosure, (ii) is receiving a ceDNA lipid particle (or pharmaceutical composition comprising aceDNA lipid particle) according to the described disclosure; or (iii) has received a ceDNA lipid particle (or pharmaceutical composition comprising a ceDNA lipid particle) according to the described disclosure, unless the context and usage of the phrase indicates otherwise.

As used herein, the term “suppress,” “decrease,” “interfere,” “inhibit” and/or “reduce” (and like terms) generally refers to the act of reducing, either directly or indirectly, a concentration, level, function, activity, or behavior relative to the natural, expected, or average, or relative to a control condition.

As used herein, the term “systemic delivery” is meant to refer to delivery of lipid particles that leads to a broad biodistribution of an active agent such as an interfering RNA (e.g., siRNA) within an organism. Some techniques of administration can lead to the systemic delivery of certain agents, but not others. Systemic delivery means that a useful, preferably therapeutic, amount of an agent is exposed to most parts of the body. To obtain broad biodistribution generally requires a blood lifetime such that the agent is not rapidly degraded or cleared (such as by first pass organs (liver, lung, etc.) or by rapid, nonspecific cell binding) before reaching a disease site distal to the site of administration. Systemic delivery of lipid particles (e.g., lipid nanoparticles) can be by any means known in the art including, for example, intravenous, subcutaneous, and intraperitoneal. In a preferred embodiment, systemic delivery of lipid particles (e.g., lipid nanoparticles) is by intravenous delivery.

As used herein, the terms “therapeutic amount”, “therapeutically effective amount”, an “amount effective”, or “pharmaceutically effective amount” of an active agent (e.g., a ceDNA lipid particle as described herein) are used interchangeably to refer to an amount that is sufficient to provide the intended benefit of treatment. However, dosage levels are based on a variety of factors, including the type of injury, the age, weight, sex, medical condition of the patient, the severity of the condition, the route of administration, and the particular active agent employed. Thus, the dosage regimen may vary widely, but can be determined routinely by a physician using standard methods. Additionally, the terms “therapeutic amount”, “therapeutically effective amounts” and “pharmaceutically effective amounts” include prophylactic or preventative amounts of the compositions of the described disclosure. In prophylactic or preventative applications of the described disclosure, pharmaceutical compositions or medicaments are administered to a patient susceptible to, or otherwise at risk of, a disease, disorder or condition in an amount sufficient to eliminate or reduce the risk, lessen the severity, or delay the onset of the disease, disorder or condition, including biochemical, histologic and/or behavioral symptoms of the disease, disorder or condition, its complications, and intermediate pathological phenotypes presenting during development of the disease, disorder or condition. It is generally preferred that a maximum dose be used, that is, the highest safe dose according to some medical judgment. The terms “dose” and “dosage” are used interchangeably herein.

As used herein the term “therapeutic effect” refers to a consequence of treatment, the results of which are judged to be desirable and beneficial. A therapeutic effect can include, directly or indirectly, the arrest, reduction, or elimination of a disease manifestation. A therapeutic effect can also include, directly or indirectly, the arrest reduction or elimination of the progression of a disease manifestation.

For any therapeutic agent described herein therapeutically effective amount may be initially determined from preliminary in vitro studies and/or animal models. A therapeutically effective dose may also be determined from human data. The applied dose may be adjusted based on the relative bioavailability and potency of the administered compound. Adjusting the dose to achieve maximal efficacy based on the methods described above and other well-known methods is within the capabilities of the ordinarily skilled artisan. General principles for determining therapeutic effectiveness, which may be found in Chapter 1 of Goodman and Gilman's The Pharmacological Basis of Therapeutics, 10th Edition, McGraw-Hill (New York) (2001), incorporated herein by reference, are summarized below.

Pharmacokinetic principles provide a basis for modifying a dosage regimen to obtain a desired degree of therapeutic efficacy with a minimum of unacceptable adverse effects. In situations where the drug's plasma concentration can be measured and related to therapeutic window, additional guidance for dosage modification can be obtained.

As used herein, the terms “treat,” “treating,” and/or “treatment” include abrogating, substantially inhibiting, slowing or reversing the progression of a condition, substantially ameliorating clinical symptoms of a condition, or substantially preventing the appearance of clinical symptoms of a condition, obtaining beneficial or desired clinical results. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously asymptomatic for the disorder(s).

Beneficial or desired clinical results, such as pharmacologic and/or physiologic effects include, but are not limited to, preventing the disease, disorder or condition from occurring in a subject that may be predisposed to the disease, disorder or condition but does not yet experience or exhibit symptoms of the disease (prophylactic treatment), alleviation of symptoms of the disease, disorder or condition, diminishment of extent of the disease, disorder or condition, stabilization (i.e., not worsening) of the disease, disorder or condition, preventing spread of the disease, disorder or condition, delaying or slowing of the disease, disorder or condition progression, amelioration or palliation of the disease, disorder or condition, and combinations thereof, as well as prolonging survival as compared to expected survival if not receiving treatment.

Beneficial or desired clinical results, such as pharmacologic and/or physiologic effects include, but are not limited to, preventing the disease, disorder or condition from occurring in a subject that may be predisposed to the disease, disorder or condition but does not yet experience or exhibit symptoms of the disease (prophylactic treatment), alleviation of symptoms of the disease, disorder or condition, diminishment of extent of the disease, disorder or condition, stabilization (i.e., not worsening) of the disease, disorder or condition, preventing spread of the disease, disorder or condition, delaying or slowing of the disease, disorder or condition progression, amelioration or palliation of the disease, disorder or condition, and combinations thereof, as well as prolonging survival as compared to expected survival if not receiving treatment.

As used herein, the term “combination therapy” refers to treatment regimens for a clinical indication that comprise two or more therapeutic agents. Thus, the term refers to a therapeutic regimen in which a first therapy comprising a first composition (e.g., active ingredient) is administered in conjunction with a second therapy comprising a second composition (active ingredient) to a patient, intended to treat the same or overlapping disease or clinical condition. The first and second compositions may both act on the same cellular target, or discrete cellular targets. The phrase “in conjunction with,” in the context of combination therapies, means that therapeutic effects of a first therapy overlaps temporarily and/or spatially with therapeutic effects of a second therapy in the subject receiving the combination therapy. Thus, the combination therapies may be formulated as a single formulation for concurrent administration, or as separate formulations, for sequential administration of the therapies.

As used herein, the term “alkyl” refers to a saturated monovalent hydrocarbon radical of 1 to 20 carbon atoms (i.e., C₁₋₂₀ alkyl). “Monovalent” means that alkyl has one point of attachment to the remainder of the molecule. In one embodiment, the alkyl has 1 to 12 carbon atoms (i.e., C₁₋₁₂ alkyl) or 1 to 10 carbon atoms (i.e., C₁₋₁₀ alkyl). In one embodiment, the alkyl has 1 to 8 carbon atoms (i.e., C₁₋₈ alkyl), 1 to 7 carbon atoms (i.e., C₁₋₇ alkyl), 1 to 6 carbon atoms (i.e., C₁₋₆ alkyl), 1 to 4 carbon atoms (i.e., C₁₋₄ alkyl), or 1 to 3 carbon atoms (i.e., C₁₋₃ alkyl). Examples include, but are not limited to, methyl, ethyl, 1-propyl, 2-propyl, 1-butyl, 2-methyl-1-propyl, 2-butyl, 2-methyl-2-propyl, 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, 1-heptyl, 1-octyl, and the like. A linear or branched alkyl, such as a “linear or branched C₁₋₆ alkyl,” “linear or branched C₁₋₄ alkyl,” or “linear or branched C₁₋₃ alkyl” means that the saturated monovalent hydrocarbon radical is a linear or branched chain. As used herein, the term “linear” as referring to aliphatic hydrocarbon chains means that the chain is unbranched.

The term “alkylene” as used herein refers to a saturated divalent hydrocarbon radical of 1 to 20 carbon atoms (i.e., C₁₋₂₀ alkylene), examples of which include, but are not limited to, those having the same core structures of the alkyl groups as exemplified above. “Divalent” means that the alkylene has two points of attachment to the remainder of the molecule. In one embodiment, the alkylene has 1 to 12 carbon atoms (i.e., C₁₋₁₂ alkylene) or 1 to 10 carbon atoms (i.e., C₁₋₁₀ alkylene). In one embodiment, the alkylene has 1 to 8 carbon atoms (i.e., C₁₋ₛ alkylene), 1 to 7 carbon atoms (i.e., C₁₋₇ alkylene), 1 to 6 carbon atoms (i.e., C₁₋₆ alkylene), 1 to 4 carbon atoms (i.e., C₁₋₄ alkylene), 1 to 3 carbon atoms (i.e., C₁₋₃ alkylene), ethylene, or methylene. A linear or branched alkylene, such as a “linear or branched C₁₋₆ alkylene,” “linear or branched C₁₋₄ alkylene,” or “linear or branched C₁₋₃ alkylene” means that the saturated divalent hydrocarbon radical is a linear or branched chain.

The term “alkenyl” refers to straight or branched aliphatic hydrocarbon radical with one or more (e.g., one or two) carbon-carbon double bonds, wherein the alkenyl radical includes radicals having “cis” and “trans” orientations, or by an alternative nomenclature, “E” and “Z” orientations.

“Alkenylene” as used herein refers to aliphatic divalent hydrocarbon radical of 2 to 20 carbon atoms (i.e., C₂₋₂₀ alkenylene) with one or two carbon-carbon double bonds, wherein the alkenylene radical includes radicals having “cis” and “trans” orientations, or by an alternative nomenclature, “E” and “Z” orientations. “Divalent” means that alkenylene has two points of attachment to the remainder of the molecule. In one embodiment, the alkenylene has 2 to 12 carbon atoms (i.e., C₂₁₆ alkenylene), 2 to 10 carbon atoms (i.e., C₂-o alkenylene). In one embodiment, the alkenylene has 2 to four carbon atoms (C₂₋₄). Examples include, but are not limited to, ethylenylene or vinylene (—CH═CH—), allyl (—CH₂CH═CH—), and the like. A linear or branched alkenylene, such as a “linear or branched C₂₆ alkenylene,” “linear or branched C₂₋₄ alkenylene,” or “linear or branched C₂₋₃ alkenylene” means that the unsaturated divalent hydrocarbon radical is a linear or branched chain.

“Cycloalkylene” as used herein refers to a divalent saturated carbocyclic ring radical having 3 to 12 carbon atoms as a monocyclic ring, or 7 to 12 carbon atoms as a bicyclic ring. “Divalent” means that the cycloalkylene has two points of attachment to the remainder of the molecule. In one embodiment, the cycloalkylene is a 3- to 7-membered monocyclic or 3- to 6-membered monocyclic.

Examples of monocyclic cycloalkyl groups include, but are not limited to, cyclopropylene, cyclobutylene, cyclopentylene, cyclohexylene, cycloheptylene, cyclooctylene, cyclononylene, cyclodecylene, cycloundecylene, cyclododecylene, and the like. In one embodiment, the cycloalkylene is cyclopropylene.

The terms “heterocycle,” “heterocyclyl,” heterocyclic and “heterocyclic ring” are used interchangeably herein and refer to a cyclic group which contains at least one N atom has a heteroatom and optionally 1-3 additional heteroatoms selected from N and S, and are non-aromatic (i.e., partially or fully saturated). It can be monocyclic or bicyclic (bridged or fused). Examples of heterocyclic rings include, but are not limited to, aziridinyl, diaziridinyl, thiaziridinyl, azetidinyl, diazetidinyl, triazetidinyl, thiadiazetidinyl, thiazetidinyl, pyrrolidinyl, pyrazolidinyl, imidazolinyl, isothiazolidinyl, thiazolidinyl, piperidinyl, piperazinyl, hexahydropyrimidinyl, azepanyl, azocanyl, and the like. The heterocycle contains 1 to 4 heteroatoms, which may be the same or different, selected from N and S. In one embodiment, the heterocycle contains 1 to 3 N atoms. In another embodiment, the heterocycle contains 1 or 2 N atoms. In another embodiment, the heterocycle contains 1 N atom. A “4- to 8-membered heterocyclyl” means a radical having from 4 to 8 atoms (including 1 to 4 heteroatoms selected from N and S, or 1 to 3 N atoms, or 1 or 2 N atoms, or 1 N atom) arranged in a monocyclic ring. A “5- or 6-membered heterocyclyl” means a radical having from 5 or 6 atoms (including 1 to 4 heteroatoms selected from N and S, or 1 to 3 N atoms, or 1 or 2 N atoms, or 1 N atom) arranged in a monocyclic ring. The term “heterocycle” is intended to include all the possible isomeric forms. Heterocycles are described in Paquette, Leo A., Principles of Modern Heterocyclic Chemistry (W. A. Benjamin, New York, 1968), particularly Chapters 1, 3, 4, 6, 7, and 9; The Chemistry of Heterocyclic Compounds, A Series of Monographs (John Wiley & Sons, New York, 1950 to present), in particular Volumes 13, 14, 16, 19, and 28; and J. Am. Chem. Soc. (1960) 82:5566. The heterocyclyl groups may be carbon (carbon-linked) or nitrogen (nitrogen-linked) attached to the rest of the molecule where such is possible.

If a group is described as being “optionally substituted,” the group may be either (1) not substituted, or (2) substituted. If a carbon of a group is described as being optionally substituted with one or more of a list of substituents, one or more of the hydrogen atoms on the carbon (to the extent there are any) may separately and/or together be replaced with an independently selected optional substituent.

Suitable substituents for an alkyl, alkylene, alkenylene, cycloalkylene, and heterocyclyl, are those which do not significantly adversely affect the biological activity of the bifunctional compound. Unless otherwise specified, exemplary substituents for these groups include linear, branched or cyclic alkyl, alkenyl or alkynyl having from 1 to 10 carbon atoms, aryl, heteroaryl, heterocyclyl, halogen, guanidinium [—NH(C═NH)NH₂], —OR₁₀₀, NR₁₀₁R₁₀₂, —NO₂, —NR₁₀₁COR₁₀₂, —SR₁₀₀, a sulfoxide represented by —SOR₁₀₁, a sulfone represented by —SO₂R₁₀₁, a sulfonate —SO₃M, a sulfate —OSO₃M, a sulfonamide represented by —SO₂NR₁₀₁R₁₀₂, cyano, an azido, —COR₁₀₁, —OCOR₁₀₁, —OCONR₁₀₁R₁₀₂ and a polyethylene glycol unit (—OCH₂CH₂)ₙR₁₀₁ wherein M is H or a cation (such as Na⁺ or K⁺); R₁₀₁, R₁₀₂ and R₁₀₃ are each independently selected from H, linear, branched or cyclic alkyl, alkenyl or alkynyl having from 1 to 10 carbon atoms, a polyethylene glycol unit (—OCH₂CH₂)n-R₁₀₄, wherein n is an integer from 1 to 24, an aryl having from 6 to 10 carbon atoms, a heterocyclic ring having from 3 to 10 carbon atoms and a heteroaryl having 5 to 10 carbon atoms; and R¹⁰⁴ is H or a linear or branched alkyl having 1 to 4 carbon atoms, wherein the alkyl, alkenyl, alkynyl, aryl, heteroaryl and heterocyclyl in the groups represented by R₁₀₀, R₁₀₁, R₁₀₂, R₁₀₃ and R₁₀₄ are optionally substituted with one or more (e.g., 2, 3, 4, 5, 6 or more) substituents independently selected from halogen, —OH, -CN, —NO₂, and unsubstituted linear or branched alkyl having 1 to 4 carbon atoms. Preferably, the substituent for the optionally substituted alkyl, alkylene, alkenylene, cycloalkylene, and heterocyclyl described above is selected from the group consisting of halogen, —CN, —NR₁₀₁R₁₀₂, —CF₃, —OR₁₀₀, aryl, heteroaryl, heterocyclyl, -SR₁₀₁, —SOR₁₀₁, —SO₂R₁₀₁, and —SO₃M. Alternatively, the suitable substituent is selected from the group consisting of halogen, —OH, —NO₂, —CN, C₁₋₄ alkyl, —OR₁₀₀, NR₁₀₁R₁₀₂, —NR₁₀₁COR₁₀₂, —SR₁₀₀, —SO₂R₁₀₁, —SO₂NR₁₀₁R₁₀₂, —COR₁₀₁, —OCOR₁₀₁, and —OCONR₁₀₁R₁₀₂, wherein R₁₀₀, R₁₀₁, and R₁₀₂ are each independently —H or C₁₋₄ alkyl.

“Halogen” as used herein refers to F, Cl, Br or I. “Cyano” is -CN.

“Amine” or “amino” as used herein interchangeably refers to a functional group that contains a basic nitrogen atom with a lone pair.

The term “pharmaceutically acceptable salt” as used herein refers to pharmaceutically acceptable organic or inorganic salts of an ionizable lipid of the disclosure. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate,” ethanesulfonate, benzenesulfonate, p-toluenesulfonate, pamoate (i.e., 1,1′-methylene-bis-(2-hydroxy-3-naphthoate)) salts, alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

Groupings of alternative elements or embodiments of the disclosure disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the disclosure.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior disclosure or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting. It should be understood that this disclosure is not limited in any manner to the particular methodology, protocols, and reagents, etc., described herein and as such can vary. The terminology used herein is for the purpose of describing particular embodiments only and is not intended to limit the scope of the present disclosure, which is defined solely by the claims.

### II. Lipid Nanoparticle Compositions

Provided herein are pharmaceutical compositions comprising a lipid nanoparticle (LNP) and a therapeutic nucleic acid (TNA), wherein the LNP comprises a single-chain variable fragment (scFv), linked to the LNP. The scFv is directed against an antigen present on the surface of a cell. The term “linked” encompasses chemical conjugation, adsorption (physisorption and/or chemisorption). The types of bonds encompassed by the term “linked” are covalent interactions and noncovalent interactions (e.g., hydrogen bonds, van der Waal bonds, ionic bonds, and hydrophobic bonds). According to some embodiments, the scFv is linked to the LNP via covalent conjugation. According to some embodiments, the scFv is linked to the LNP via maleimide linkage. It is a finding of the present disclosure that maleimide conjugation of scFv to LNP resulted in more robust conjugation to the LNP compared to other thiol based cross-linking methods, such as PDS conjugation, and importantly maintained LNP size and integrity.

Accordingly, provided herein are pharmaceutical compositions comprising a lipid nanoparticle (LNP) and a therapeutic nucleic acid (TNA), wherein the LNP comprises a single-chain variable fragment (scFv) linked to the LNP, wherein the scFv is directed against an antigen present on the surface of a cell, and at least one pharmaceutically acceptable excipient, wherein the scFv is covalently linked to the LNP via a non-cleavable linker. According to some embodiments, the non-cleavable linker is a maleimide-containing linker.

Also provided herein are pharmaceutical compositions comprising a lipid nanoparticle (LNP) and a therapeutic nucleic acid (TNA), wherein the LNP comprises a single-chain variable fragment (scFv) linked to the LNP, wherein the scFv is directed against an antigen present on the surface of a cell, and at least one pharmaceutically acceptable excipient, wherein the scFv is covalently linked to the LNP via a cleavable linker.

The LNPs described herein provides numerous therapeutic advantages, including a smaller size that can encapsulate large, therapeutic nucleic acid molecules. It is an advantageous feature of the present disclosure that the scFv LNPs as described herein are useful for targeting any cell or tissue that actively expresses the antigen present on the surface of a cell to which the scFv is directed. According to some embodiments, the cell is a tumor cell. According to some embodiments, the cell is a liver cell (hepatocyte).

According to some embodiments, the antigen is a tumor-associated antigen (TAA) or a tumor-selective antigen (TSA). A “tumor-associated antigen” or TAA is an antigen that is expressed on tumors. A “tumor-selective antigen” or TSA is an antigen that is expressed selectively on tumors. In one embodiment, the antigen is human epidermal growth factor receptor 2 (HER2).

In one embodiment, TAA expression can be restricted to the tumor cell population alone, expressed by all tumor cells, and expressed on the tumor cell surface. Other antigens are overexpressed on tumor cells, but may be found on normal cells at lower levels of expression and thus are tumor-selective antigens (TSA). In addition, some tumor antigens arise as “passenger mutations”, i.e., are non-essential antigens expressed by tumor cells that have defective control over DNA repair, thus accumulating mutations in diverse proteins. Some tumor antigens are proteins that are produced by tumor cells that elicit an immune response; particularly T-cell mediated immune responses.

According to some embodiments, the TAA or TSA is selected from the group consisting of glioma-associated antigen, carcinoembryonic antigen (CEA), R-human chorionic gonadotropin, alphafetoprotein (AFP), lectin-reactive AFP, thyroglubilin, RAGE-1, MN-CA IX, human telomerase reverse transcriptase, RU1, RU2 (AS), intestinal carboxylesterase, mut hsp70-2, M-CSF, prostase, prostate-specific antigen (PSA), PAP, NY-ESO-1, LAGE-1a, p53, prostein, PSMA, HER2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin, EphA2, HER2, GD2, Glypican-3, 5T4, 8H9, ανβ6 integrin, BCMA, B7-H3, B7-H6, CAIX, CA9, CD19, CD20, CD22, kappa light chain, CD30, CD33, CD38, CD44, CD44v6, CD44v7/8, CD70, CD123, CD138, CD171, CEA, CSPG4, EGFR, EGFRvIII, EGP2, EGP40, EPCAM, ERBB3, ERBB4, ErbB3/4, FAP, FAR, FBP, fetal AchR, Folate Receptor a, GD2, GD3, HLA-AI MAGE A1, HLA-A2, IL11Ra, ILI3Ra2, KDR, Lambda, Lewis-Y, MCSP, Mesothelin, Muc1, Muc16, NCAM, NKG2D ligands, NY-ESO-1, PRAME, PSCA, PSC1, PSMA, ROR1, SURVIVIN, TAG72, TEM1, TEM8, VEGRR2, carcinoembryonic antigen, HMW-MAA, and VEGF receptors. Other exemplary antigens that can be used are antigens that are present with in the extracellular matrix of tumors, such as oncofetal variants of fibronectin, tenascin, or necrotic regions of tumors.

Additional tumor-selective molecules can be used include any membrane protein or biomarker that is expressed or overexpressed in tumor cells including, but not limited to, integrins (e.g., integrin ανβ3, α5β1), EGF Receptor Family (e.g., EGFR2, Erbb2/HER2/neu, Erbb3, Erbb4), proteoglycans (e.g., heparan sulfate proteoglycans), disialogangliosides (e.g., GD2, GD3), B7-H3 (aka CD276), cancer antigen 125 (CA-125), epithelial cell adhesion molecule (EpCAM), vascular endothelial growth factor receptors 1 and 2 (VEGFR-1, VEGFR-2), CD52, carcinoembryonic antigen (CEA), tumor associated glycoproteins (e.g., TAG-72), cluster of differentiation 19 (CD19), CD20, CD22, CD30, CD33, CD40, CD44, CD74, CD152, mucin 1 (MUC1), tumor necrosis factor receptors (e.g., TRAIL-R2), insulin-like growth factor receptors, folate receptor a, transmembrane glycoprotein NMB (GPNMB), C-C chemokine receptors (e.g., CCR4), prostate specific membrane antigen (PSMA), recepteur d'origine nantais (RON) receptor, cytotoxic T-lymphocyte antigen 4 (CTLA4), and other tumor specific receptors or antigens.

The Cancer Antigenic Peptide Database is a publically available database (caped.icp.ucl.ac.be) that compiles information of human tumor antigens, including the peptide sequence and its position in the protein sequence. According to some embodiments, the scFv is directed to a tumor associated antigen set forth in the Cancer Antigenic Peptide Database.

According to some embodiments, a scFv binds to a tumor antigen associated with a hematologic malignancy. In some embodiments, a scFv binds to a tumor antigen associated with a solid tumor.

The majority of antibody fragments currently being developed in the clinic are for oncological applications. In addition to the generic characteristics of antibody fragments that make them attractive as immunotherapies, e.g., their small size, which grants them superior tissue and tumor penetration compared to a conventional mAb, and the lack of an Fc domain that reduces non-specific activation of innate immune cells, there are many mechanisms of action that are unique to a specific format.

While oncology is a major area in which antibody fragments have become a prominent class of therapeutic molecules, there are several other disease areas in which antibody fragments are being evaluated.

Autoimmune diseases are chronic and potentially life-threatening, and antibody therapies are extremely expensive because they usually require intensive, life-long treatment. The lower production costs of antibody fragments and potential reduced immunogenicity due to their small size renders the use of antibody fragments with half-life extension moieties as a viable alternative to full-length antibodies. Furthermore, like for cancer immunotherapies, the development of antibody fragments for the treatment of autoimmune diseases has been growing at a fast pace and there are numerous possibilities for bispecific targeting.

One of the first antibody fragments to be marketed for an autoimmune disease indication was Certolizumab pegol (CIMZIA®), a pegylated Fab targeting TNF developed by UCB (Belgium), approved by the FDA for the treatment of Crohn's disease in 2008. It has subsequently been approved for rheumatoid arthritis, psoriatic arthritis, and ankylosing spondylitis. Two other Fabs are in clinical trials: FR104 (OSE/Janssen) against CD28 in phase II for RA, and Dapirolizumab, an anti-CD40L Fab developed by UCB in phase II for SLE.

One scFv format currently being evaluated in climical trials for the treatment of RA is Dekavil or F8IL10 (Philogen). It is a fully human fusion protein composed of the vascular targeting scFv antibody F8 fused to the cytokine interleukin-10. A number of other immunocytokines fused to scFvs are also in preclinical development.

Antibody fragments such as Fabs and scFvs have been shown to be able to penetrate the cornea and pass into the eye and achieve clinically useful concentrations in the anterior chamber over a reasonable time-span following topical administration (Thiel et al. Clin. Exp. Immunol. 2002.). The most common eye disorder treated with antibodies or antibody fragments is age-related macular degeneration (AMD), which is the leading cause of irreversible blindness in people aged 50 years or older, in the developed world. For AMD, the antibody fragments are applied directly to the eye via the intravitreal route. Ranibizumab (LUCENTIS®) is an anti-angiogenic monoclonal antibody fragment targeting VEGF-A, derived from the same parental mouse antibody as bevacizumab. It was approved in 2006 for wet AMD and subsequently in 2012 and 2015 for diabetic macular oedema and diabetic retinopathy, respectively. Brolucizumab (Alcon/Novartis) is a scFv targeting VEGF that is in phase III for wet AMD.

According to some embodiments, the scFv comprises SEQ ID NO: 1.

(SEQ ID NO: 1)
EVQLVESGGGLVQPGGSLRLSCAASGFNIDDTYIHWVRQAPGKGLEWVA
RIYPTNGYTRYADSVKGRFTISADTSKNTAYLQMNSLRAEDTAVYYCSR
WGGDGFYAMDVWGQGTLVTVSSGGGGSGGGGSGGGGSDIQMTQSPSS
LSASVGDRVTITCRASQDVNTAVAWYQQKPGKAPKLLIYSADFLYSGVP
SRFSGSRSGTDFTLTISSLQPEDFATYYCQQHYTTPPTFGQGTKVEIK

According to some embodiments, the scFv comprises an amino acid sequence that is at least 85% identical to SEQ ID NO: 1. According to some embodiments, the scFv comprises an amino acid sequence that is at least 90% identical to SEQ ID NO: 1. According to some embodiments, the scFv comprises an amino acid sequence that is at least 95% identical to SEQ ID NO: 1. According to some embodiments, the scFv comprises an amino acid sequence that is at least 96% identical to SEQ ID NO: 1. According to some embodiments, the scFv comprises an amino acid sequence that is at least 97% identical to SEQ ID NO: 1. According to some embodiments, the scFv comprises an amino acid sequence that is at least 98% identical to SEQ ID NO: 1. According to some embodiments, the scFv comprises an amino acid sequence that is at least 99% identical to SEQ ID NO: 1. According to some embodiments, the scFv consists of SEQ ID NO: 1.

According to some embodiments, the scFv comprises SEQ ID NO: 2. SEQ ID NO:2 contains a myc (bold underlined) tag and a His (italic) tag with a c-terminal cysteine required for maleimide conjugation.

(SEQ ID NO: 2)
EVQLVESGGGLVQPGGSLRLSCAASGFNIDDTYIHWVRQAPGKGLEWVA
RIYPTNGYTRYADSVKGRFTISADTSKNTAYLQMNSLRAEDTAVYYCSR
WGGDGFYAMDVWGQGTLVTVSSGGGGSGGGGSGGGGSDIQMTQSPSS
LSASVGDRVTITCRASQDVNTAVAWYQQKPGKAPKLLIYSADFLYSGVP
SRFSGSRSGTDFTLTISSLQPEDFATYYCQQHYTTPPTFGQGTKVEIK
EQKLISEEDLHHHHHHC

According to some embodiments, the scFv comprises an amino acid sequence that is at least 85% identical to SEQ ID NO: 2. According to some embodiments, the scFv comprises an amino acid sequence that is at least 90% identical to SEQ ID NO: 2. According to some embodiments, the scFv comprises an amino acid sequence that is at least 95% identical to SEQ ID NO: 2. According to some embodiments, the scFv comprises an amino acid sequence that is at least 96% identical to SEQ ID NO: 2. According to some embodiments, the scFv comprises an amino acid sequence that is at least 97% identical to SEQ ID NO: 2. According to some embodiments, the scFv comprises an amino acid sequence that is at least 98% identical to SEQ ID NO: 2. According to some embodiments, the scFv comprises an amino acid sequence that is at least 99% identical to SEQ ID NO: 2. According to some embodiments, the scFv consists of SEQ ID NO: 2.

According to some embodiments, the scFv comprises SEQ ID NO: 3. SEQ ID NO:3 comprises the same scFV core sequence as SEQ ID NO:1 but with an N-terminal His (italic) tag and a c-terminal LLQGA polypeptide (bold and underlined) to facilitate transglutaminase-mediated conjugation.

(SEQ ID NO: 3)
HHHHHHEVQLVESGGGLVQPGGSLRLSCAASGFNIDDTYIHWVRQAPGK
GLEWVARIYPTNGYTRYADSVKGRFTISADTSKNTAYLQMNSLRAEDTA
VYYCSRWGGDGFYAMDVWGQGTLVTVSSGGGGSGGGGSGGGGSDIQM
TQSPSSLSASVGDRVTITCRASQDVNTAVAWYQQKPGKAPKLLIYSADFL
YSGVPSRFSGSRSGTDFTLTISSLQPEDFATYYCQQHYTTPPTFGQGTKV
EIKLLQGA

According to some embodiments, the scFv comprises an amino acid sequence that is at least 85% identical to SEQ ID NO: 3. According to some embodiments, the scFv comprises an amino acid sequence that is at least 90% identical to SEQ ID NO: 3. According to some embodiments, the scFv comprises an amino acid sequence that is at least 95% identical to SEQ ID NO: 3. According to some embodiments, the scFv comprises an amino acid sequence that is at least 96% identical to SEQ ID NO: 3. According to some embodiments, the scFv comprises an amino acid sequence that is at least 97% identical to SEQ ID NO: 3. According to some embodiments, the scFv comprises an amino acid sequence that is at least 98% identical to SEQ ID NO: 3. According to some embodiments, the scFv comprises an amino acid sequence that is at least 99% identical to SEQ ID NO: 3. According to some embodiments, the scFv consists of SEQ ID NO: 3.

According to some embodiments, the LNP comprises a cationic lipid, a sterol or a derivative thereof, a non-cationic lipid, or a PEGylated lipid.

### A. Cationic Lipids

In some embodiments, the lipid nanoparticle having mean diameter of 20-74 nm comprises a cationic lipid. In some embodiments, the cationic lipid is, e.g., a non-fusogenic cationic lipid. By a “non-fusogenic cationic lipid” is meant a cationic lipid that can condense and/or encapsulate the nucleic acid cargo, such as ceDNA, but does not have, or has very little, fusogenic activity.

In some embodiments, the cationic lipid is described in the international and U.S. patent application publications listed below in Table 1, and determined to be non-fusogenic, as measured, for example, by a membrane-impermeable fluorescent dye exclusion assay, e.g., the assay described in the Examples section herein. Contents of all of these patent documents international and U.S. patent application publications listed below in Table 1 are incorporated herein by reference in their entireties.

TABLE 1
Exemplary patent documents describing cationic or ionizable lipids

| International Patent | U.S. Patent Application |
|---|---|
| Application Publication No. | Publication No. |
| WO2015/095340 | US2016/0311759 |
| WO2015/199952 | US2015/0376115 |
| WO2018/011633 | US2016/0151284 |
| WO2017/049245 | US2017/0210697 |
| WO2015/061467 | US2015/0140070 |
| WO2012/040184 | US2013/0178541 |
| WO2012/000104 | US2013/0303587 |
| WO2015/074085 | US2015/0141678 |
| WO2016/081029 | US2015/0239926 |
| WO2017/004143 | US2016/0376224 |
| WO2017/075531 | US2017/0119904 |
| WO2017/117528 | |
| WO2011/022460 | US2012/0149894 |
| WO2013/148541 | US2015/0057373 |
| WO2013/116126 | |
| WO2011/153120 | US2013/0090372 |
| WO2012/044638 | US2013/0274523 |
| WO2012/054365 | US2013/0274504 |
| WO2011/090965 | US2013/0274504 |
| WO2013/016058 | |
| WO2012/162210 | |
| WO2008/042973 | US2009/0023673 |
| WO2010/129709 | US2012/0128760 |
| WO2010/144740 | US201/003241240 |
| WO2012/099755 | US2014/0200257 |
| WO2013/049328 | US2015/0203446 |
| WO2013/086322 | US2018/0005363 |
| WO2013/086373 | US2014/0308304 |
| WO2011/071860 | US2013/0338210 |
| WO2009/132131 | |
| WO2010/048536 | |
| WO2010/088537 | US2012/0101148 |
| WO2010/054401 | US2012/0027796 |
| WO2010/054406 | |
| WO2010/054405 | |
| WO2010/054384 | US2012/0058144 |
| WO2012/016184 | US2013/0323269 |
| WO2009/086558 | US2011/0117125 |
| WO2010/042877 | US2011/0256175 |
| WO2011/000106 | US2012/0202871 |
| WO2011/000107 | US2011/0076335 |
| WO2005/120152 | US2006/0083780 |
| WO2011/141705 | US2013/0123338 |
| WO2013/126803 | US2015/0064242 |
| WO2006/07712 | US2006/0051405 |
| WO2011/038160 | US2013/0065939 |
| WO2005/121348 | US2006/0008910 |
| WO2011/066651 | US2003/0022649 |
| WO2009/127060 | US2010/0130588 |
| WO2011/141704 | US2013/0116307 |
| WO2006/069782 | US2010/0062967 |
| WO2012/031043 | US2013/0202684 |
| WO2013/006825 | US2014/0141070 |
| WO2013/033563 | US2014/0255472 |
| WO2013/089151 | US2014/0039032 |
| WO2017/099823 | US2018/0028664 |
| WO2015/095346 | US2016/0317458 |
| WO2013/086354 | US2013/0195920 |

In some embodiments, the cationic lipid is selected from the group consisting of N-[1-(2,3-dioleyloxy)propyll-N,N,N-trimethylammonium chloride (DOTMA); N-[1-(2,3-dioleoyloxy)propyll-N,N,N-trimethylammonium chloride (DOTAP); 1,2-dioleoyl-sn-glycero -3-ethylphosphocholine (DOEPC); 1,2-dilauroyl-sn-glycero-3-ethylphosphocholine (DLEPC); 1,2-dimyristoyl-sn-glycero-3-ethylphosphocholine (DMEPC); 1,2-dimyristoleoyl—sn-glycero-3-ethylphosphocholine (14:1), N1-[12-((1S)-1-[(3-aminopropyl)amino]-4-[Idi(3-amino-propyl) aminolbutylc arboxamidoiethy11-3,4-di[loleyloxy]-benzamide(MVLS); Dioctadecylamido-glycylspermine (DOGS); 3b-[IN-(N′,N′-dimethylaminoethyl)carb amoyl]cholesterol (DC-Chol); Dioctadecyldimethylammonium Bromide (DDAB); a Saint lipid (e.g., SAINT-2, N-methyl-4-(dioleyl)methylpyridinium); 1,2-dimyristyloxypropy1-3-dimethylhydroxyethylammonium bromide (DMRIE); 1,2-dioleoy1-3-dimethyl-hydroxyethyl ammonium bromide (DORIE); 1,2-dioleoyloxypropy1-3-dimethylhydroxyethyl ammonium chloride (DORI); Di-alkylated Amino Acid (DILA2) (e.g., C18:1-norArg -C16); Dioleyldimethylammonium chloride (DODAC); 1-palmitoyl-2-oleoyl-sn-glycero-3-ethylpho sphocholine (POEPC); and 1,2-dimyristoleoyl-sn-glycero-3-ethylphosphocholine (MOEPC). In some variations, the condensing agent, e.g. a cationic lipid, is a lipid such as, e.g., Dioctadecyldimethylammonium bromide (DDAB), 1,2-dilinoleyloxy-3-dimethylaminopropane (DLinDMA), 2,2-dilinoleyl-4-(2dimethylaminoethyl)-[1,31-dioxolane (DLin-KC2-DMA), heptatriaconta-6,9,28,31-tetraen-19-yl-4-(dimethylamino)butanoate (DLin-MC3-DMA), 1,2-Dioleoyloxy-3-dimethylaminopropane (DODAP), 1,2-Dioleyloxy-3-dimethylaminopropane (DODMA), Morpholinocholesterol (Mo-CHOL), (R)-5-(dimethylamino)pentane-1,2-diyl dioleate hydrochloride (DODAPen-C1), (R)-5-guanidinopentane-1,2-diy1 dioleate hydrochloride (DOPen-G), (R)-N,N,N-trimethyl-4,5-bis(oleoyloxy)pentan-1-aminium chloride(DOTAPen). In some embodiments, the condensing lipid is DOTAP.

### Ionizable Lipids

According to some embodiments, also provided herein are pharmaceutical compositions containing LNPs comprising an ionizable lipid and a therapeutic nucleic acid like non-viral vector (e.g., ceDNA). Such LNPs can be used to deliver, e.g., the pharmaceutical composition comprising a lipid nanoparticle (LNP) and a therapeutic nucleic acid (TNA), wherein the LNP comprises a scFv, linked to the LNP, as described herein, to a target site of interest (e.g., cell, tissue, organ, and the like).

Exemplary ionizable lipids are described in International Patent Application Publication Nos. WO2015/095340, WO2015/199952, WO2018/011633, WO2017/049245, WO2015/061467, WO2012/040184, WO2012/000104, WO2015/074085, WO2016/081029, WO2017/004143, WO2017/075531, WO2017/117528, WO2011/022460, WO2013/148541, WO2013/116126, WO2011/153120, WO2012/044638, WO2012/054365, WO2011/090965, WO2013/016058, WO2012/162210, WO2008/042973, WO2010/129709, WO2010/144740, WO2012/099755, WO2013/049328, WO2013/086322, WO2013/086373, WO2011/071860, WO2009/132131, WO2010/048536, WO2010/088537, WO2010/054401, WO2010/054406, WO2010/054405, WO2010/054384, WO2012/016184, WO2009/086558, WO2010/042877, WO2011/000106, WO2011/000107, WO2005/120152, WO2011/141705, WO2013/126803, WO2006/007712, WO2011/038160, WO2005/121348, WO2011/066651, WO2009/127060, WO2011/141704, WO2006/069782, WO2012/031043, WO2013/006825, WO2013/033563, WO2013/089151, WO2017/099823, WO2015/095346, and WO2013/086354, and US Patent Application Publication Nos. US2016/0311759, US2015/0376115, US2016/0151284, US2017/0210697, US2015/0140070, US2013/0178541, US2013/0303587, US2015/0141678, US2015/0239926, US2016/0376224, US2017/0119904, US2012/0149894, US2015/0057373, US2013/0090372, US2013/0274523, US2013/0274504, US2013/0274504, US2009/0023673, US2012/0128760, US2010/0324120, US2014/0200257, US2015/0203446, US2018/0005363, US2014/0308304, US2013/0338210, US2012/0101148, US2012/0027796, US2012/0058144, US2013/0323269, US2011/0117125, US2011/0256175, US2012/0202871, US2011/0076335, US2006/0083780, US2013/0123338, US2015/0064242, US2006/0051405, US2013/0065939, US2006/0008910, US2003/0022649, US2010/0130588, US2013/0116307, US2010/0062967, US2013/0202684, US2014/0141070, US2014/0255472, US2014/0039032, US2018/0028664, US2016/0317458, and US2013/0195920, the contents of all of which are incorporated herein by reference in their entirety.

In some embodiments, the ionizable lipid is MC3 (6Z,9Z,28Z,31Z)-heptatriaconta-6,9,28,31-tetraen-19-yl-4-(dimethylamino) butanoate (DLin-MC3-DMA or MC3) having the following structure:

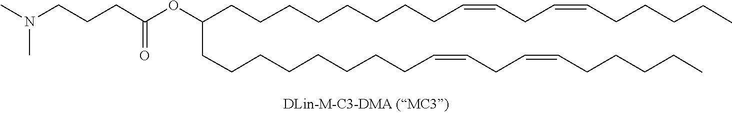 [SMILES: CCCCC/C=C\C/C=C\CCCCCCCCC(CCCCCCCC/C=C\C/C=C\CCCCC)OC(=O)CCCN(C)C]

The lipid DLin-MC3-DMA is described in Jayaraman et al., Angew. Chem. Int. Ed Engl. (2012), 51(34): 8529-8533, content of which is incorporated herein by reference in its entirety.

In some embodiments, the ionizable lipid is the lipid ATX-002 as described in WO2015/074085, the contents of which is incorporated herein by reference in its entirety.

In some embodiments, the ionizable lipid is (13Z,16Z)-N,N-dimethyl-3-nonyldocosa-13,16-dien-1-amine (Compound 32), as described in WO2012/040184, the contents of which is incorporated herein by reference in its entirety.

In some embodiments, the ionizable lipid is Compound 6 or Compound 22 as described in WO2015/199952, the contents of which is incorporated herein by reference in its entirety.

### Formula (I)

According to some embodiments, the cationic lipids are represented by Formula (I):

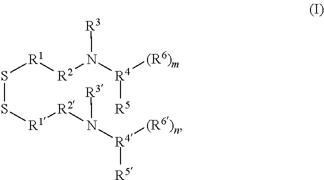

or a pharmaceutically acceptable salt thereof, wherein:
R¹ and R¹′ are each independently C₁₋₃ alkylene;
R² and R^{2′} are each independently linear or branched C₁₋₆ alkylene, or C₃₋₆ cycloalkylene;
R³ and R^{3′} are each independently optionally substituted C₁₋₆ alkyl or optionally substituted C₃₋₆ cycloalkyl;
or alternatively, when R² is branched C₁₋₆ alkylene and when R³ is C₁₋₆ alkyl, R² and R³, taken together with their intervening N atom, form a 4- to 8-membered heterocyclyl;
or alternatively, when R^{2′} is branched C₁₋₆ alkylene and when R^{3′} is C₁₋₆ alkyl, R^{2′} and R^{3′}, taken together with their intervening N atom, form a 4- to 8-membered heterocyclyl;
R⁴ and R^{4′} are each independently —CH, —CH₂CH, or —(CH₂)₂CH;
R⁵ and R^{5′} are each independently hydrogen, C₁₋₂₀ alkylene or C₂₋₂₀ alkenylene;
R⁶ and R^{6′}, for each occurrence, are independently C₁₋₂₀ alkylene, C₃₋₂₀ cycloalkylene, or C₂₂o alkenylene; and
m and n are each independently an integer selected from 1, 2, 3, 4, and 5.

According to some embodiments of any of the aspects or embodiments herein, R² and R^{2′} are each independently C₁₋₃ alkylene.

According to some embodiments of any of the aspects or embodiments herein, the linear or branched C₁₋₃ alkylene represented by R¹ or R″, the linear or branched C₁₋₆ alkylene represented by R² or R^{2′}, and the optionally substituted linear or branched C₁₋₆ alkyl are each optionally substituted with one or more halo and cyano groups.

According to some embodiments of any of the aspects or embodiments herein, R¹ and R² taken together are C₁₋₃ alkylene and R^{1′} and R^{2′} taken together are C₁₋₃ alkylene, e.g., ethylene.

According to some embodiments of any of the aspects or embodiments herein, R³ and R^{3′} are each independently optionally substituted C₁₋₃ alkyl, e.g., methyl.

According to some embodiments of any of the aspects or embodiments herein, R⁴ and R^{4′} are each -CH.

According to some embodiments of any of the aspects or embodiments herein, R² is optionally substituted branched C₁₋₆ alkylene; and R² and R³, taken together with their intervening N atom, form a 5- or 6-membered heterocyclyl. According to some embodiments of any of the aspects or embodiments herein, R^{2′} is optionally substituted branched C₁₋₆ alkylene; and R^{2′} and R³, taken together with their intervening N atom, form a 5- or 6-membered heterocyclyl, such as pyrrolidinyl or piperidinyl.

According to some embodiments of any of the aspects or embodiments herein, R⁴ is —C(R^{a})₂CR^{a}, or —[C(R^{a})₂]₂CR^{a} and R^{a} is C₁₋₃ alkyl; and R³ and R⁴, taken together with their intervening N atom, form a 5- or 6-membered heterocyclyl. According to some embodiments of any of the aspects or embodiments herein, R^{4′} is —C(R^{a})₂CR^{a}, or —[C(R^{a})₂]₂CR^{a} and R^{a} is C₁₋₃ alkyl; and R^{3′} and R^{4′}, taken together with their intervening N atom, form a 5- or 6-membered heterocyclyl, such as pyrrolidinyl or piperidinyl.

According to some embodiments of any of the aspects or embodiments herein, R⁵ and R^{5′} are each independently C₁₋₁₀ alkylene or C₂₋₁₀ alkenylene. In one embodiment, R⁵ and R^{5′} are each independently C₁₋₈ alkylene or C₁₋₆ alkylene.

According to some embodiments of any of the aspects or embodiments herein, R⁶ and R^{6′}, for each occurrence, are independently C₁₋₁₀ alkylene, C₃₋₁₀ cycloalkylene, or C₂₋₁₀ alkenylene. In one embodiment, C₁₋₆ alkylene, C₃₋₆ cycloalkylene, or C₂₋₆alkenylene. In one embodiment the C₃₋₁₀ cycloalkylene or the C₃₋₆ cycloalkylene is cyclopropylene. According to some embodiments of any of the aspects or embodiments herein, m and n are each 3.

According to some embodiments of any of the aspects or embodiments herein, the cationic lipid is selected from any one of the lipids in Table 2 or a pharmaceutically acceptable salt thereof.

TABLE 2
Exemplary cationic lipids of Formula (I)

| Lipid . | |
|---|---|
| No | Structure and Name |
| 1 | 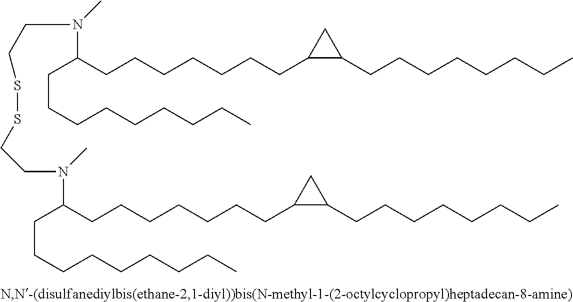 [SMILES: CCCCCCCCCC(CCCCCCCC1CC1CCCCCCCC)N(C)CCSSCCN(C)C(CCCCCCCCC)CCCCCCCC1CC1CCCCCCCC] |
| 2 | 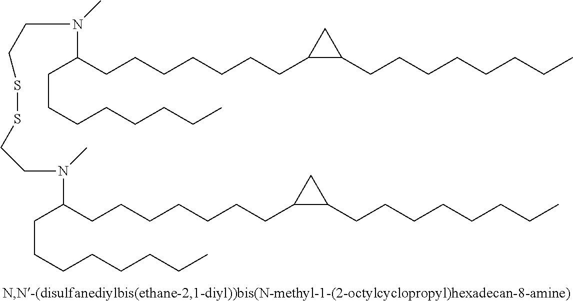 [SMILES: CCCCCCCCC1CC1CCCCCCCC(CCCCCCCC)N(C)CCSSCCN(C)C(CCCCCCCC)CCCCCCCC1CC1CCCCCCCC] |
| 3 | 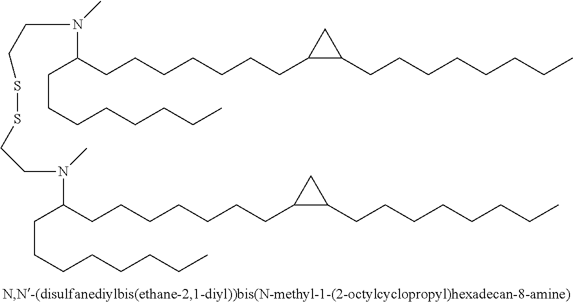 [SMILES: CCCCCCCCC1CC1CCCCCCCC(CCCCCCCC)N(C)CCSSCCN(C)C(CCCCCCCC)CCCCCCCC1CC1CCCCCCCC] |
| 4 | 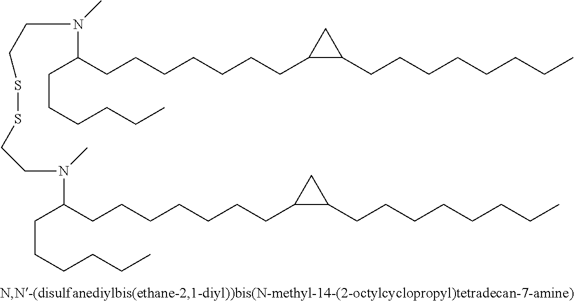 [SMILES: CCCCCCCCC1CC1CCCCCCCC(CCCCCC)N(C)CCSSCCN(C)C(CCCCCC)CCCCCCCC1CC1CCCCCCCC] |
| 5 | 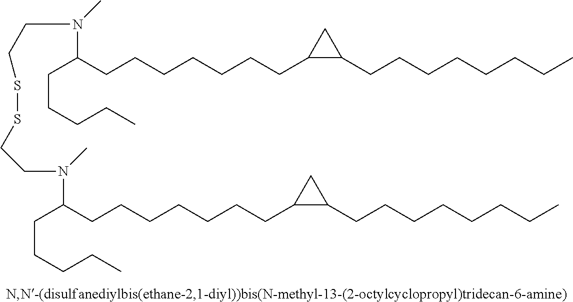 [SMILES: CCCCCCCCC1CC1CCCCCCCC(CCCCC)N(C)CCSSCCN(C)C(CCCCC)CCCCCCCC1CC1CCCCCCCC] |
| 6 | 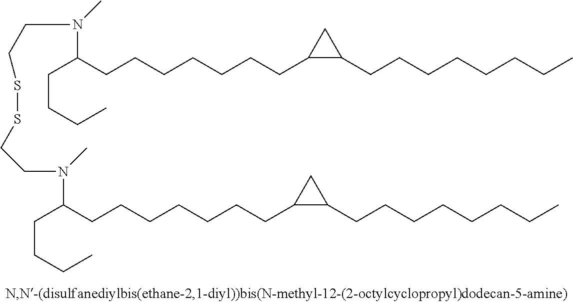 [SMILES: CCCCCCCCC1CC1CCCCCCCC(CCCC)N(C)CCSSCCN(C)C(CCCC)CCCCCCCC1CC1CCCCCCCC] |
| 7 | 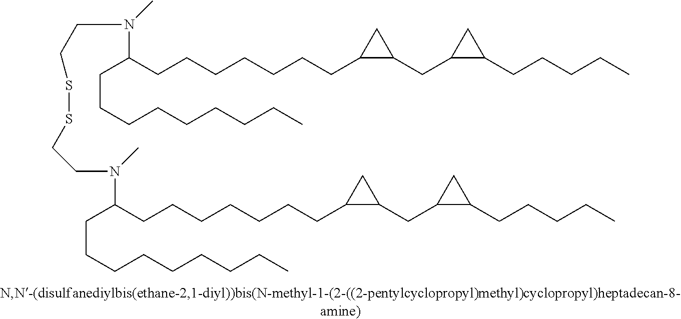 [SMILES: CCCCCCCCCC(CCCCCCCC1CC1CC1CC1CCCCC)N(C)CCSSCCN(C)C(CCCCCCCCC)CCCCCCCC1CC1CC1CC1CCCCC] |
| 8 | 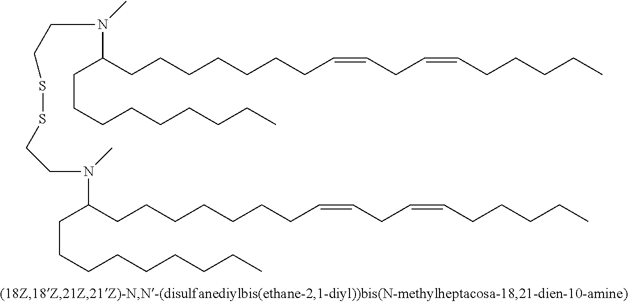 [SMILES: CCCCC/C=C\C/C=C\CCCCCCCC(CCCCCCCCC)N(C)CCSSCCN(C)C(CCCCCCC/C=C\C/C=C\CCCCC)CCCCCCCCC] |
| 9 | 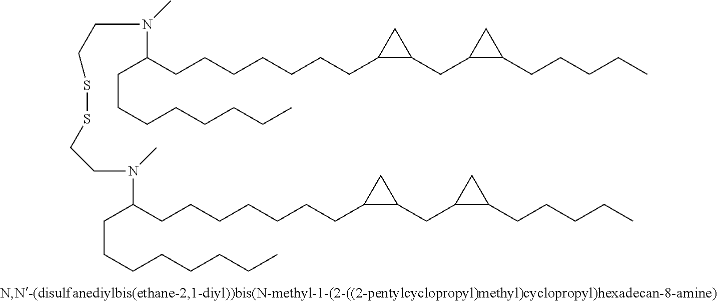 [SMILES: CCCCCCCCC(CCCCCCCC1CC1CC1CC1CCCCC)N(C)CCSSCCN(C)C(CCCCCCCC)CCCCCCCC1CC1CC1CC1CCCCC] |
| 10 | 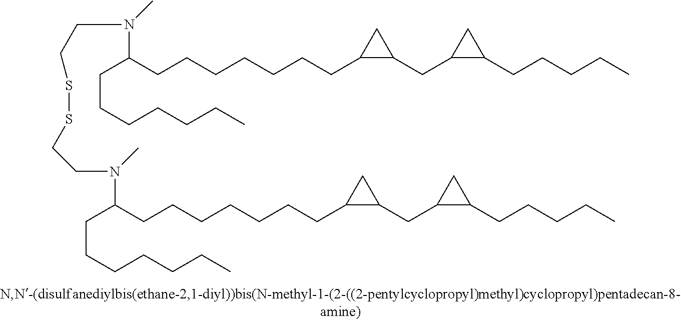 [SMILES: CCCCCCCC(CCCCCCCC1CC1CC1CC1CCCCC)N(C)CCSSCCN(C)C(CCCCCCC)CCCCCCCC1CC1CC1CC1CCCCC] |
| 11 | 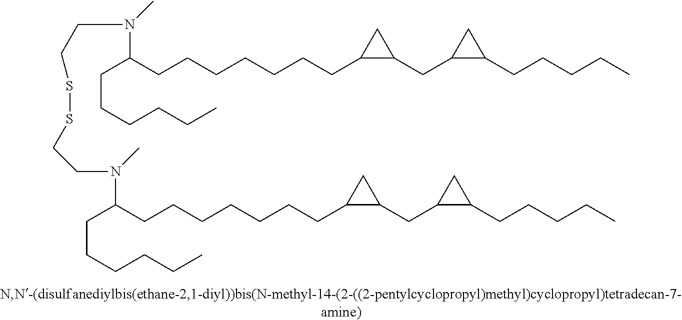 [SMILES: CCCCCCC(CCCCCCCC1CC1CC1CC1CCCCC)N(C)CCSSCCN(C)C(CCCCCC)CCCCCCCC1CC1CC1CC1CCCCC] |
| 12 | 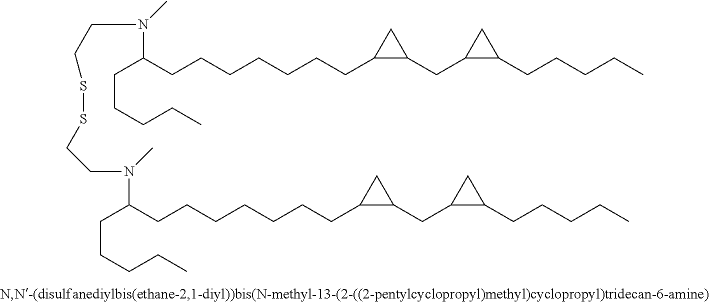 [SMILES: CCCCCC1CC1CC1CC1CCCCCCCC(CCCCC)N(C)CCSSCCN(C)C(CCCCC)CCCCCCCC1CC1CC1CC1CCCCC] |
| 13 | 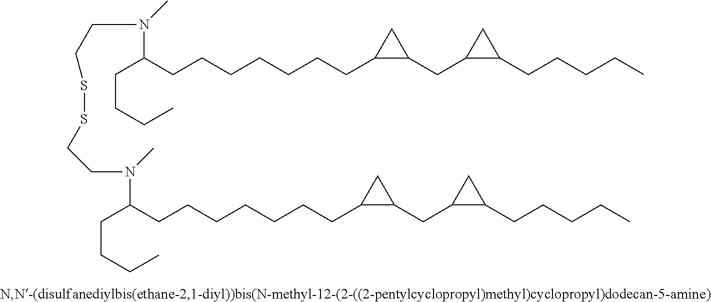 [SMILES: CCCCCC1CC1CC1CC1CCCCCCCC(CCCC)N(C)CCSSCCN(C)C(CCCC)CCCCCCCC1CC1CC1CC1CCCCC] |
| 14 | 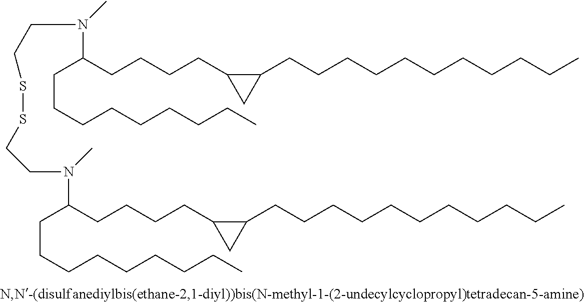 [SMILES: CCCCCCCCCCCC1CC1CCCCC(CCCCCCCCC)N(C)CCSSCCN(C)C(CCCCCCCCC)CCCCC1CC1CCCCCCCCCCC] |
| 15 | 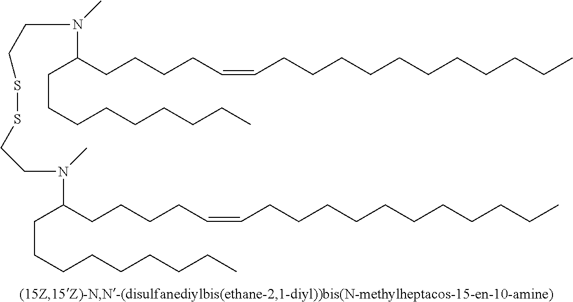 [SMILES: CCCCCCCCCCC/C=C\CCCCC(CCCCCCCCC)N(C)CCSSCCN(C)C(CCCC/C=C\CCCCCCCCCCC)CCCCCCCCC] |
| 16 | 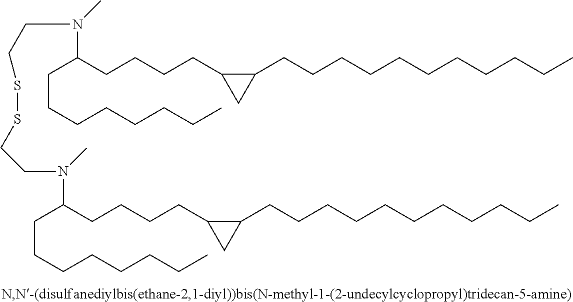 [SMILES: CCCCCCCCCCCC1CC1CCCCC(CCCCCCCC)N(C)CCSSCCN(C)C(CCCCCCCC)CCCCC1CC1CCCCCCCCCCC] |
| 17 | 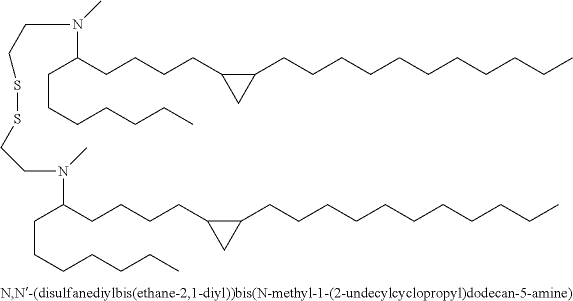 [SMILES: CCCCCCCCCCCC1CC1CCCCC(CCCCCCC)N(C)CCSSCCN(C)C(CCCCCCC)CCCCC1CC1CCCCCCCCCCC] |
| 18 | 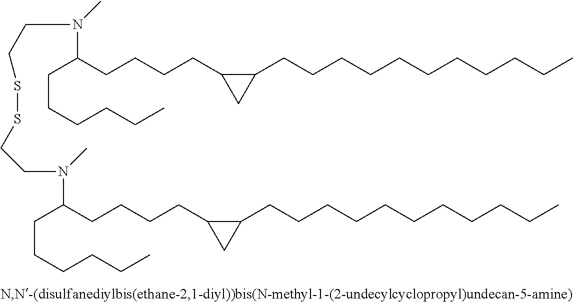 [SMILES: CCCCCCCCCCCC1CC1CCCCC(CCCCCC)N(C)CCSSCCN(C)C(CCCCCC)CCCCC1CC1CCCCCCCCCCC] |
| 19 | 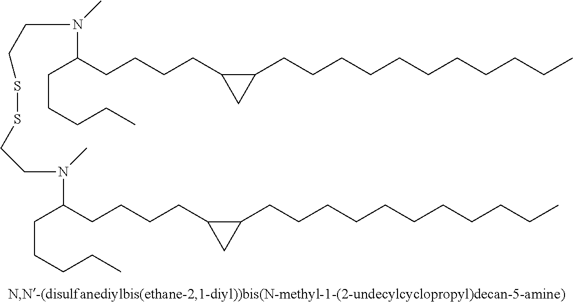 [SMILES: CCCCCCCCCCCC1CC1CCCCC(CCCCC)N(C)CCSSCCN(C)C(CCCCC)CCCCC1CC1CCCCCCCCCCC] |
| 20 | 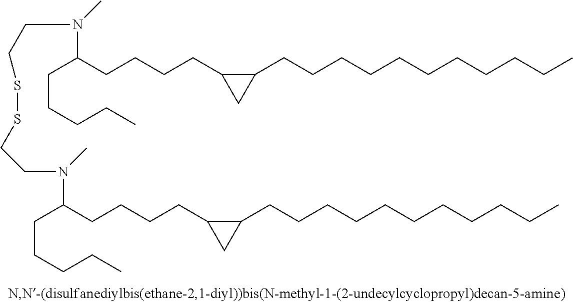 [SMILES: CCCCCCCCCCCC1CC1CCCCC(CCCCC)N(C)CCSSCCN(C)C(CCCCC)CCCCC1CC1CCCCCCCCCCC] |
| 21 | 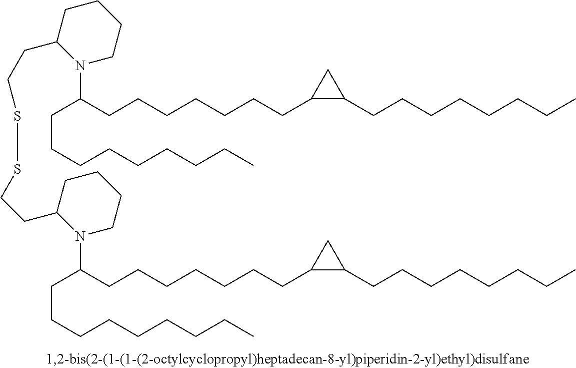 [SMILES: CCCCCCCCCC(CCCCCCCC1CC1CCCCCCCC)N1CCCCC1CCSSCCC1CCCCN1C(CCCCCCCCC)CCCCCCCC1CC1CCCCCCCC] |
| 22 | 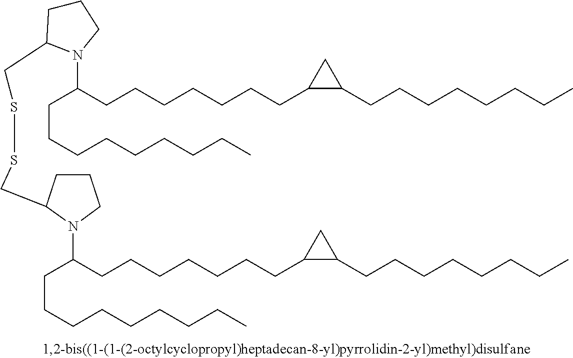 [SMILES: CCCCCCCCCC(CCCCCCCC1CC1CCCCCCCC)N1CCCC1CSSCC1CCCN1C(CCCCCCCCC)CCCCCCCC1CC1CCCCCCCC] |
| 23 | 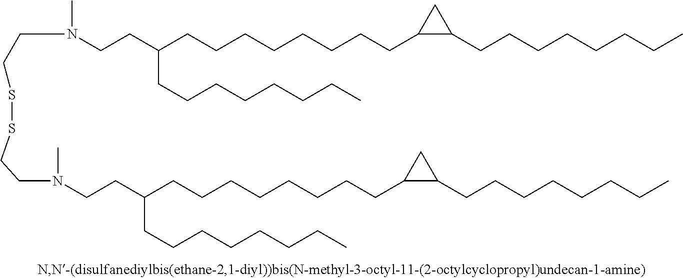 [SMILES: CCCCCCCCC(CCCCCCCCC1CC1CCCCCCCC)CCN(C)CCSSCCN(C)CCC(CCCCCCCC)CCCCCCCCC1CC1CCCCCCCC] |
| 24 | 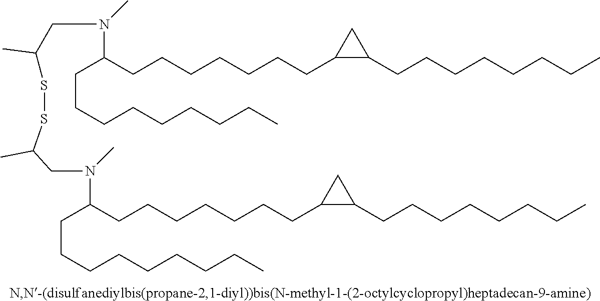 [SMILES: CCCCCCCCCC(CCCCCCCC1CC1CCCCCCCC)N(C)CC(C)SSC(C)CN(C)C(CCCCCCCCC)CCCCCCCC1CC1CCCCCCCC] |
| 25 | 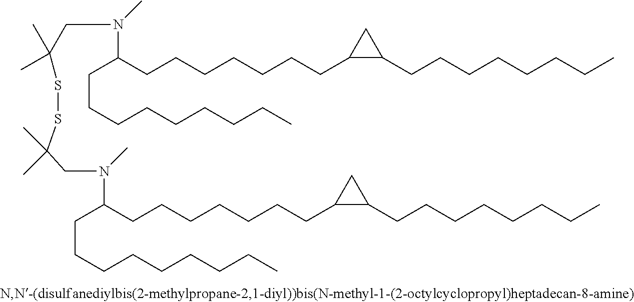 [SMILES: CCCCCCCCCC(CCCCCCCC1CC1CCCCCCCC)N(C)CC(C)(C)SSC(C)(C)CN(C)C(CCCCCCCCC)CCCCCCCC1CC1CCCCCCCC] |
| 26 | 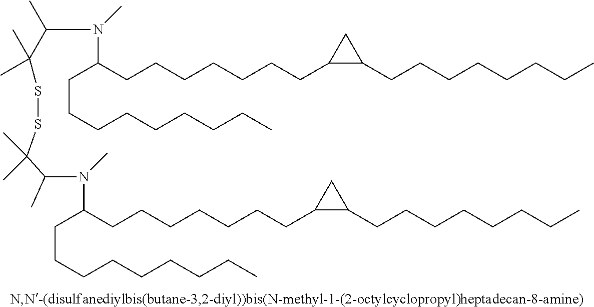 [SMILES: CCCCCCCCCC(CCCCCCCC1CC1CCCCCCCC)N(C)C(C)C(C)(C)SSC(C)(C)C(C)N(C)C(CCCCCCCCC)CCCCCCCC1CC1CCCCCCCC] |
| 27 | 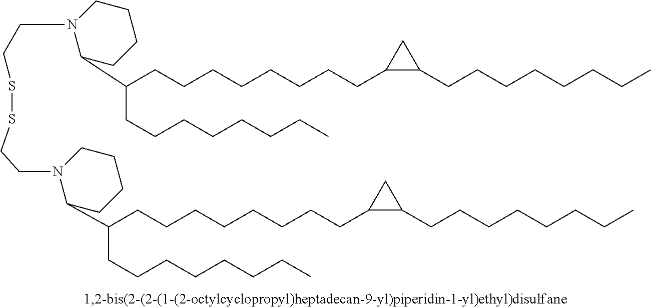 [SMILES: CCCCCCCCC1CC1CCCCCCCCC(CCCCCCCC)C1CCCCN1CCSSCCN1CCCCC1C(CCCCCCCC)CCCCCCCCC1CC1CCCCCCCC] |
| 28 | 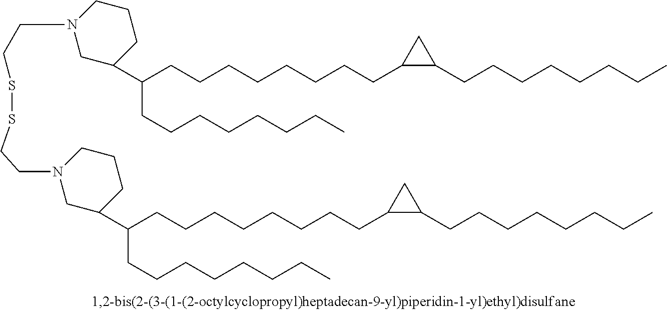 [SMILES: CCCCCCCCC(CCCCCCCCC1CC1CCCCCCCC)C1CCCN(CCSSCCN2CCCC(C(CCCCCCCC)CCCCCCCCC3CC3CCCCCCCC)C2)C1] |
| 29 | 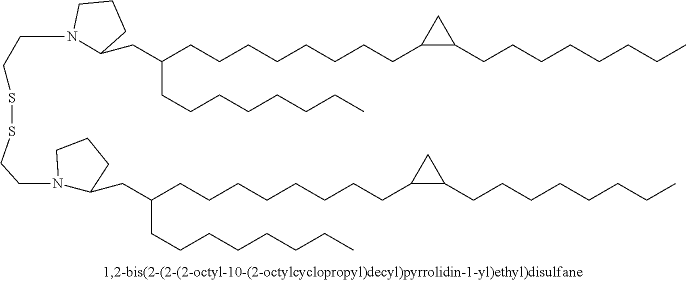 [SMILES: CCCCCCCCC(CCCCCCCCC1CC1CCCCCCCC)CC1CCCN1CCSSCCN1CCCC1CC(CCCCCCCC)CCCCCCCCC1CC1CCCCCCCC] |
| 30 | 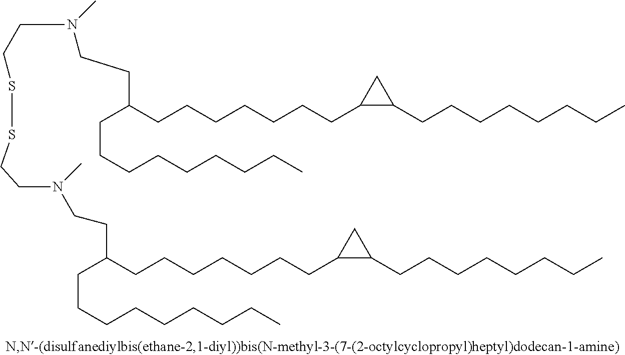 [SMILES: CCCCCCCCCC(CCCCCCCC1CC1CCCCCCCC)CCN(C)CCSSCCN(C)CCC(CCCCCCCCC)CCCCCCCC1CC1CCCCCCCC] |
| 31 | 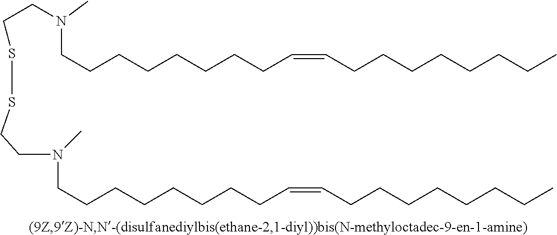 [SMILES: CCCCCCCC/C=C\CCCCCCCCN(C)CCSSCCN(C)CCCCCCCC/C=C\CCCCCCCC] |
| 32 | 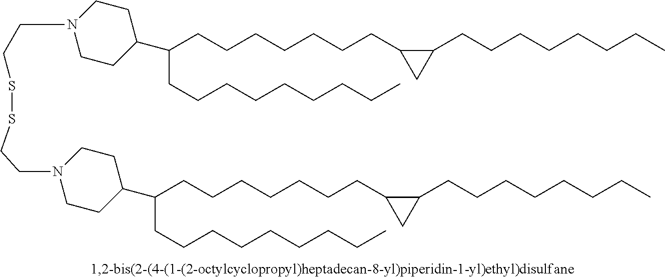 [SMILES: CCCCCCCCCC(CCCCCCCC1CC1CCCCCCCC)C1CCN(CCSSCCN2CCC(C(CCCCCCCCC)CCCCCCCC3CC3CCCCCCCC)CC2)CC1] |
| 33 | 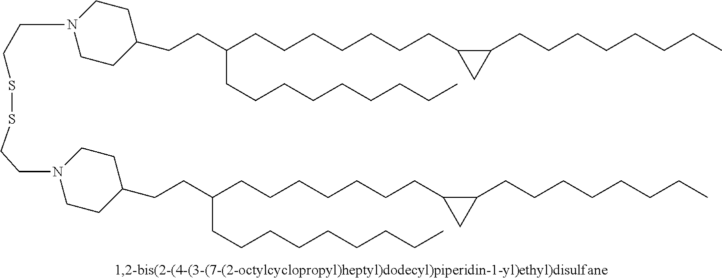 [SMILES: CCCCCCCCCC(CCCCCCCC1CC1CCCCCCCC)CCC1CCN(CCSSCCN2CCC(CCC(CCCCCCCCC)CCCCCCCC3CC3CCCCCCCC)CC2)CC1] |
| 34 | 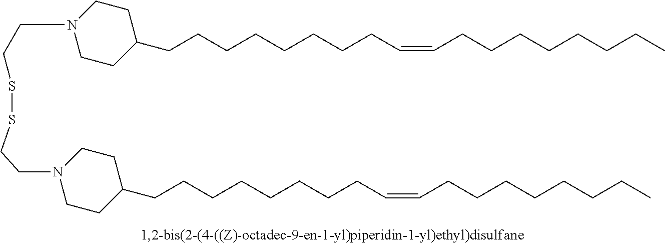 [SMILES: CCCCCCCC/C=C\CCCCCCCCC1CCN(CCSSCCN2CCC(CCCCCCCC/C=C\CCCCCCCC)CC2)CC1] |
| 35 | 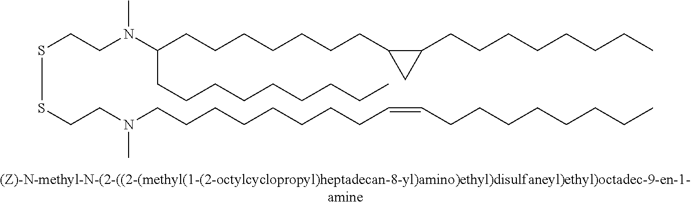 [SMILES: CCCCCCCC/C=C\CCCCCCCCN(C)CCSSCCN(C)C(CCCCCCCCC)CCCCCCCC1CC1CCCCCCCC] |
| 36 | 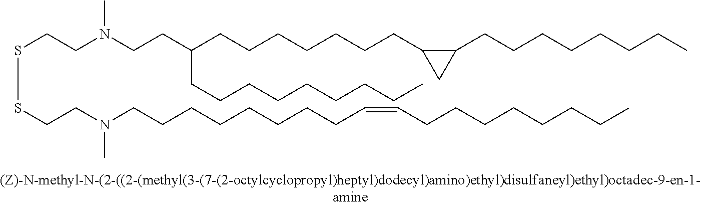 [SMILES: CCCCCCCC/C=C\CCCCCCCCN(C)CCSSCCN(C)CCC(CCCCCCCCC)CCCCCCCC1CC1CCCCCCCC] |
| 37 | 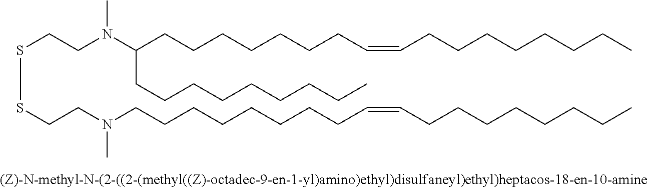 [SMILES: CCCCCCCC/C=C\CCCCCCCCN(C)CCSSCCN(C)C(CCCCCCC/C=C\CCCCCCCC)CCCCCCCCC] |
| 38 | 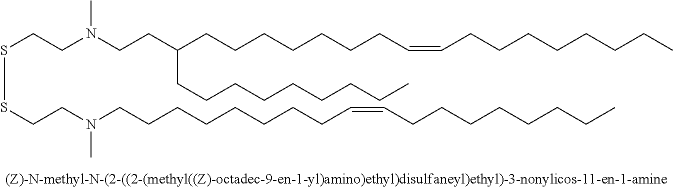 [SMILES: CCCCCCCC/C=C\CCCCCCCCN(C)CCSSCCN(C)CCC(CCCCCCC/C=C\CCCCCCCC)CCCCCCCCC] |
| 39 | 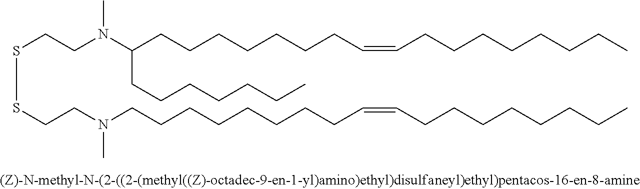 [SMILES: CCCCCCCC/C=C\CCCCCCCCN(C)CCSSCCN(C)C(CCCCCCC)CCCCCCC/C=C\CCCCCCCC] |
| 40 | 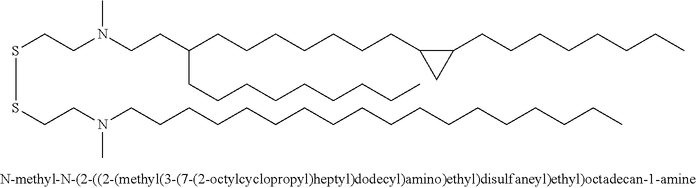 [SMILES: CCCCCCCCCCCCCCCCCCN(C)CCSSCCN(C)CCC(CCCCCCCCC)CCCCCCCC1CC1CCCCCCCC] |
| 41 | 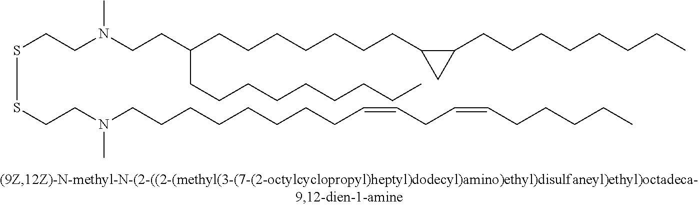 [SMILES: CCCCC/C=C\C/C=C\CCCCCCCCN(C)CCSSCCN(C)CCC(CCCCCCCCC)CCCCCCCC1CC1CCCCCCCC] |
| 42 | 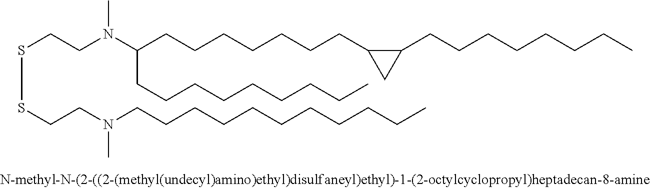 [SMILES: CCCCCCCCCCCN(C)CCSSCCN(C)C(CCCCCCCCC)CCCCCCCC1CC1CCCCCCCC] |
| 43 | 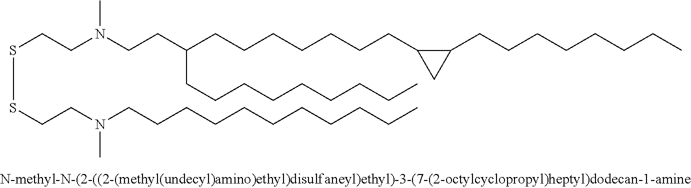 [SMILES: CCCCCCCCCCCN(C)CCSSCCN(C)CCC(CCCCCCCCC)CCCCCCCC1CC1CCCCCCCC] |
| 44 | 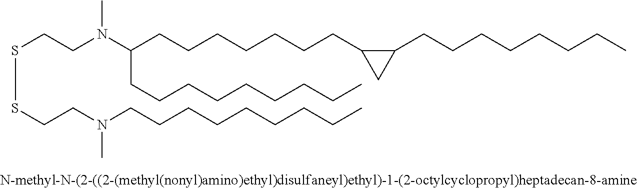 [SMILES: CCCCCCCCCC(CCCCCCCC1CC1CCCCCCCC)N(C)CCSSCCN(C)CCCCCCCCC] |
| 45 | 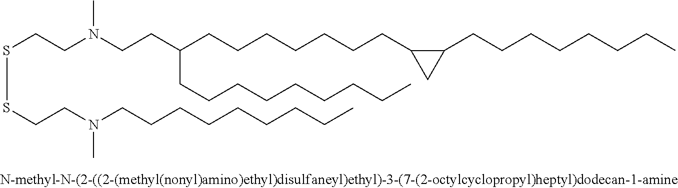 [SMILES: CCCCCCCCCC(CCCCCCCC1CC1CCCCCCCC)CCN(C)CCSSCCN(C)CCCCCCCCC] |
| 46 | 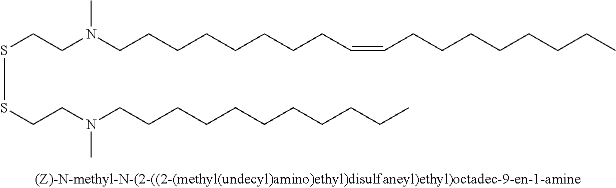 [SMILES: CCCCCCCC/C=C\CCCCCCCCN(C)CCSSCCN(C)CCCCCCCCCCC] |
| 47 | 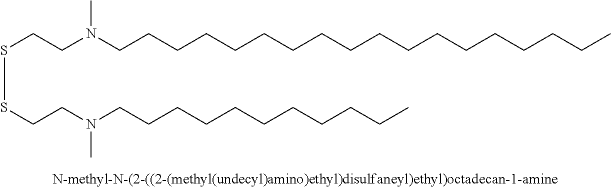 [SMILES: CCCCCCCCCCCCCCCCCCN(C)CCSSCCN(C)CCCCCCCCCCC] |
| 48 | 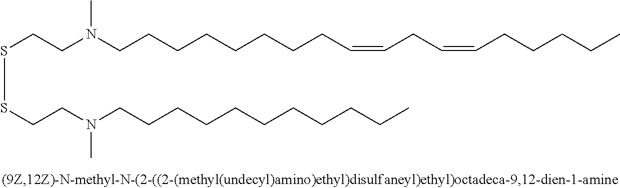 [SMILES: CCCCC/C=C\C/C=C\CCCCCCCCN(C)CCSSCCN(C)CCCCCCCCCCC] |
| 49 | 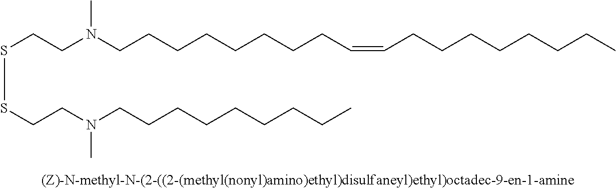 [SMILES: CCCCCCCC/C=C\CCCCCCCCN(C)CCSSCCN(C)CCCCCCCCC] |
| 50 | 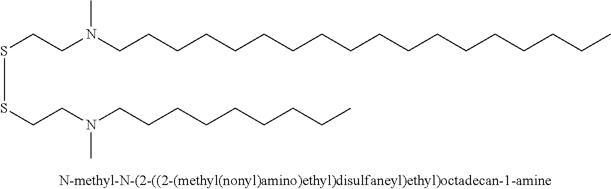 [SMILES: CCCCCCCCCCCCCCCCCCN(C)CCSSCCN(C)CCCCCCCCC] |
| 51 | 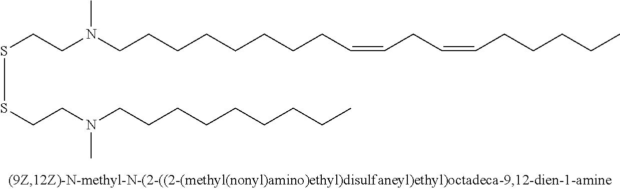 [SMILES: CCCCC/C=C\C/C=C\CCCCCCCCN(C)CCSSCCN(C)CCCCCCCCC] |

### Formula (II)

In some aspects, the cationic lipids are of the Formula (II):

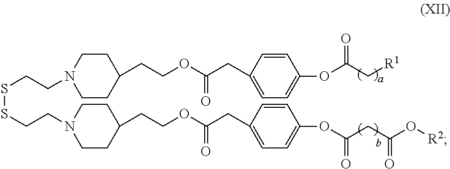

or a pharmaceutically acceptable salt thereof, wherein:
a is an integer ranging from 1 to 20 (e.g., a is 1, 2, 3, 4, 5, 6, 7, 8, 9, 10, 11, 12, 13, 14, 15, 16, 17, 18, 19, or 20);
b is an integer ranging from 2 to 10 (e.g., b is 2, 3, 4, 5, 6, 7, 8, 9, or 10);
R¹ is absent or is selected from (C₂-C₂₀)alkenyl, —C(O)O(C₂-C₂₀)alkyl, and cyclopropyl substituted with (C₂-C₂₀)alkyl; and
R² is (C₂-C₂₀)alkyl.

In a second chemical embodiment, the cationic lipid of the Formula (II) is of the Formula (XIII):

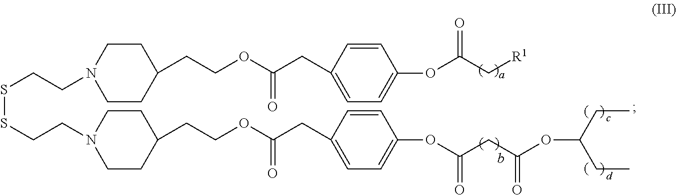

or a pharmaceutically acceptable salt thereof, wherein c and d are each independently integers ranging from 1 to 8 (e.g., 1, 2, 3, 4, 5, 6, 7, or 8), and wherein the remaining variables are as described for Formula (XII).

In a third chemical embodiment, c and d in the cationic lipid of Formula (II) or (III) are each independently integers ranging from 2 to 8, 3 to 8, 3 to 7, 3 to 6, 3 to 5, 4 to 8, 4 to 7, 4 to 6, 5 to 8, 5 to 7, or 6 to 8, wherein the remaining variables are as described for Formula (XII).

In a fourth chemical embodiment, c in the cationic lipid of Formula (II) or (III) is 2, 3, 4, 5, 6, 7, or 8, wherein the remaining variables are as described for Formula (XII) or the second or third chemical embodiment. Alternatively, as part of a fourth chemical embodiment, c and d in the cationic lipid of Formula (XII) or (XIII) or a pharmaceutically acceptable salt thereof are each independently 1, 3, 5, or 7, wherein the remaining variables are as described for Formula (XII) or the second or third chemical embodiment.

In a fifth chemical embodiment, d in the cationic lipid of Formula (II) or (III) is 2, 3, 4, 5, 6, 7, or 8, wherein the remaining variables are as described for Formula (II) or the second or third or fourth chemical embodiment. Alternatively, as part of a fourth chemical embodiment, at least one of c and d in the cationic lipid of Formula (II) or (III) or a pharmaceutically acceptable salt thereof is 7, wherein the remaining variables are as described for Formula (II) or the second or third or fourth chemical embodiment.

In a sixth chemical embodiment, the cationic lipid of Formula (II) or (III) is of the Formula (IV):

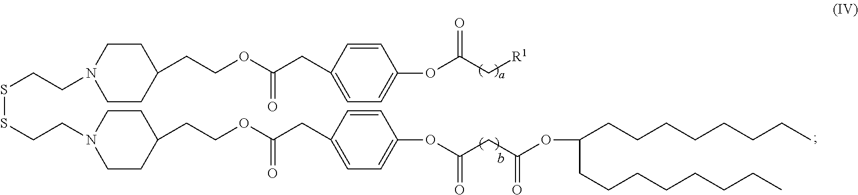

or a pharmaceutically acceptable salt thereof, wherein the remaining variables are as described for Formula (I).

In a seventh chemical embodiment, b in the cationic lipid of Formula (II), (III), or (IV) is an integer ranging from 3 to 9, wherein the remaining variables are as described for Formula (II), or the second, third, fourth or fifth chemical embodiment. Alternatively, as part of a seventh chemical embodiment, b in the cationic lipid of Formula (II), (III), or (IV) is an integer ranging from 3 to 8, 3 to 7, 3 to 6, 3 to 5, 4 to 9, 4 to 8, 4 to 7, 4 to 6, 5 to 9, 5 to 8, 5 to 7, 6 to 9, 6 to 8, or 7 to 9, wherein the remaining variables are as described for Formula (II), or the second, third, fourth or fifth chemical embodiment. In another alternative, as part of a seventh chemical embodiment, b in the cationic lipid of Formula (II), (III), or (IV) is 3, 4, 5, 6, 7, 8, or 9, wherein the remaining variables are as described for Formula (XII), or the second, third, fourth or fifth chemical embodiment.

In an eighth chemical embodiment, a in the cationic lipid of Formula (II), (III), or (IV) is an integer ranging from 2 to 18, wherein the remaining variables are as described for Formula (II), or the second, third, fourth, fifth, or seventh chemical embodiment. Alternatively, as part of an eighth embodiment, a in the cationic lipid of Formula (II), (III), or (IV) is an integer ranging from 2 to 18, 2 to 17, 2 to 16, 2 to 15, 2 to 14, 2 to 13, 2 to 12, 2 to 11, 2 to 10, 2 to 9, 2 to 8, 2 to 7, 2 to 6, 2 to 5, 2 to 4, 3 to 18, 3 to 17, 3 to 16, 3 to 15, 3 to 14, 3 to 13, 3 to 12, 3 to 11, 3 to 10, 3 to 9, 3 to 8, 3 to 7, 3 to 6, 3 to 5, 4 to 18, 4 to 17, 4 to 16, 4 to 15, 4 to 14, 4 to 13, 4 to 12, 4 to 11, 4 to 10, 4 to 9, 4 to 8, 4 to 7, 4 to 6, 5 to 18, 5 to 17, 5 to 16, 5 to 15, 5 to 14, 5 to 13, 5 to 12, 5 to 11, 5 to 10, 5 to 9, 25 to 8, 5 to 7, 6 to 18, 6 to 17, 6 to 16, 6 to 15, 6 to 14, 6 to 13, 6 to 12, 6 to 11, 6 to 10, 6 to 9, 6 to 8, 7 to 18, 7 to 17, 7 to 16, 7 to 15, 7 to 14, 7 to 13, 7 to 12, 7 to 11, 7 to 10, 7 to 9, 8 to 18, 8 to 17, 8 to 16, 8 to 15, 8 to 14, 8 to 13, 8 to 12, 8 to 11, 8 to 10, 9 to 18, 9 to 17, 9 to 16, 9 to 15, 9 to 14, 9 to 13, 9 to 12, 9 to 11, 10 to 18, 10 to 17, 10 to 16, 10 to 15, 10 to 14, 10 to 13, 11 to 18, 11 to 17, 11 to 16, 11 to 15, 11 to 14, 11 to 13, 12 to 18, 12 to 17, 12 to 16, 12 to 15, 12 to 14, 13 to 18, 13 to 17, 13 to 16, 13 to 15, 14 to 18, 14 to 17, 14 to 16, 15 to 18, 15 to 17, or 16 to 18, wherein the remaining variables are as described for Formula (II), or the second, third, fourth, fifth, or seventh chemical embodiment. In another alternative, as part of an eighth embodiment, a in the cationic lipid of Formula (II), (III), or (IV) is 2, 3, 4, 5, 6, 7, 8, 9, 10, 11, 12, 13, 14, 15, 16, 17, or 18, wherein the remaining variables are as described for Formula (II), or the second, third, fourth, fifth, or seventh chemical embodiment.

In a ninth chemical embodiment, R¹ in the cationiclipid of Formula (II), (III), or (IV) or a pharmaceutically acceptable salt thereof is absent or is selected from (C₅-C₁₅)alkenyl, —C(O)O(C₄-C₁₈)alkyl, and cyclopropyl substituted with (C₄-C₁₆)alkyl, wherein the remaining variables are as described for Formula (II), (III), or (IV) or the second, third, fourth, fifth, seventh, or eighth chemical embodiment. Alternatively, as part of a ninth chemical embodiment, R¹ in the cationic lipid of Formula (II), (III), or (IV) or a pharmaceutically acceptable salt thereof is absent or is selected from (C₅-C₁₅)alkenyl, —C(O)O(C₄-C₁₆)alkyl, and cyclopropyl substituted with (C₄-C₁₆)alkyl, wherein the remaining variables are as described for Formula (II), (III), or (IV) or the second, third, fourth, fifth, seventh, or eighth chemical embodiment. Alternatively, as part of a ninth chemical embodiment, R¹ in the cationic lipid of Formula (II), (III), or (IV) or a pharmaceutically acceptable salt thereof is absent or is selected from (C₅-C₁₂)alkenyl, —C(O)O(C₄-C₁₂)alkyl, and cyclopropyl substituted with (C₄-C₁₂)alkyl, wherein the remaining variables are as described for Formula (II), (III), or (IV) or the second, third, fourth, fifth, seventh, or eighth chemical embodiment. In another alternative, as part of a ninth chemical embodiment, R¹ in the cationic lipid of Formula (II), (III), or (IV) or a pharmaceutically acceptable salt thereof is absent or is selected from (C₅-C₁₀)alkenyl, —C(O)O(C₄-C₁₀)alkyl, and cyclopropyl substituted with (C₄-C₁₀)alkyl, wherein the remaining variables are as described for Formula (II), (III), or (IV) or the second, third, fourth, fifth, seventh, or eighth chemical embodiment.

In a tenth chemical embodiment, R¹ is C₁₀ alkenyl, wherein the remaining variables are as described in any one of the foregoing embodiments.

In an eleventh chemical embodiment, the alkyl in C(O)O(C₂-C₂₀)alkyl, —C(O)O(C₄-C₁₅)alkyl, —C(O)O(C₄-C₁₂)alkyl, or —C(O)O(C₄-C₁₀)alkyl of R¹ in the cationic lipid of Formula (II), (III), or (IV) or a pharmaceutically acceptable salt thereof is an unbranched alkyl, wherein the remaining variables are as described in any one of the foregoing embodiments. In one chemical embodiment, R¹ is —C(O)O(C₉ alkyl). Alternatively, in an eleventh chemical embodiment, the alkyl in —C(O)O(C₄-C₁₈)alkyl, —C(O)O(C₄-C₁₂)alkyl, or —C(O)O(C₄-C₁₀)alkyl of R¹ in the cationic lipid of Formula (II), (III), or (IV) or a pharmaceutically acceptable salt thereof is a branched alkyl, wherein the remaining variables are as described in any one of the foregoing chemical embodiments. In one chemical embodiment, R¹ is —C(O)O(C₁₇ alkyl), wherein the remaining variables are as described in any one of the foregoing chemical embodiments.

In a twelfth chemical embodiment, R¹ in the cationic lipid of Formula (II), (III), or (IV) or a pharmaceutically acceptable salt thereof is selected from any group listed in Table 3 below, wherein the wavy bond in each of the groups indicates the point of attachment of the group to the rest of the lipid molecule, and wherein the remaining variables are as described for Formula (II), (III), or (IV) or the second, third, fourth, fifth, seventh, or eighth chemical embodiment. The present disclosure further contemplates the combination of any one of the R¹ groups in Table 4 with any one of the R²groups in Table 5, wherein the remaining variables are as described for Formula (II), (III), or (IV) or the second, third, fourth, fifth, seventh, or eighth chemical embodiment.

TABLE 3
Exemplary R¹ groups in Formula (II), (III), or (IV)

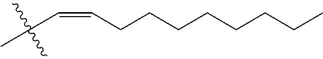 [SMILES: */C=C\CCCCCCCC]

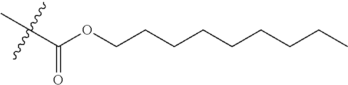 [SMILES: *C(=O)OCCCCCCCCC]

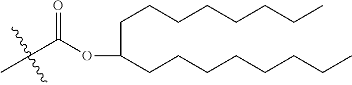 [SMILES: *C(=O)OC(CCCCCCCC)CCCCCCCC]

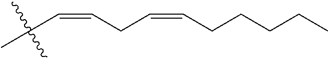 [SMILES: */C=C\C/C=C\CCCCC]

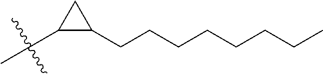 [SMILES: *C1CC1CCCCCCCC]

In a thirteenth chemical embodiment, R² in the cationic lipid of Formula (II) or a pharmaceutically acceptable salt thereof is selected from any group listed in Table 4 below, wherein the wavy bond in each of the groups indicates the point of attachment of the group to the rest of the lipid molecule, and wherein the remaining variables are as described for Formula (II), or the seventh, eighth, ninth, tenth, or eleventh chemical embodiment.

TABLE 4
Exemplary R² groups in Formula (II)

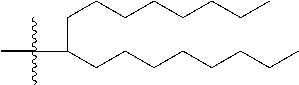 [SMILES: *C(CCCCCCCC)CCCCCCCC]

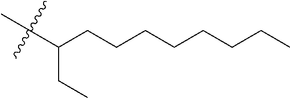 [SMILES: *C(CC)CCCCCCCC]

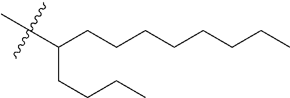 [SMILES: *C(CCCC)CCCCCCCC]

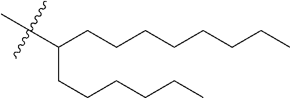 [SMILES: *C(CCCCCC)CCCCCCCC]

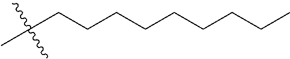 [SMILES: *CCCCCCCCC]

Specific examples are provided in Table 5 the exemplification section below and are included as part of a fourteenth chemical embodiment herein of cationic lipids of Formula (II). Pharmaceutically acceptable salts as well as ionized and neutral forms are also included.

TABLE 5
Exemplary cationic lipids of Formula (II), (III), or (IV)

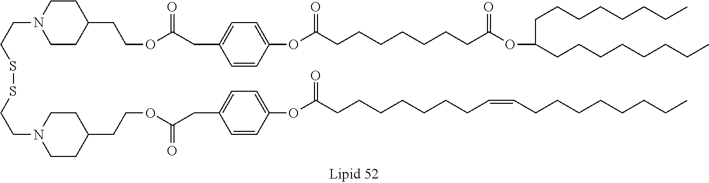 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCCC(=O)OC(CCCCCCCC)CCCCCCCC)cc4)CC3)CC2)cc1]

1-(heptadecan-9-yl) 9-(4-(2-(2-(1-(2-((2-(4-(2-(2-(4-(oleoyloxy)phenyl)acetoxy)ethyl)piperidin-1-
yl)ethyl)disulfaneyl)ethyl)piperidin-4-yl)ethoxy)-2-oxoethyl)phenyl) nonanedioate

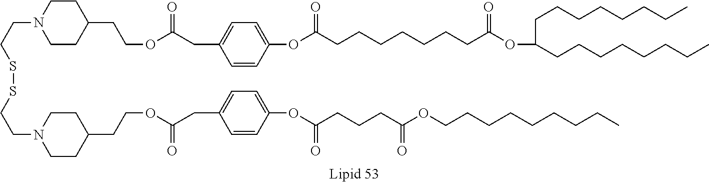 [SMILES: CCCCCCCCCOC(=O)CCCC(=O)Oc1ccc(CC(=O)OCCC2CCN(CCSSCCN3CCC(CCOC(=O)Cc4ccc(OC(=O)CCCCCCCC(=O)OC(CCCCCCCC)CCCCCCCC)cc4)CC3)CC2)cc1]

1-(heptadecan-9-yl) 9-(4-(2-(2-(1-(2-((2-(4-(2-(2-(4-((5-(nonyloxy)-5-
oxopentanoyl)oxy)phenyl)acetoxy)ethyl) piperidin-1-yl)ethyl)disulfaneyl)ethyl) piperidin-4-
yl)ethoxy)-2-oxoethyl)phenyl) nonanedioate

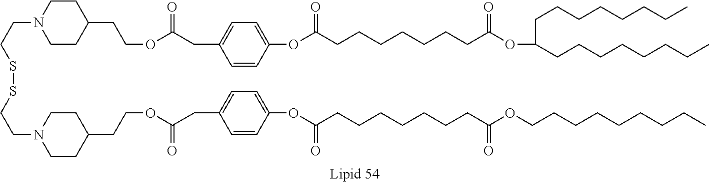 [SMILES: CCCCCCCCCOC(=O)CCCCCCCC(=O)Oc1ccc(CC(=O)OCCC2CCN(CCSSCCN3CCC(CCOC(=O)Cc4ccc(OC(=O)CCCCCCCC(=O)OC(CCCCCCCC)CCCCCCCC)cc4)CC3)CC2)cc1]

1-(heptadecan-9-yl) 9-(4-(2-(2-(1-(2-((2-(4-(2-(2-(4-((9-(nonyloxy)-9-
oxononanoyl)oxy)phenyl)acetoxy)ethyl)piperidin-1-yl)ethyl)disulfaneyl)ethyl)piperidin-4-
yl)ethoxy)-2-oxoethyl)phenyl) nonanedioate

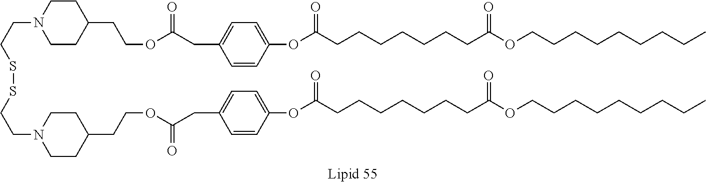 [SMILES: CCCCCCCCCOC(=O)CCCCCCCC(=O)Oc1ccc(CC(=O)OCCC2CCN(CCSSCCN3CCC(CCOC(=O)Cc4ccc(OC(=O)CCCCCCCC(=O)OCCCCCCCCC)cc4)CC3)CC2)cc1]

1-(heptadecan-9-yl) 9-(4-(2-(2-(1-(2-((2-(4-(2-(2-(4-((5-(nonyloxy)-5-
oxopentanoyl)oxy)phenyl)acetoxy)ethyl)
piperidin-1-yl)ethyl)disulfaneyl)ethyl)piperidin-4-yl)ethoxy)-2-oxoethyl)phenyl) nonanedioate

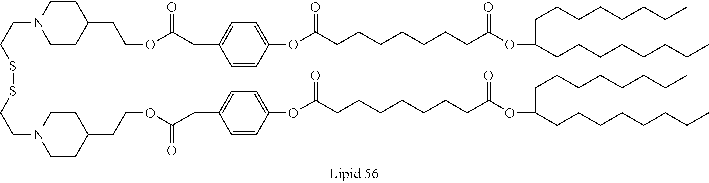 [SMILES: CCCCCCCCC(CCCCCCCC)OC(=O)CCCCCCCC(=O)Oc1ccc(CC(=O)OCCC2CCN(CCSSCCN3CCC(CCOC(=O)Cc4ccc(OC(=O)CCCCCCCC(=O)OC(CCCCCCCC)CCCCCCCC)cc4)CC3)CC2)cc1]

O′1,O1-((((((disulfanediylbis(ethane-2,1-diyl))bis(piperidine-1,4-diyl))bis(ethane-2,1-
diyl))bis(oxy))bis(2-oxoethane-2,1-diyl))bis(4,1-phenylene)) 9,9′-di(heptadecan-9-yl)
di(nonanedioate)

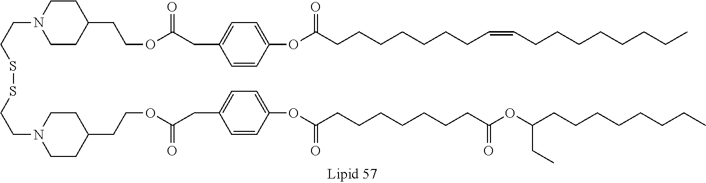 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCCC(=O)OC(CC)CCCCCCCC)cc4)CC3)CC2)cc1]

1-(4-(2-(2-(1-(2-((2-(4-(2-(2-(4-(oleoyloxy)phenyl)acetoxy)ethyl)piperidin-1-
yl)ethyl)disulfaneyl)ethyl)piperidin-4-yl)ethoxy)-2-oxoethyl)phenyl) 9-(undecan-3-yl)
nonanedioate

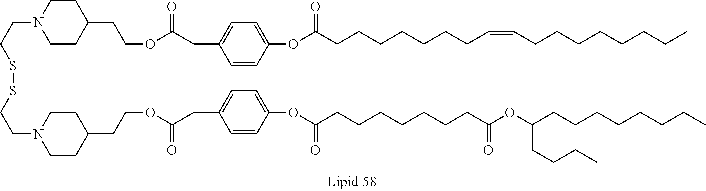 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCCC(=O)OC(CCCC)CCCCCCCC)cc4)CC3)CC2)cc1]

1-(4-(2-(2-(1-(2-((2-(4-(2-(2-(4-(oleoyloxy)phenyl)acetoxy)ethyl)piperidin-1-
yl)ethyl)disulfaneyl)ethyl)piperidin-4-yl)ethoxy)-2-oxoethyl)phenyl) 9-(tridecan-5-yl)
nonanedioate

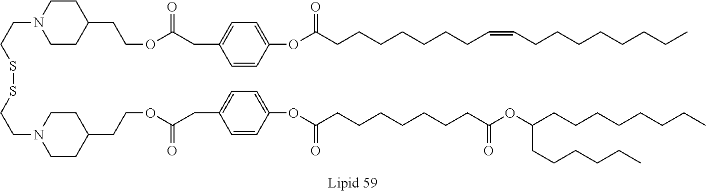 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCCC(=O)OC(CCCCCC)CCCCCCCC)cc4)CC3)CC2)cc1]

1-(4-(2-(2-(1-(2-((2-(4-(2-(2-(4-(oleoyloxy)phenyl)acetoxy)ethyl)piperidin-1-
yl)ethyl)disulfaneyl)ethyl)piperidin-4-yl)ethoxy)-2-oxoethyl)phenyl) 9-(pentadecan-7-yl)
nonanedioate

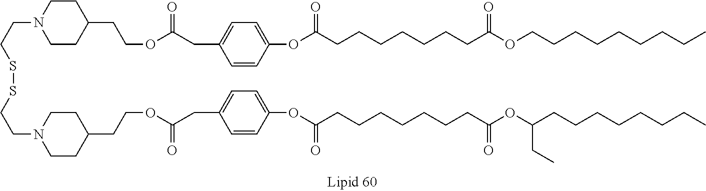 [SMILES: CCCCCCCCCOC(=O)CCCCCCCC(=O)Oc1ccc(CC(=O)OCCC2CCN(CCSSCCN3CCC(CCOC(=O)Cc4ccc(OC(=O)CCCCCCCC(=O)OC(CC)CCCCCCCC)cc4)CC3)CC2)cc1]

1-nonyl 9-(4-(2-oxo-2-(2-(1-(2-((2-(4-(2-(2-(4-((9-oxo-9-(undecan-3-
yloxy)nonanoyl)oxy)phenyl)acetoxy)ethyl)piperidin-1-yl)ethyl)disulfaneyl)ethyl)piperidin-4-
yl)ethoxy)ethyl)phenyl) nonanedioate

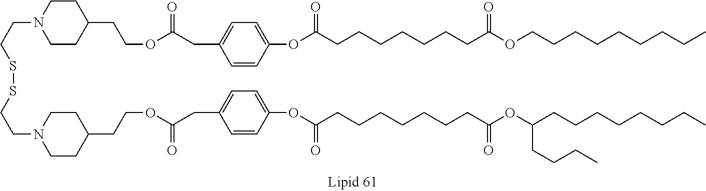 [SMILES: CCCCCCCCCOC(=O)CCCCCCCC(=O)Oc1ccc(CC(=O)OCCC2CCN(CCSSCCN3CCC(CCOC(=O)Cc4ccc(OC(=O)CCCCCCCC(=O)OC(CCCC)CCCCCCCC)cc4)CC3)CC2)cc1]

1-nonyl 9-(4-(2-oxo-2-(2-(1-(2-((2-(4-(2-(2-(4-((9-oxo-9-(tridecan-5-
yloxy)nonanoyl)oxy)phenyl)acetoxy)ethyl)piperidin-1-yl)ethyl)disulfaneyl)ethyl)piperidin-4-
yl)ethoxy)ethyl)phenyl) nonanedioate

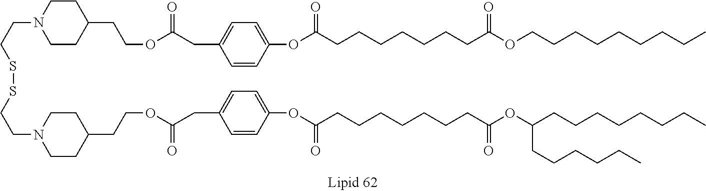 [SMILES: CCCCCCCCCOC(=O)CCCCCCCC(=O)Oc1ccc(CC(=O)OCCC2CCN(CCSSCCN3CCC(CCOC(=O)Cc4ccc(OC(=O)CCCCCCCC(=O)OC(CCCCCC)CCCCCCCC)cc4)CC3)CC2)cc1]

1-nonyl 9-(4-(2-oxo-2-(2-(1-(2-((2-(4-(2-(2-(4-((9-oxo-9-(pentadecan-7-
yloxy)nonanoyl)oxy)phenyl)acetoxy)ethyl)piperidin-1-yl)ethyl)disulfaneyl)ethyl)piperidin-4-
yl)ethoxy)ethyl)phenyl) nonanedioate

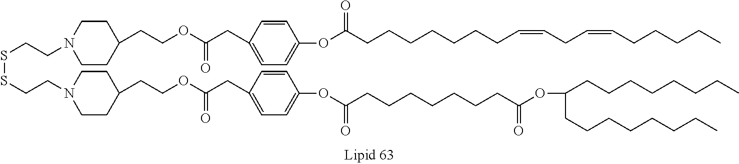 [SMILES: CCCCC/C=C\C/C=C\CCCCCCCC(=O)Oc1ccc(CC(=O)OCCC2CCN(CCSSCCN3CCC(CCOC(=O)Cc4ccc(OC(=O)CCCCCCCC(=O)OC(CCCCCCCC)CCCCCCCC)cc4)CC3)CC2)cc1]

1-(heptadecan-9-yl) 9-(4-(2-(2-(1-(2-((2-(4-(2-(2-(4-(((9Z,12Z)-octadeca-9,12-
dienoyl)oxy)phenyl)acetoxy)ethyl)piperidin-1-yl)ethyl)disulfaneyl)ethyl)piperidin-4-yl)ethoxy)-2-
oxoethyl)phenyl) nonanedioate

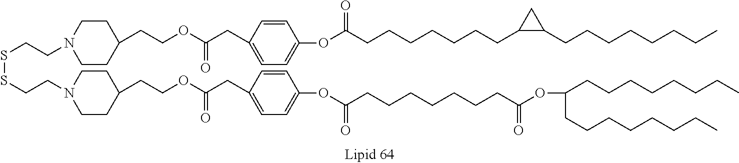 [SMILES: CCCCCCCCC(CCCCCCCC)OC(=O)CCCCCCCC(=O)Oc1ccc(CC(=O)OCCC2CCN(CCSSCCN3CCC(CCOC(=O)Cc4ccc(OC(=O)CCCCCCCC5CC5CCCCCCCC)cc4)CC3)CC2)cc1]

1-(heptadecan-9-yl) 9-(4-(2-(2-(1-(2-((2-(4-(2-(2-(4-((8-(2-
octylcyclopropyl)octanoyl)oxy)phenyl)acetoxy)ethyl)piperidin-1-
yl)ethyl)disulfaneyl)ethyl)piperidin-4-yl)ethoxy)-2-oxoethyl)phenyl) nonanedioate

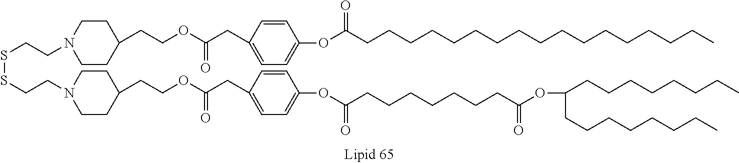 [SMILES: CCCCCCCCCCCCCCCCCC(=O)Oc1ccc(CC(=O)OCCC2CCN(CCSSCCN3CCC(CCOC(=O)Cc4ccc(OC(=O)CCCCCCCC(=O)OC(CCCCCCCC)CCCCCCCC)cc4)CC3)CC2)cc1]

1-(heptadecan-9-yl) 9-(4-(2-oxo-2-(2-(1-(2-((2-(4-(2-(2-(4-
(stearoyloxy)phenyl)acetoxy)ethyl)piperidin-1-yl)ethyl)disulfaneyl)ethyl)piperidin-4-
yl)ethoxy)ethyl)phenyl) nonanedioate

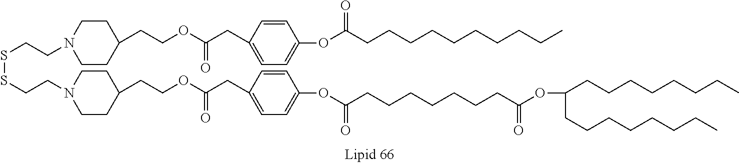 [SMILES: CCCCCCCCCCC(=O)Oc1ccc(CC(=O)OCCC2CCN(CCSSCCN3CCC(CCOC(=O)Cc4ccc(OC(=O)CCCCCCCC(=O)OC(CCCCCCCC)CCCCCCCC)cc4)CC3)CC2)cc1]

1-(heptadecan-9-yl) 9-(4-(2-oxo-2-(2-(1-(2-((2-(4-(2-(2-(4-
(undecanoyloxy)phenyl)acetoxy)ethyl)piperidin-1-yl)ethyl)disulfaneyl)ethyl)piperidin-4-
yl)ethoxy)ethyl)phenyl) nonanedioate

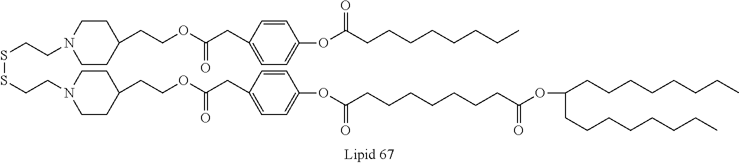 [SMILES: CCCCCCCCC(=O)Oc1ccc(CC(=O)OCCC2CCN(CCSSCCN3CCC(CCOC(=O)Cc4ccc(OC(=O)CCCCCCCC(=O)OC(CCCCCCCC)CCCCCCCC)cc4)CC3)CC2)cc1]

1-(heptadecan-9-yl) 9-(4-(2-(2-(1-(2-((2-(4-(2-(2-(4-(nonanoyloxy)phenyl)acetoxy)ethyl)piperidin-
1-yl)ethyl)disulfaneyl)ethyl)piperidin-4-yl)ethoxy)-2-oxoethyl)phenyl) nonanedioate

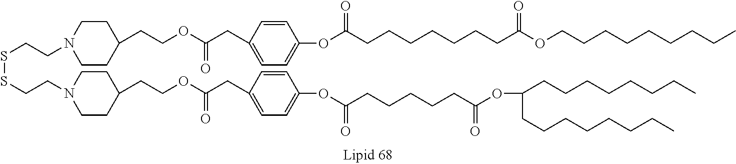 [SMILES: CCCCCCCCCOC(=O)CCCCCCCC(=O)Oc1ccc(CC(=O)OCCC2CCN(CCSSCCN3CCC(CCOC(=O)Cc4ccc(OC(=O)CCCCCC(=O)OC(CCCCCCCC)CCCCCCCC)cc4)CC3)CC2)cc1]

1-nonyl 9-(4-(2-(2-(1-(2-((2-(4-(2-(2-(4-((9-((3-octylundecyl)oxy)-9-
oxononanoyl)oxy)phenyl)acetoxy)ethyl)piperidin-1-yl)ethyl)disulfaneyl)ethyl)piperidin-4-
yl)ethoxy)-2-oxoethyl)phenyl) nonanedioate

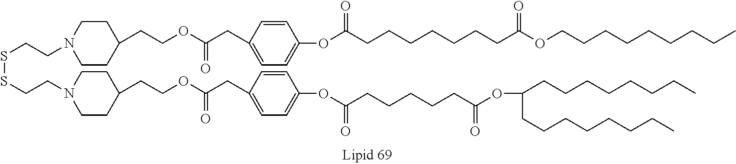 [SMILES: CCCCCCCCCOC(=O)CCCCCCCC(=O)Oc1ccc(CC(=O)OCCC2CCN(CCSSCCN3CCC(CCOC(=O)Cc4ccc(OC(=O)CCCCCC(=O)OC(CCCCCCCC)CCCCCCCC)cc4)CC3)CC2)cc1]

1-(4-(2-(2-(1-(2-((2-(4-(2-(2-(4-((7-(heptadecan-9-yloxy)-7-
oxoheptanoyl)oxy)phenyl)acetoxy)ethyl)piperidin-1-yl)ethyl)disulfaneyl)ethyl)piperidin-4-
yl)ethoxy)-2-oxoethyl)phenyl) 9-nonyl nonanedioate

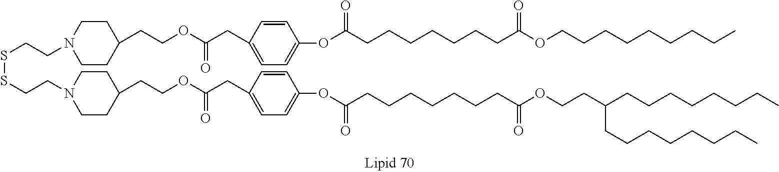 [SMILES: CCCCCCCCCOC(=O)CCCCCCCC(=O)Oc1ccc(CC(=O)OCCC2CCN(CCSSCCN3CCC(CCOC(=O)Cc4ccc(OC(=O)CCCCCCCC(=O)OCCC(CCCCCCCC)CCCCCCCC)cc4)CC3)CC2)cc1]

1-nonyl 9-(4-(2-(2-(1-(2-((2-(4-(2-(2-(4-((9-((3-octylundecyl)oxy)-9-
oxononanoyl)oxy)phenyl)acetoxy)ethyl)piperidin-1-yl)ethyl)disulfaneyl)ethyl)piperidin-4-
yl)ethoxy)-2-oxoethyl)phenyl) nonanedioate

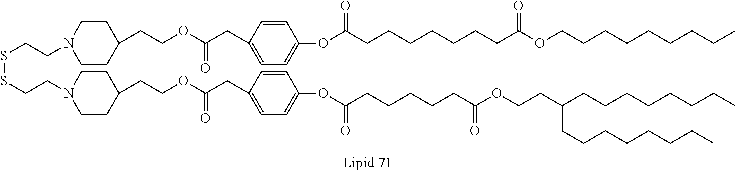 [SMILES: CCCCCCCCCOC(=O)CCCCCCCC(=O)Oc1ccc(CC(=O)OCCC2CCN(CCSSCCN3CCC(CCOC(=O)Cc4ccc(OC(=O)CCCCCC(=O)OCCC(CCCCCCCC)CCCCCCCC)cc4)CC3)CC2)cc1]

1-nonyl 9-(4-(2-(2-(1-(2-((2-(4-(2-(2-(4-((7-((3-octylundecyl)oxy)-7-
oxoheptanoyl)oxy)phenyl)acetoxy)ethyl)piperidin-1-yl)ethyl)disulfaneyl)ethyl)piperidin-4-
yl)ethoxy)-2-oxoethyl)phenyl) nonanedioate

### Formula (V)

In some aspects, the cationic lipids are of the Formula (V):

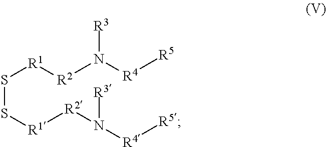

or a pharmaceutically acceptable salt thereof, wherein:
R¹ and R^{1′} are each independently (C₁-C₆)alkylene optionally substituted with one or more groups selected from R^{a};
R² and R^{2′} are each independently (C₁-C₂)alkylene;
R³ and R^{3′} are each independently (C₁-C₆)alkyl optionally substituted with one or more groups selected from R^{b};
or alternatively, R² and R³ and/or R^{2′} and R^{3′} are taken together with their intervening N atom to form a 4- to 7-membered heterocyclyl;
R⁴ and R^{4′} are each a (C₂-C₆)alkylene interrupted by —C(O)O—;
R⁵ and R^{5′} are each independently a (C₂-C₃₀)alkyl or (C₂-C₃₀)alkenyl, each of which are optionally interrupted with —C(O)O— or (C₃-C₆)cycloalkyl; and
R^{a} and R^{b} are each halo or cyano.

In a second chemical aspect, R¹ and R^{1′} in the cationic lipids of the Formula (V) each independently (C₁-C₆)alkylene, wherein the remaining variables are as described above for Formula (V). Alternatively, as part of a second chemical aspect, R¹ and R^{1′} in the cationic lipids of the Formula (V) each independently (C₁-C₃)alkylene, wherein the remaining variables are as described above for Formula (V).

In a third chemical aspect, the cationic lipids of the Formula (V) are of the Formula (VI):

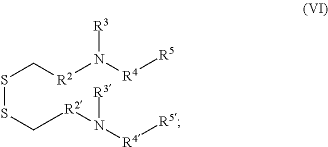

or a pharmaceutically acceptable salt thereof, wherein the remaining variables are as described above for Formula (V).

In a fourth chemical aspect, the cationic lipids of the Formula (V) are of the Formula (VII) or (VIII):

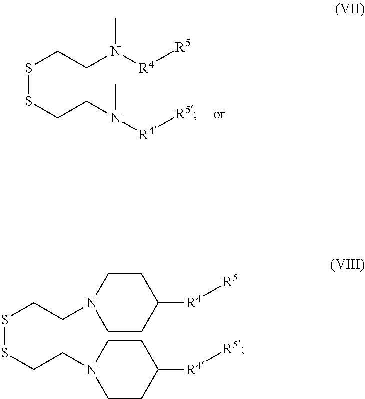

or a pharmaceutically acceptable salt thereof, wherein the remaining variables are as described above for Formula (V).

In a fifth chemical aspect, the cationic lipids of the Formula (V) are of the Formula (IX) or (VI):

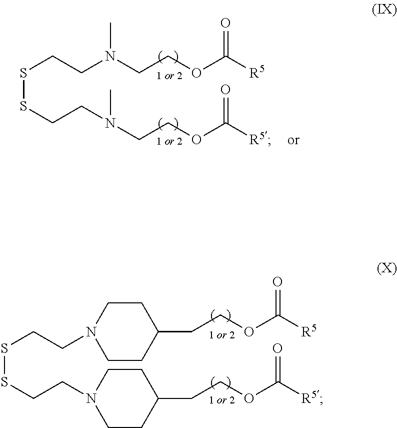

or a pharmaceutically acceptable salt thereof, wherein the remaining variables are as described above for Formula (V).

In a sixth chemical aspect, the cationic lipids of the Formula (V) are of the Formula (XI), (XII), (XIII), or (XIV):

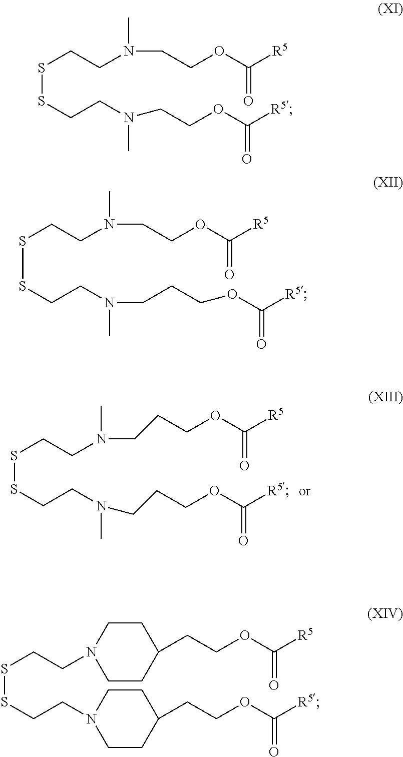

or a pharmaceutically acceptable salt thereof, wherein the remaining variables are as described above for Formula (XV).

In a seventh chemical aspect, at least one of R⁵ and R^{5′} in the cationic lipid of Formula (V), (VI), (VII), (VIII), (IX), (X), (XI), (XII), (XIII), or (XIV) is a branched alkyl or branched alkenyl (number of carbon atoms as described above for Formula (V), (VI), (VII), (VIII), (IX), (X), (XI), (XII), (XIII), or (XIV)). In another alternative, as part of a seventh chemical aspect, one of R⁵ and R^{5′} in the cationic lipid of Formula (V), (VI), (VII), (VIII), (IX), (X), (XI), (XII), (XIII), or (XIV) is a branched alkyl or branched alkenyl. In another alternative, as part of a seventh chemical aspect, R⁵ in the cationic lipid of Formula (V), (VI), (VII), (VIII), (IX), (X), (XI), (XII), (XIII), or (XIV) is a branched alkyl or branched alkenyl. In another alternative, as part of a seventh chemical aspect, R^{5′} in the cationic lipid of Formula (V), (VI), (VII), (VIII), (IX), (X), (XI), (XII), (XIII), or (XIV) is a branched alkyl or branched alkenyl.

In an eighth chemical aspect, R⁵ in the cationic lipid of Formula (V), (VI), (VII), (VIII), (IX), (X), (XI), (XII), (XIII), or (XIV) is a (C₆-C₂₆)alkyl or (C₆-C₂₆)alkenyl, each of which are optionally interrupted with —C(O)O— or (C₃-C₆)cycloalkyl, wherein the remaining variables are as described above for Formula (I). Alternatively, as part of a seventh chemical aspect, R⁵ in the cationic lipid of Formula (V), (VI), (VII), (VIII), (IX), (X), (XI), (XII), (XIII), or (XIV) is a (C₆-C₂₆)alkyl or (C₆-C₂₆)alkenyl, each of which are optionally interrupted with —C(O)O— or (C₃-C₅)cycloalkyl, wherein the remaining variables are as described above for Formula (V). In another alternative, as part of an eighth chemical aspect, R⁵ in the cationic lipid of Formula (V), (VI), (VII), (VIII), (IX), (X), (XI), (XII), (XIII), or (XIV) is a (C₇-C₂₆)alkyl or (C₇-C₂₆)alkenyl, each of which are optionally interrupted with —C(O)O— or (C₃-C₅)cycloalkyl, wherein the remaining variables are as described above for Formula (V). In another alternative, as part of an eighth chemical aspect, R⁵ in the cationic lipid of Formula (V), (VI), (VII), (VIII), (IX), (X), (XI), (XII), (XIII), or (XIV) is a (C₅-C₂₆)alkyl or (Cs-C₂₆)alkenyl, each of which are optionally interrupted with —C(O)O— or (C₃-C₅)cycloalkyl, wherein the remaining variables are as described above for Formula (V). In another alternative, as part of an eighth chemical aspect, R⁵ in the cationic lipid of Formula (V), (VI), (VII), (VIII), (IX), (X), (XI), (XII), (XIII), or (XIV) is a (C₆-C₂₄)alkyl or (C₆-C₂₄)alkenyl, each of which are optionally interrupted with —C(O)O— or cyclopropyl, wherein the remaining variables are as described above for Formula (V). In another alternative, as part of an eighth chemical aspect, R⁵ in the cationic lipid of Formula (V), (VI), (VII), (VIII), (IX), (X), (XI), (XII), (XIII), or (XIV) is a (C₅-C₂₄)alkyl or (C₅-C₂₄)alkenyl, wherein said (C₅-C₂₄)alkyl is optionally interrupted with —C(O)O— or cyclopropyl, wherein the remaining variables are as described above for Formula (V). In another alternative, as part of an eighth chemical aspect, R⁵ the cationic lipid of Formula (V), (VI), (VII), (VIII), (IX), (X), (XI), (XII), (XIII), or (XIV) is a (C₅-C₁₀)alkyl, wherein the remaining variables are as described above for Formula (V). In another alternative, as part of an eighthchemical aspect, R⁵ in the cationiclipid of Formula (V), (VI), (VII), (VIII), (IX), (X), (XI), (XII), (XIII), or (XIV) is a (C₁₄-C₁₆)alkyl interrupted with cyclopropyl, wherein the remaining variables are as described above for Formula (V). In another alternative, as part of an eighth chemical aspect, R⁵ in the cationic lipid of Formula (V), (VI), (VII), (VIII), (IX), (X), (XI), (XII), (XIII), or (XIV) is a (C₁₀-C₂₄)alkyl interrupted with —C(O)O—, wherein the remaining variables are as described above for Formula (V). In another alternative, as part of an eighth chemical aspect, R⁵ in the cationic lipid of Formula (V), (VI), (VII), (VIII), (IX), (X), (XI), (XII), (XIII), or (XIV) is a (C₁₆-C₁₅)alkenyl, wherein the remaining variables are as described above for Formula (V). In another alternative, as part of an eighth chemical aspect, R⁵ in the cationic lipid of Formula (V), (VI), (VII), (VIII), (IX), (X), (XI), (XII), (XIII), or (XIV) is —(CH₂)₃C(O)O(CH₂)₈CH₃, —(CH₂)₅C(O)O(CH₂)₈CH₃,—(CH₂)₇C(O)O(CH₂)₈CH₃, —(CH₂)₇C(O)OCH[(CH₂)₇CH₃]2, —(CH₂)₇-C₃H6-(CH₂)₇CH₃, —(CH₂)₇CH₃, —(CH₂)₉CH₃,—(CH₂)₁₆CH₃, —(CH₂)₇CH═CH(CH₂)₇CH₃, or —(CH₂)₇CH═CHCH₂CH═CH(CH₂)₄CH₃, wherein the remaining variables are as described above for Formula (XV).

In a ninth chemical aspect, R^{5′} in the cationic lipid of Formula (V), (VI), (VII), (VIII), (IX), (X), (XI), (XII), (XIII), or (XIV) is a (C₁₅-C₂₈)alkyl interrupted with —C(O)O—, wherein the remaining variables are as described above for Formula (V) or the eighth chemical aspect. Alternatively, as part of a ninth chemical aspect, R^{5′} in the cationic lipid of Formula (V), (VI), (VII), (VIII), (IX), (X), (XI), (XII), (XIII), or (XIV) is a (C₁₇-C₂₈)alkyl interrupted with —C(O)O—, wherein the remaining variables are as described above for Formula (V) or the eighth chemical aspect. In another alternative, as part of a ninth embodiment, R^{5′} in the cationic lipid of Formula (V), (VI), (VII), (VIII), (IX), (X), (XI), (XII), (XIII), or (XIV) is a (C₁₉-C₂₈)alkyl interrupted with —C(O)O—, wherein the remaining variables are as described above for Formula (V) or the eighth chemical aspect. In another alternative, as part of a ninth chemical aspect, R^{5′} in the cationic lipid of Formula (V), (VI), (VII), (VIII), (IX), (X), (XI), (XII), (XIII), or (XIV) is a (C₁₇-C₂₆)alkyl interrupted with —C(O)O—, wherein the remaining variables are as described above for Formula (V) or the eighth chemical aspect. In another alternative, as part of a ninth embodiment, R^{5′} in the cationic lipid of Formula (V), (VI), (VII), (VIII), (IX), (X), (XI), (XII), (XIII), or (XIV) is a (C₁₉-C₂₆)alkyl interrupted with —C(O)O—, wherein the remaining variables are as described above for Formula (V) or the eighth chemical aspect. In another alternative, as part of a ninth chemical aspect, R^{5′} in the cationic lipid of Formula (V), (VI), (VII), (VIII), (IX), (X), (XI), (XII), (XIII), or (XIV) is a (C₂₀-C₂₆)alkyl interrupted with —C(O)O—, wherein the remaining variables are as described above for Formula (V) or the eighth chemical aspect. In another alternative, as part of a ninth embodiment, R^{5′} is a (C₂₂-C₂₄)alkyl interrupted with —C(O)O—, wherein the remaining variables are as described above for Formula (V) or the eighth chemical aspect. In another alternative, as part of a ninth embodiment, R^{1′} is —(CH₂)₅C(O)OCH[(CH₂)₇CH₃]2, -(CH₂)₇C(O)OCH[(CH₂)₇CH₃]2, —(CH₂)₅C(O)OCH(CH₂)₂[(CH₂)₇CH₃]2, or -(CH₂)₇C(O)OCH(CH₂)₂[(CH₂)₇CH₃]2, wherein the remaining variables are as described above for Formula (V) or the eighth chemical aspect.

In another aspect, the cationic lipid of Formula (V), (VI), (VIII), (VIII), (IX), (X), (XII), (XIII), or (XIV) may be selected from any of the following lipids in Table 6 or a pharmaceutically acceptable salt thereof.

TABLE 6
Exemplary cationic lipids of Formula (V), (VI), (VIII), (VIII), (IX), (X), (XII), (XIII), or (XIV)

| Lipid No. | Lipid Structure and Name |
|---|---|
| 72 | 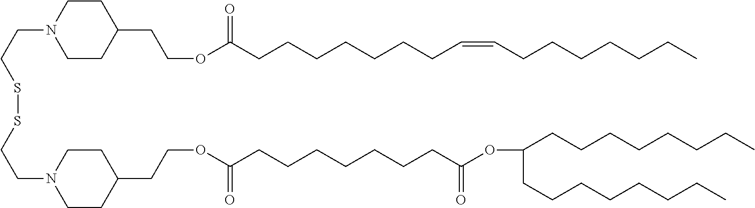 [SMILES: CCCCCCC/C=C\CCCCCCCC(=O)OCCC1CCN(CCSSCCN2CCC(CCOC(=O)CCCCCCCC(=O)OC(CCCCCCCC)CCCCCCCC)CC2)CC1] |
| | (Z)-1-(2-(1-(2-((2-(4-(2-(heptadec-9-enoyloxy)ethyl)piperidin-1- |
| | yl)ethyl)disulfaneyl)ethyl)piperidin-4-yl)ethyl) 9-(heptadecan-9-yl) |
| | nonanedioate |
| 73 | 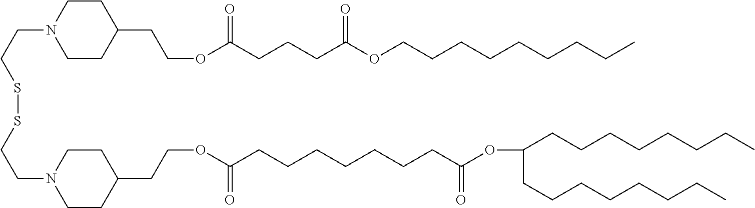 [SMILES: CCCCCCCCCOC(=O)CCCC(=O)OCCC1CCN(CCSSCCN2CCC(CCOC(=O)CCCCCCCC(=O)OC(CCCCCCCC)CCCCCCCC)CC2)CC1] |
| | 1-(heptadecan-9-yl) 9-(2-(1-(2-((2-(4-(2-((5-(nonyloxy)-5- |
| | oxopentanoyl)oxy)ethyl)piperidin-1-yl)ethyl)disulfaneyl)ethyl)piperidin-4- |
| | yl)ethyl) nonanedioate |
| 74 | 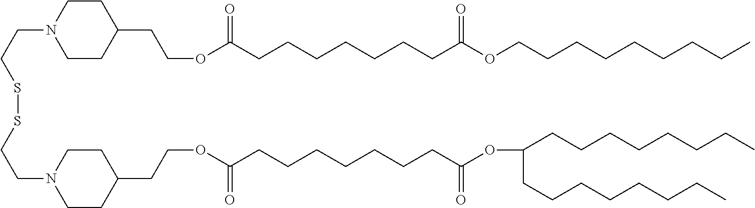 [SMILES: CCCCCCCCCOC(=O)CCCCCCCC(=O)OCCC1CCN(CCSSCCN2CCC(CCOC(=O)CCCCCCCC(=O)OC(CCCCCCCC)CCCCCCCC)CC2)CC1] |
| | 1-(heptadecan-9-yl) 9-(2-(1-(2-((2-(4-(2-((9-(nonyloxy)-9- |
| | oxononanoyl)oxy)ethyl)piperidin-1-yl)ethyl)disulfaneyl)ethyl)piperidin-4- |
| | yl)ethyl) nonanedioate |
| 75 | 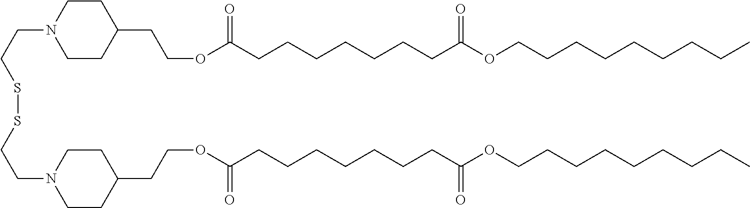 [SMILES: CCCCCCCCCOC(=O)CCCCCCCC(=O)OCCC1CCN(CCSSCCN2CCC(CCOC(=O)CCCCCCCC(=O)OCCCCCCCCC)CC2)CC1] |
| | O′1,O1-(((disulfanediylbis(ethane-2,1-diyl))bis(piperidine-1,4- |
| | diyl))bis(ethane-2,1-diyl)) 9,9′-dinonyl di(nonanedioate) |
| 76 | 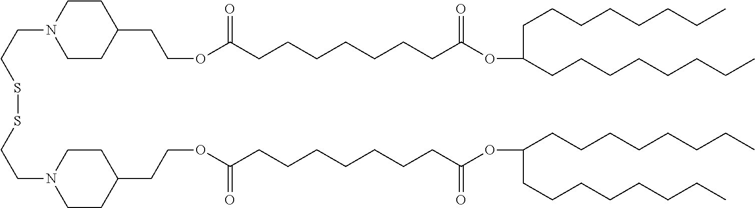 [SMILES: CCCCCCCCC(CCCCCCCC)OC(=O)CCCCCCCC(=O)OCCC1CCN(CCSSCCN2CCC(CCOC(=O)CCCCCCCC(=O)OC(CCCCCCCC)CCCCCCCC)CC2)CC1] |
| | O′1,O1-(((disulfanediylbis(ethane-2,1-diyl))bis(piperidine-1,4- |
| | diyl))bis(ethane-2,1-diyl)) 9,9′-di(heptadecan-9-yl) di(nonanedioate) |

### Formula (XV)

In some aspects, the cationic lipids are of the Formula (XV):

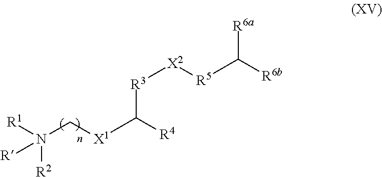

or a pharmaceutically acceptable salt thereof, wherein:
R′ is absent, hydrogen, or C₁-C₆ alkyl; provided that when R′ is hydrogen or C₁-C₆ alkyl, the nitrogen atom to which R′, R¹, and R² are all attached is protonated;
R¹ and R² are each independently hydrogen, C₁-C₆ alkyl, or C₂-C₆ alkenyl;
R³ is C₁-C₁₂ alkylene or C₂-C₁₂ alkenylene;
R⁴ is C₁-C₁₆unbranched alkyl, C₂-C₁₆unbranched alkenyl, or

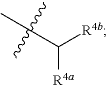

wherein:
R^{4a} and R^{4b} are each independently C₁-C₁₆unbranched alkyl or C₂-C₁₆unbranched alkenyl;
R⁵ is absent, C₁-C₅ alkylene, or C₂-C₅ alkenylene;
R^{6a} and R^{6b} are each independently C₇-C₁₆ alkyl or C₇-C₁₆ alkenyl; provided that the total number of carbon atoms in R^{6a} and R^{6b} as combined is greater than 15;
X¹ and X² are each independently —OC(═O)—, —SC(═O)—, —OC(═S)—, —C(═O)O—, —C(═O)S—, —S—S—, —C(R^{a})═N—, —N═C(R^{a})—, —C(R^{a})═NO—, —O—N═C(R^{a})—, —C(═O)NR^{a}—, —NR^{a}C(═O)—, —NR^{a}C(═O)NR^{a}—, —OC(═O)O—, —OSi(R^{a})₂O—, —C(═O)(CR^{a}₂)C(═O)O—, or OC(═O)(CR^{a}₂)C(═O)—; wherein:
   R^{a}, for each occurrence, is independently hydrogen or C₁-C₆ alkyl; and
n is an integer selected from 1, 2, 3, 4, 5, and 6.

In a second embodiment, in the cationic lipid according to the first embodiment, or a pharmaceutically acceptable salt thereof, X¹ and X² are the same; and all other remaining variables are as described for Formula (V) or the first embodiment.

In a third embodiment, in the cationic lipid according to the first or second embodiment, or a pharmaceutically acceptable salt thereof, X¹ and X² are each independently —OC(═O)—, —SC(═O)—, —OC(═S)—, —C(═O)O—, —C(═O)S—, or —S—S—; or X¹ and X² are each independently —C(═O)O—, —C(═O)S—, or —S—S—; or X¹ and X² are each independently —C(═O)O- or —S—S—; and all other remaining variables are as described for Formula V or any one of the preceding embodiments.

In a fourth embodiment, the cationic lipid of the present disclosure is represented by Formula (XVI):

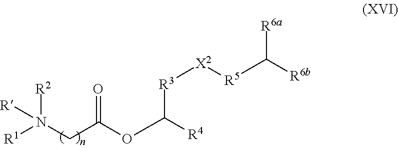

or a pharmaceutically acceptable salt thereof, wherein n is an integer selected from 1, 2, 3, and 4; and all other remaining variables are as described for Formula (XV) or any one of the preceding embodiments.

In a fifth embodiment, the cationic lipid of the present disclosure is represented by Formula (XVII):

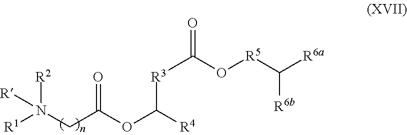

or a pharmaceutically acceptable salt thereof, wherein n is an integer selected from 1, 2, and 3; and all other remaining variables are as described for Formula (XV), Formula (XVI) or any one of the preceding embodiments.

In a sixth embodiment, the cationic lipid of the present disclosure is represented by Formula (XVIII):

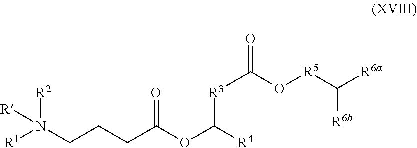

or a pharmaceutically acceptable salt thereof; and all other remaining variables are as described for Formula (XV), Formula (XVI), Formula (XVII) or any one of the preceding embodiments.

In a seventh embodiment, in the cationic lipid according to Formula (XV), Formula (XVI), Formula (XVII), Formula (XVIII) or any one of the preceding embodiments, or a pharmaceutically acceptable salt thereof, R¹ and R² are each independently hydrogen, C₁-C₆ alkyl or C₂-C₆ alkenyl, or C₁-C₅ alkyl or C₂-C₅ alkenyl, or C₁-C₄ alkyl or C₂-C₄ alkenyl, or C₆ alkyl, or C₅ alkyl, or C₄ alkyl, or C₃ alkyl, or C₂ alkyl, or C₁ alkyl, or C₆ alkenyl, or C₅ alkenyl, or C₄ alkenyl, or C₃ alkenyl, or C₂ alkenyl; and all other remaining variables are as described for Formula (XV), Formula (XVI), Formula (XVII), Formula (XVIII) or any one of the preceding embodiments.

In an eighth embodiment, the cationic lipid of the present disclosure is represented by Formula (XIX):

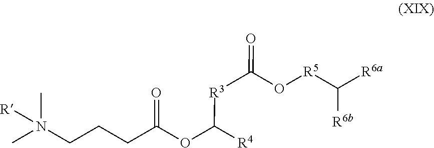

or a pharmaceutically acceptable salt thereof; and all other remaining variables are as described for Formula (XV), Formula (XVI), Formula (XVII), Formula (XVIII) or any one of the preceding embodiments.

In a ninth embodiment, in the cationic lipid according to Formula (XV), Formula (XVI), Formula (XVII), Formula (XVIII), Formula (XIX) or any one of the preceding embodiments, or a pharmaceutically acceptable salt thereof, R³ is C₁-C₉ alkylene or C₂-C₉ alkenylene, C₁-C₇ alkylene or C₂-C₇ alkenylene, C₁-C₅ alkylene or C₂-C₅ alkenylene, or C₂-C₅ alkylene or C₂-C₅ alkenylene, or C₃-C₇ alkylene or C₃-C₇ alkenylene, or C₅-C₇ alkylene or C₅-C₇ alkenylene; or R³ is C₁₂ alkylene, C₁₁alkylene, C₁₀ alkylene, C₉ alkylene, or C₅ alkylene, or C₇ alkylene, or C₆ alkylene, or C₅ alkylene, or C₄ alkylene, or C₃ alkylene, or C₂ alkylene, or C₁ alkylene, or C₁₂ alkenylene, C₁₁ alkenylene, C₁₀ alkenylene, C₉ alkenylene, or C₈ alkenylene, or C₇ alkenylene, or C₆ alkenylene, or C₅ alkenylene, or C₄ alkenylene, or C₃ alkenylene, or C₂ alkenylene; and all other remaining variables are as described for Formula (XV), Formula (XVI), Formula (XVII), Formula (XVIII), Formula (XIX) or any one of the preceding embodiments.

In a tenth embodiment, in the cationic lipid according to Formula (XV), Formula (XVI), Formula (XVII), Formula (XVIII), Formula (XIX) or any one of the preceding embodiments, or a pharmaceutically acceptable salt thereof, R⁵ is absent, C₁-C₆ alkylene, or C₂-C₆ alkenylene; or R⁵ is absent, C₁-C₄ alkylene, or C₂-C₄ alkenylene; or R⁵ is absent; or R⁵ is C₈ alkylene, C₇ alkylene, C₆ alkylene, C₅ alkylene, C₄ alkylene, C₃ alkylene, C₂ alkylene, C₁ alkylene, C₈ alkenylene, C₇ alkenylene, C₆ alkenylene, C₈ alkenylene, C₄ alkenylene, C₃ alkenylene, or C₂ alkenylene; and all other remaining variables are as described for Formula (XV), Formula (XVI), Formula (XVII), Formula (XVIII), Formula (XIX) or any one of the preceding embodiments.

In an eleventh embodiment, in the cationic lipid according to Formula (XV), Formula (XVI), Formula (XVII), Formula (XVIII), Formula (XIX) or any one of the preceding embodiments, or a pharmaceutically acceptable salt thereof, R⁴ is C₁-C₁₄unbranched alkyl, C₂-C₁₄unbranched alkenyl, or

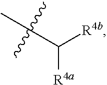

wherein R^{4a} and R^{4b} are each independently C₁-C₁₂unbranched alkyl or C₂-C₁₂ unbranched alkenyl; or R⁴ is C₂-C₁₂unbranched alkyl or C₂-C₁₂unbranched alkenyl; or R₄ is C₅-C₇ unbranched alkyl or C₅-C₇unbranched alkenyl; or R⁴ is C₁₆unbranched alkyl, C₁₅unbranched alkyl, C₁₄unbranched alkyl, C₁₃unbranched alkyl, C₁₂unbranched alkyl, C₁₁ unbranched alkyl, C₁₀ unbranched alkyl, C₉unbranched alkyl, C₈ unbranched alkyl, C₇unbranched alkyl, C₆ unbranched alkyl, C₈ unbranched alkyl, C₄unbranched alkyl, C₃unbranched alkyl, C₂unbranched alkyl, C₁unbranched alkyl, C₁₆unbranched alkenyl, C₁₅unbranched alkenyl, C₁₄unbranched alkenyl, C₁₃ unbranched alkenyl, C₁₂unbranched alkenyl, C₁₁ unbranched alkenyl, C₁₀ unbranched alkenyl, C₉ unbranched alkenyl, C₈ unbranched alkenyl, C₇unbranched alkenyl, C₆ unbranched alkenyl, C₅ unbranched alkenyl, C₄unbranched alkenyl, C₃unbranched alkenyl, or C₂ alkenyl; or R⁴ is

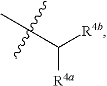

wherein R^{4a} and R^{4b} are each independently C₂-C₁₀ unbranched alkyl or C₂-C₁₀ unbranched alkenyl; or R⁴ is

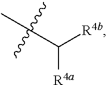

wherein R^{4a} and R^{4b} are each independently C₁₆unbranched alkyl, C₁₅ unbranched alkyl, C₁₄ unbranched alkyl, C₁₃ unbranched alkyl, C₁₂unbranched alkyl, C₁₁unbranched alkyl, C₁₀unbranched alkyl, C₉unbranched alkyl, C₈ unbranched alkyl, C₇unbranched alkyl, C₆ unbranched alkyl, C₈ unbranched alkyl, C₄unbranched alkyl, C₃unbranched alkyl, C₂ alkyl, C₁ alkyl, C₁₆unbranched alkenyl, C₁₅unbranched alkenyl, C₁₄unbranched alkenyl, C₁₃unbranched alkenyl, C₁₂unbranched alkenyl, C₁₁ unbranched alkenyl, C₁₀unbranched alkenyl, C₉unbranched alkenyl, C₈ unbranched alkenyl, C₇unbranched alkenyl, C₆ unbranched alkenyl, C₈ unbranched alkenyl, C₄unbranched alkenyl, C₃unbranched alkenyl, or C₂ alkenyl; and all other remaining variables are as described for Formula (XV), Formula (XVI), Formula (XVII), Formula (XVIII), Formula (XIX) or any one of the preceding embodiments.

In a twelfth embodiment, in the cationic lipid according to Formula (XV), Formula (XVI), Formula (XVII), Formula (XVIII), Formula (XIX) or any one of the preceding embodiments, or a pharmaceutically acceptable salt thereof, R^{6a} and R^{6b} are each independently C₆-C₁₄ alkyl or C₆-C₁₄ alkenyl; or R^{6a} and R^{6b} are each independently C₈-C₁₂ alkyl or C₈-C₁₂ alkenyl; or R^{6a} and R^{6b} are each independently C₁₆ alkyl, C₁₅ alkyl, C₁₄ alkyl, C₁₃ alkyl, C₁₂ alkyl, C₁₁ alkyl, C₁₀ alkyl, C₉ alkyl, C₈ alkyl, C₇ alkyl, C₁₆ alkenyl, C₁₅ alkenyl, C₁₄ alkenyl, C₁₃ alkenyl, C₁₂ alkenyl, C₁₁ alkenyl, C₁₀ alkenyl, C₉ alkenyl, C₈ alkenyl, or C₇ alkenyl; provided that the total number of carbon atoms in R^{6a} and R^{6b} as combined is greater than 15; and all other remaining variables are as described for Formula (XV), Formula (XVI), Formula (XVII), Formula (XVIII), Formula (XIX) or any one of the preceding embodiments.

In a thirteenth embodiment, in the cationic lipid according to Formula (XV), Formula (XVI), Formula (XVII), Formula (XVIII), Formula (XIX) or any one of the preceding embodiments, or a pharmaceutically acceptable salt thereof, R^{6a} and R^{6b} contain an equal number of carbon atoms with each other; or R^{6a} and R^{6b} are the same; or R^{6a} and R^{6b} are both C₁₆ alkyl, C₁₅ alkyl, C₁₄ alkyl, C₁₃ alkyl, C₁₂ alkyl, C₁₁ alkyl, C₁₁ alkyl, C₉ alkyl, C₈ alkyl, C₇ alkyl, C₁₆ alkenyl, C₁₅ alkenyl, C₁₄ alkenyl, C₁₃ alkenyl, C₁₂ alkenyl, C₁₁ alkenyl, C₁₀ alkenyl, C₉ alkenyl, C₈ alkenyl, or C₇ alkenyl; provided that the total number of carbon atoms in R^{6a} and R^{6b} as combined is greater than 15; and all other remaining variables are as described for Formula (XV), Formula (XVI), Formula (XVII), Formula (XVIII), Formula (XIX) or any one of the preceding embodiments.

In a fourteenth embodiment, in the cationic lipid according to Formula (XV), Formula (XVI), Formula (XVII), Formula (XVIII), Formula (XIX) or any one of the preceding embodiments, or a pharmaceutically acceptable salt thereof, R^{6a} and R^{6b} as defined in any one of the preceding embodiments each contain a different number of carbon atoms with each other; or the number of carbon atoms R^{6a} and R^{6b} differs by one or two carbon atoms; or the number of carbon atoms R^{6a} and R^{6b} differs by one carbon atom; or R^{6a} is C₇ alkyl and R^{6a} is C₈ alkyl, R^{6a} is C₈ alkyl and R^{6a} is C₇ alkyl, R^{6a} is C₈ alkyl and R^{6a} is C₉ alkyl, R^{6a} is C₉ alkyl and R^{6a} is C₈ alkyl, R^{6a} is C₉ alkyl and R^{6a} is C₁₀ alkyl, R^{6a} is C₁₁ alkyl and R^{6a} is C₉ alkyl, R^{6a} is C₁₁ alkyl and R^{6a} is C₁₁ alkyl, R^{6a} is C₁, alkyl and R^{6a} is C₁₀ alkyl, R^{6a} is C₁, alkyl and R^{6a} is C₁₂ alkyl, R^{6a} is C₁₂ alkyl and R^{6a} is C₁₁ alkyl, R^{6a} is C₇ alkyl and R^{6a} is C₉ alkyl, R^{6a} is C₉ alkyl and R^{6a} is C₇ alkyl, R^{6a} is C₈ alkyl and R^{6a} is C₁₁ alkyl, R^{6a} is C₁₀ alkyl and R^{6a} is C₈ alkyl, R^{6a} is C₉ alkyl and R^{6a} is C₁₁ alkyl, R^{6a} is C₁ alkyl and R^{6a} is C₉ alkyl, R^{6a} is C₁₀ alkyl and R^{6a} is C₁₂ alkyl, R⁶ is C₁₂ alkyl and R^{6a} is C₁₁alkyl, R^{6a} is C₁₃ alkyl and R^{6a} is C₁₃ alkyl, or R^{6a} is C₁₃ alkyl and R^{6a} is C₁h alkyl, etc.; and all other remaining variables are as described for Formula I, Formula II, Formula III, Formula IV, Formula V or any one of the preceding embodiments.

In one embodiment, the cationic lipid of the present disclosure or the cationic lipid of Formula (XV), Formula (XVI), Formula (XVII), Formula (XVIII), or Formula (XIX) is any one lipid selected from the lipids in Table 7 or a pharmaceutically acceptable salt thereof:

TABLE 7
Exemplary lipids of Formula (XV), Formula (XVI), Formula (XVII), Formula (XVIII), Formula (XIX)

| Lipid | |
|---|---|
| No. | Lipid Structure and Name |
| 77 | 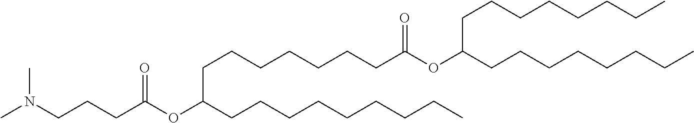 [SMILES: CCCCCCCCCC(CCCCCCCC(=O)OC(CCCCCCCC)CCCCCCCC)OC(=O)CCCN(C)C] |
| | heptadecan-9-yl 9-((4-(dimethylamino)butanoyl)oxy)octadecanoate |
| 78 | 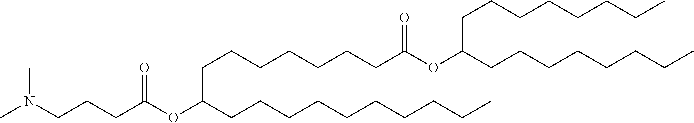 [SMILES: CCCCCCCCCCC(CCCCCCCC(=O)OC(CCCCCCCC)CCCCCCCC)OC(=O)CCCN(C)C] |
| | heptadecan-9-yl 9-((4-(dimethylamino)butanoyl)oxy)nonadecanoate |
| 79 | 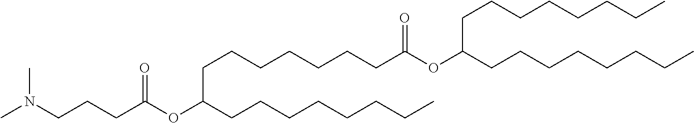 [SMILES: CCCCCCCCC(CCCCCCCC)OC(=O)CCCCCCCC(CCCCCCCC)OC(=O)CCCN(C)C] |
| | heptadecan-9-yl 9-((4-(dimethylamino)butanoyl)oxy)heptadecanoate |
| 80 | 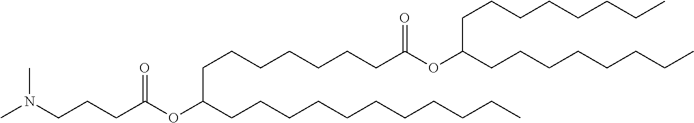 [SMILES: CCCCCCCCCCCC(CCCCCCCC(=O)OC(CCCCCCCC)CCCCCCCC)OC(=O)CCCN(C)C] |
| | heptadecan-9-yl 9-((4-(dimethylamino)butanoyl)oxy)icosanoate |
| 81 | 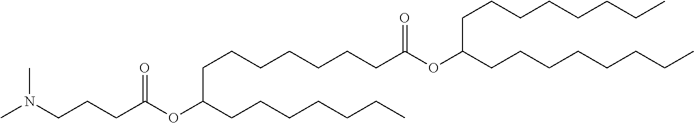 [SMILES: CCCCCCCCC(CCCCCCCC)OC(=O)CCCCCCCC(CCCCCCC)OC(=O)CCCN(C)C] |
| | heptadecan-9-yl 9-((4-(dimethylamino)butanoyl)oxy)hexadecanoate |
| 82 | 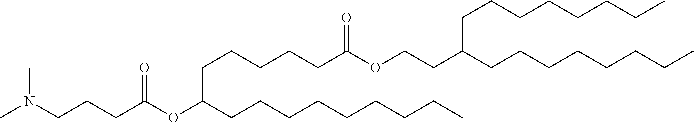 [SMILES: CCCCCCCCCC(CCCCCC(=O)OCCC(CCCCCCCC)CCCCCCCC)OC(=O)CCCN(C)C] |
| | 3-octylundecyl 7-((4-(dimethylamino)butanoyl)oxy)hexadecanoate |
| 83 | 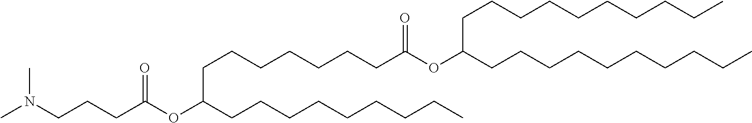 [SMILES: CCCCCCCCCCC(CCCCCCCCCC)OC(=O)CCCCCCCC(CCCCCCCCC)OC(=O)CCCN(C)C] |
| | henicosan-11-yl 9-((4-(dimethylamino)butanoyl)oxy)octadecanoate |
| 84 | 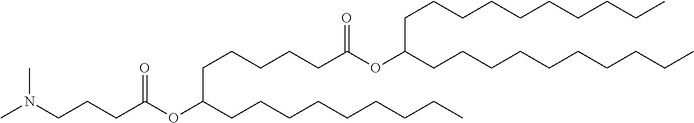 [SMILES: CCCCCCCCCCC(CCCCCCCCCC)OC(=O)CCCCCC(CCCCCCCCC)OC(=O)CCCN(C)C] |
| | henicosan-11-yl 7-((4-(dimethylamino)butanoyl)oxy)hexadecanoate |
| 85 | 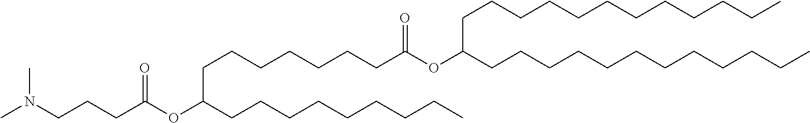 [SMILES: CCCCCCCCCCCCC(CCCCCCCCCCCC)OC(=O)CCCCCCCC(CCCCCCCCC)OC(=O)CCCN(C)C] |
| | pentacosan-13-yl 9-((4-(dimethylamino)butanoyl)oxy)octadecanoate |
| 86 | 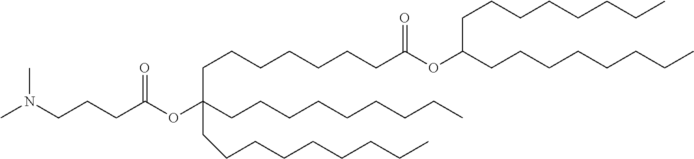 [SMILES: CCCCCCCCCC(CCCCCCCCC)(CCCCCCCC(=O)OC(CCCCCCCC)CCCCCCCC)OC(=O)CCCN(C)C] |
| | heptadecan-9-yl 9-((4-(dimethylamino)butanoyl)oxy)-9-nonyloctadecanoate |
| 87 | 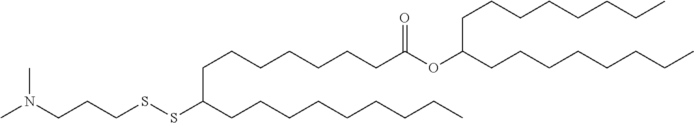 [SMILES: CCCCCCCCCC(CCCCCCCC(=O)OC(CCCCCCCC)CCCCCCCC)SSCCCN(C)C] |
| | heptadecan-9-yl 9-((3-(dimethylamino)propyl)disulfaneyl)octadecanoate |

### Formula (XX)

In some aspects, the cationic lipids are of the Formula (XX):

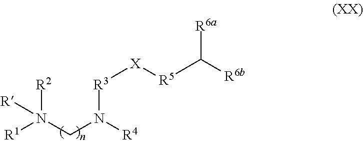

or a pharmaceutically acceptable salt thereof, wherein:
R′ is absent, hydrogen, or C₁-C₃ alkyl; provided that when R′ is hydrogen or C₁-C₃ alkyl, the nitrogen atom to which R′, R¹, and R² are all attached is protonated;
R¹ and R² are each independently hydrogen or C₁-C₃ alkyl;
R³ is C₃-C₁₀ alkylene or C₃-C₁₀ alkenylene;
R⁴ is C₁-C₁₆unbranched alkyl, C₂-C₁₆unbranched alkenyl, or

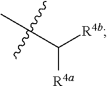

wherein:
R^{4a} and R^{4b} are each independently C₁-C₁₆unbranched alkyl or C₂-C₁₆unbranched alkenyl;
R⁵ is absent, C₁-C₆ alkylene, or C₂-C₆ alkenylene;
R^{6a} and R^{6b} are each independently C₇-C₁₄ alkyl or C₇-C₁₄ alkenyl;
X is —OC(═O)—, —SC(═O)—, —OC(═S)—, —C(═O)O—, —C(═O)S—, —S—S—, —C(R^{a})═N—, —N═C(R^{a})—, —C(R^{a})═NO—, —O—N═C(R^{a})—, —C(═O)NR^{a}—, —NR^{a}C(═O)—, —NR^{a}C(═O)NR^{a}—, —OC(═O)O—, —OSi(R^{a})₂O—, —C(═O)(CR^{a}₂)C(═O)O—, or OC(═O)(CR^{a}₂)C(═O)—; wherein:
   R^{a}, for each occurrence, is independently hydrogen or C₁-C₆ alkyl; and
n is an integer selected from 1, 2, 3, 4, 5, and 6.

In a second embodiment, in the cationic lipid according to the first embodiment, or a pharmaceutically acceptable salt thereof, X is —OC(═O)—, —SC(═O)—, —OC(═S)—, —C(═O)O—, —C(═O)S—, or —S—S—; and all other remaining variables are as described for Formula I or the first embodiment.

In a third embodiment, the cationic lipid of the present disclosure is represented by Formula (XXI):

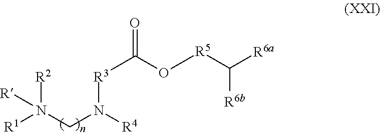

or a pharmaceutically acceptable salt thereof, wherein n is an integer selected from 1, 2, 3, and 4; and all other remaining variables are as described for Formula (XX) or any one of the preceding embodiments. In an alternative third embodiment, n is an integer selected from 1, 2, and 3; and all other remaining variables are as described for Formula (XX) or any one of the preceding embodiments.

In a fourth embodiment, the cationic lipid of the present disclosure is represented by Formula (XXII):

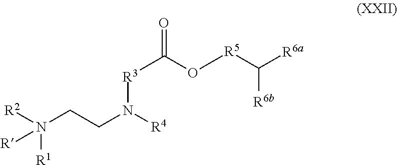

or a pharmaceutically acceptable salt thereof; and all other remaining variables are as described for Formula (XX), Formula (XXI) or any one of the preceding embodiments.

In a fifth embodiment, in the cationic lipid according to the first embodiment, or a pharmaceutically acceptable salt thereof, R¹ and R² are each independently hydrogen or C₁-C₂ alkyl, or C₂-C₃ alkenyl; or R′, R¹, and R² are each independently hydrogen, C₁-C₂ alkyl; and all other remaining variables are as described for Formula (XX), Formula (XXI) or any one of the preceding embodiments.

In a sixth embodiment, the cationic lipid of the present disclosure is represented by Formula (XXII):

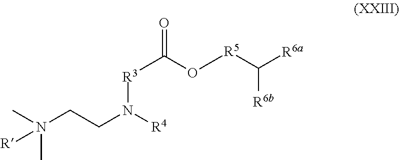

or a pharmaceutically acceptable salt thereof; and all other remaining variables are as described for Formula (XX), Formula (XXI), Formula (XXII) or any one of the preceding embodiments.

In a seventh embodiment, in the cationic lipid according to Formula (XX), Formula (XXI), Formula (XXII), Formula (XXIII) or any one of the preceding embodiments, or a pharmaceutically acceptable salt thereof, R⁵ is absent or C₁-C₈ alkylene; or R⁵ is absent, C₁-C₆ alkylene, or C₂-C₆ alkenylene; or R⁵ is absent, C₁-C₄ alkylene, or C₂-C₄ alkenylene; or R⁵ is absent; or R⁵ is C₈ alkylene, C₇ alkylene, C₆ alkylene, C₅ alkylene, C₄ alkylene, C₃ alkylene, C₂ alkylene, C₁ alkylene, C₈ alkenylene, C₇ alkenylene, C₆ alkenylene, C₅ alkenylene, C₄ alkenylene, C₃ alkenylene, or C₂ alkenylene; and all other remaining variables are as described for Formula (XX), Formula (XXI), Formula (XXII), Formula (XXIII) or any one of the preceding embodiments.

In an eighth embodiment, the cationic lipid of the present disclosure is represented by Formula (XXIV):

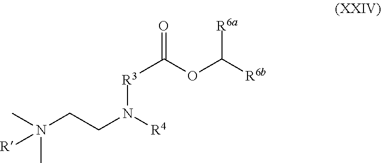

or a pharmaceutically acceptable salt thereof; and all other remaining variables are as described for Formula (XX), Formula (XXI), Formula (XXII), Formula (XXIII) or any one of the preceding embodiments.

In a ninth embodiment, in the cationic lipid according to Formula (XX), Formula (XXI), Formula (XXII), Formula (XXIII), Formula (XXIV) or any one of the preceding embodiments, or a pharmaceutically acceptable salt thereof, R⁴ is C₁-C₁₄unbranched alkyl, C₂-C₁₄unbranched alkenyl, or

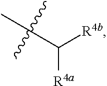

wherein R^{4a} and R^{4b} are each independently C₁-C₁₂unbranched alkyl or C₂-C₁₂ unbranched alkenyl; or R⁴ is C₂-C₁₂unbranched alkyl or C₂-C₁₂unbranched alkenyl; or R⁴ is C₅-C₁₂ unbranched alkyl or C₈-C₁₂unbranched alkenyl; or R⁴ is C₁₆unbranched alkyl, C₁₅ unbranched alkyl, C₁₄unbranched alkyl, C₁₃unbranched alkyl, C₁₂unbranched alkyl, C₁₁ unbranched alkyl, C₁₀ unbranched alkyl, C₉unbranched alkyl, C₈ unbranched alkyl, C₇unbranched alkyl, C₆ unbranched alkyl, C₈ unbranched alkyl, C₄unbranched alkyl, C₃unbranched alkyl, C₂unbranched alkyl, C₁ unbranched alkyl, C₁₆unbranched alkenyl, C₁₅unbranched alkenyl, C₁₄unbranched alkenyl, C₁₃ unbranched alkenyl, C₁₂unbranched alkenyl, C₁₁ unbranched alkenyl, C₁₀ unbranched alkenyl, C₉ unbranched alkenyl, C₈ unbranched alkenyl, C₇unbranched alkenyl, C₆ unbranched alkenyl, C₅ unbranched alkenyl, C₄unbranched alkenyl, C₃unbranched alkenyl, or C₂ alkenyl; or R⁴ is

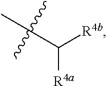

wherein R^{4a} and R^{4b} are each independently C₂-C₁₀ unbranched alkyl or C₂-C₁₀ unbranched alkenyl; or R⁴ is

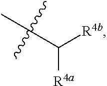

wherein R^{4a} and R^{4b} are each independently C₁₆unbranched alkyl, C₁₅ unbranched alkyl, C₁₄ unbranched alkyl, C₁₃ unbranched alkyl, C₁₂unbranched alkyl, C₁₁unbranched alkyl, C₁₀unbranched alkyl, C₉unbranched alkyl, C₈ unbranched alkyl, C₇unbranched alkyl, C₆ unbranched alkyl, C₈ unbranched alkyl, C₄unbranched alkyl, C₃unbranched alkyl, C₂ alkyl, C₁ alkyl, C₁₆unbranched alkenyl, C₁₅unbranched alkenyl, C₁₄unbranched alkenyl, C₁₃unbranched alkenyl, C₁₂unbranched alkenyl, C₁₁ unbranched alkenyl, C₁₀unbranched alkenyl, C₉unbranched alkenyl, C₈ unbranched alkenyl, C₇unbranched alkenyl, C₆ unbranched alkenyl, C₈ unbranched alkenyl, C₄unbranched alkenyl, C₃unbranched alkenyl, or C₂ alkenyl; and all other remaining variables are as described for Formula (XX), Formula (XXI), Formula (XXII), Formula (XXIII), Formula (XXIV) or any one of the preceding embodiments.

In a tenth embodiment, in the cationic lipid according to Formula (XX), Formula (XXI), Formula (XXII), Formula (XXIII), Formula (XXIV) or any one of the preceding embodiments, or a pharmaceutically acceptable salt thereof, R³ is C₃-C₅ alkylene or C₃-C₅ alkenylene, C₃-C₇ alkylene or C₃-C₇ alkenylene, or C₃-C₅ alkylene or C₃-C₅ alkenylene,; or R³ is C₈ alkylene, or C₇ alkylene, or C₆ alkylene, or C₅ alkylene, or C₄ alkylene, or C₃ alkylene, or C₁ alkylene, or C₈ alkenylene, or C₇ alkenylene, or C₆ alkenylene, or C₈ alkenylene, or C₄ alkenylene, or C₃ alkenylene; and all other remaining variables are as described for Formula Formula (XX), Formula (XXI), Formula (XXII), Formula (XXIII), Formula (XXIV) or any one of the preceding embodiments.

In an eleventh embodiment, in the cationic lipid according to Formula (XX), Formula (XXI), Formula (XXII), Formula (XXIII), Formula (XXIV) or any one of the preceding embodiments, or a pharmaceutically acceptable salt thereof, R^{6a} and R^{6b} are each independently C₇-C₁₂ alkyl or C₇-C₁₂ alkenyl; or R^{6a} and R^{6b} are each independently C₅-C₁₀ alkyl or C₅-C₁₀ alkenyl; or R^{6a} and R^{6b} are each independently C₁₂ alkyl, C₁₁ alkyl, C₁₀ alkyl, C₉ alkyl, C₈ alkyl, C₇ alkyl, C₁₂ alkenyl, C₁₁ alkenyl, C₁₀ alkenyl, C₉ alkenyl, C₈ alkenyl, or C₇ alkenyl; and all other remaining variables are as described for Formula (XX), Formula (XXI), Formula (XXII), Formula (XXIII), Formula (XXIV) or any one of the preceding embodiments.

In a twelfth embodiment, in the cationic lipid according to Formula (XX), Formula (XXI), Formula (XXII), Formula (XXIII), Formula (XXIV) or any one of the preceding embodiments, or a pharmaceutically acceptable salt thereof, R^{6a} and R^{6b} contain an equal number of carbon atoms with each other; or R^{6a} and R^{6b} are the same; or R^{6a} and R^{6b} are both C₁₂ alkyl, C₁₁ alkyl, C₁₀ alkyl, C₉ alkyl, Cs alkyl, C₇ alkyl, C₁₂ alkenyl, C₁₁ alkenyl, C₁₁ alkenyl, C₉ alkenyl, C₈ alkenyl, or C₇ alkenyl; and all other remaining variables are as described for Formula (XX), Formula (XXI), Formula (XXII), Formula (XXIII), Formula (XXIV) or any one of the preceding embodiments.

In a thirteenth embodiment, in the cationic lipid according to Formula (XX), Formula (XXI), Formula (XXII), Formula (XXIII), Formula (XXIV) or any one of the preceding embodiments, or a pharmaceutically acceptable salt thereof, R^{6a} and R^{6b} as defined in any one of the preceding embodiments each contain a different number of carbon atoms with each other; or the number of carbon atoms R^{6a} and R^{6b} differs by one or two carbon atoms; or the number of carbon atoms R^{6a} and R^{6b} differs by one carbon atom; or R^{6a} is C₇ alkyl and R^{6a} is C₈ alkyl, R^{6a} is C₈ alkyl and R^{6a} is C₇ alkyl, R^{6a} is C₈ alkyl and R^{6a} is C₉ alkyl, R^{6a} is C₉ alkyl and R^{6a} is C₈ alkyl, R^{6a} is C₉ alkyl and R^{6a} is C₁₀ alkyl, R^{6a} is C₁₁ alkyl and R^{6a} is C₉ alkyl, R^{6a} is C₁₁ alkyl and R^{6a} is C₁₁ alkyl, R^{6a} is C₁₁ alkyl and R^{6a} is C₁₀ alkyl, R^{6a} is C₁₁ alkyl and R^{6a} is C₁₂ alkyl, R^{6a} is C₁₂ alkyl and R^{6a} is C₁₁ alkyl, R^{6a} is C₇ alkyl and R^{6a} is C₉ alkyl, R^{6a} is C₉ alkyl and R^{6a} is C₇ alkyl, R^{6a} is C₈ alkyl and R^{6a} is C₁₁ alkyl, R^{6a} is C₁₀ alkyl and R^{6a} is C₈ alkyl, R⁶ is C₉ alkyl and R^{6a} is C₁₁ alkyl, R^{6a} is C₁₁ alkyl and R^{6a} is C₉ alkyl, R⁶ is C₁₀ alkyl and R^{6a} is C₁₂ alkyl, R^{6a} is C₁₂ alkyl and R^{6a} is C₁₀ alkyl, etc.; and all other remaining variables are as described for Formula (XX), Formula (XXI), Formula (XXII), Formula (XXIII), Formula (XXIV) or any one of the preceding embodiments.

In a fourteenth embodiment, in the cationic lipid according to Formula (XX), Formula (XXI), Formula (XXII), Formula (XXIII), Formula (XXIV) or any one of the preceding embodiments, or a pharmaceutically acceptable salt thereof, R′ is absent; and all other remaining variables are as described for Formula (XX), Formula (XXI), Formula (XXII), Formula (XXIII), Formula (XXIV) or any one of the preceding embodiments.

In one embodiment, the cationic lipid of the present disclosure or the cationic lipid of Formula (XX), Formula (XXI), Formula (XXII), Formula (XXIII), Formula (XXIV) is any one lipid selected from the lipids in Table 8 or a pharmaceutically acceptable salt thereof:

TABLE 8
Exemplary lipids of Formula (XX), Formula (XXI), Formula (XXII), Formula (XXIII), Formula (XXIV)

| Lipid | |
|---|---|
| No. | Lipid Structure and Name |
| 88 | 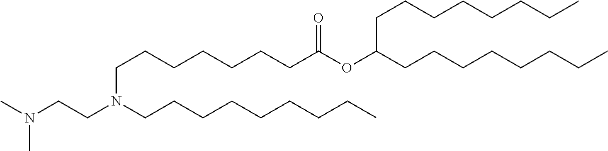 [SMILES: CCCCCCCCCN(CCCCCCCC(=O)OC(CCCCCCCC)CCCCCCCC)CCN(C)C] |
| | heptadecan-9-yl 8-((2-(dimethylamino)ethyl)(nonyl)amino)octanoate |
| 89 | 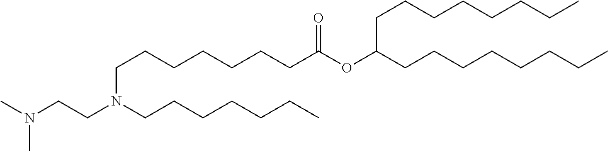 [SMILES: CCCCCCCCC(CCCCCCCC)OC(=O)CCCCCCCN(CCCCCCC)CCN(C)C] |
| | heptadecan-9-yl 8-((2-(dimethylamino)ethyl)(heptyl)amino)octanoate |
| 90 | 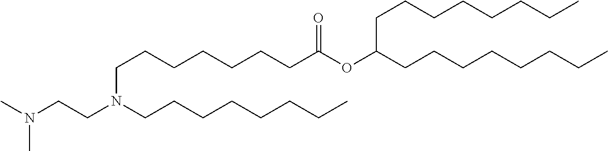 [SMILES: CCCCCCCCC(CCCCCCCC)OC(=O)CCCCCCCN(CCCCCCCC)CCN(C)C] |
| | heptadecan-9-yl 8-((2-(dimethylamino)ethyl)(octyl)amino)octanoate |
| 91 | 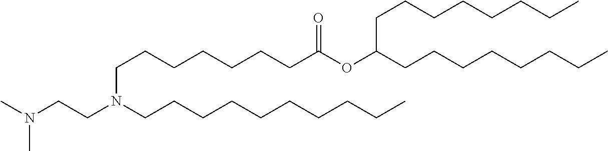 [SMILES: CCCCCCCCCCN(CCCCCCCC(=O)OC(CCCCCCCC)CCCCCCCC)CCN(C)C] |
| | heptadecan-9-yl 8-(decyl(2-(dimethylamino)ethyl)amino)octanoate |
| 92 | 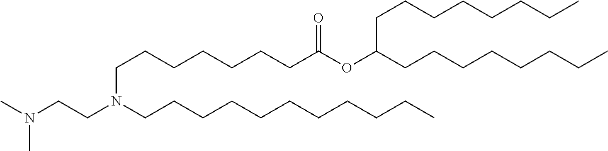 [SMILES: CCCCCCCCCCCN(CCCCCCCC(=O)OC(CCCCCCCC)CCCCCCCC)CCN(C)C] |
| | heptadecan-9-yl 8-((2-(dimethylamino)ethyl)(undecyl)amino)octanoate |
| 93 | 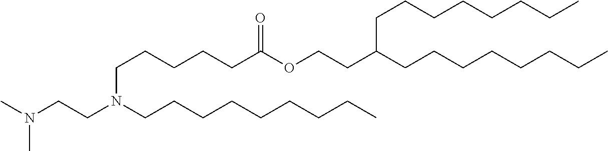 [SMILES: CCCCCCCCCN(CCCCCC(=O)OCCC(CCCCCCCC)CCCCCCCC)CCN(C)C] |
| | 3-octylundecyl 6-((2-(dimethylamino)ethyl)(nonyl)amino)hexanoate |
| 94 | 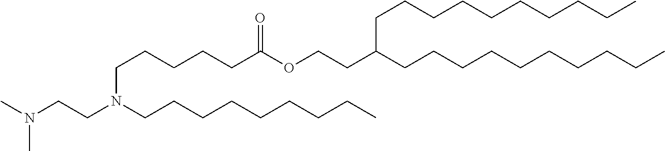 [SMILES: CCCCCCCCCCC(CCCCCCCCCC)CCOC(=O)CCCCCN(CCCCCCCCC)CCN(C)C] |
| | 3-decyltridecyl 6-((2-(dimethylamino)ethyl)(nonyl)amino)hexanoate |
| 95 | 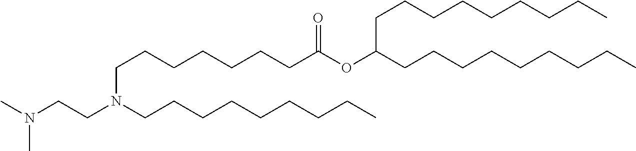 [SMILES: CCCCCCCCCC(CCCCCCCCC)OC(=O)CCCCCCCN(CCCCCCCCC)CCN(C)C] |
| | nonadecan-10-yl 8-((2-(dimethylamino)ethyl)(nonyl)amino)octanoate |
| 96 | 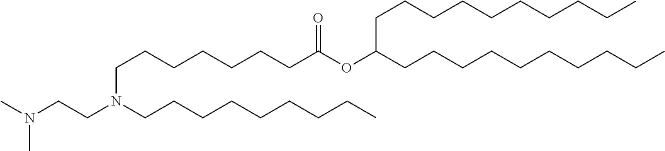 [SMILES: CCCCCCCCCCC(CCCCCCCCCC)OC(=O)CCCCCCCN(CCCCCCCCC)CCN(C)C] |
| | henicosan-11-yl 8-((2-(dimethylamino)ethyl)(nonyl)amino)octanoate |
| 97 | 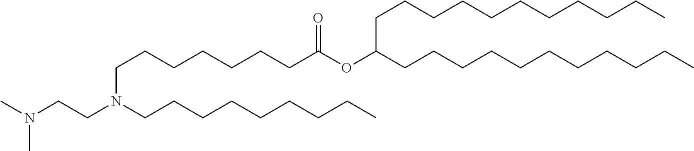 [SMILES: CCCCCCCCCCCC(CCCCCCCCCCC)OC(=O)CCCCCCCN(CCCCCCCCC)CCN(C)C] |
| | tricosan-12-yl 8-((2-(dimethylamino)ethyl)(nonyl)amino)octanoate |
| 98 | 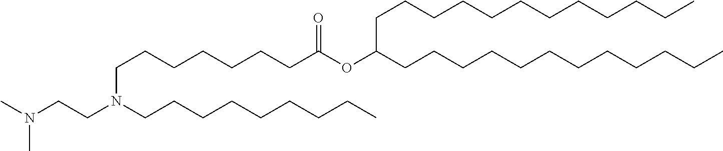 [SMILES: CCCCCCCCCCCCC(CCCCCCCCCCCC)OC(=O)CCCCCCCN(CCCCCCCCC)CCN(C)C] |
| | pentacosan-13-yl 8-((2-(dimethylamino)ethyl)(nonyl)amino)octanoate |

Specific examples are provided in the exemplification section below and are included as part of the cationic or ionizable lipids described herein. Pharmaceutically acceptable salts as well as neutral forms are also included.

### Cleavable Lipids

According to some embodiments, provided herein are pharmaceutical compositions comprising a lipid nanoparticle (LNP) and a therapeutic nucleic acid (TNA), wherein the LNP comprises a scFv (e.g., wherein the scFv is directed against an antigen present on the surface of a cell), linked to the LNP, via a cleavable lipid that can be used to deliver the capsid-free, non-viral DNA vector to a target site of interest (e.g., cell, tissue, organ, and the like). As used herein, the term “cleavable lipid” refers to a cationic lipid comprising a disulfide bond (“SS”) cleavable unit. In one embodiment, SS-cleavable lipids comprise a tertiary amine, which responds to an acidic compartment (e.g., an endosome or lysosome) for membrane destabilization and a disulfide bond that can cleave in a reductive environment (e.g., the cytoplasm). SS-cleavable lipids may include SS-cleavable and pH-activated lipid-like materials, such as ss-OP lipids, ssPalm lipids, ss-M lipids, ss-E lipids, ss-EC lipids, ss-LC lipids and ss-OC lipids, etc.

According to some embodiments, SS-cleavable lipids are described in International Patent Application Publication No. WO2019188867, incorporated by reference in its entirety herein.

According to some embodiments, the LNPs described herein range in size from about 20 to about 70 nm in mean diameter, for example, a mean diameter of from about 20 nm to about 70 nm, about 25 nm to about 70 nm, from about 30 nm to about 70 nm, from about 35 nm to about 70 nm, from about 40 nm to about 70 nm, from about 45 nm to about 80 nm, from about 50 nm to about 70 nm, from about 60 nm to about 70 nm, from about 65 nm to about 70 nm, or about 20 nm, about 25 nm, about 30 nm, about 35 nm, about 40 nm, about 45 nm, about 50 nm, about 55 nm, about 60 nm, about 65 nm, about 70 nm. According to some embodiments, the mean diameter of the LNPs is about 50 nm to about 70 nm. which is significantly smaller and therefore advantageous in targeting and circumventing immune responses. Moreover, the LNPs described herein can encapsulate greater than about 60% to about 90% of double stranded DNA, like ceDNA. According to some embodiments, the LNPs described herein can encapsulate greater than about 60% of double stranded DNA, like ceDNA, greater than about 65% of double stranded DNA, like ceDNA, greater than about 70% of double stranded DNA, like ceDNA, greater than about 75% of double stranded DNA, like ceDNA, greater than about 80% of double stranded DNA, like ceDNA, greater than about 85% of double stranded DNA, like ceDNA, or greater than about 90% of double stranded DNA, like ceDNA.

The lipid particles (e.g., LNPs comprising a scFv (e.g., wherein the scFv is directed against an antigen present on the surface of a cell), linked to the LNP) described herein can advantageously be used to increase delivery of nucleic acids (e.g., ceDNA, mRNA) to target cells/tissues compared to LNPs produced by other processes, and compared to other lipids, e.g., ionizable cationic lipids. Thus, the lipid particles (e.g., LNPs comprising a scFv (e.g., wherein the scFv is directed against an antigen present on the surface of a cell), linked to the LNP) described herein provided maximum nucleic acid delivery compared to lipid particles prepared by processes and methods known in the art. Although the mechanism has not yet been determined, and without being bound by theory, it is thought that the lipid particles (e.g., LNPs comprising a scFv (e.g., wherein the scFv is directed against an antigen present on the surface of a cell), linked to the LNP) to hepatocytes escaping phagocytosis from and more efficient trafficking to the nucleus. Another advantage of the lipid particles (e.g., LNPs comprising a scFv (e.g., wherein the scFv is directed against an antigen present on the surface of a cell), linked to the LNP) described herein is better tolerability compared to other lipids, e.g., ionizable cationic lipids, e.g., MC3.

In one embodiment, a cleavable lipid may comprise three components: an amine head group, a linker group, and a hydrophobic tail(s). In one embodiment, the cleavable lipid comprises one or more phenyl ester bonds, one of more tertiary amino groups, and a disulfide bond. The tertiary amine groups provide pH responsiveness and induce endosomal escape, the phenyl ester bonds enhance the degradability of the structure (self-degradability) and the disulfide bond cleaves in a reductive environment.

In one embodiment, the cleavable lipid is an ss-OP lipid. In one embodiment, an ss-OP lipid comprises the structure shown in Formula A below:

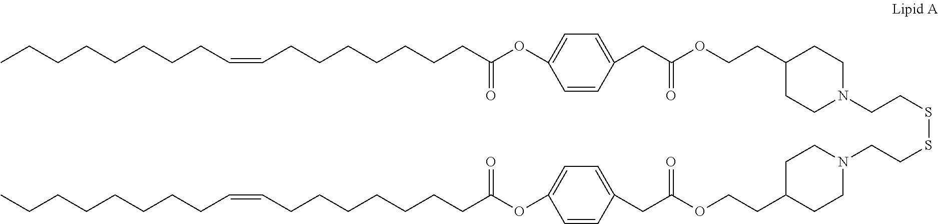 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCC/C=C\CCCCCCCC)cc4)CC3)CC2)cc1]

In one embodiment, the SS-cleavable lipid is an SS-cleavable and pH-activated lipid-like material (ssPalm). ssPalm lipids are well known in the art. For example, see Togashi et al., Journal of Controlled Release, 279 (2018) 262-270, the entire contents of which are incorporated herein by reference. In one embodiment, the ssPalm is an ssPalmM lipid comprising the structure of Lipid B.

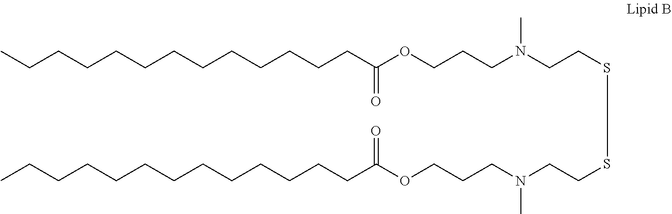 [SMILES: CCCCCCCCCCCCCC(=O)OCCCN(C)CCSSCCN(C)CCCOC(=O)CCCCCCCCCCCCC]

In one embodiment, the ssPalmE lipid is a ssPalmE-P4-C2 lipid comprising the structure of Lipid C.

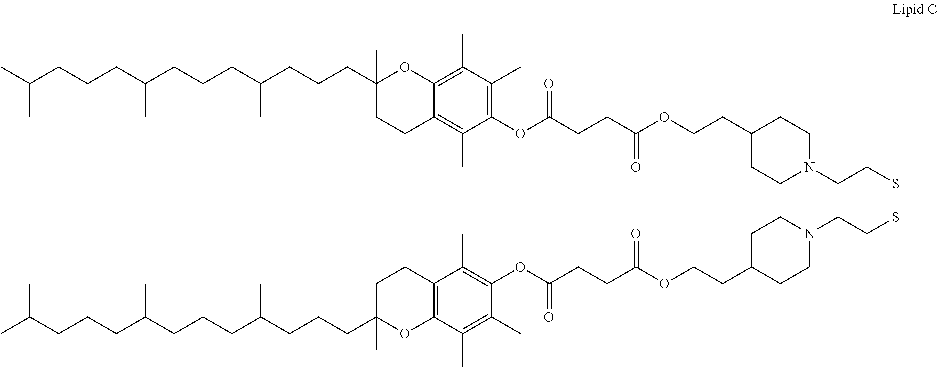

In one embodiment, the ssPalmE lipid is a ssPalmE-Paz4-C2 lipid, comprising the structure of Lipid D.

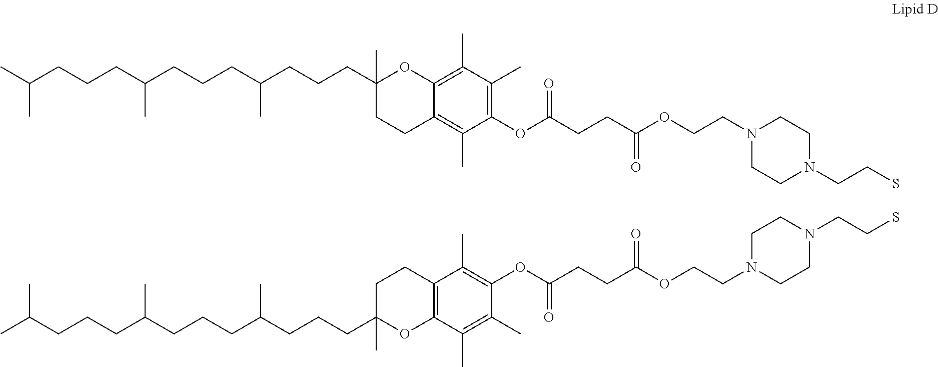

In one embodiment, the cleavable lipid is an ss-M lipid. In one embodiment, an ss-M lipid comprises the structure shown in Lipid E below:

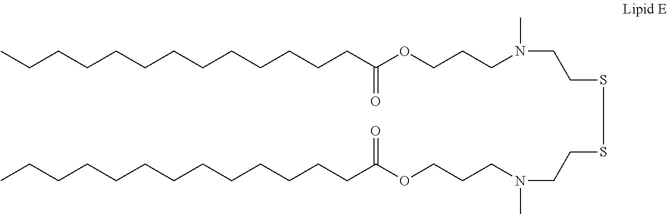 [SMILES: CCCCCCCCCCCCCC(=O)OCCCN(C)CCSSCCN(C)CCCOC(=O)CCCCCCCCCCCCC]

In one embodiment, the cleavable lipid is an ss-E lipid. In one embodiment, an ss-E lipid comprises the structure shown in Lipid F below:

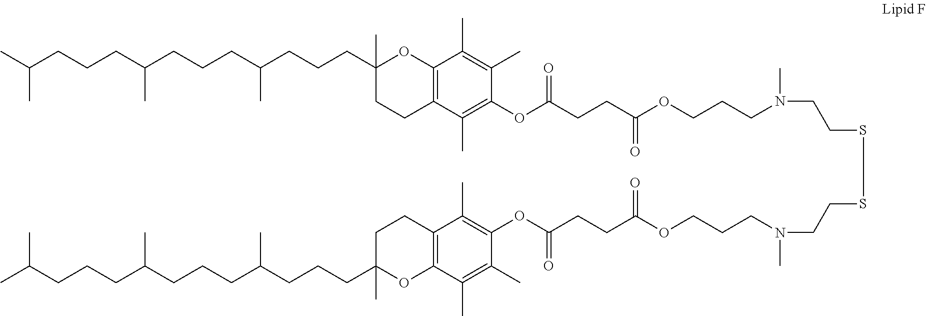 [SMILES: Cc1c(C)c2c(c(C)c1OC(=O)CCC(=O)OCCCN(C)CCSSCCN(C)CCCOC(=O)CCC(=O)Oc1c(C)c(C)c3c(c1C)CCC(C)(CCCC(C)CCCC(C)CCCC(C)C)O3)CCC(C)(CCCC(C)CCCC(C)CCCC(C)C)O2]

In one embodiment, the cleavable lipid is an ss-EC lipid In one embodiment, an ss-EC lipid comprises the structure shown in Lipid G below:

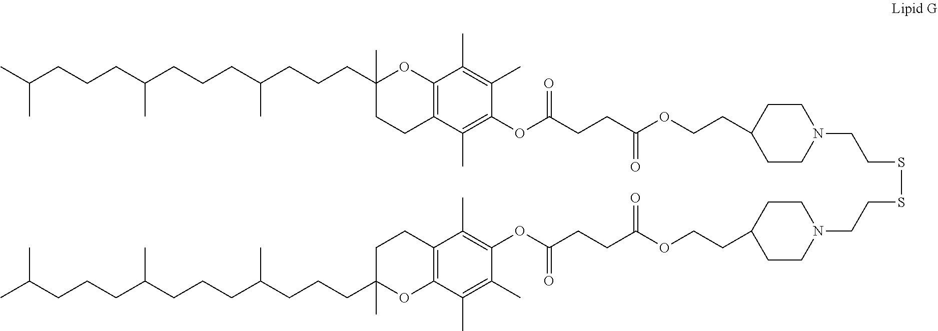 [SMILES: Cc1c(C)c2c(c(C)c1OC(=O)CCC(=O)OCCC1CCN(CCSSCCN3CCC(CCOC(=O)CCC(=O)Oc4c(C)c(C)c5c(c4C)CCC(C)(CCCC(C)CCCC(C)CCCC(C)C)O5)CC3)CC1)CCC(C)(CCCC(C)CCCC(C)CCCC(C)C)O2]

In one embodiment, the cleavable lipid is an ss-LC lipid. In one embodiment, an ss-LC lipid comprises the structure shown in Lipid H below:

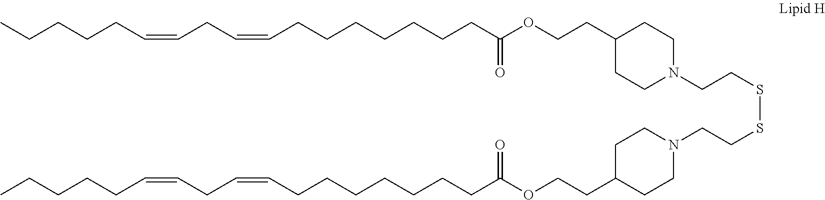 [SMILES: CCCCC/C=C\C/C=C\CCCCCCCC(=O)OCCC1CCN(CCSSCCN2CCC(CCOC(=O)CCCCCCC/C=C\C/C=C\CCCCC)CC2)CC1]

In one embodiment, the cleavable lipid is an ss-OC lipid. In one embodiment, an ss-OC lipid comprises the structure shown in Lipid J below:

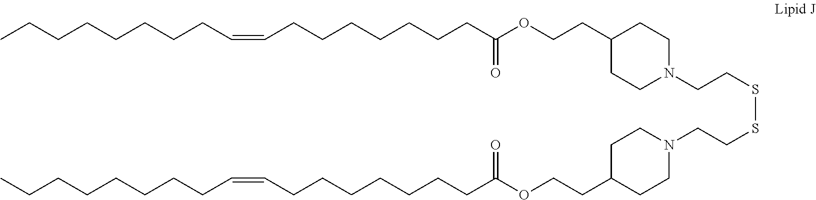 [SMILES: CCCCCCCC/C=C\CCCCCCCC(=O)OCCC1CCN(CCSSCCN2CCC(CCOC(=O)CCCCCCC/C=C\CCCCCCCC)CC2)CC1]

In one embodiment, a lipid particle (e.g., LNPs comprising a scFv (e.g., wherein the scFv is directed against an antigen present on the surface of a cell), linked to the LNP) formulation is made and loaded with ceDNA obtained by the process as disclosed in International Patent Application No. PCT/US2018/050042, filed on Sep. 7, 2018, which is incorporated by reference in its entirety herein. This can be accomplished by high energy mixing of ethanolic lipids with aqueous ceDNA at low pH which protonates the lipid and provides favorable energetics for ceDNA/lipid association and nucleation of particles. The particles can be further stabilized through aqueous dilution and removal of the organic solvent. The particles can be concentrated to the desired level. In one embodiment, the disclosure provides a ceDNA lipid particle comprising a lipid of Formula I prepared by a process as described in Example 2 of U.S. Provisional Application No. 63/194,620.

Generally, the lipid particles (e.g., LNPs comprising a scFv (e.g., wherein the scFv is directed against an antigen present on the surface of a cell), linked to the LNP) are prepared at a total lipid to ceDNA (mass or weight) ratio of from about 10:1 to 60:1. In some embodiments, the lipid to ceDNA ratio (mass/mass ratio; w/w ratio) can be in the range of from about 1:1 to about 60:1, from about 1:1 to about 55:1, from about 1:1 to about 50:1, from about 1:1 to about 45:1, from about 1:1 to about 40:1, from about 1:1 to about 35:1, from about 1:1 to about 30:1, from about 1:1 to about 25:1, from about 10:1 to about 14:1, from about 3:1 to about 15:1, from about 4:1 to about 10:1, from about 5:1 to about 9:1, about 6:1 to about 9:1; from about 30:1 to about 60:1. According to some embodiments, the lipid particles (e.g., LNPs comprising a scFv (e.g., wherein the scFv is directed against an antigen present on the surface of a cell), linked to the LNP) are prepared at a ceDNA (mass or weight) to total lipid ratio of about 60:1. According to some embodiments, the lipid particles (e.g., LNPs comprising a scFv (e.g., wherein the scFv is directed against an antigen present on the surface of a cell), linked to the LNP) are prepared at a ceDNA (mass or weight) to total lipid ratio of about 30:1. The amounts of lipids and ceDNA can be adjusted to provide a desired N/P ratio, for example, N/P ratio of 3, 4, 5, 6, 7, 8, 9, 10 or higher. Generally, the lipid particle formulation's overall lipid content can range from about 5 mg/ml to about 30 mg/mL.

In some embodiments, the lipid nanoparticle comprises an agent for condensing and/or encapsulating nucleic acid cargo, such as ceDNA. Such an agent is also referred to as a condensing or encapsulating agent herein. Without limitations, any compound known in the art for condensing and/or encapsulating nucleic acids can be used as long as it is non-fusogenic. In other words, an agent capable of condensing and/or encapsulating the nucleic acid cargo, such as ceDNA, but having little or no fusogenic activity. Without wishing to be bound by theory, a condensing agent may have some fusogenic activity when not condensing/encapsulating a nucleic acid, such as ceDNA, but a nucleic acid encapsulating lipid nanoparticle formed with said condensing agent can be non-fusogenic.

According to some embodiments, the LNPs comprising a scFv (e.g., wherein the scFv is directed against an antigen present on the surface of a cell), linked to the LNP described herein can encapsulate greater than about 60% of rigid double stranded DNA, like ceDNA, greater than about 65% of rigid double stranded DNA, like ceDNA, greater than about 70% of rigid double stranded DNA, like ceDNA, greater than about 75% of rigid double stranded DNA, like ceDNA, greater than about 80% of rigid double stranded DNA, like ceDNA,n greater than about 85% of rigid double stranded DNA, like ceDNA, or greater than about 90% of rigid double stranded DNA, like ceDNA.

The cationic lipid is typically employed to condense the nucleic acid cargo, e.g., ceDNA at low pH and to drive membrane association and fusogenicity. Generally, catonic lipids are lipids comprising at least one amino group that is positively charged or becomes protonated under acidic conditions, for example at pH of 6.5 or lower. Cationic lipids may also be ionizable lipids, e.g., ionizable cationic lipids. By a “non-fusogenic cationic lipid” is meant a cationic lipid that can condense and/or encapsulate the nucleic acid cargo, such as ceDNA, but does not have, or has very little, fusogenic activity.

In one embodiment, the cationic lipid can comprise 20-90% (mol) of the total lipid present in the lipid particles (e.g., lipid nanoparticles). For example, cationic lipid molar content can be 20-70% (mol), 30-60% (mol), 40-60% (mol), 40-55% (mol) or 45-55% (mol) of the total lipid present in the lipid particle (e.g., lipid nanoparticles). In some embodiments, cationic lipid comprises from about 50 mol % to about 90 mol % of the total lipid present in the lipid particles (e.g., LNPs comprising a scFv (e.g., wherein the scFv is directed against an antigen present on the surface of a cell), linked to the LNP).

In one embodiment, the SS-cleavable lipid is not MC3 (6Z,9Z,28Z,3 lZ)-heptatriaconta-6,9,28,31-tetraen-19-yl-4-(dimethylamino)butanoate (DLin-MC3-DMA or MC3). DLin-MC3-DMA is described in Jayaraman et al., Angew. Chem. Int. Ed Engl. (2012), 51(34): 8529-8533, the contents of which is incorporated herein by reference in its entirety. The structure of D-Lin-MC3-DMA (MC3) is shown below as Lipid K:

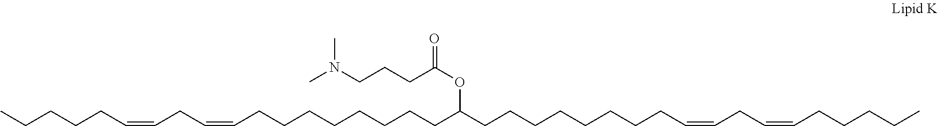 [SMILES: CCCCC/C=C\C/C=C\CCCCCCCCC(CCCCCCCC/C=C\C/C=C\CCCCC)OC(=O)CCCN(C)C]

In one embodiment, the cleavable lipid is not the lipid ATX-002. The lipid ATX-002 is described in WO2015/074085, the content of which is incorporated herein by reference in its entirety.

In one embodiment, the cleavable lipid is not (13Z.16Z)-/V,/V-dimethyl-3-nonyldocosa-13,16-dien-1-amine (Compound 32). Compound 32 is described in WO2012/040184, the contents of which is incorporated herein by reference in its entirety. In one embodiment, the cleavable lipid is not Compound 6 or Compound 22. Compounds 6 and 22 are described in WO2015/199952, the content of which is incorporated herein by reference in its entirety.

Non-limiting examples of cationic lipids include SS-cleavable and pH-activated lipid-like material-OP (ss-OP; Formula I), SS-cleavable and pH-activated lipid-like material-M (SS-M; Formula V), SS-cleavable and pH-activated lipid-like material-E (SS-E; Formula VI), SS-cleavable and pH-activated lipid-like material-EC (SS-EC; Formula VII), SS-cleavable and pH-activated lipid-like material-LC (SS-LC; Formula VIII), SS-cleavable and pH-activated lipid-like material-OC (SS-OC; Formula IX), polyethylenimine, polyamidoamine (PAMAM) starburst dendrimers, Lipofectin (a combination of DOTMA and DOPE), Lipofectase, LIPOFECTAMINE™ (e.g., LIPOFECTAMINE™ 2000), DOPE, Cytofectin (Gilead Sciences, Foster City, Calif.), and Eufectins (JBL, San Luis Obispo, Calif.). Exemplary cationic liposomes can be made from N-[1-(2,3-dioleoloxy)-propyl]-N,N,N-trimethylammonium chloride (DOTMA), N-[1-(2,3-dioleoloxy)-propyl]-N,N,N-trimethylammonium methylsulfate (DOTAP), 3b-[N-(N′,N′-dimethylaminoethane)carbamoyl]cholesterol (DC-Chol), 2,3,-dioleyloxy-N-[2(sperminecarboxamido)ethyl]-N,N -dimethyl-1-propanaminium trifluoroacetate (DOSPA), 1,2-dimyristyloxypropyl-3-dimethyl-hydroxyethyl ammonium bromide; and dimethyldioctadecylammonium bromide (DDAB). Nucleic acids (e.g., ceDNA or CELiD) can also be complexed with, e.g., poly (L-lysine) or avidin and lipids can, or cannot, be included in this mixture, e.g., steryl-poly (L-lysine).

In one embodiment, the cationic lipid is ss-OP of Formula I. In another embodiment, the cationic lipid SS-PAZ of Formula II.

In one embodiment, a ceDNA vector as disclosed herein is delivered using a cationic lipid described in U.S. Pat. No. 8,158,601, or a polyamine compound or lipid as described in U.S. Pat. No. 8,034,376.

### B. Non-cationic Lipids

In one embodiment, the lipid particles (e.g., LNPs comprising a scFv (e.g., wherein the scFv is directed against an antigen present on the surface of a cell), linked to the LNP) can further comprise a non-cationic lipid. The non-cationic lipid can serve to increase fusogenicity and also increase stability of the LNP during formation. Non-cationic lipids include amphipathic lipids, neutral lipids and anionic lipids. Accordingly, the non-cationic lipid can be a neutral uncharged, zwitterionic, or anionic lipid. Non-cationic lipids are typically employed to enhance fusogenicity.

Exemplary non-cationic lipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), 1,2-dilauroyl-sn-glycero-3-pho sphoethanolamine (DLPE); 1,2-diphytanoyl-sn-glycero-3-phosphoethanolamine (DPHyPE); lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid,cerebrosides, dicetylphosphate, lysophosphatidylcholine, dilinoleoylphosphatidylcholine, or mixtures thereof. It is to be understood that other diacylphosphatidylcholine and diacylphosphatidylethanolamine phospholipids can also be used. The acyl groups in these lipids are preferably acyl groups derived from fatty acids having C₁₀-C₂₄ carbon chains, e.g., lauroyl, myristoyl, palmitoyl, stearoyl, or oleoyl.

Other examples of non-cationic lipids suitable for use in the lipid particles (e.g., 1 LNPs comprising a scFv (e.g., wherein the scFv is directed against an antigen present on the surface of a cell), linked to the LNP) include nonphosphorous lipids such as, e.g., stearylamine, dodecylamine, hexadecylamine, acetyl palmitate, glycerolricinoleate, hexadecyl stereate, isopropyl myristate, amphoteric acrylic polymers, triethanolamine-lauryl sulfate, alkyl-aryl sulfate polyethyloxylated fatty acid amides, dioctadecyldimethyl ammonium bromide, ceramide, sphingomyelin, and the like.

In one embodiment, the non-cationic lipid is a phospholipid. In one embodiment, the non-cationic lipid is selected from the group consisting of DSPC, DPPC, DMPC, DOPC, POPC, DOPE, and SM. In some embodiments, the non-cationic lipid is DSPC. In other embodiments, the non-cationic lipid is DOPC. In other embodiments, the non-cationic lipid is DOPE.

In some embodiments, the non-cationic lipid can comprise 0-20% (mol) of the total lipid present in the lipid nanoparticle. In some embodiments, the non-cationic lipid content is 0.5-15% (mol) of the total lipid present in the lipid particle (e.g., lipid nanoparticle). In some embodiments, the non-cationic lipid content is 5-12% (mol) of the total lipid present in the lipid particle (e.g., lipid nanoparticle). In some embodiments, the non-cationic lipid content is 5-10% (mol) of the total lipid present in the lipid particle (e.g., lipid nanoparticle). In one embodiment, the non-cationic lipid content is about 6% (mol) of the total lipid present in the lipid particle (e.g., lipid nanoparticle). In one embodiment, the non-cationic lipid content is about 7.0% (mol) of the total lipid present in the lipid particle (e.g., lipid nanoparticle). In one embodiment, the non-cationic lipid content is about 7.5% (mol) of the total lipid present in the lipid particle (e.g., lipid nanoparticle). In one embodiment, the non-cationic lipid content is about 8.0% (mol) of the total lipid present in the lipid particle (e.g., lipid nanoparticle). In one embodiment, the non-cationic lipid content is about 9.0% (mol) of the total lipid present in the lipid particle (e.g., lipid nanoparticle). In some embodiments, the non-cationic lipid content is about 10% (mol) of the total lipid present in the lipid particle (e.g., lipid nanoparticle). In one embodiment, the non-cationic lipid content is about 11% (mol) of the total lipid present in the lipid particle (e.g., lipid nanoparticle).

Exemplary non-cationic lipids are described in International Patent Application Publication No. WO2017/099823 and US Patent Application Publication No. US2018/0028664, the contents of both of which are incorporated herein by reference in their entirety.

In one embodiment, the lipid particles (e.g., lipid nanoparticles) can further comprise a component, such as a sterol, to provide membrane integrity and stability of the lipid particle. In one embodiment, an exemplary sterol that can be used in the lipid particle is cholesterol, or a derivative thereof. Non-limiting examples of cholesterol derivatives include polar analogues such as 5α-cholestanol, 50-coprostanol, cholesteryl-(2′-hydroxy)-ethyl ether, cholesteryl-(4′-hydroxy)-butyl ether, and 6-ketocholestanol; non-polar analogues such as 5α-cholestane, cholestenone, 5α-cholestanone, 50-cholestanone, and cholesteryl decanoate; and mixtures thereof. In some embodiments, the cholesterol derivative is a polar analogue such as cholesteryl-(4′-hydroxy)-butyl ether. In some embodiments, cholesterol derivative is cholestryl hemisuccinate (CHEMS).

Exemplary cholesterol derivatives are described in International Patent Application Publication No. WO2009/127060 and U.S. Patent Application Publication No. US2010/0130588, contents of both of which are incorporated herein by reference in their entirety.

In one embodiment, the component providing membrane integrity, such as a sterol, can comprise 0-50% (mol) of the total lipid present in the lipid particle (e.g., lipid nanoparticle). In some embodiments, such a component is 20-50% (mol) of the total lipid content of the lipid particle (e.g., lipid nanoparticle). In some embodiments, such a component is 30-40% (mol) of the total lipid content of the lipid particle (e.g., lipid nanoparticle). In some embodiments, such a component is 35-45% (mol) of the total lipid content of the lipid particle (e.g., lipid nanoparticle). In some embodiments, such a component is 38-42% (mol) of the total lipid content of the lipid particle (e.g., lipid nanoparticle).

In one embodiment, the lipid particle (e.g., lipid nanoparticle) can further comprise a polyethylene glycol (PEG) or a conjugated lipid molecule. Generally, these are used to inhibit aggregation of lipid particle (e.g., lipid nanoparticle) and/or provide steric stabilization. Exemplary conjugated lipids include, but are not limited to, PEG-lipid conjugates, polyoxazoline (POZ)-lipid conjugates, polyamide -lipid conjugates (such as ATTA-lipid conjugates), cationic-polymer lipid (CPL) conjugates, and mixtures thereof. In some embodiments, the conjugated lipid molecule is a PEGylated lipid, for example, a (methoxy polyethylene glycol)-conjugated lipid. In some other embodiments, the PEGylated lipid is PEG₂₀₀₀-DMG (dimyristoylglycerol).

Exemplary PEGylated lipids include, but are not limited to, PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di(tetradecanoyloxy)propyl-1-0-(w-methoxy(polyethoxy)ethyl) butanedioate (PEG-S-DMG)), PEG dialkoxypropylcarbam, N-(carbonyl-methoxypoly ethylene glycol 2000)-1,2-distearoyl-sn-glycero-3-phosphoethanolamine sodium salt, or a mixture thereof. Additional exemplary PEG-lipid conjugates are described, for example, in U.S. Pat. Nos. 5,885,613, 6,287,591, US2003/0077829, US2003/0077829, US2005/0175682, US2008/0020058, US2011/0117125, US2010/0130588, US2016/0376224, and US2017/0119904, the contents of all of which are incorporated herein by reference in their entirety.

In one embodiment, the PEG-DAA PEGylated lipid can be, for example, PEG-dilauryloxypropyl, PEG-dimyristyloxypropyl, PEG-dipalmityloxypropyl, or PEG-distearyloxypropyl. The PEG-lipid can be one or more of PEG-DMG, PEG-dilaurylglycerol, PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol (1-[8′-(Cholest-5-en-3[beta]-oxy)carboxamido-3′,6′-dioxaoctanyl]carbamoyl-[omega]-methyl-poly(ethylene glycol), PEG-DMB (3,4-Ditetradecoxylbenzyl-[omega]-methyl-poly(ethylene glycol) ether), and 1,2-dimyristoyl-sn-glycero-3-phosphoethanolamine-N-[methoxy(polyethylene glycol)-2000]. In one embodiment, the PEG-lipid can be selected from the group consisting of PEG-DMG, 1,2-dimyristoyl-sn-glycero-3-phosphoethanolamine-N-[methoxy(polyethylene glycol)-2000],

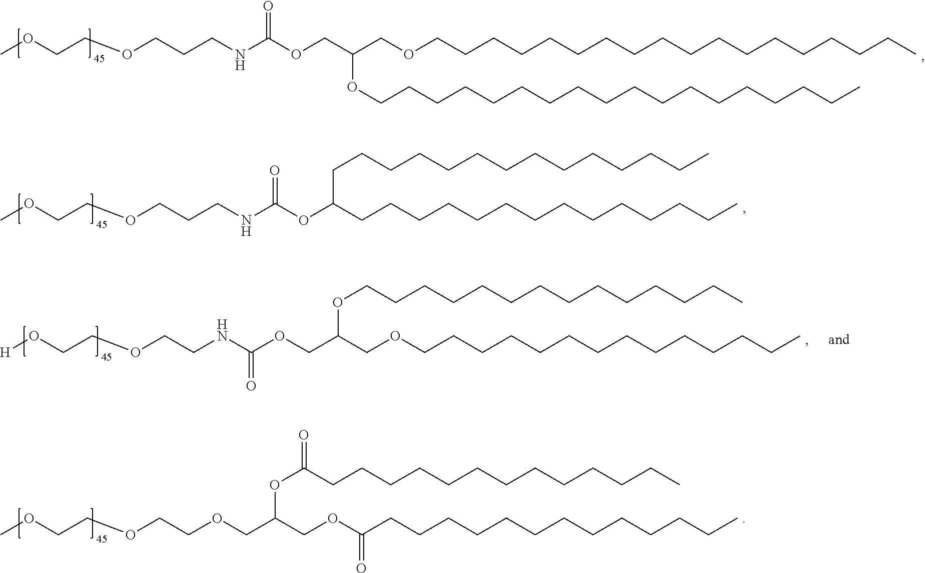

In some embodiments, the PEGylated lipid is selected from the group consisting N-(Carbonyl-methoxypolyethyleneglycoln)-1,2-dimyristoyl-sn-glycero-3-phosphoethanolamine (DMPE-PEG., where n is 350, 500, 750, 1000 or 2000), N-(Carbonyl-methoxypolyethyleneglycoln)-1,2-distearoyl-sn-glycero-3-phosphoethanolamine (DSPE-PEG., where n is 350, 500, 750, 1000 or 2000), DSPE-polyglycelin-cyclohexyl-carboxylic acid, DSPE-polyglycelin-2-methylglutar-carboxylic acid, 1,2-Distearoyl-sn-Glycero-3-Phosphoethanolamine (DSPE) conjugated Polyethylene Glycol (DSPE-PEG-OH), polyethylene glycol-dimyristolglycerol (PEG-DMG), polyethylene glycol-distearoyl glycerol (PEG-DSG), or N-octanoyl-sphingosine-1-{succinyl[methoxy(polyethylene glycol)200011 (C8 PEG2000 Ceramide). In some examples of DMPE-PEG., where n is 350, 500, 750, 1000 or 2000, the PEG-lipid is N-(Carbonyl-methoxypolyethyleneglycol 2000)-1,2-dimyristoyl-sn-glycero-3-phosphoethanolamine (DMPE-PEG 2,000). In some examples of DSPE-PEG,. where n is 350, 500, 750, 1000 or 2000, the PEG-lipid is N-(Carbonyl-methoxypolyethyleneglycol 2000)-1,2-distearoyl-sn-glycero-3-phosphoethanolamine (DSPE-PEG 2,000). In some embodiments, the PEG-lipid is DSPE-PEG-OH. In some preferred embodiments, the PEG-lipid is PEG-DMG.

In some embodiments, the conjugated lipid, e.g., PEGylated lipid, includes a tissue-specific targeting ligand, e.g., first or second targeting ligand. For example, PEG-DMG conjugated with a GalNAc ligand.

In one embodiment, lipids conjugated with a molecule other than a PEG can also be used in place of PEG-lipid. For example, polyoxazoline (POZ)-lipid conjugates, polyamide-lipid conjugates (such as ATTA-lipid conjugates), and cationic -polymer lipid (CPL) conjugates can be used in place of or in addition to the PEG-lipid. Exemplary conjugated lipids, i.e., PEG-lipids, (POZ)-lipid conjugates, ATTA-lipid conjugates and cationic polymer-lipids are described in the International Patent Application Publication Nos. WO 1996/010392, WO1998/051278, WO2002/087541, WO2005/026372, WO2008/147438, WO2009/086558, WO2012/000104, WO2017/117528, WO2017/099823, WO2015/199952, WO2017/004143, WO2015/095346, WO2012/000104, WO2012/000104, and WO2010/006282, U.S. Patent Application Publication Nos. US2003/0077829, US2005/0175682, US2008/0020058, US2011/0117125, US2013/0303587, US2018/0028664, US2015/0376115, US2016/0376224, US2016/0317458, US2013/0303587, US2013/0303587, and US20110123453, and U.S. Pat. Nos. U.S. Pat. Nos. 5,885,613, 6,287,591, 6,320,017, and 6,586,559, the contents of all of which are incorporated herein by reference in their entireties.

In some embodiments, the PEGylated lipid can comprise 0-20% (mol) of the total lipid present in the lipid nanoparticle. In some embodiments, PEGylated lipid content is 0.5-10% (mol). In some embodiments, PEGylated lipid content is 1-5% (mol). In some embodiments, PEGylated lipid content is 2-4% (mol). In some embodiments, PEGylated lipid content is 2-3% (mol). In one embodiment, PEGylated lipid content is about 2% (mol). In one embodiment, PEGylated lipid content is about 2.5% (mol). In some embodiments, PEGylated lipid content is about 3% (mol). In one embodiment, PEGylated lipid content is about 3.5% (mol). In one embodiment, PEGylated lipid content is about 4% (mol).

It is understood that molar ratios of the cationic lipid, e.g., ionizable cationic lipid, with the non-cationic-lipid, sterol, and PEGylated lipid can be varied as needed. For example, the lipid particle (e.g., lipid nanoparticle) can comprise 30-70% cationic lipid by mole or by total weight of the composition, 0-60% cholesterol by mole or by total weight of the composition, 0-30% non-cationic lipid by mole or by total weight of the composition and 2-5% PEGylated lipid by mole or by total weight of the composition. In one embodiment, the composition comprises 40-60% cationic lipid by mole or by total weight of the composition, 30-50% cholesterol by mole or by total weight of the composition, 5-15% non-cationic lipid by mole or by total weight of the composition and 2-5% PEG or the conjugated lipid by mole or by total weight of the composition. In one embodiment, the composition is 40-60% cationic lipid by mole or by total weight of the composition, 30-40% cholesterol by mole or by total weight of the composition, and 5-10% non-cationic lipid, by mole or by total weight of the composition and 2-5% PEGylated lipid by mole or by total weight of the composition. The composition may contain 60-70% cationic lipid by mole or by total weight of the composition, 25-35% cholesterol by mole or by total weight of the composition, 5-10% non-cationic lipid by mole or by total weight of the composition and 2-5% PEGylated lipid by mole or by total weight of the composition. The composition may also contain up to 45-55% cationic lipid by mole or by total weight of the composition, 35-45% cholesterol by mole or by total weight of the composition, 2 to 15% non-cationic lipid by mole or by total weight of the composition, and 2-5% PEGylated lipid by mole or by total weight of the composition. The formulation may also be a lipid nanoparticle formulation, for example comprising 8-30% cationic lipid by mole or by total weight of the composition, 5-15% non-cationic lipid by mole or by total weight of the composition, and 0-40% cholesterol by mole or by total weight of the composition; 4-25% cationic lipid by mole or by total weight of the composition, 4-25% non-cationic lipid by mole or by total weight of the composition, 2 to 25% cholesterol by mole or by total weight of the composition, 10 to 35% conjugate lipid by mole or by total weight of the composition, and 5% cholesterol by mole or by total weight of the composition; or 2-30% cationic lipid by mole or by total weight of the composition, 2-30% non-cationic lipid by mole or by total weight of the composition, 1 to 15% cholesterol by mole or by total weight of the composition, 2 to 35% PEGylated lipid by mole or by total weight of the composition, and 1-20% cholesterol by mole or by total weight of the composition; or even up to 90% cationic lipid by mole or by total weight of the composition and 2-10% non-cationic lipids by mole or by total weight of the composition, or even 100% cationic lipid by mole or by total weight of the composition.

In some embodiments, the lipid particle formulation comprises cationic lipid, non-cationic phospholipid, cholesterol and a PEGylated lipid (conjugated lipid) in a molar ratio of about 50:9:38.5:2.5.

In one embodiment, the lipid particle (e.g., lipid nanoparticle) formulation comprises cationic lipid, non-cationic phospholipid, cholesterol and a PEGylated lipid (conjugated lipid) in a molar ratio of about 50:7:40:3.

In other aspects, the disclosure provides for a lipid nanoparticle formulation comprising phospholipids, lecithin, phosphatidylcholine and phosphatidylethanolamine.

In one embodiment, the lipid particle (e.g., lipid nanoparticle) comprises cationic lipid, non-cationic lipid (e.g. phospholipid), a sterol (e.g., cholesterol) and a PEGylated lipid (conjugated lipid), where the molar ratio of lipids ranges from 20 to 70 mole percent for the cationic lipid, with a target of 30-60, the mole percent of non-cationic lipid ranges from 0 to 30, with a target of 0 to 15, the mole percent of sterol ranges from 20 to 70, with a target of 30 to 50, and the mole percent of PEGylated lipid (conjugated lipid) ranges from 1 to 6, with a target of 2 to 5.

Lipid nanoparticles (LNPs) comprising ceDNA are disclosed in International Patent Application No. PCT/US2018/050042, filed on Sep. 7, 2018, which is incorporated herein in its entirety and envisioned for use in the methods and compositions as disclosed herein.

Lipid particle (e.g., lipid nanoparticle) size can be determined by quasi-elastic light scattering using a Malvern Zetasizer Nano ZS (Malvern, UK). According to some embodiments, LNP mean diameter as determined by light scattering is less than about 75 nm or less than about 70 nm.

According to some embodiments, LNP mean diameter as determined by light scattering is between about 50 nm to about 75 nm or about 50 nm to about 70 nm.

The pKa of formulated cationic lipids can be correlated with the effectiveness of the LNPs for delivery of nucleic acids (see Jayaraman et al, Angewandte Chemie, International Edition (2012), 51(34), 8529-8533; Semple et al., Nature Biotechnology 28, 172-176 (2010), both of which are incorporated by reference in their entireties). In one embodiment, the pKa of each cationic lipid is determined in lipid nanoparticles using an assay based on fluorescence of 2-(p-toluidino)-6-napthalene sulfonic acid (TNS). Lipid nanoparticles comprising of cationic lipid/DSPC/cholesterol/PEG-lipid (50/10/38.5/1.5 mol %) in PBS at a concentration of 0.4 mM total lipid can be prepared using the in-line process as described herein and elsewhere. TNS can be prepared as a 100 mM stock solution in distilled water. Vesicles can be diluted to 24 mM lipid in 2 mL of buffered solutions containing, 10 mM HEPES, 10 mM MES, 10 mM ammonium acetate, 130 mM NaCl, where the pH ranges from 2.5 to 11. An aliquot of the TNS solution can be added to give a final concentration of 1 mM and following vortex mixing fluorescence intensity is measured at room temperature in a SLM Aminco Series 2 Luminescence Spectrophotometer using excitation and emission wavelengths of 321 nm and 445 nm. A sigmoidal best fit analysis can be applied to the fluorescence data and the pKa is measured as the pH giving rise to half-maximal fluorescence intensity.

In one embodiment, relative activity can be determined by measuring luciferase expression in the liver 4 hours following administration via tail vein injection. The activity is compared at a dose of 0.3 and 1.0 mg ceDNA/kg and expressed as ng luciferase/g liver measured 4 hours after administration.

Without limitations, a lipid particle (e.g., lipid nanoparticle) of the disclosure includes a lipid formulation that can be used to deliver a capsid-free, non-viral DNA vector to a target site of interest (e.g., cell, tissue, organ, and the like). Generally, the lipid particle (e.g., lipid nanoparticle) comprises capsid-free, non-viral DNA vector and a cationic lipid or a salt thereof.

In one embodiment, the lipid particle (e.g., lipid nanoparticle) comprises a cationic lipid/non-cationic-lipid/sterol/conjugated lipid at a molar ratio of 50:10:38.5:1.5. In one embodiment, the disclosure provides for a lipid particle (e.g., lipid nanoparticle) formulation comprising phospholipids, lecithin, phosphatidylcholine and phosphatidylethanolamine.

III. Closed-ended DNA (ceDNA) Vectors

Embodiments of the disclosure are based on methods and compositions comprising closed-ended linear duplexed (ceDNA) vectors that can express a transgene (e.g. a therapeutic nucleic acid (TNA)). The ceDNA vectors as described herein have no packaging constraints imposed by the limiting space within the viral capsid. ceDNA vectors represent a viable eukaryotically-produced alternative to prokaryote-produced plasmid DNA vectors, as opposed to encapsulated AAV genomes. This permits the insertion of control elements, e.g., regulatory switches as disclosed herein, large transgenes, multiple transgenes etc. ceDNA vectors preferably have a linear and continuous structure rather than a non-continuous structure. The linear and continuous structure is believed to be more stable from attack by cellular endonucleases, as well as less likely to be recombined and cause mutagenesis. Thus, a ceDNA vector in the linear and continuous structure is a preferred embodiment. The continuous, linear, single strand intramolecular duplex ceDNA vector can have covalently bound terminal ends, without sequences encoding AAV capsid proteins. These ceDNA vectors are structurally distinct from plasmids (including ceDNA plasmids described herein), which are circular duplex nucleic acid molecules of bacterial origin. The complimentary strands of plasmids may be separated following denaturation to produce two nucleic acid molecules, whereas in contrast, ceDNA vectors, while having complimentary strands, are a single DNA molecule and therefore even if denatured, it is likely to remain a single molecule. In some embodiments, ceDNA vectors can be produced without DNA base methylation of prokaryotic type, unlike plasmids. Therefore, the ceDNA vectors and ceDNA-plasmids are different both in term of structure (in particular, linear versus circular) and also in view of the methods used for producing and purifying these different objects, and also in view of their DNA methylation which is of prokaryotic type for ceDNA-plasmids and of eukaryotic type for the ceDNA vector.

Provided herein are non-viral, capsid-free ceDNA molecules with covalently closed ends (ceDNA). These non-viral capsid free ceDNA molecules can be produced in permissive host cells from an expression construct (e.g., a ceDNA-plasmid, a ceDNA-bacmid, a ceDNA-baculovirus, or an integrated cell-line) containing a heterologous gene (e.g., a transgene, in particular a therapeutic transgene) positioned between two different inverted terminal repeat (ITR) sequences, where the ITRs are different with respect to each other. In some embodiments, one of the ITRs is modified by deletion, insertion, and/or substitution as compared to a wild-type ITR sequence (e.g. AAV ITR); and at least one of the ITRs comprises a functional terminal resolution site (trs) and a Rep binding site. The ceDNA vector is preferably duplex, e.g., self-complementary, over at least a portion of the molecule, such as the expression cassette (e.g., ceDNA is not a double stranded circular molecule). The ceDNA vector has covalently closed ends, and thus is resistant to exonuclease digestion (e.g. exonuclease I or exonuclease III), e.g. for over an hour at 37° C.

In one aspect, a ceDNA vector comprises, in the 5′ to 3′ direction: a first adeno-associated virus (AAV) inverted terminal repeat (ITR), a nucleotide sequence of interest (for example an expression cassette as described herein) and a second AAV ITR. In one embodiment, the first ITR (5′ ITR) and the second ITR (3′ ITR) are asymmetric with respect to each other -that is, they have a different 3D-spatial configuration from one another. As an exemplary embodiment, the first ITR can be a wild-type ITR and the second ITR can be a mutated or modified ITR, or vice versa, where the first ITR can be a mutated or modified ITR and the second ITR a wild-type ITR. In one embodiment, the first ITR and the second ITR are both modified but are different sequences, or have different modifications, or are not identical modified ITRs, and have different 3D spatial configurations. Stated differently, a ceDNA vector with asymmetric ITRs have ITRs where any changes in one ITR relative to the WT-ITR are not reflected in the other ITR; or alternatively, where the asymmetric ITRs have a the modified asymmetric ITR pair can have a different sequence and different three-dimensional shape with respect to each other.

In one embodiment, a ceDNA vector comprises, in the 5′ to 3′ direction: a first adeno-associated virus (AAV) inverted terminal repeat (ITR), a nucleotide sequence of interest (for example an expression cassette as described herein) and a second AAV ITR, where the first ITR (5′ ITR) and the second ITR (3′ ITR) are symmetric, or substantially symmetrical with respect to each other -that is, a ceDNA vector can comprise ITR sequences that have a symmetrical three-dimensional spatial organization such that their structure is the same shape in geometrical space, or have the same A, C-C′ and B-B' loops in 3D space. In such an embodiment, a symmetrical ITR pair, or substantially symmetrical ITR pair can be modified ITRs (e.g., mod-ITRs) that are not wild-type ITRs. A mod-ITR pair can have the same sequence which has one or more modifications from wild-type ITR and are reverse complements (inverted) of each other. In one embodiment, a modified ITR pair are substantially symmetrical as defined herein, that is, the modified ITR pair can have a different sequence but have corresponding or the same symmetrical three-dimensional shape. In some embodiments, the symmetrical ITRs, or substantially symmetrical ITRs can be wild type (WT-ITRs) as described herein. That is, both ITRs have a wild-type sequence, but do not necessarily have to be WT-ITRs from the same AAV serotype. In one embodiment, one WT-ITR can be from one AAV serotype, and the other WT-ITR can be from a different AAV serotype. In such an embodiment, a WT-ITR pair are substantially symmetrical as defined herein, that is, they can have one or more conservative nucleotide modification while still retaining the symmetrical three-dimensional spatial organization.

The wild-type or mutated or otherwise modified ITR sequences provided herein represent DNA sequences included in the expression construct (e.g., ceDNA-plasmid, ceDNA Bacmid, ceDNA-baculovirus) for production of the ceDNA vector. Thus, ITR sequences actually contained in the ceDNA vector produced from the ceDNA-plasmid or other expression construct may or may not be identical to the ITR sequences provided herein as a result of naturally occurring changes taking place during the production process (e.g., replication error).

In one embodiment, a ceDNA vector described herein comprising the expression cassette with a transgene which is a therapeutic nucleic acid sequence, can be operatively linked to one or more regulatory sequence(s) that allows or controls expression of the transgene. In one embodiment, the polynucleotide comprises a first ITR sequence and a second ITR sequence, wherein the nucleotide sequence of interest is flanked by the first and second ITR sequences, and the first and second ITR sequences are asymmetrical relative to each other, or symmetrical relative to each other.

In one embodiment, an expression cassette is located between two ITRs comprised in the following order with one or more of: a promoter operably linked to a transgene, a posttranscriptional regulatory element, and a polyadenylation and termination signal. In one embodiment, the promoter is regulatable -inducible or repressible. The promoter can be any sequence that facilitates the transcription of the transgene. In one embodiment the promoter is a CAG promoter, or variation thereof. The posttranscriptional regulatory element is a sequence that modulates expression of the transgene, as a non-limiting example, any sequence that creates a tertiary structure that enhances expression of the transgene which is a therapeutic nucleic acid sequence.

In one embodiment, the posttranscriptional regulatory element comprises WPRE. In one embodiment, the polyadenylation and termination signal comprise BGHpolyA. Any cis regulatory element known in the art, or combination thereof, can be additionally used e.g., SV40 late polyA signal upstream enhancer sequence (USE), or other posttranscriptional processing elements including, but not limited to, the thymidine kinase gene of herpes simplex virus, or hepatitis B virus (HBV). In one embodiment, the expression cassette length in the 5′ to 3′ direction is greater than the maximum length known to be encapsidated in an AAV virion. In one embodiment, the length is greater than 4.6 kb, or greater than 5 kb, or greater than 6 kb, or greater than 7 kb. Various expression cassettes are exemplified herein.

In one embodiment, the expression cassette can comprise more than 4000 nucleotides, 5000 nucleotides, 10,000 nucleotides or 20,000 nucleotides, or 30,000 nucleotides, or 40,000 nucleotides or 50,000 nucleotides, or any range between about 4000-10,000 nucleotides or 10,000-50,000 nucleotides, or more than 50,000 nucleotides. In some embodiments, the expression cassette can comprise a transgene which is a therapeutic nucleic acid sequence in the range of 500 to 50,000 nucleotides in length. In one embodiment, the expression cassette can comprise a transgene which is a therapeutic nucleic acid sequence in the range of 500 to 75,000 nucleotides in length. In one embodiment, the expression cassette can comprise a transgene which is a therapeutic nucleic acid sequence in the range of 500 to 10,000 nucleotides in length. In one embodiment, the expression cassette can comprise a transgene which is a therapeutic nucleic acid sequence in the range of 1000 to 10,000 nucleotides in length. In one embodiment, the expression cassette can comprise a transgene which is a therapeutic nucleic acid sequence in the range of 500 to 5,000 nucleotides in length. The ceDNA vectors do not have the size limitations of encapsidated AAV vectors, and thus enable delivery of a large-size expression cassette to the host. In one embodiment, the ceDNA vector is devoid of prokaryote-specific methylation.

In one embodiment, the rigid therapeutic nucleic acid can be a plasmid.

In one embodiment, the ceDNA vectors disclosed herein are used for therapeutic purposes (e.g., for medical, diagnostic, or veterinary uses) or immunogenic polypeptides.

The expression cassette can comprise any transgene which is a therapeutic nucleic acid sequence. In certain embodiments, the ceDNA vector comprises any gene of interest in the subject, which includes one or more polypeptides, peptides, ribozymes, peptide nucleic acids, siRNAs, RNAis, antisense oligonucleotides, antisense polynucleotides, antibodies, antigen binding fragments, or any combination thereof.

In one embodiment, the ceDNA expression cassette can include, for example, an expressible exogenous sequence (e.g., open reading frame) that encodes a protein that is either absent, inactive, or insufficient activity in the recipient subject or a gene that encodes a protein having a desired biological or a therapeutic effect. In one embodiment, the exogenous sequence such as a donor sequence can encode a gene product that can function to correct the expression of a defective gene or transcript. In one embodiment, the expression cassette can also encode corrective DNA strands, encode polypeptides, sense or antisense oligonucleotides, or RNAs (coding or non-coding; e.g., siRNAs, shRNAs, micro-RNAs, and their antisense counterparts (e.g., antagoMiR)). In one embodiment, expression cassettes can include an exogenous sequence that encodes a reporter protein to be used for experimental or diagnostic purposes, such as b-lactamase,b-galactosidase (LacZ), alkaline phosphatase, thymidine kinase, green fluorescent protein (GFP), chloramphenicol acetyltransferase (CAT), luciferase, and others well known in the art.

Accordingly, the expression cassette can include any gene that encodes a protein, polypeptide or RNA that is either reduced or absent due to a mutation or which conveys a therapeutic benefit when overexpressed is considered to be within the scope of the disclosure. The ceDNA vector may comprise a template or donor nucleotide sequence used as a correcting DNA strand to be inserted after a double-strand break (or nick) provided by a nuclease. The ceDNA vector may include a template nucleotide sequence used as a correcting DNA strand to be inserted after a double-strand break (or nick) provided by a guided RNA nuclease, meganuclease, or zinc finger nuclease.

### IV. Therapeutic Nucleic Acids

Aspects of the present disclosure generally provide compositions (e.g., pharmaceutical compositions) comprising a lipid nanoparticle (LNP) and a therapeutic nucleic acid (TNA), wherein the LNP comprises a scFv, linked to the LNP. According to embodiments, the disclosure provides pharmaceutical compositions comprising a lipid nanoparticle (LNP) and a therapeutic nucleic acid (TNA), wherein the LNP comprises a scFv, linked to the LNP, wherein the scFv is directed against an antigen present on the surface of a cell, and wherein the scFv is linked to the LNP by a maleimide conjugation.

Illustrative therapeutic nucleic acids of the present disclosure can include, but are not limited to, minigenes, plasmids, minicircles, small interfering RNA (siRNA), microRNA (miRNA), antisense oligonucleotides (ASO), ribozymes, closed ended double stranded DNA (e.g., ceDNA, CELiD, linear covalently closed DNA (“ministring”), doggybone™, protelomere closed ended DNA, or dumbbell linear DNA), dicer-substrate dsRNA, small hairpin RNA (shRNA), asymmetrical interfering RNA (aiRNA), microRNA (miRNA), mRNA, tRNA, rRNA, and DNA viral vectors, viral RNA vector, and any combination thereof.

siRNA or miRNA that can downregulate the intracellular levels of specific proteins through a process called RNA interference (RNAi) are also contemplated by the present disclosure to be nucleic acid therapeutics. After siRNA or miRNA is introduced into the cytoplasm of a host cell, these double-stranded RNA constructs can bind to a protein called RISC. The sense strand of the siRNA or miRNA is removed by the RISC complex. The RISC complex, when combined with the complementary mRNA, cleaves the mRNA and release the cut strands. RNAi is by inducing specific destruction of mRNA that results in downregulation of a corresponding protein.

Antisense oligonucleotides (ASO) and ribozymes that inhibit mRNA translation into protein can be nucleic acid therapeutics. For antisense constructs, these single stranded deoxy nucleic acids have a complementary sequence to the sequence of the target protein mRNA, and Watson -capable of binding to the mRNA by Crick base pairing. This binding prevents translation of a target mRNA, and/or triggers RNaseH degradation of the mRNA transcript. As a result, the antisense oligonucleotide has increased specificity of action (i.e., down-regulation of a specific disease-related protein).

In any of the aspects and embodiments provided herein, the therapeutic nucleic acid can be a therapeutic RNA. Said therapeutic RNA can be an inhibitor of mRNA translation, agent of RNA interference (RNAi), catalytically active RNA molecule (ribozyme), transfer RNA (tRNA) or an RNA that binds an mRNA transcript (ASO), protein or other molecular ligand (aptamer). In any of the methods provided herein, the agent of RNAi can be a double-stranded RNA, single-stranded RNA, micro RNA, short interfering RNA, short hairpin RNA, or a triplex-forming oligonucleotide.

### Denatured Therapeutic Nucleic Acids

Aspects of the present disclosure further provide pharmaceutical compositions comprising lipid particles (e.g., compositions (e.g., pharmaceutical compositions), comprising a lipid nanoparticle (LNP) and a therapeutic nucleic acid (TNA), wherein the LNP comprises a scFv (e.g., wherein the scFv is directed against an antigen present on the surface of a cell), linked to the LNP) and a denatured therapeutic nucleic acid (TNA), where TNA is as defined above.

In one embodiment, the denatured TNA is a closed ended DNA (ceDNA). The term “denatured therapeutic nucleic acid” refers to a partially or fully TNA where the conformation has changed from the standard B-form structure. The conformational changes may include changes in the secondary structure (i.e., base pair interactions within a single nucleic acid molecule) and/or changes in the tertiary structure (i.e., double helix structure). Without being bound by theory, it was thought that TNA treated with an alcohol/water solution or pure alcohol solvent results in the denaturation of the nucleic acid to a conformation that enhances encapsulation efficiency by LNP and produces LNP formulations having a smaller diameter size (i.e., smaller than 75 nm, for example, the mean size of about 68 to 74 nm in diameter). All LNP mean diameter sizes and size ranges described herein apply to LNPs containing a denatured TNA.

When DNA is in an aqueous environment, it has a B-form structure with 10.4 base pairs in each complete helical turn. If this aqueous environment is gradually changed by adding a moderately less polar alcohol such as methanol, the twist of the helix relaxes, whereby the DNA changes smoothly into a form with only 10.2 base pairs per helical turn, as visualized by circular dichroism (CD) spectroscopy. In one embodiment, the denatured TNA in a pharmaceutical composition provided herein has a 10.2-form structure.

In contrast to this behavior, if the water is replaced with a slightly less polar alcohol such as ethanol, the same kind of conformational change will occur only until about 65% of the water is replaced with ethanol. At this point, the DNA abruptly changes to the A-form structure which has a more tightly-twisted helix containing 11 base pairs per helical turn, as visualized by CD. In one embodiment, the denatured TNA in a pharmaceutical composition provided herein has an A-form structure.

According to some embodiments, the denatured TNA in a pharmaceutical composition provided herein has a rod-like structure when visualized under transmission electron microscopy (TEM). According to some embodiments, the denatured TNA in a pharmaceutical composition provided herein has a circular-like structure when visualized under transmission electron microscopy (TEM). Comparatively, TNA that has not been denatured has a strand-like structure.

V. Production of a ceDNA Vector

Embodiments of the disclosure are based on compositions comprising a lipid nanoparticle (LNP) and a therapeutic nucleic acid (TNA). The ceDNA vectors as described herein have no packaging constraints imposed by the limiting space within the viral capsid. ceDNA vectors represent a viable eukaryotically-produced alternative to prokaryote-produced plasmid DNA vectors, as opposed to encapsulated AAV genomes. This permits the insertion of control elements, e.g., regulatory switches as disclosed herein, large transgenes, multiple transgenes etc.

Methods for the production of a ceDNA vector as described herein comprising an asymmetrical ITR pair or symmetrical ITR pair as defined herein is described in section IV of PCT/US 18/49996 filed Sep. 7, 2018, which is incorporated herein in its entirety by reference. As described herein, the ceDNA vector can be obtained, for example, by the process comprising the steps of: a) incubating a population of host cells (e.g. insect cells) harboring the polynucleotide expression construct template (e.g., a ceDNA-plasmid, a ceDNA-Bacmid, and/or a ceDNA-baculovirus), which is devoid of viral capsid coding sequences, in the presence of a Rep protein under conditions effective and for a time sufficient to induce production of the ceDNA vector within the host cells, and wherein the host cells do not comprise viral capsid coding sequences; and b) harvesting and isolating the ceDNA vector from the host cells. The presence of Rep protein induces replication of the vector polynucleotide with a modified ITR to produce the ceDNA vector in a host cell.

The following is provided as a non-limiting example.

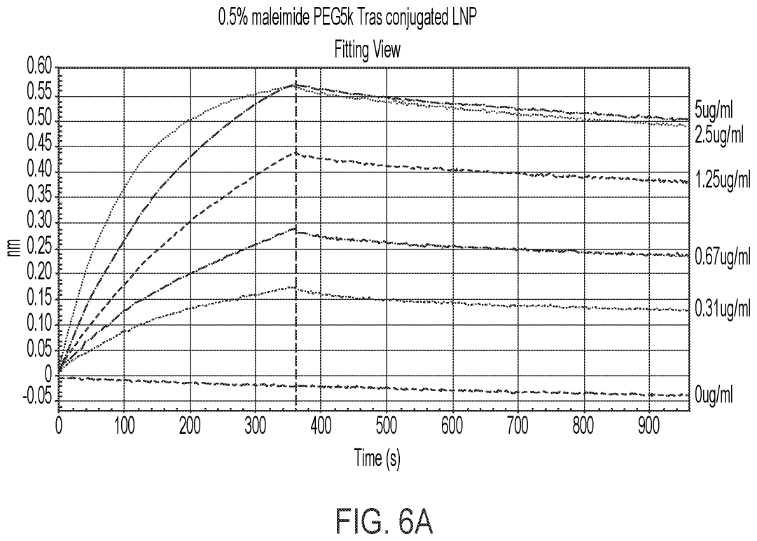

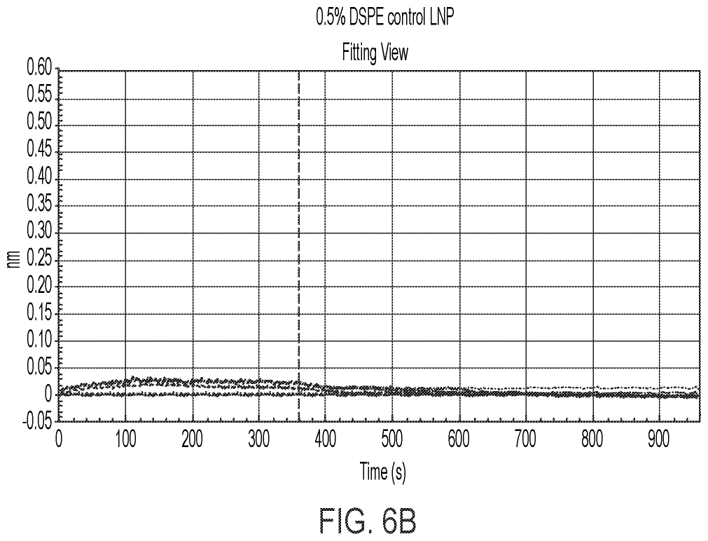

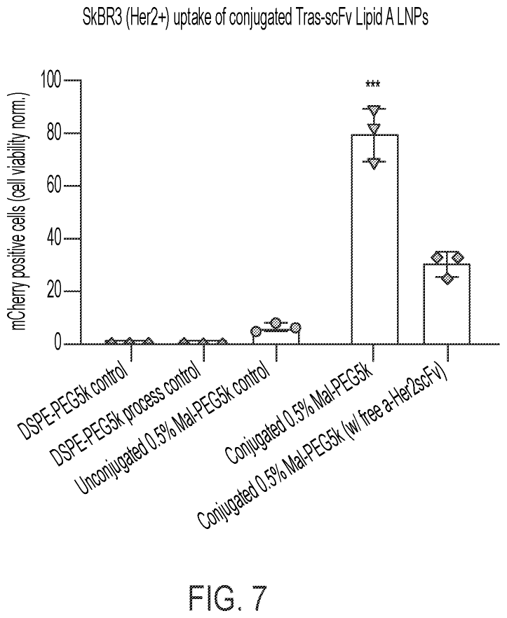

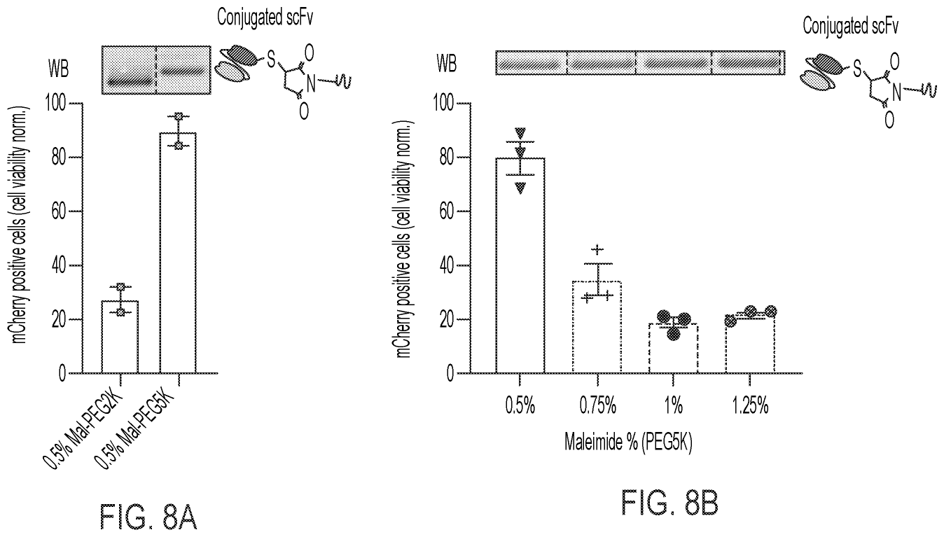

According to some embodiments, synthetic ceDNA is produced via excision from a double-stranded DNA molecule. Synthetic production of the ceDNA vectors is described in Examples 2-6 of International Application PCT/US19/14122, filed Jan. 18, 2019, which is incorporated herein in its entirety by reference. One exemplary method of producing a ceDNA vector using a synthetic method that involves the excision of a double-stranded DNA molecule. In brief, a ceDNA vector can be generated using a double stranded DNA construct, e.g., see FIGS. 7A-8E of PCT/US19/14122. In some embodiments, the double stranded DNA construct is a ceDNA plasmid, e.g., see, e.g., FIG. 6 in International patent application PCT/US2018/064242, filed Dec. 6, 2018).

In some embodiments, a construct to make a ceDNA vector comprises additional components to regulate expression of the transgene, for example, regulatory switches, to regulate the expression of the transgene, or a kill switch, which can kill a cell comprising the vector.

A molecular regulatory switch is one which generates a measurable change in state in response to a signal. Such regulatory switches can be usefully combined with the ceDNA vectors described herein to control the output of expression of the transgene. In some embodiments, the ceDNA vector comprises a regulatory switch that serves to fine tune expression of the transgene. For example, it can serve as a biocontainment function of the ceDNA vector. In some embodiments, the switch is an “ON/OFF” switch that is designed to start or stop (i.e., shut down) expression of the gene of interest in the ceDNA vector in a controllable and regulatable fashion. In some embodiments, the switch can include a “kill switch” that can instruct the cell comprising the synthetic ceDNA vector to undergo cell programmed death once the switch is activated. Exemplary regulatory switches encompassed for use in a ceDNA vector can be used to regulate the expression of a transgene, and are more fully discussed in International application PCT/US18/49996, which is incorporated herein in its entirety by reference and described herein.

Another exemplary method of producing a ceDNA vector using a synthetic method that involves assembly of various oligonucleotides, is provided in Example 3 of PCT/US19/14122, where a ceDNA vector is produced by synthesizing a 5′ oligonucleotide and a 3′ ITR oligonucleotide and ligating the ITR oligonucleotides to a double-stranded polynucleotide comprising an expression cassette. FIG. 11B of PCT/US19/14122, incorporated by reference in its entirety herein, shows an exemplary method of ligating a 5′ ITR oligonucleotide and a 3′ ITR oligonucleotide to a double stranded polynucleotide comprising an expression cassette.

An exemplary method of producing a ceDNA vector using a synthetic method is provided in Example 4 of PCT/US19/14122, incorporated by reference in its entirety herein, and uses a single-stranded linear DNA comprising two sense ITRs which flank a sense expression cassette sequence and are attached covalently to two antisense ITRs which flank an antisense expression cassette, the ends of which single stranded linear DNA are then ligated to form a closed-ended single-stranded molecule. One non-limiting example comprises synthesizing and/or producing a single-stranded DNA molecule, annealing portions of the molecule to form a single linear DNA molecule which has one or more base-paired regions of secondary structure, and then ligating the free 5′ and 3′ ends to each other to form a closed single-stranded molecule.

In yet another aspect, the disclosure provides for host cell lines that have stably integrated the DNA vector polynucleotide expression template (ceDNA template) described herein, into their own genome for use in production of the non-viral DNA vector. Methods for producing such cell lines are described in Lee, L. et al. (2013) Plos One 8(8): e69879, which is herein incorporated by reference in its entirety. For example, the Rep protein is added to host cells at an MOI of 3. In one embodiment, the host cell line is an invertebrate cell line, preferably insect Sf9 cells. When the host cell line is a mammalian cell line, preferably 293 cells the cell lines can have polynucleotide vector template stably integrated, and a second vector, such as herpes virus can be used to introduce Rep protein into cells, allowing for the excision and amplification of ceDNA in the presence of Rep.

Any promoter can be operably linked to the heterologous nucleic acid (e.g. reporter nucleic acid or therapeutic transgene) of the vector polynucleotide. The expression cassette can contain a synthetic regulatory element, such as CAG promoter. The CAG promoter comprises (i) the cytomegalovirus (CMV) early enhancer element, (ii) the promoter, the first exon and the first intron of the chicken beta actin gene, and (ii) the splice acceptor of the rabbit beta globin gene. Alternatively, expression cassette can contain an Alpha-i-antitrypsin (AAT) promoter, a liver specific (LPi) promoter, or Human elongation factor-1 alpha (EF1-α) promoter. In some embodiments, the expression cassette includes one or more constitutive promoters, for example, the retroviral Rous sarcoma virus (RSV) LTR promoter (optionally with the RSV enhancer), cytomegalovirus (CMV) immediate early promoter (optionally with the CMV enhancer). Alternatively, an inducible or repressible promoter, a native promoter for a transgene, a tissue-specific promoter, or various promoters known in the art can be used. Suitable transgenes for gene therapy are well known to those of skill in the art.

The capsid-free ceDNA vectors can also be produced from vector polynucleotide expression constructs that further comprise cis-regulatory elements, or combination of cis regulatory elements, a non-limiting example include a woodchuck hepatitis virus posttranscriptional regulatory element (WPRE) and BGH polyA, or e.g., beta-globin polyA. Other posttranscriptional processing elements include, e.g., the thymidine kinase gene of herpes simplex virus, or hepatitis B virus (HBV). The expression cassettes can include any poly-adenylation sequence known in the art or a variation thereof, such as a naturally occurring isolated from bovine BGHpA or a virus SV40 pA, or synthetic.

Some expression cassettes can also include SV40 late polyA signal upstream enhancer (USE) sequence. The USE can be used in combination with SV40 pA or heterologous poly-A signal.

The time for harvesting and collecting DNA vectors described herein from the cells can be selected and optimized to achieve a high-yield production of the ceDNA vectors. For example, the harvest time can be selected in view of cell viability, cell morphology, cell growth, etc. In one embodiment, cells are grown under sufficient conditions and harvested a sufficient time after baculoviral infection to produce DNA-vectors) but before the majority of cells start to die because of the viral toxicity. The DNA-vectors can be isolated using plasmid purification kits such as Qiagen Endo-Free Plasmid kits. Other methods developed for plasmid isolation can be also adapted for DNA-vectors. Generally, any nucleic acid purification methods can be adopted.

The DNA vectors can be purified by any means known to those of skill in the art for purification of DNA. In one embodiment, ceDNA vectors are purified as DNA molecules. In another embodiment, the ceDNA vectors are purified as exosomes or microparticles.

In one embodiment, the capsid free non-viral DNA vector comprises or is obtained from a plasmid comprising a polynucleotide template comprising in this order: a first adeno-associated virus (AAV) inverted terminal repeat (ITR), a nucleotide sequence of interest (for example an expression cassette of an exogenous DNA) and a modified AAV ITR, wherein said template nucleic acid molecule is devoid of AAV capsid protein coding. In a further embodiment, the nucleic acid template of the disclosure is devoid of viral capsid protein coding sequences (i.e., it is devoid of AAV capsid genes but also of capsid genes of other viruses). In addition, in a particular embodiment, the template nucleic acid molecule is also devoid of AAV Rep protein coding sequences. Accordingly, in a preferred embodiment, the nucleic acid molecule of the disclosure is devoid of both functional AAV cap and AAV rep genes.

In one embodiment, ceDNA can include an ITR structure that is mutated with respect to the wild type AAV2 ITR disclosed herein, but still retains an operable RBE, TRS and RBE′ portion.

ceDNA Plasmid

A ceDNA-plasmid is a plasmid used for later production of a ceDNA vector. In one embodiment, a ceDNA-plasmid can be constructed using known techniques to provide at least the following as operatively linked components in the direction of transcription: (1) a modified 5′ ITR sequence; (2) an expression cassette containing a cis-regulatory element, for example, a promoter, inducible promoter, regulatory switch, enhancers and the like; and (3) a modified 3′ ITR sequence, where the 3′ ITR sequence is symmetric relative to the 5′ ITR sequence. In some embodiments, the expression cassette flanked by the ITRs comprises a cloning site for introducing an exogenous sequence. The expression cassette replaces the rep and cap coding regions of the AAV genomes.

In one embodiment, a ceDNA vector is obtained from a plasmid, referred to herein as a “ceDNA-plasmid” encoding in this order: a first adeno-associated virus (AAV) inverted terminal repeat (ITR), an expression cassette comprising a transgene, and a mutated or modified AAV ITR, wherein said ceDNA-plasmid is devoid of AAV capsid protein coding sequences. In alternative embodiments, the ceDNA-plasmid encodes in this order: a first (or 5′) modified or mutated AAV ITR, an expression cassette comprising a transgene, and a second (or 3′) modified AAV ITR, wherein said ceDNA-plasmid is devoid of AAV capsid protein coding sequences, and wherein the 5′ and 3′ ITRs are symmetric relative to each other. In alternative embodiments, the ceDNA-plasmid encodes in this order: a first (or 5′) modified or mutated AAV ITR, an expression cassette comprising a transgene, and a second (or 3′) mutated or modified AAV ITR, wherein said ceDNA-plasmid is devoid of AAV capsid protein coding sequences, and wherein the 5′ and 3′ modified ITRs have the same modifications (i.e., they are inverse complement or symmetric relative to each other).

In one embodiment, the ceDNA-plasmid system is devoid of viral capsid protein coding sequences (i.e., it is devoid of AAV capsid genes but also of capsid genes of other viruses). In one embodiment, the ceDNA-plasmid is also devoid of AAV Rep protein coding sequences. In one embodiment, ceDNA-plasmid is devoid of functional AAV cap and AAV rep genes GG-3′ for AAV2) plus a variable palindromic sequence allowing for hairpin formation. In one embodiment, a ceDNA-plasmid of the present disclosure can be generated using natural nucleotide sequences of the genomes of any AAV serotypes well known in the art. In one embodiment, the ceDNA-plasmid backbone is derived from the AAV1, AAV2, AAV3, AAV4, AAVS, AAV 5, AAV7, AAV8, AAV9, AAV 10, AAV 11, AAV 12, AAVrh8, AAVrhlO, AAV-DJ, and AAV-DJ8 genome, e.g., NCBI: NC 002077; NC 001401; NC001729; NC001829; NC006152; NC 006260; NC 006261; Kotin and Smith, The Springer Index of Viruses, available at the URL maintained by Springer. In one embodiment, the ceDNA-plasmid backbone is derived from the AAV2 genome. In one embodiment, the ceDNA-plasmid backbone is a synthetic backbone genetically engineered to include at its 5′ and 3′ ITRs derived from one of these AAV genomes.

In one embodiment, a ceDNA-plasmid can optionally include a selectable or selection marker for use in the establishment of a ceDNA vector-producing cell line. In one embodiment, the selection marker can be inserted downstream (i.e., 3′) of the 3′ ITR sequence. In another embodiment, the selection marker can be inserted upstream (i.e., 5′) of the 5′ ITR sequence. Appropriate selection markers include, for example, those that confer drug resistance. Selection markers can be, for example, a blasticidin S-resistance gene, kanamycin, geneticin, and the like.

### VI. Preparation of Lipid Particles

Lipid particles (e.g., lipid nanoparticles) can form spontaneously upon mixing of ceDNA and the lipid(s). Depending on the desired particle size distribution, the resultant nanoparticle mixture can be extruded through a membrane (e.g., 100 nrn cut-off) using, for example, a thermobarrel extruder, such as Lipex Extruder (Northern Lipids, Inc). In some cases, the extrusion step can be omitted. Ethanol removal and simultaneous buffer exchange can be accomplished by, for example, dialysis or tangential flow filtration. In one embodiment, the lipid nanoparticles are formed as described in Example 3 described in U.S. Provisional Application No. 63/194,620.

Generally, lipid particles (e.g., lipid nanoparticles) can be formed by any method known in the art. For example, the lipid particles (e.g., lipid nanoparticles) can be prepared by the methods described, for example, in US2013/0037977, US2010/0015218, US2013/0156845, US2013/0164400, US2012/0225129, and US2010/0130588, content of each of which is incorporated herein by reference in its entirety. In some embodiments, lipid particles (e.g., lipid nanoparticles) can be prepared using a continuous mixing method, a direct dilution process, or an in-line dilution process. The processes and apparatuses for apparatuses for preparing lipid nanoparticles using direct dilution and in-line dilution processes are described in US2007/0042031, the content of which is incorporated herein by reference in its entirety. The processes and apparatuses for preparing lipid nanoparticles using step-wise dilution processes are described in US2004/0142025, the content of which is incorporated herein by reference in its entirety.

According to some embodiments, the disclosure provides for an LNP comprising a DNA vector, including a ceDNA vector as described herein and an ionizable lipid. For example, a lipid nanoparticle formulation that is made and loaded with therapeutic nucleic acid like ceDNA obtained by the process as disclosed in International Patent Application No. PCT/US2018/050042, filed on Sep. 7, 2018, which is incorporated by reference in its entirety herein.

In one embodiment, the lipid particles (e.g., lipid nanoparticles) can be prepared by an impinging jet process. Generally, the particles are formed by mixing lipids dissolved in alcohol (e.g., ethanol) with ceDNA dissolved in a buffer, e.g., a citrate buffer, a sodium acetate buffer, a sodium acetate and magnesium chloride buffer, a malic acid buffer, a malic acid and sodium chloride buffer, or a sodium citrate and sodium chloride buffer. The mixing ratio of lipids to ceDNA can be about 45-55% lipid and about 65-45% ceDNA.

The lipid solution can contain a cationic lipid (e.g., an ionizable cationic lipid), a non-cationic lipid (e.g., a phospholipid, such as DSPC, DOPE, and DOPC), PEG or PEG conjugated molecule (e.g., PEG-lipid), and a sterol (e.g., cholesterol) at a total lipid concentration of 5-30 mg/mL, more likely 5-15 mg/mL, most likely 9-12 mg/mL in an alcohol, e.g., in ethanol. In the lipid solution, mol ratio of the lipids can range from about 25-98% for the cationic lipid, preferably about 35-65%; about 0-15% for the non-ionic lipid, preferably about 0-12%; about 0-15% for the PEG or PEG conjugated lipid molecule, preferably about 1-6%; and about 0-75% for the sterol, preferably about 30-50%.

The ceDNA solution can comprise the ceDNA at a concentration range from 0.3 to 1.0 mg/mL, preferably 0.3-0.9 mg/mL in buffered solution, with pH in the range of 3.5-5.

For forming the LNPs, in one exemplary but nonlimiting embodiment, the two liquids are heated to a temperature in the range of about 15-40° C., preferably about 30-40° C., and then mixed, for example, in an impinging jet mixer, instantly forming the LNP. The mixing flow rate can range from 10-600 m/min. The tube ID can have a range from 0.25 to 1.0 mm and a total flow rate from 10-600 m/min. The combination of flow rate and tubing ID can have the effect of controlling the particle size of the LNPs between 30 and 200 nm. The solution can then be mixed with a buffered solution at a higher pH with a mixing ratio in the range of 1:1 to 1:3 vol:vol, preferably about 1:2 vol:vol. If needed this buffered solution can be at a temperature in the range of 15-40° C. or 30-40° C. The mixed LNPs can then undergo an anion exchange filtration step. Prior to the anion exchange, the mixed LNPs can be incubated for a period of time, for example 30 mins to 2 hours. The temperature during incubating can be in the range of 15-40° C. or 30-40° C. After incubating the solution is filtered through a filter, such as a 0.8 μm filter, containing an anion exchange separation step. This process can use tubing IDs ranging from 1 mm ID to 5 mm ID and a flow rate from 10 to 2000 mL/min.

After formation, the LNPs can be concentrated and diafiltered via an ultrafiltration process where the alcohol is removed and the buffer is exchanged for the final buffer solution, for example, phosphate buffered saline (PBS) at about pH 7, e.g., about pH 6.9, about pH 7.0, about pH 7.1, about pH 7.2, about pH 7.3, or about pH 7.4.

The ultrafiltration process can use a tangential flow filtration format (TFF) using a membrane nominal molecular weight cutoff range from 30-500 kD. The membrane format is hollow fiber or flat sheet cassette. The TFF processes with the proper molecular weight cutoff can retain the LNP in the retentate and the filtrate or permeate contains the alcohol; citrate buffer and final buffer wastes. The TFF process is a multiple step process with an initial concentration to a ceDNA concentration of 1-3 mg/mL. Following concentration, the LNPs solution is diafiltered against the final buffer for 10-20 volumes to remove the alcohol and perform buffer exchange. The material can then be concentrated an additional 1-3-fold. The concentrated LNP solution can be sterile filtered.

### VII. Pharmaceutical Compositions and Formulations

Provided herein is a pharmaceutical composition comprising a lipid nanoparticle (LNP) and a therapeutic nucleic acid (TNA), wherein the LNP comprises a scFv linked to the LNP, and at least one pharmaceutically acceptable excipient. According to one aspect, provided herein are pharmaceutical compositions comprising a lipid nanoparticle (LNP) and a therapeutic nucleic acid (TNA), wherein the LNP comprises a single-chain variable fragment (scFv) linked to the LNP, wherein the scFv is directed against an antigen present on the surface of a cell, and at least one pharmaceutically acceptable excipient, wherein the scFv is covalently linked to the LNP via a non-cleavable linker. According to one aspect, provided herein are pharmaceutical compositions comprising a lipid nanoparticle (LNP) and a therapeutic nucleic acid (TNA), wherein the LNP comprises a single-chain variable fragment (scFv) linked to the LNP, wherein the scFv is directed against an antigen present on the surface of a cell, and at least one pharmaceutically acceptable excipient, wherein the scFv is covalently linked to the LNP via a cleavable linker. According to one aspect, provided herein are pharmaceutical compositions comprising a lipid nanoparticle (LNP) and a therapeutic nucleic acid (TNA), wherein the LNP comprises a single-chain variable fragment (scFv) linked to the LNP, wherein the scFv is directed against an antigen present on the surface of a cell, and at least one pharmaceutically acceptable excipient, wherein the scFv is non-covalently linked to the LNP.

According to some embodiments, the TNA (e.g., ceDNA) is encapsulated in the lipid. In one embodiment, the TNA (e.g., ceDNA) lipid particles (e.g., lipid nanoparticles) are provided with full encapsulation, partial encapsulation of the therapeutic nucleic acid. In one embodiment, the nucleic acid therapeutics is fully encapsulated in the lipid particles (e.g., lipid nanoparticles) to form a nucleic acid containing lipid particle. In one embodiment, the nucleic acid may be encapsulated within the lipid portion of the particle, thereby protecting it from enzymatic degradation.

Depending on the intended use of the lipid particles (e.g., lipid nanoparticles), the proportions of the components can vary and the delivery efficiency of a particular formulation can be measured using, for example, an endosomal release parameter (ERP) assay.

In one embodiment, the lipid particles (e.g., lipid nanoparticles) may be conjugated with other moieties to prevent aggregation. Such lipid conjugates include, but are not limited to, PEG-lipid conjugates such as, e.g., PEG coupled to dialkyloxypropyls (e.g., PEG-DAA conjugates), PEG coupled to diacylglycerols (e.g., PEG-DAG conjugates), PEG coupled to cholesterol, PEG coupled to phosphatidylethanolamines, and PEG conjugated to ceramides (see, e.g., U.S. Pat. No. 5,885,613), cationic PEG lipids, polyoxazoline (POZ)-lipid conjugates (e.g., POZ-DAA conjugates; see, e.g., U.S. Provisional Application No. 61/294,828, filed Jan. 13, 2010, and U.S. Provisional Application No. 61/295,140, filed Jan. 14, 2010), polyamide oligomers (e.g., ATTA-lipid conjugates), and mixtures thereof. Additional examples of POZ-lipid conjugates are described in PCT Publication No. WO 2010/006282. PEG or POZ can be conjugated directly to the lipid or may be linked to the lipid via a linker moiety. Any linker moiety suitable for coupling the PEG or the POZ to a lipid can be used including, e.g., non-ester containing linker moieties and ester-containing linker moieties. In certain preferred embodiments, non-ester containing linker moieties, such as amides or carbamates, are used. The disclosures of each of the above patent documents are herein incorporated by reference in their entirety for all purposes.

In one embodiment, the TNA (e.g., ceDNA) can be complexed with the lipid portion of the particle or encapsulated in the lipid position of the lipid particle (e.g., lipid nanoparticle). In one embodiment, the TNA can be fully encapsulated in the lipid position of the lipid particle (e.g., lipid nanoparticle), thereby protecting it from degradation by a nuclease, e.g., in an aqueous solution. In one embodiment, the TNA in the lipid particle (e.g., lipid nanoparticle) is not substantially degraded after exposure of the lipid particle (e.g., lipid nanoparticle) to a nuclease at 37° C. for at least about 20, 30, 45, or 60 minutes. In some embodiments, the TNA in the lipid particle (e.g., lipid nanoparticle) is not substantially degraded after incubation of the particle in serum at 37° C. for at least about 30, 45, or 60 minutes or at least about 2, 3, 4, 5, 6, 7, 8, 9, 10, 12, 14, 16, 18, 20, 22, 24, 26, 28, 30, 32, 34, or 36 hours.

In one embodiment, the lipid particles (e.g., lipid nanoparticles) are substantially non-toxic to a subject, e.g., to a mammal such as a human.

In one embodiment, a pharmaceutical composition comprising a therapeutic nucleic acid of the present disclosure may be formulated in lipid particles (e.g., lipid nanoparticles). In some embodiments, the lipid particle comprising a therapeutic nucleic acid can be formed from a cationic lipid. In some other embodiments, the lipid particle comprising a therapeutic nucleic acid can be formed from non-cationic lipid. In a preferred embodiment, the lipid particle of the disclosure is a nucleic acid containing lipid particle, which is formed from a cationic lipid comprising a therapeutic nucleic acid selected from the group consisting of mRNA, antisense RNA and oligonucleotide, ribozymes, aptamer, interfering RNAs (RNAi), Dicer-substrate dsRNA, small hairpin RNA (shRNA), asymmetrical interfering RNA (aiRNA), microRNA (miRNA), minicircle DNA, minigene, viral DNA (e.g., Lentiviral or AAV genome) or non-viral synthetic DNA vectors, closed-ended linear duplex DNA (ceDNA/CELiD), plasmids, bacmids, doggybone™ DNA vectors, minimalistic immunological-defined gene expression (MIDGE)-vector, nonviral ministring DNA vector (linear-covalently closed DNA vector), or dumbbell-shaped DNA minimal vector (“dumbbell DNA”).

In another preferred embodiment, the lipid particle of the disclosure is a nucleic acid containing lipid particle, which is formed from a non-cationic lipid, and optionally a conjugated lipid that prevents aggregation of the particle.

In one embodiment, the lipid particle formulation is an aqueous solution. In one embodiment, the lipid particle (e.g., lipid nanoparticle) formulation is a lyophilized powder.

According to some aspects, the disclosure provides for a lipid particle formulation further comprising one or more pharmaceutical excipients. In one embodiment, the lipid particle (e.g., lipid nanoparticle) formulation further comprises sucrose, tris, trehalose and/or glycine.

In one embodiment, the lipid particles (e.g., lipid nanoparticles) disclosed herein can be incorporated into pharmaceutical compositions suitable for administration to a subject for in vivo delivery to cells, tissues, or organs of the subject. Typically, the pharmaceutical composition comprises the TNA (e.g., ceDNA) lipid particles (e.g., lipid nanoparticles) disclosed herein and a pharmaceutically acceptable carrier. In one embodiment, the TNA (e.g., ceDNA) lipid particles (e.g., lipid nanoparticles) of the disclosure can be incorporated into a pharmaceutical composition suitable for a desired route of therapeutic administration (e.g., parenteral administration). Passive tissue transduction via high pressure intravenous or intraarterial infusion, as well as intracellular injection, such as intranuclear microinjection or intracytoplasmic injection, are also contemplated. Pharmaceutical compositions for therapeutic purposes can be formulated as a solution, microemulsion, dispersion, liposomes, or other ordered structure suitable for high TNA (e.g., ceDNA) vector concentration. Sterile injectable solutions can be prepared by incorporating the TNA (e.g., ceDNA) vector compound in the required amount in an appropriate buffer with one or a combination of ingredients enumerated above, as required, followed by filtered sterilization.

A lipid particle as disclosed herein can be incorporated into a pharmaceutical composition suitable for topical, systemic, intra-amniotic, intrathecal, intracranial, intraarterial, intravenous, intralymphatic, intraperitoneal, subcutaneous, tracheal, intra-tissue (e.g., intramuscular, intracardiac, intrahepatic, intrarenal, intracerebral), intrathecal, intravesical, conjunctival (e.g., extra-orbital, intraorbital, retroorbital, intraretinal, subretinal, choroidal, sub-choroidal, intrastromal, intracameral and intravitreal), intracochlear, and mucosal (e.g., oral, rectal, nasal) administration. Passive tissue transduction via high pressure intravenous or intraarterial infusion, as well as intracellular injection, such as intranuclear microinjection or intracytoplasmic injection, are also contemplated.

Pharmaceutically active compositions comprising TNA (e.g., ceDNA) lipid particles (e.g., lipid nanoparticles) can be formulated to deliver a transgene in the nucleic acid to the cells of a recipient, resulting in the therapeutic expression of the transgene therein. The composition can also include a pharmaceutically acceptable carrier.

Pharmaceutical compositions for therapeutic purposes typically must be sterile and stable under the conditions of manufacture and storage. The composition can be formulated as a solution, microemulsion, dispersion, liposomes, or other ordered structure suitable to high TNA (e.g., ceDNA) vector concentration. Sterile injectable solutions can be prepared by incorporating the ceDNA vector compound in the required amount in an appropriate buffer with one or a combination of ingredients enumerated above, as required, followed by filtered sterilization.

In one embodiment, lipid particles (e.g., lipid nanoparticles) are solid core particles that possess at least one lipid bilayer. In one embodiment, the lipid particles (e.g., lipid nanoparticles) have a non-bilayer structure, i.e., a non-lamellar (i.e., non-bilayer) morphology. Without limitations, the non-bilayer morphology can include, for example, three dimensional tubes, rods, cubic symmetries, etc. The non-lamellar morphology (i.e., non-bilayer structure) of the lipid particles (e.g., lipid nanoparticles) can be determined using analytical techniques known to and used by those of skill in the art. Such techniques include, but are not limited to, Cryo-Transmission Electron Microscopy (“Cryo-TEM”), Differential Scanning calorimetry (“DSC”), X-Ray Diffraction, and the like. For example, the morphology of the lipid particles (lamellar vs. non-lamellar) can readily be assessed and characterized using, e.g., Cryo-TEM analysis as described in US2010/0130588, the content of which is incorporated herein by reference in its entirety.

In one embodiment, the lipid particles (e.g., lipid nanoparticles) having a non-lamellar morphology are electron dense.

In one embodiment, the disclosure provides for a lipid particle (e.g., lipid nanoparticle) that is either unilamellar or multilamellar in structure. In some aspects, the disclosure provides for a lipid particle (e.g., lipid nanoparticle) formulation that comprises multi-vesicular particles and/or foam-based particles. By controlling the composition and concentration of the lipid components, one can control the rate at which the lipid conjugate exchanges out of the lipid particle and, in turn, the rate at which the lipid particle (e.g., lipid nanoparticle) becomes fusogenic. In addition, other variables including, for example, pH, temperature, or ionic strength, can be used to vary and/or control the rate at which the lipid particle (e.g., lipid nanoparticle) becomes fusogenic. Other methods which can be used to control the rate at which the lipid particle (e.g., lipid nanoparticle) becomes fusogenic will be apparent to those of ordinary skill in the art based on this disclosure. It will also be apparent that by controlling the composition and concentration of the lipid conjugate, one can control the lipid particle size.

In one embodiment, the pKa of formulated cationic lipids can be correlated with the effectiveness of the LNPs for delivery of nucleic acids (see Jayaraman et al., Angewandte Chemie, International Edition (2012), 51(34), 8529-8533; Semple et al., Nature Biotechnology 28, 172-176 (2010), both of which are incorporated by reference in their entireties). In one embodiment, the preferred range of pKa is −5 to ˜ 7. In one embodiment, the pKa of the cationic lipid can be determined in lipid particles (e.g., lipid nanoparticles) using an assay based on fluorescence of 2-(p-toluidino)-6-napthalene sulfonic acid (TNS).

In one embodiment, encapsulation of TNA (e.g., ceDNA) in lipid particles (e.g., lipid nanoparticles) can be determined by performing a membrane-impermeable fluorescent dye exclusion assay, which uses a dye that has enhanced fluorescence when associated with nucleic acid, for example, an Oligreen® assay or PicoGreen® assay. Generally, encapsulation is determined by adding the dye to the lipid particle formulation, measuring the resulting fluorescence, and comparing it to the fluorescence observed upon addition of a small amount of nonionic detergent. Detergent-mediated disruption of the lipid bilayer releases the encapsulated TNA (e.g., ceDNA), allowing it to interact with the membrane-impermeable dye. Encapsulation of ceDNA can be calculated as E=(Io-I)/Io, where I and Io refer to the fluorescence intensities before and after the addition of detergent.

According to some embodiments, for ophthalmic delivery, interfering RNA-ligand conjugates and nanoparticle-ligand conjugates may be combined with ophthalmologically acceptable preservatives, co-solvents, surfactants, viscosity enhancers, penetration enhancers, buffers, sodium chloride, or water to form an aqueous, sterile ophthalmic suspension or solution.

Unit Dosage In one embodiment, the pharmaceutical compositions can be presented in unit dosage form. A unit dosage form will typically be adapted to one or more specific routes of administration of the pharmaceutical composition. In some embodiments, the unit dosage form is adapted for administration by inhalation. In some embodiments, the unit dosage form is adapted for administration by a vaporizer. In some embodiments, the unit dosage form is adapted for administration by a nebulizer. In some embodiments, the unit dosage form is adapted for administration by an aerosolizer. In some embodiments, the unit dosage form is adapted for oral administration, for buccal administration, or for sublingual administration. In some embodiments, the unit dosage form is adapted for intravenous, intramuscular, or subcutaneous administration. In some embodiments, the unit dosage form is adapted for intrathecal or intracerebroventricular administration. In some embodiments, the pharmaceutical composition is formulated for topical administration. The amount of active ingredient which can be combined with a carrier material to produce a single dosage form will generally be that amount of the compound which produces a therapeutic effect.

### VIII. Methods of Treatment

The pharmaceutical compositions comprising a lipid nanoparticle (LNP) and a therapeutic nucleic acid (TNA), wherein the LNP comprises a scFv (e.g., wherein the scFv is directed against an antigen present on the surface of a cell), linked to the LNP, as described herein, can be used to introduce a nucleic acid sequence (e.g., a TNA) in a cell to treat or prevent a disease or disorder. According to some embodiments, the pharmaceutical compositions may be used in a diagnostic method.

Provided herein are methods of treating a disease or disorder in a subject comprising introducing into a target cell in need thereof (for example, a muscle cell or tissue, or other affected cell type) of the subject a therapeutically effective amount of pharmaceutical composition comprising a lipid nanoparticle (LNP) and a therapeutic nucleic acid (TNA), wherein the LNP comprises a scFv, linked to the LNP, wherein the scFv is directed against an antigen present on the surface of a cell, wherein the cell is a tumor cell.

Thus, according to some aspects, the pharmaceutical compositions described herein may be used in a method of treating cancer. According to other aspects, the pharmaceutical compositions described herein may be used in a method of preventing cancer or preventing the reoccurrence of cancer.

As used herein, the term “cancer” refers to any of various malignant neoplasms characterized by the proliferation of anaplastic cells that tend to invade surrounding tissue and metastasize to new body sites and also refers to the pathological condition characterized by such malignant neoplastic growths. Cancers may be localized (e.g., solid tumors) or systemic. In the context of the present disclosure, the term “localized” (as in “localized tumor”) refers to anatomically isolated or isolatable abnormalities, such as solid malignancies, as opposed to systemic disease. Certain cancers, such as certain leukemia (e.g., myelofibrosis) and multiple myeloma, for example, may have both a localized component (for instance the bone marrow) and a systemic component (for instance circulating blood cells) to the disease. In some embodiments, cancers may be systemic, such as hematological malignancies. Cancers that may be treated according to the present disclosure include but are not limited to, all types of lymphomas/leukemias, carcinomas and sarcomas, such as those cancers or tumors found in the anus, bladder, bile duct, bone, brain, breast, cervix, colon/rectum, endometrium, esophagus, eye, gallbladder, head and neck, liver, kidney, larynx, lung, mediastinum (chest), mouth, ovaries, pancreas, penis, prostate, skin, small intestine, stomach, spinal marrow, tailbone, testicles, thyroid and uterus. Types of carcinomas which may be treated by the methods of the present disclosure include, but are not limited to, papilloma/carcinoma, choriocarcinoma, endodermal sinus tumor, teratoma, adenoma/adenocarcinoma, melanoma, fibroma, lipoma, leiomyoma, rhabdomyoma, mesothelioma, angioma, osteoma, chondroma, glioma, lymphoma/leukemia, squamous cell carcinoma, small cell carcinoma, large cell undifferentiated carcinomas, basal cell carcinoma and sinonasal undifferentiated carcinoma. Types of sarcomas include, but are not limited to, soft tissue sarcoma such as alveolar soft part sarcoma, angiosarcoma, dermatofibrosarcoma, desmoid tumor, desmoplastic small round cell tumor, extraskeletal chondrosarcoma, extraskeletal osteosarcoma, fibrosarcoma, hemangiopericytoma, hemangiosarcoma, Kaposi's sarcoma, leiomyosarcoma, liposarcoma, lymphangiosarcoma, lymphosarcoma, malignant fibrous histiocytoma, neurofibrosarcoma, rhabdomyosarcoma, synovial sarcoma, and Askin's tumor, Ewing's sarcoma (primitive neuroectodermal tumor), malignant hemangioendothelioma, malignant schwannoma, osteosarcoma, and chondrosarcoma.

The TNA (e.g., ceDNA) lipid nanoparticles can be administered via any suitable route as described herein and known in the art. In one embodiment, the target cells are in a human subject.

Provided herein are methods for providing a subject in need thereof with a diagnostically- or therapeutically-effective amount of the pharmaceutical composition comprising a LNP and a TNA, wherein the LNP comprises a scFv, wherein the scFv is directed against an antigen present on the surface of a cell, linked to the LNP, as described herein, the method comprising providing to a cell, tissue or organ of a subject in need thereof, an amount of the pharmaceutical composition comprising a LNP and a TNA, wherein the LNP comprises a scFv, wherein the scFv is directed against an antigen present on the surface of a cell, linked to the LNP, as described herein; and for a time effective to enable expression of the transgene from the ceDNA vector thereby providing the subject with a diagnostically- or a therapeutically-effective amount of the pharmaceutical composition comprising a LNP and a TNA, wherein the LNP comprises a scFv, wherein the scFv is directed against an antigen present on the surface of a cell, linked to the LNP, as described herein. In one embodiment, the subject is human.

Provided herein are methods comprising using the pharmaceutical composition comprising a LNP and a TNA, wherein the LNP comprises a scFv, wherein the scFv is directed against an antigen present on the surface of a cell, linked to the LNP, as described herein, for treating or reducing one or more symptoms of a disease or disease states. There are a number of inherited diseases in which defective genes are known, and typically fall into two classes: deficiency states, usually of enzymes, which are generally inherited in a recessive manner, and unbalanced states, which may involve regulatory or structural proteins, and which are typically but not always inherited in a dominant manner. For deficiency state diseases, the pharmaceutical composition comprising a LNP and a TNA, wherein the LNP comprises a scFv, wherein the scFv is directed against an antigen present on the surface of a cell, linked to the LNP, as described herein, can be used to deliver transgenes to bring a normal gene into affected tissues for replacement therapy, as well, in some embodiments, to create animal models for the disease using antisense mutations. As used herein, a disease state is treated by partially or wholly remedying the deficiency or imbalance that causes the disease or makes it more severe.

In general, the pharmaceutical composition comprising a LNP and a TNA, wherein the LNP comprises a scFv, wherein the scFv is directed against an antigen present on the surface of a cell), linked to the LNP, as described herein can be used to deliver any transgene in accordance with the description above to treat, prevent, or ameliorate the symptoms associated with any disorder related to gene expression. Illustrative disease states include, but are not-limited to: cystic fibrosis (and other diseases of the lung), hemophilia A, hemophilia B, thalassemia, anemia and other blood disorders, AIDS, Alzheimer's disease, Parkinson's disease, Huntington's disease, amyotrophic lateral sclerosis, epilepsy, and other neurological disorders, cancer, diabetes mellitus, muscular dystrophies (e.g., Duchenne, Becker), Hurler's disease, adenosine deaminase deficiency, metabolic defects, retinal degenerative diseases (and other diseases of the eye), mitochondriopathies (e.g., Leber's hereditary optic neuropathy (LHON), Leigh syndrome, and subacute sclerosing encephalopathy), myopathies (e.g., facioscapulohumeral myopathy (FSHD) and cardiomyopathies), diseases of solid organs (e.g., brain, liver, kidney, heart), and the like. In some embodiments, the ceDNA vectors as disclosed herein can be advantageously used in the treatment of individuals with metabolic disorders (e.g., ornithine transcarbamylase deficiency).

In one embodiment, the pharmaceutical composition comprising a LNP and a TNA, wherein the LNP comprises a scFv, wherein the scFv is directed against an antigen present on the surface of a cell), linked to the LNP, as described herein can be used to treat, ameliorate, and/or prevent a disease or disorder caused by mutation in a gene or gene product. Exemplary diseases or disorders that can be treated with ceDNA vectors (e.g., the pharmaceutical composition comprising a LNP and a TNA, wherein the LNP comprises a scFv, wherein the scFv is directed against an antigen present on the surface of a cell), linked to the LNP, as described herein) include, but are not limited to, cancers and tumors, metabolic diseases or disorders (e.g., Fabry disease, Gaucher disease, phenylketonuria (PKU), glycogen storage disease); urea cycle diseases or disorders (e.g., ornithine transcarbamylase (OTC) deficiency); lysosomal storage diseases or disorders (e.g., metachromatic leukodystrophy (MLD), mucopolysaccharidosis Type II (MPSII; Hunter syndrome)); liver diseases or disorders (e.g., progressive familial intrahepatic cholestasis (PFIC); blood diseases or disorders (e.g., hemophilia (A and B), thalassemia, and anemia); and genetic diseases or disorders (e.g., cystic fibrosis).

In one embodiment, the pharmaceutical composition comprising a LNP and a TNA, wherein the LNP comprises a scFv, wherein the scFv is directed against an antigen present on the surface of a cell, linked to the LNP, as described herein, may be employed to deliver a heterologous nucleotide sequence in situations in which it is desirable to regulate the level of transgene expression (e.g., transgenes encoding hormones or growth factors, as described herein).

In one embodiment, the pharmaceutical composition comprising a LNP and a TNA, wherein the LNP comprises a scFv, wherein the scFv is directed against an antigen present on the surface of a cell, linked to the LNP, as described herein, can be used to correct an abnormal level and/or function of a gene product (e.g., an absence of, or a defect in, a protein) that results in the disease or disorder. The ceDNA vectors in lipid nanoparticles as described herein can produce a functional protein and/or modify levels of the protein to alleviate or reduce symptoms resulting from, or confer benefit to, a particular disease or disorder caused by the absence or a defect in the protein. For example, treatment of OTC deficiency can be achieved by producing functional OTC enzyme; treatment of hemophilia A and B can be achieved by modifying levels of Factor VIII, Factor IX, and Factor X; treatment of PKU can be achieved by modifying levels of phenylalanine hydroxylase enzyme; treatment of Fabry or Gaucher disease can be achieved by producing functional alpha galactosidase or beta glucocerebrosidase, respectively; treatment of MFD or MPSII can be achieved by producing functional arylsulfatase A or iduronate-2-sulfatase, respectively; treatment of cystic fibrosis can be achieved by producing functional cystic fibrosis transmembrane conductance regulator; treatment of glycogen storage disease can be achieved by restoring functional G6Pase enzyme function; and treatment of PFIC can be achieved by producing functional ATP8B1, ABCB11, ABCB4, or TJP2 genes.

In one embodiment, the pharmaceutical composition comprising a LNP and a TNA, wherein the LNP comprises a scFv, wherein the scFv is directed against an antigen present on the surface of a cell, linked to the LNP, as described herein, can be used to provide an RNA-based therapeutic to a cell in vitro or in vivo. Examples of RNA-based therapeutics include, but are not limited to, mRNA, antisense RNA and oligonucleotides, ribozymes, aptamers, interfering RNAs (RNAi), Dicer-substrate dsRNA, small hairpin RNA (shRNA), asymmetrical interfering RNA (aiRNA), microRNA (miRNA). For example, in one embodiment, the ceDNA vectors (e.g., ceDNA vector lipid particles (e.g., lipid nanoparticles) as described herein) can be used to provide an antisense nucleic acid to a cell in vitro or in vivo. For example, where the transgene is a RNAi molecule, expression of the antisense nucleic acid or RNAi in the target cell diminishes expression of a particular protein by the cell. Accordingly, transgenes which are RNAi molecules or antisense nucleic acids may be administered to decrease expression of a particular protein in a subject in need thereof. Antisense nucleic acids may also be administered to cells in vitro to regulate cell physiology, e.g., to optimize cell or tissue culture systems.

In one embodiment, the pharmaceutical composition comprising a LNP and a TNA, wherein the LNP comprises a scFv, wherein the scFv is directed against an antigen present on the surface of a cell, linked to the LNP, as described herein, can be used to provide a DNA-based therapeutic to a cell in vitro or in vivo. Examples of DNA-based therapeutics include, but are not limited to, minicircle DNA, minigene, viral DNA (e.g., Lentiviral or AAV genome) or non-viral synthetic DNA vectors, closed-ended linear duplex DNA (ceDNA/CELiD), plasmids, bacmids, doggybone™ DNA vectors, minimalistic immunological-defined gene expression (MIDGE)-vector, nonviral ministring DNA vector (linear-covalently closed DNA vector), or dumbbell-shaped DNA minimal vector (“dumbbell DNA”).

In one embodiment, exemplary transgenes encoded by the TNA such as ceDNA vector include, but are not limited to: lysosomal enzymes (e.g., hexosaminidase A, associated with Tay-Sachs disease, or iduronate sulfatase, associated, with Hunter Syndrome/MPS II), erythropoietin, angiostatin, endostatin, superoxide dismutase, globin, leptin, catalase, tyrosine hydroxylase, as well as cytokines (e.g., a interferon, b-interferon, interferon-g, interleukin-2, interleukin-4, interleukin 12, granulocyte-macrophage colony stimulating factor, lymphotoxin, and the like), peptide growth factors and hormones (e.g., somatotropin, insulin, insulin-like growth factors 1 and 2, platelet derived growth factor (PDGF), epidermal growth factor (EGF), fibroblast growth factor (FGF), nerve growth factor (NGF), neurotrophic factor-3 and 4, brain-derived neurotrophic factor (BDNF), glial derived growth factor (GDNF), transforming growth factor-a and -b, and the like), receptors (e.g., tumor necrosis factor receptor). In some exemplary embodiments, the transgene encodes a monoclonal antibody specific for one or more desired targets. In some exemplary embodiments, more than one transgene is encoded by the ceDNA vector. In some exemplary embodiments, the transgene encodes a fusion protein comprising two different polypeptides of interest. In some embodiments, the transgene encodes an antibody, including a full-length antibody or antibody fragment, as defined herein. In some embodiments, the antibody is an antigen-binding domain or an immunoglobulin variable domain sequence, as that is defined herein. Other illustrative transgene sequences encode suicide gene products (thymidine kinase, cytosine deaminase, diphtheria toxin, cytochrome P450, deoxycytidine kinase, and tumor necrosis factor), proteins conferring resistance to a drug used in cancer therapy, and tumor suppressor gene products.

### Administration

In one embodiment, the pharmaceutical compositions comprising a lipid nanoparticle (LNP) and a therapeutic nucleic acid (TNA), as described herein, can be administered to an organism for transduction of cells in vivo. In one embodiment, the TNA can be administered to an organism for transduction of cells ex vivo.

Generally, administration is by any of the routes normally used for introducing a molecule into ultimate contact with blood or tissue cells. Suitable methods of administering such nucleic acids are available and well known to those of skill in the art, and, although more than one route can be used to administer a particular composition, a particular route can often provide a more immediate and more effective reaction than another route. Exemplary modes of administration of the pharmaceutical composition comprising a LNP and a TNA, wherein the LNP comprises a scFv, wherein the scFv is directed against an antigen present on the surface of a cell, linked to the LNP, as described herein includes oral, rectal, transmucosal, intranasal, inhalation (e.g., via an aerosol), buccal (e.g., sublingual), vaginal, intrathecal, intraocular, transdermal, intraendothelial, in utero (or in ovo), parenteral (e.g., intravenous, subcutaneous, intradermal, intracranial, intramuscular [including administration to skeletal, diaphragm and/or cardiac muscle], intrapleural, intracerebral, and intraarticular), topical (e.g., to both skin and mucosal surfaces, including airway surfaces, and transdermal administration), intralymphatic, and the like, as well as direct tissue or organ injection (e.g., to liver, eye, skeletal muscle, cardiac muscle, diaphragm muscle or brain).

Administration of the pharmaceutical composition comprising a LNP and a TNA, wherein the LNP comprises a scFv, wherein the scFv is directed against an antigen present on the surface of a cell, linked to the LNP, as described herein can be to any site in a subject, including, without limitation, a site selected from the group consisting of the brain, a skeletal muscle, a smooth muscle, the heart, the diaphragm, the airway epithelium, the liver, the kidney, the spleen, the pancreas, the skin, and the eye.

In one embodiment, administration of the pharmaceutical composition comprising a LNP and a TNA, wherein the LNP comprises a scFv, wherein the scFv is directed against an antigen present on the surface of a cell, linked to the LNP, as described herein is to a tumor (e.g., in or near a tumor or a lymph node).

The most suitable route in any given case will depend on the nature and severity of the condition being treated, ameliorated, and/or prevented and on the nature of the particular ceDNA (e.g., ceDNA lipid nanoparticles) as described herein that is being used. Additionally, ceDNA permits one to administer more than one transgene in a single vector, or multiple ceDNA vectors (e.g., a ceDNA cocktail).

In one embodiment, administration of the ceDNA vectors (e.g., the pharmaceutical composition comprising a LNP and a TNA, wherein the LNP comprises a scFv, wherein the scFv is directed against an antigen present on the surface of a cell, linked to the LNP, as described herein) to skeletal muscle includes but is not limited to administration to skeletal muscle in the limbs (e.g., upper arm, lower arm, upper leg, and/or lower leg), back, neck, head (e.g., tongue), thorax, abdomen, pelvis/perineum, and/or digits. The ceDNA vectors (e.g., the pharmaceutical composition comprising a LNP and a TNA, wherein the LNP comprises a scFv, wherein the scFv is directed against an antigen present on the surface of a cell, linked to the LNP, as described herein) can be delivered to skeletal muscle by intravenous administration, intra-arterial administration, intraperitoneal administration, limb perfusion, (optionally, isolated limb perfusion of a leg and/or arm; see, e.g., Arruda et al., (2005) Blood 105: 3458-3464), and/or direct intramuscular injection. In particular embodiments, the ceDNA vector (e.g., a ceDNA vector lipid particle as described herein) is administered to a limb (arm and/or leg) of a subject (e.g., a subject with muscular dystrophy such as DMD) by limb perfusion, optionally isolated limb perfusion (e.g., by intravenous or intra-articular administration. In one embodiment, the ceDNA vector (e.g., the pharmaceutical composition comprising a LNP and a TNA, wherein the LNP comprises a scFv, wherein the scFv is directed against an antigen present on the surface of a cell, linked to the LNP, as described herein) can be administered without employing “hydrodynamic” techniques.

Administration of the ceDNA vectors (e.g., the pharmaceutical composition comprising a LNP and a TNA, wherein the LNP comprises a scFv, wherein the scFv is directed against an antigen present on the surface of a cell, linked to the LNP, as described herein) to cardiac muscle includes administration to the left atrium, right atrium, left ventricle, right ventricle and/or septum. The ceDNA vectors (e.g., the pharmaceutical composition comprising a LNP and a TNA, wherein the LNP comprises a scFv, wherein the scFv is directed against an antigen present on the surface of a cell, linked to the LNP, as described herein) can be delivered to cardiac muscle by intravenous administration, intra-arterial administration such as intra-aortic administration, direct cardiac injection (e.g., into left atrium, right atrium, left ventricle, right ventricle), and/or coronary artery perfusion. Administration to diaphragm muscle can be by any suitable method including intravenous administration, intra-arterial administration, and/or intra-peritoneal administration. Administration to smooth muscle can be by any suitable method including intravenous administration, intra-arterial administration, and/or intra-peritoneal administration. In one embodiment, administration can be to endothelial cells present in, near, and/or on smooth muscle.

In one embodiment, ceDNA vectors (e.g., the pharmaceutical composition comprising a LNP and a TNA, wherein the LNP comprises a scFv, wherein the scFv is directed against an antigen present on the surface of a cell, linked to the LNP, as described herein) are administered to skeletal muscle, diaphragm muscle and/or cardiac muscle (e.g., to treat, ameliorate, and/or prevent muscular dystrophy or heart disease (e.g., PAD or congestive heart failure).

ceDNA vectors (e.g., the pharmaceutical composition comprising a LNP and a TNA, wherein the LNP comprises a scFv, wherein the scFv is directed against an antigen present on the surface of a cell, linked to the LNP, as described herein) can be administered to the CNS (e.g., to the brain or to the eye). The ceDNA vectors (e.g., the pharmaceutical composition comprising a LNP and a TNA, wherein the LNP comprises a scFv, wherein the scFv is directed against an antigen present on the surface of a cell, linked to the LNP, as described herein) may be introduced into the spinal cord, brainstem (medulla oblongata, pons), midbrain (hypothalamus, thalamus, epithalamus, pituitary gland, substantia nigra, pineal gland), cerebellum, telencephalon (corpus striatum, cerebrum including the occipital, temporal, parietal and frontal lobes, cortex, basal ganglia, hippocampus and portaamygdala), limbic system, neocortex, corpus striatum, cerebrum, and inferior colliculus. The ceDNA vectors (e.g., the pharmaceutical composition comprising a LNP and a TNA, wherein the LNP comprises a scFv, wherein the scFv is directed against an antigen present on the surface of a cell, linked to the LNP, as described herein) may also be administered to different regions of the eye such as the retina, cornea and/or optic nerve. The ceDNA vectors (e.g., the pharmaceutical composition comprising a LNP and a TNA, wherein the LNP comprises a scFv, wherein the scFv is directed against an antigen present on the surface of a cell, linked to the LNP, as described herein) may be delivered into the cerebrospinal fluid (e.g., by lumbar puncture). The ceDNA vectors (e.g., ceDNA vector lipid particles (e.g., lipid nanoparticles) as described herein) may further be administered intravascularly to the CNS in situations in which the blood-brain barrier has been perturbed (e.g., brain tumor or cerebral infarct).

In one embodiment, the ceDNA vectors (e.g., the pharmaceutical composition comprising a LNP and a TNA, wherein the LNP comprises a scFv, wherein the scFv is directed against an antigen present on the surface of a cell, linked to the LNP, as described herein) can be administered to the desired region(s) of the CNS by any route known in the art, including but not limited to, intrathecal, intra-ocular, intracerebral, intraventricular, intravenous (e.g., in the presence of a sugar such as mannitol), intranasal, intra-aural, intra-ocular (e.g., intra-vitreous, sub-retinal, anterior chamber) and peri-ocular (e.g., sub-Tenon's region) delivery as well as intramuscular delivery with retrograde delivery to motor neurons.

According to some embodiment, the ceDNA vectors (e.g., the pharmaceutical composition comprising a LNP and a TNA, wherein the LNP comprises a scFv, wherein the scFv is directed against an antigen present on the surface of a cell, linked to the LNP, as described herein) is administered in a liquid formulation by direct injection (e.g., stereotactic injection) to the desired region or compartment in the CNS. According to other embodiments, the ceDNA vectors (e.g., ceDNA vector lipid particles (e.g., the pharmaceutical composition comprising a LNP and a TNA, wherein the LNP comprises a scFv, wherein the scFv is directed against an antigen present on the surface of a cell, linked to the LNP, as described herein) can be provided by topical application to the desired region or by intra-nasal administration of an aerosol formulation. Administration to the eye may be by topical application of liquid droplets. As a further alternative, the ceDNA vector can be administered as a solid, slow-release formulation (see, e.g., U.S. Pat. No. 7,201,898, incorporated by reference in its entirety herein). In one embodiment, the ceDNA vectors (e.g., the pharmaceutical composition comprising a LNP and a TNA, wherein the LNP comprises a scFv, wherein the scFv is directed against an antigen present on the surface of a cell, linked to the LNP, as described herein) can used for retrograde transport to treat, ameliorate, and/or prevent diseases and disorders involving motor neurons (e.g., amyotrophic lateral sclerosis (ALS); spinal muscular atrophy (SMA), etc.). For example, the ceDNA vectors (e.g., the pharmaceutical composition comprising a LNP and a TNA, wherein the LNP comprises a scFv, wherein the scFv is directed against an antigen present on the surface of a cell, linked to the LNP, as described herein) can be delivered to muscle tissue from which it can migrate into neurons.

In one embodiment, repeat administrations of the therapeutic product can be made until the appropriate level of expression has been achieved. Thus, in one embodiment, a therapeutic nucleic acid can be administered and re-dosed multiple times. For example, the therapeutic nucleic acid can be administered on day 0. Following the initial treatment at day 0, a second dosing (re-dose) can be performed in about 1 week, about 2 weeks, about 3 weeks, about 4 weeks, about 5 weeks, about 6 weeks, about 7 weeks, about 8 weeks, or about 3 months, about 4 months, about 5 months, about 6 months, about 7 months, about 8 months, about 9 months, about 10 months, about 11 months, or about 1 year, about 2 years, about 3 years, about 4 years, about 5 years, about 6 years, about 7 years, about 8 years, about 9 years, about 10 years, about 11 years, about 12 years, about 13 years, about 14 years, about 15 years, about 16 years, about 17 years, about 18 years, about 19 years, about 20 years, about 21 years, about 22 years, about 23 years, about 24 years, about 25 years, about 26 years, about 27 years, about 28 years, about 29 years, about 30 years, about 31 years, about 32 years, about 33 years, about 34 years, about 35 years, about 36 years, about 37 years, about 38 years, about 39 years, about 40 years, about 41 years, about 42 years, about 43 years, about 44 years, about 45 years, about 46 years, about 47 years, about 48 years, about 49 years or about 50 years after the initial treatment with the therapeutic nucleic acid.

In one embodiment, one or more additional compounds can also be included. Those compounds can be administered separately or the additional compounds can be included in the lipid particles (e.g., lipid nanoparticles) of the disclosure. In other words, the lipid particles (e.g., lipid nanoparticles) can contain other compounds in addition to the ceDNA or at least a second ceDNA, different than the first. Without limitations, other additional compounds can be selected from the group consisting of small or large organic or inorganic molecules, monosaccharides, disaccharides, trisaccharides, oligosaccharides, polysaccharides, peptides, proteins, peptide analogs and derivatives thereof, peptidomimetics, nucleic acids, nucleic acid analogs and derivatives, an extract made from biological materials, or any combinations thereof.

In one embodiment, the one or more additional compound can be a therapeutic agent. The therapeutic agent can be selected from any class suitable for the therapeutic objective. Accordingly, the therapeutic agent can be selected from any class suitable for the therapeutic objective. The therapeutic agent can be selected according to the treatment objective and biological action desired.

For example, in one embodiment, if the ceDNA within the LNP is useful for treating cancer, the additional compound can be an anti-cancer agent (e.g., a chemotherapeutic agent, a targeted cancer therapy (including, but not limited to, a small molecule, an antibody, or an antibody-drug conjugate). According to some embodiments, the additional compound is a checkpoint inhibitor.

In one embodiment, if the LNP containing the ceDNA is useful for treating an infection, the additional compound can be an antimicrobial agent (e.g., an antibiotic or antiviral compound). In one embodiment, if the LNP containing the ceDNA is useful for treating an immune disease or disorder, the additional compound can be a compound that modulates an immune response (e.g., an immunosuppressant, immunostimulatory compound, or compound modulating one or more specific immune pathways). In one embodiment, different cocktails of different lipid particles containing different compounds, such as a ceDNA encoding a different protein or a different compound, such as a therapeutic may be used in the compositions and methods of the disclosure. In one embodiment, the additional compound is an immune modulating agent. For example, the additional compound is an immunosuppressant. In some embodiments, the additional compound is immunostimulatory.

### REFERENCES

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior disclosure or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting. It should be understood that this disclosure is not limited in any manner to the particular methodology, protocols, and reagents, etc., described herein and as such can vary. The terminology used herein is for the purpose of describing particular embodiments only and is not intended to limit the scope of the present disclosure, which is defined solely by the claims.

### EXAMPLES

The following examples are provided by way of illustration not limitation.

### Example 1: Validation of Targeting Ligand

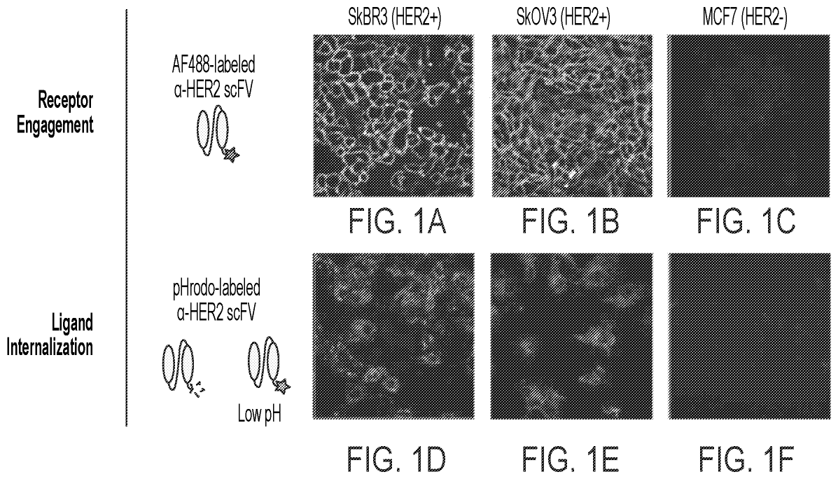

As proof of concept, HER2 scFv binding and internalization in cell-based assays in vitro was carried out. FIGS. 1A-1F shows trastuzumab-derived α-HER2 scFv shows clear HER2-specific membrane targeting and internalization in vitro. Alexa-fluor 488 labeled anti-HER2 scFv was used to show HER2 receptor engagement in Sk-BR3 and Sk-OV3 HER2 expressing (HER2+) cell lines (FIGS. 1A and 1B), but not in MCF7 cells (FIG. 1C), which do not express HER2 receptor (HER2-). A second immunofluorescent label (pHrhodo) was used to demonstrate ligand internalization. As shown in FIGS. 1D and 1E, Sk-BR3 and Sk-OV3 cells that express the HER2 receptor showed ligand internalization, while the MCF7 HER2-cell line did not (FIG. 1F).

### Example 2: Formulation of LNPs with α-HER2 scFv

This example describes the preparation of LNPs that present α-HER2 scFv (SEQ ID NO:1) on their surface. As described herein, enhanced uptake was demonstrated with HER2 scFvs LNPs prepared by maleimide chemistry. scFV sequences for HER2 targeting are shown below:

SEQ ID NO: 1
(SEQ ID NO: 1)
EVQLVESGGGLVQPGGSLRLSCAASGFNIDDTYIHWVRQAPGKGLEWVA
RIYPTNGYTRYADSVKGRFTISADTSKNTAYLQMNSLRAEDTAVYYCSR
WGGDGFYAMDVWGQGTLVTVSSGGGGSGGGGSGGGGSDIQMTQSPSS
LSASVGDRVTITCRASQDVNTAVAWYQQKPGKAPKLLIYSADFLYSGVP
SRFSGSRSGTDFTLTISSLQPEDFATYYCQQHYTTPPTFGQGTKVEIK

SEQ ID NO:2 contains a myc (bold underlined) tag and a His (italic) tag with a c-terminal cysteine required for maleimide conjugation. This sequence was used in scFV for the PDS conjugation.

(SEQ ID NO: 2)
EVQLVESGGGLVQPGGSLRLSCAASGFNIDDTYIHWVRQAPGKGLEWVA
RIYPTNGYTRYADSVKGRFTISADTSKNTAYLQMNSLRAEDTAVYYCSR
WGGDGFYAMDVWGQGTLVTVSSGGGGSGGGGSGGGGSDIQMTQSPSS
LSASVGDRVTITCRASQDVNTAVAWYQQKPGKAPKLLIYSADFLYSGVP
SRFSGSRSGTDFTLTISSLQPEDFATYYCQQHYTTPPTFGQGTKVEIK
EQKLISEEDLHHHHHHC

SEQ ID NO:3 was used for transglutaminase-mediated conjugation, has the same scFV core sequence as SEQ ID NO:1 but with an N-terminal His (italic) tag and a c-terminal LLQGA polypeptide (bold and underlined) to facilitate transglutaminase-mediated conjugation.

(SEQ ID NO: 3)
EHHHHHHEVQLVESGGGLVQPGGSLRLSCAASGFNIDDTYIHWVRQAPGK
GLEWVARIYPTNGYTRYADSVKGRFTISADTSKNTAYLQMNSLRAEDTAV
YYCSRWGGDGFYAMDVWGQGTLVTVSSGGGGSGGGGSGGGGSDIQMTQSP
SSLSASVGDRVTITCRASQDVNTAVAWYQQKPGKAPKLLIYSADFLYSGV
PSRFSGSRSGTDFTLTISSLQPEDFATYYCQQHYTTPPTFGQGTKVEIK
LLQGA

Evaluation of Maleimide Chemistry for scFv-LNPs

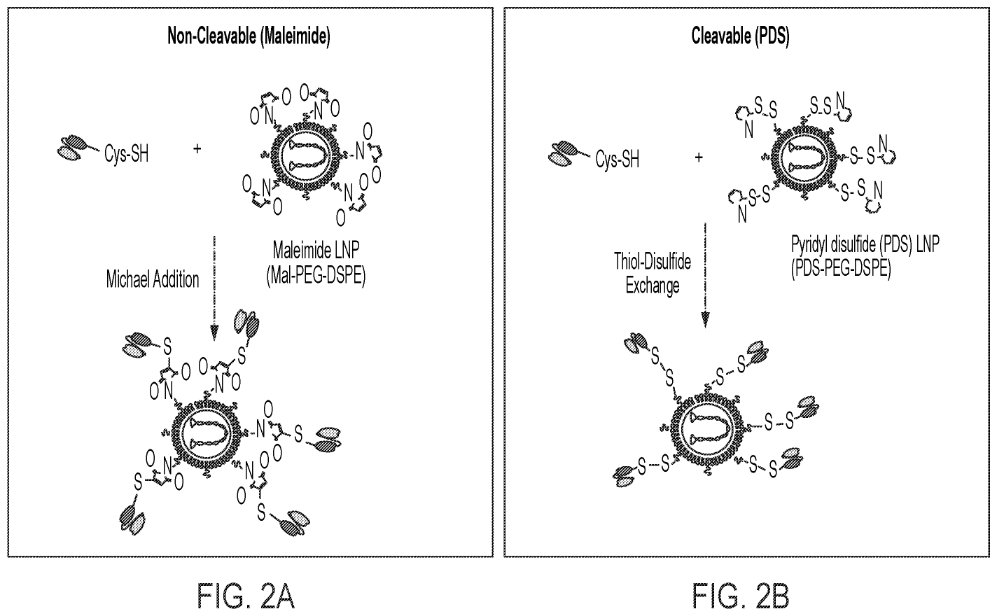

Exemplary primary routes of conjugation using thiol-based crosslinking are shown in FIG. 2A and FIG. 2B. Maleimide (non-cleavable) linkage is shown in FIG. 2A. Pyridyl disulfide or PDS (cleavable) linkage is shown in FIG. 2B.

The conjugation protocol for PDS chemistry was performed as follows. The scFvs were reduced with a 50 molar excess of TCEP for 2 hours at 37° C. After reduction, TCEP was removed from scFvs using G-25 spin columns (237 μg scFv/column). The scFvs were then incubated with LNPs formulated with Lipid A and DSPE-PEG-OPSS (Nanosoft Polymers, Winston-Salem, NC, USA) of different mole percentages (0.1%, 0.5%) and PEG lengths (2k, 5k) for 2 hours at 25° C. The ratio of scFV/PDS is 0.05. Following reaction, unreacted scFvs were removed by dialysis in 300 kD MWCO membranes overnight. To determine if scFvs were conjugated to the LNPs, SDS-PAGE was performed and Western blots against the HER2 scFv were performed.

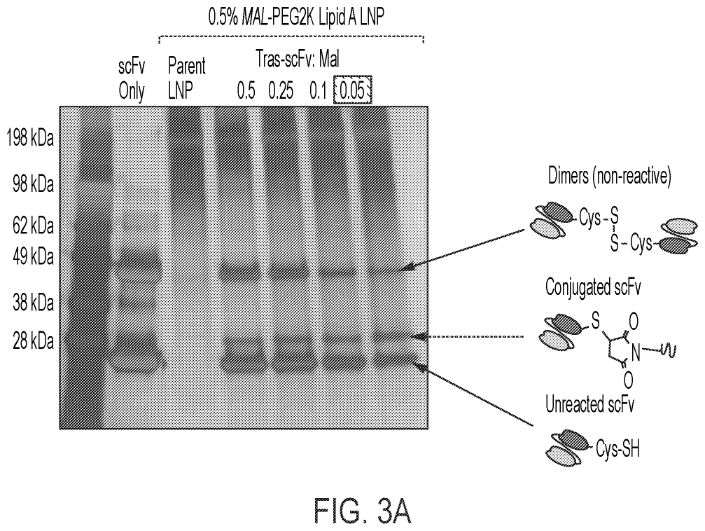

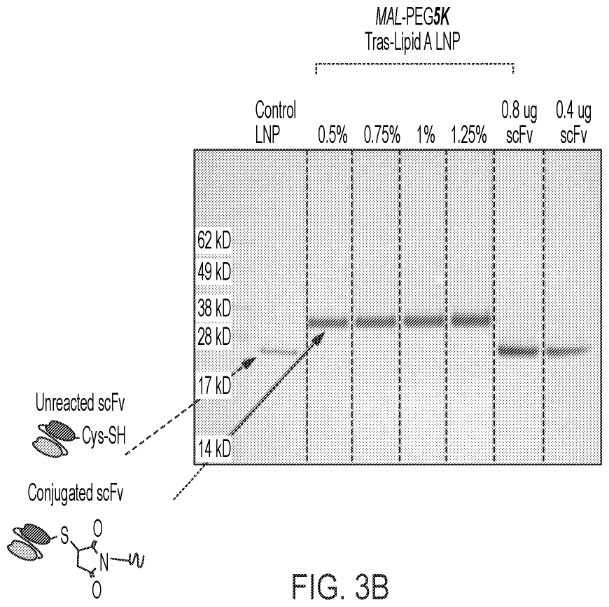

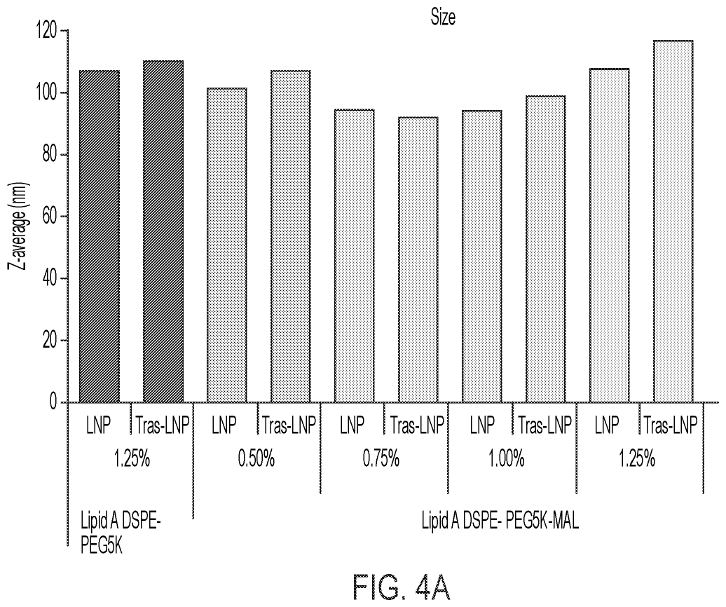

The conjugation protocol for maleimide chemistry was performed as follows. The scFvs were reduced with a 50 molar excess of TCEP for 2 hours at 37° C. After reduction, TCEP was removed from scFvs using G-25 spin columns (237 μg scFv/column). The scFvs were then incubated with LNPs formulated with Lipid A and DSPE-PEG-maleimide of different mole percentages (0.1%, 0.5%, 0.75%, 1%, 1.25%) and PEG lengths (2k, 5k) for 2 hours at 25° C. The ratio of scFV/Maleimide is 0.05. Following reaction, unreacted scFvs were removed by dialysis in 300 kD MWCO membranes overnight. To determine if scFvs were conjugated to the LNPs, SDS-PAGE was performed and Western blots against the HER2 scFv were performed. It was found that the scFv-LNP conjugation process demonstrated excellent conjugation yield and LNP particle stability. For example, the results of the conjugation process that included an initial TCEP reduction, fresh maleimide-conjugated LNP preparation, 0.5% MAL-PEG2K, and decreasing scFv:Mal molar equivalents of 0.5:1, 0.25:1, 0.1:1, and 0.05:1 are shown in FIG. 3A. Next, the PEG chain length was increased and the dialysis step was used to remove unreacted scFv without disrupting the particle size and stability. The results are shown in FIG. 3B.

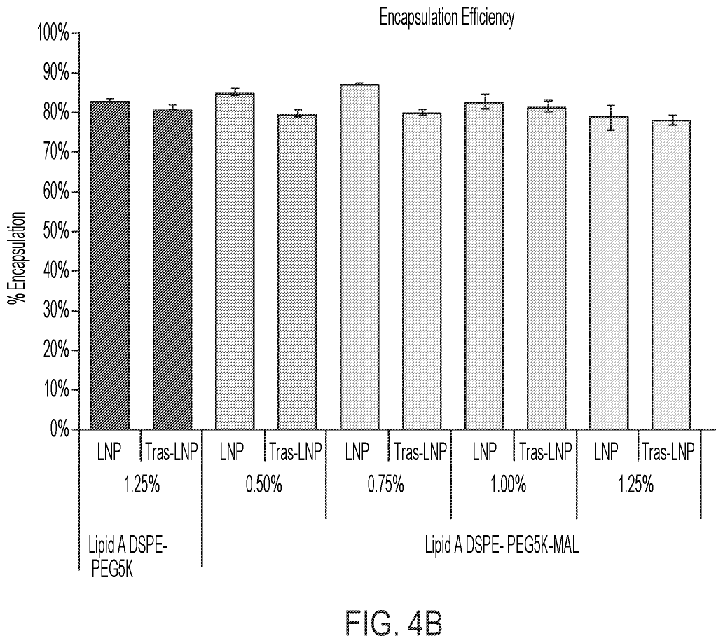

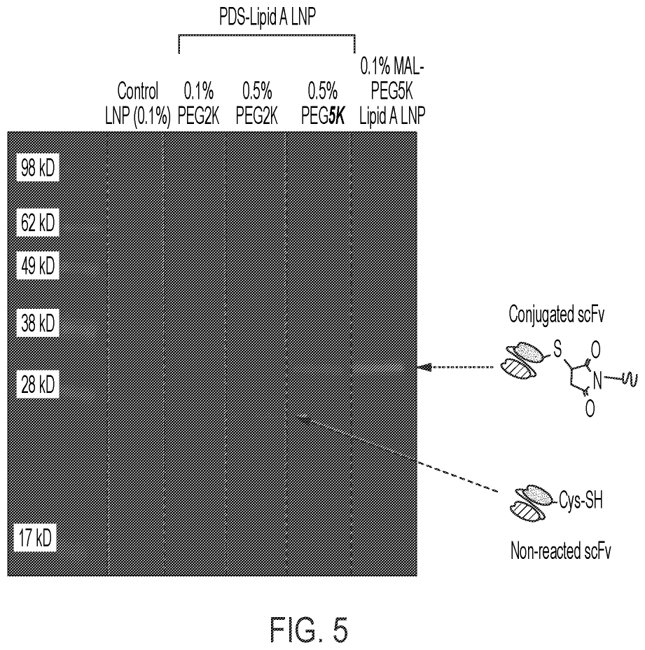

FIG. 4A and FIG. 4B show that LNP size and integrity, as measured by encapsulation efficiency (EE), were maintained post-scFv conjugation (±10 nm). FIG. 5 shows that the maleimide conjugation process resulted in robust conjugation, while PDS conjugation process was equivalent or slightly weaker. This was a surprising and unexpected result, because maleimides are generally susceptible to hydrolysis in aqueous environments and especially at higher pH values, which can affect conjugation efficiency of polypeptides to LNPs, while PDS chemistry does not typically present this challenge.

Next, confirmation of ligand function on the LNP was carried out. FIG. 6A shows that only Tras-scFv conjugated LNPs showed HER2 engagement, compared to DSPE control LNP in FIG. 6B, confirming ligand function on the LNP. FIG. 7 shows that maleimide conjugated LNPs demonstrated HER2-specific, enhanced cell uptake. Specifically, FIG. 7 demonstrates that uptake of conjugated Tras-scFv Lipid A LNPs (mCherry) was mediated by HER2. Finally, FIG. 8A and FIG. 8B show that ligand presentation on the LNP surface significantly affected biological activity. The graph in FIG. 8A compares LNP uptake (mCherry) in maleimide-conjugated LNPs, where the PEG chain length was either 2000 Da (PEG2K) or 5000 Da (PEG5K), normalized to cell viability. As shown in FIG. 8A, maleimide-conjugated LNPs having PEG5K showed greater biological activity, as assessed by cellular uptake of LNPs. The graph in FIG. 8B shows that a dose-dependent decrease in LNP uptake (mCherry) was observed as the maleimide concentration (as conjugated to PEG5K) was increased from 0.5% to 1.25%.

The results presented herein demonstrated in vitro binding and internalization of the scFv-LNPs, described an optimal and efficient maleimide covalent conjugation with minimal effects on LNP particle size and stability, have confirmed the ligand function on the LNP, and demonstrated receptor specific cell internalization and delivery of the TNA cargo. These results strongly suggest the effectiveness of the described scFv-LNPs in targeted tumor uptake in vivo with minimal off-target tissue (e.g., liver, spleen) uptake after systemic administration.

1. A pharmaceutical composition comprising:
a lipid nanoparticle (LNP), wherein the LNP comprises a cationic lipid, a sterol, a non-cationic lipid, and a PEG5000 PEGylated lipid;
a therapeutic nucleic acid (TNA):
and at least one pharmaceutically acceptable excipient; wherein the LNP comprises a single-chain variable fragment (scFv) linked to the LNP; and wherein the scFv is directed against an antigen present on the surface of a cell.

2. The pharmaceutical composition of claim 1, wherein the scFv is covalently linked to the LNP or the scFv is chemically conjugated to the LNP.

3. (canceled)

4. The pharmaceutical composition of claim 2, wherein the scFv is chemically conjugated to the LNP via a non-cleavable linker or wherein the scFV is chemically conjugated to the LNP via a cleavable linker.

5. The pharmaceutical composition of claim 4, wherein:
the non-cleavable linker is a maleimide-containing linker, or
the cleavable linker is a pyridyldisulfide (PDS)-containing linker.

6. (canceled)

7. (canceled)

8. The pharmaceutical composition of claim 1, wherein the scFv is linked to the LNP via transglutaminase-mediated conjugation.

9. The pharmaceutical composition of claim 1, wherein the antigen is a tumor-associated antigen (TAA) or a tumor-specific antigen (TSA).

10-14. (canceled)

15. The pharmaceutical composition of claim 1, wherein the TNA is encapsulated in the LNP.

16. The pharmaceutical composition of claim 1, wherein the TNA is selected from the group consisting of a minigenes, a plasmid, a minicircle, a small interfering RNA (siRNA), a microRNA (miRNA), an antisense oligonucleotide (ASO), a ribozyme, a closed-ended (ceDNA), a ministring DNA, a doggybone™ DNA, a protelomere closed ended DNA, a dumbbell linear DNA, a dicer-substrate dsRNA, a small hairpin RNA (shRNA), an asymmetrical interfering RNA (aiRNA), a microRNA (miRNA), a mRNA, a tRNA, a rRNA, a DNA viral vector, a viral RNA vector, a non-viral vector and any combination thereof.

17-21. (canceled)

22. The pharmaceutical composition of claim 1, wherein the pharmaceutical composition is administered to a subject; wherein the subject is a human patient in need of treatment with LNP encapsulated with TNA.

23. (canceled)

24. The pharmaceutical composition of claim 1, wherein:
the composition is targeted to a cell expressing the cell-surface antigen for which the scFv is directed, or
wherein the composition is targeted to tumor cells: or
wherein the composition is targeted to liver cells: or
wherein the composition is targeted to hepatocytes in the liver.

25-27. (canceled)

28. The pharmaceutical composition of claim 1, wherein:
the cationic lipid is represented by Formula (I), 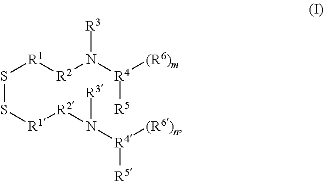
or a pharmaceutically acceptable salt thereof, wherein:
R¹ and R^{1′} are each independently optionally substituted linear or branched C₁₋₃ alkylene;
R² and R^{2′} are each independently optionally substituted linear or branched C₁₋₆ alkylene;
R³ and R^{3′} are each independently optionally substituted linear or branched C₁₋₆ alkyl;
or alternatively, when R² is optionally substituted branched C₁₋₆ alkylene, R² and R³, taken together with their intervening N atom, form a 4- to 8-membered heterocyclyl;
or alternatively, when R^{2′} is optionally substituted branched C₁₋₆ alkylene, R^{2′} and R^{3′}, taken together with their intervening N atom, form a 4- to 8-membered heterocyclyl;
R⁴ and R^{4′} are each independently —CR^{a}, —C(R^{a})₂CR^{a}, or —[C(R^{a})₂]₂CR^{a};
R^{a}, for each occurrence, is independently H or C₁₋₃ alkyl;
or alternatively, when R⁴ is —C(R^{a})₂CR^{a}, or —[C(R^{a})₂]₂CR^{a} and when R^{a} is C₁₋₃ alkyl, R³ and R⁴, taken together with their intervening N atom, form a 4- to 8-membered heterocyclyl;
or alternatively, when R^{4′} is —C(R^{a})₂CR^{a}, or —[C(R^{a})₂]₂CR^{a} and when R^{a} is C₁₋₃ alkyl, R^{3′} and R^{4′}, taken together with their intervening N atom, form a 4- to 8-membered heterocyclyl;
R⁵ and R^{5′} are each independently hydrogen, C₁₋₂₀ alkylene or C₂₋₂₀ alkenylene;
R⁶ and R^{6′}, for each occurrence, are independently C₁₋₂₀ alkylene, C₃₋₂₀ cycloalkylene, or C₂₋₂₀ alkenylene; and
m and n are each independently an integer selected from 1, 2, 3, 4, and 5: or the cationic lipid is represented by Formula (II): 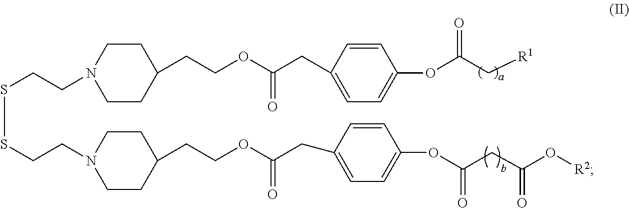
or a pharmaceutically acceptable salt thereof, wherein:
a is an integer ranging from 1 to 20;
b is an integer ranging from 2 to 10;
R¹ is absent or is selected from (C₂-C₂₀)alkenyl, —C(O)O(C₂-C₂₀)alkyl, and cyclopropyl substituted with (C₂-C₂₀)alkyl; and
R² is (C₂-C₂₀)alkyl; or
the cationic lipid is represented by the Formula (V): 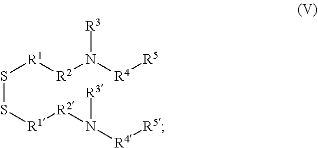
or a pharmaceutically acceptable salt thereof, wherein:
R¹ and R^{1′} are each independently (C₁-C₆)alkylene optionally substituted with one or more groups selected from R^{a};
R² and R^{2′} are each independently (C₁-C₂)alkylene;
R³ and R^{3′} are each independently (C₁-C₆)alkyl optionally substituted with one or more groups selected from R^{b}:
or alternatively, R² and R³ and/or R^{2′} and R^{3′} are taken together with their intervening N atom to form a 4- to 7-membered heterocyclyl;
R⁴ and R^{4′} are each a (C₂-C₆)alkylene interrupted by —C(O)O—;
R⁵ and R^{5′} are each independently a (C₂-C₃₀)alkyl or (C₂-C₃₀)alkenyl, each of which are optionally interrupted with —C(O)O— or (C₃-C₆)cycloalkyl; and
R^{a} and R^{b} are each halo or cyano; or
wherein the cationic lipid is represented by Formula (XV): 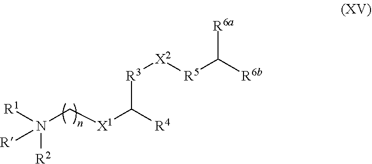
or a pharmaceutically acceptable salt thereof, wherein:
R′ is absent, hydrogen, or C₁-C₆ alkyl provided that when R′ is hydrogen or C₁-C₆ alkyl, the nitrogen atom to which R′, R¹, and R² are all attached is protonated;
R¹ and R² are each independently hydrogen, C₁-C₆ alkyl, or C₂-C₆ alkenyl;
R³ is C₁-C₁₂ alkylene or C₂-C₁₂ alkenylene;
R⁴ is C₁-C₁6 unbranched alkyl, C₂-C₁₆unbranched alkenyl, or 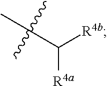
wherein:
R^{4a} and R^{4b} are each independently C₁-C₁₆ unbranched alkyl or C₂-C₁₆unbranched alkenyl;
R⁵ is absent, C₁-C₈ alkylene, or C₂-C₈ alkenylene;
R^{6a} and R^{6b} are each independently C₇-C₁₆ alkyl or C₇-C₁₆ alkenyl; provided that the total number of carbon atoms in R^{6a} and R^{6b} as combined is greater than 15;
X¹ and X² are each independently —OC(═O)—, —SC(═O)—, —OC(═S)—, —C(═O)O—, —C(═O)S—, —S—S—, —C(R^{a})═N—, —N═C(R^{a})—, —C(R^{a})═NO—, —O—N═C(R^{a})—, —C(═O)NR^{a}—, —NR^{a}C(═O)—, —NR^{a}C(═O)NR^{a}—, —OC(═O)O—, —OSi(R^{a})₂O—, —C(═O)(CR^{a}2)C(═O)O—, or OC(═O)(CR^{a}₂)C(═O)—; wherein:
R^{a}, for each occurrence, is independently hydrogen or C₁-C₆ alkyl; and
n is an integer selected from 1, 2, 3, 4, 5, and 6; or
wherein the cationic lipid is represented by Formula (XX): 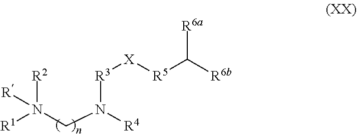
or a pharmaceutically acceptable salt thereof, wherein:
R′ is absent, hydrogen, or C₁-C₃ alkyl; provided that when R′ is hydrogen or C₁-C₃ alkyl, the nitrogen atom to which R′, R¹, and R² are all attached is protonated;
R¹ and R² are each independently hydrogen or C₁-C₃ alkyl;
R³ is C₃-C₁₀ alkylene or C₃-C₁₀ alkenylene;
R⁴ is C₁-C₁₆ unbranched alkyl, C₂-C₁₆unbranched alkenyl, or 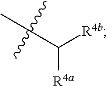
wherein:
R^{4a} and R^{4b} are each independently C₁-C₁₆ unbranched alkyl or C₂-C₁₆unbranched alkenyl;
R⁵ is absent, C₁-C₆ alkylene, or C₂-C₆ alkenylene;
R^{6a} and R^{6b} are each independently C₇-C₁4 alkyl or C₇-C₁4 alkenyl X is —OC(═O)—, —SC(═O)—, —OC(═S)—, —C(═O)O—, —C(═O)S—, —S—S—, —C(R^{a})═N—, —N═C(R^{a})—, —C(R^{a})═NO—, —O—N═C(R^{a})—, —C(═O)NR^{a}—, —NR^{a}C(═O)—, —NR^{a}C(═O)NR^{a}—, —OC(═O)O—, —OSi(R^{a})₂O—, —C(═O)(CR^{a}2)C(═O)O—, or OC(═O)(CR^{a}2)C(═O)—; wherein:
R^{a}, for each occurrence, is independently hydrogen or C₁-C₆ alkyl; and
n is an integer selected from 1, 2, 3, 4, 5, and 6; or
the cationic lipid is selected from a lipid comprising any lipid set forth in Table 2, Table 5, Table 6, Table 7, and Table 8; or
wherein the cationic lipid is a lipid having the structure: 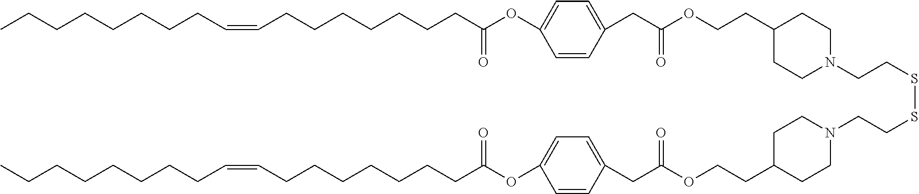 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCC/C=C\CCCCCCCC)cc4)CC3)CC2)cc1]
or a pharmaceutically acceptable salt thereof; and/or
wherein the cationic lipid is MC3 (6Z,9Z,28Z,31Z)-heptatriaconta-6,9,28,31-tetraen-19-yl-4-(dimethylamino) butanoate (DLin-MC3-DMA or MC3) having the following structure: 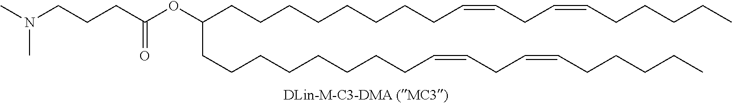 [SMILES: CCCCC/C=C\C/C=C\CCCCCCCCC(CCCCCCCC/C=C\C/C=C\CCCCC)OC(=O)CCCN(C)C]
or a pharmaceutically acceptable salt thereof.

29-35. (canceled)

36. The pharmaceutical composition of claim 1, wherein:
the sterol or a derivative thereof is a cholesterol or a beta-sitosterol;
the non-cationic lipid is selected from the group consisting of distearoyl-sn-glycero-phosphoethanolamine (DSPE), distearoylphosphatidylcholine (DSPC),
dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), 1,2-dilauroyl-sn-glycero-3-pho sphoethanolamine (DLPE); 1,2-diphytanoyl-sn-glycero-3-phosphoethanolamine (DPHyPE); lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid,cerebrosides, dicetylphosphate, lysophosphatidylcholine, dilinoleoylphosphatidylcholine, and mixtures thereof; optionally wherein the non-cationic lipid is selected from the group consisting of dioleoylphosphatidylcholine (DOPC), distearoylphosphatidylcholine (DSPC), and dioleoyl-phosphatidylethanolamine (DOPE); and/or
the PEG5000 PEGylated lipid is selected from the group consisting of PEG-dilauryloxypropyl; PEG-dimyristyloxypropyl; PEG-dipalmityloxypropyl, PEG-distearyloxypropyl; 1-(monomethoxy-polyethyleneglycol)-2,3-dimyristoylglycerol (DMG-PEG); PEG-dilaurylglycerol; PEG-dipalmitoylglycerol; PEG-disterylglycerol; PEG-dilaurylglycamide; PEG-dimyristylglycamide; PEG-dipalmitoylglycamide; PEG-disterylglycamide; (1-[8′-(Cholest-5-en-3[beta]-oxy)carboxamido-3′,6′-dioxaoctanyl1 carbamoyl-[omega]-methyl-poly(ethylene glycol) (PEG-cholesterol); 3,4-ditetradecoxylbenzyl-[omega]-methyl-poly(ethylene glycol) ether (PEG-DMB), and 1,2-dimyristoyl-sn-glycero-3-phosphoethanolamine-N-[methoxy(polyethylene glycol) (DSPE-PEG), and 1,2-distearoyl-sn-glycero-3-phosphoethanolamine-N-poly(ethylene glycol)-hydroxyl (DSPE-PEG-OH); optionally wherein the at least one PEGylated lipid is DMG-PEG5000, DSPE-PEG5000, DSPE-PEG5000-OH, or a combination thereof.

37-45. (canceled)

46. The pharmaceutical composition of claim 28, wherein:
the cationic lipid is present at a molar percentage of about 30% to about 80%;
the sterol is present at a molar percentage of about 20% to about 50%;
the non-cationic lipid is present at a molar percentage of about 2% to about 20%;
the at least one PEGylated lipid is present at a molar percentage of about 2.1% to about ° 1%;
the LNP has a total lipid to TNA ratio of about 10:1 to about 40:1 and/or the scFv are present at a total amount of about 0.02 μg/μg of TNA to about 0.1 μg/μg of TNA.

47-50. (canceled)

51. The pharmaceutical composition claim 1, further comprising dexamethasone palmitate.

52. (canceled)

53. The pharmaceutical composition of claim 1, wherein the LNP has a diameter ranging from about 40 nm to about 120 nm; a diameter of about 60 nm to about 80 nm; or a diameter of less than about 100 nm.

54. (canceled)

55. (canceled)

56. The pharmaceutical composition of claim 16, wherein the TNA comprises an expression cassette, and wherein the expression cassette comprises a promoter sequence and a transgene.

57-72. (canceled)

73. A method of treating a cancer in a subject, said method comprising administering to the subject an effective amount of the pharmaceutical composition of claim 1.

74. (canceled)

75. A method of delivering a therapeutic nucleic acid (TNA) or increasing the concentration of the TNA to a tumor in a subject, said method comprising administering to the subject an effective amount of the pharmaceutical composition of claim 1.

76. A method of delivering a therapeutic nucleic acid (TNA) or increasing the concentration of the TNA to the liver of a subject, said method comprising administering to the subject an effective amount of the pharmaceutical composition of claim 1.

77. The pharmaceutical composition of claim 1, wherein the scFv is chemically conjugated to the LNP via PEG5000.

78. The pharmaceutical composition of claim 1, wherein the PEGylated lipid to which the scFv is chemically conjugated or covalently linked is DSPE-PEG5000.


SEQUENCE LISTING (4 sequences)

SEQ ID NO: 1 - protein, 242 aa, synthetic construct
EVQLVESGGGLVQPGGSLRLSCAASGFNIDDTYIHWVRQAPGKGLEWVARIYPTNGYTRYADSVKGRFTISADTSKNTAYLQMNSLRAEDTAVYYCSRWGGDGFYAMDVWGQGTLVTVSSGGGGSGGGGSGGGGSDIQMTQSPSSLSASVGDRVTITCRASQDVNTAVAWYQQKPGKAPKLLIYSADFLYSGVPSRFSGSRSGTDFTLTISSLQPEDFATYYCQQHYTTPPTFGQGTKVEIK

SEQ ID NO: 2 - protein, 259 aa, synthetic construct
EVQLVESGGGLVQPGGSLRLSCAASGFNIDDTYIHWVRQAPGKGLEWVARIYPTNGYTRYADSVKGRFTISADTSKNTAYLQMNSLRAEDTAVYYCSRWGGDGFYAMDVWGQGTLVTVSSGGGGSGGGGSGGGGSDIQMTQSPSSLSASVGDRVTITCRASQDVNTAVAWYQQKPGKAPKLLIYSADFLYSGVPSRFSGSRSGTDFTLTISSLQPEDFATYYCQQHYTTPPTFGQGTKVEIKEQKLISEEDLHHHHHHC

SEQ ID NO: 3 - protein, 253 aa, synthetic construct
HHHHHHEVQLVESGGGLVQPGGSLRLSCAASGFNIDDTYIHWVRQAPGKGLEWVARIYPTNGYTRYADSVKGRFTISADTSKNTAYLQMNSLRAEDTAVYYCSRWGGDGFYAMDVWGQGTLVTVSSGGGGSGGGGSGGGGSDIQMTQSPSSLSASVGDRVTITCRASQDVNTAVAWYQQKPGKAPKLLIYSADFLYSGVPSRFSGSRSGTDFTLTISSLQPEDFATYYCQQHYTTPPTFGQGTKVEIKLLQGA

SEQ ID NO: 4 - protein, 5 aa, synthetic construct
LLQGA